(19) United States
(12) Patent Application Publication
(10) Pub. No.: US 2023/0265190 A1
(43) Pub. Date: Aug. 24, 2023

(54) ALLOREACTIVE IMMUNE CELL-DISTANCING DEVICE AND USES THEREOF FOR PROTECTING DONOR-DERIVED CELLS FROM ALLOREJECTION

(71) Applicant: MIGAL Galilee Research Institute Ltd, Kiryat Shmona (IL)

(72) Inventors: Gideon Gross, Moshav Almagor (IL); Hadas Weinstein-Marom, Upper Galilee (IL)

(73) Assignee: MIGAL Galilee Research Institute Ltd, Kiryat Shmona (IL)

(21) Appl. No.: 18/020,500

(22) PCT Filed: Aug. 12, 2021
(86) PCT No.: PCT/IB2021/000552

§ 371 Date: Feb. 9, 2023

Related U.S. Application Data
(60) Provisional application No. 63/064,683, filed on Aug. 12, 2020.

(51) Int. Cl. C07K 16/28; C07K 14/705; A61P 37/06

(52) U.S. Cl. CPC C07K 16/2806; C07K 14/70528; C07K 14/70589; C07K 14/70596; A61P 37/06; C07K 2319/03

(57) ABSTRACT

Provided herein are cell-distancing devices that protect cells that comprise them against host immune response when administered in a host. The disclosed cell-distancing devices engage with host immune cells and reduce their activity against cells that comprise the devices. Compositions of cell-distancing devices, cells comprising cell-distancing devices, and method of making and using such compositions are disclosed herein.

### RELATED APPLICATIONS

This application claims the benefit under 35 U.S.C. § 119(e) of U.S. Provisional Application No. 63/064,683, entitled “ALLOREACTIVE IMMUNE CELL-DISTANCING DEVICE AND USES THEREOF FOR PROTECTING DONOR-DERIVED CELLS FROM ALLOREJECTION,” filed Aug. 12, 2020, the contents of which are incorporated herein by reference in their entirety.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Aug. 12, 2021, is named G097170008WO00-SEQ-DQP.txt and is 429,673 bytes in size.

### FIELD OF THE INVENTION

The present invention relates in general to tools designed to confer resistance to allorejection on all cell types used in adoptive cell therapy and regenerative medicine, independently of donor/host HLA disparity.

### BACKGROUND OF THE INVENTION

Adoptive cell therapy (ACT), for example of cancer, using allogeneic cells (e.g., T cells or NK cells) offers critical advantages over the use of autologous cells. Numerous strategies for preparing off-the-shelf products, mostly for CAR-T cell therapy, are currently being explored (see [1-4] for review). Yet, HLA disparity between donor and host is responsible for the two major risks associated with the use of allogeneic T cells: graft-versus-host disease (GVHD), resulting from damage to nontumor host tissues inflicted by alloreactive donor T cells, and rejection of the therapeutic donor cells by alloreactive host T and NK cells. The use of donor-derived regulatory T cells (Tregs) also holds great promise in the rapidly evolving field of Treg therapy of inflammatory diseases and disorders [5-8]. While the risk of GVHD posed by adoptively transferred allogeneic effector T cells (Teffs) does not apply to donor Treg therapy (but is replaced by the risk of systemic blunting of immunity), rejection of these cells by host T cells is still a major concern that should be obviated.

Several gene-based approaches, which have been put forward for protecting antitumor donor T cells and NK cells from elimination by the host, are also applicable for Treg protection. One such approach involves the targeted disruption of β₂-microglobulin (β₂m [9]), which can be complemented by the expression of a non-classical HLA protein, such as HLA-E, to avoid an NK response against the resulting HLA-I(−) cells [10]. Targeted elimination of distinct HLA-I products, rather than generating an HLA-I-null cell population, could mitigate the prospects of an NK cell attack [11] [12]. As activated human T cells often express high levels of HLA-JI molecules, the suppression of the HLA-II transactivator (CIITA) and, consequently, HLA-II expression [13] or the knockout of selected HLA-II alleles [14] emerge as practicable strategies for evading alloreactive host CD4 T cells. An entirely different tactic attempts to protect the donor cells from lymphodepletion, which eliminates host T cells, including the anti-donor fraction. Following this rationale, investigators disrupted the CD52 gene to render donor anti-CD19, TCR α chain-knockout CAR-T cells resistant to lymphodepletion mediated by the anti-CD52 mAb alemtuzumab, thus avoiding allorejection [15]. Such double-knockout donor T cells were successfully used to treat two infants with refractory relapsed B-ALL, who were the first patients ever to undergo allogeneic CAR-T cell therapy [16]. Selective protection of donor CAR T cells from lymphodepletion could also be achieved by inactivating the deoxycytidine kinase (dCK) gene, sparing these cells from treatment with purine nucleotide analogues [17].

In the field of regenerative medicine, turning allogeneic stem cell-derived transplants ‘hypoimmunogenic’ for preventing allorejection is an active area of research (e.g., [18], and see [19] for a recent review). The different approaches pursued for achieving this goal exploit the fact that the allogeneic iPSC and ES cell-lines are readily amenable to genetic modification. FIG. 1 (taken from [19]) depicts the multitude of strategies currently explored for this purpose. These include the ablation of HLA-I and HLA-II genes for evading alloreactive host CD8 and CD4 T cells, respectively, expression of HLA-E for inhibiting NK cells, expression of PD-L1 as a pan T cell inhibitory ligand and of HLA-G for inhibiting T cells, NK cells and other immune cells.

These approaches are cumbersome, often require gene editing and most are alloreactive immune cell type-specific (i.e., CD8 or CD4 T cells, NK cells). There remains thus an unmet need for a simple and universal genetic tool designed to confer resistance to allorejection on all cell types used in ACT and/or regenerative medicine from all potentially alloreactive donor immune cells, independently of donor/host HLA disparity.

### SUMMARY OF INVENTION

Aspects of the application relate to compositions and methods for protecting therapeutic cells (e.g., engineered cells that are allogeneic) administered to a subject from host immune responses. In some aspects, cells engineered for use as a therapy are engineered to express a recombinant protein referred to as a cell-distancing device that interferes with synapse formation between the engineered cell and a host immune cell. In some aspects, the recombinant protein includes a domain that is attached to the engineered cell surface (e.g., a transmembrane domain), and a domain that binds to a protein in the synapse of a host immune cell (e.g., a host T-cell). In some aspects, the recombinant protein includes a spacer domain between the domain that attaches to the cell and binding domains such that the recombinant protein interferes with immune synapse formation between the engineered cell and the host immune cell. In some aspects, the spacer domain is an elongation domain that distances membranes of the engineered and host immune cells from each other in a way that interferes with synapse formation.

In some aspects, the present invention provides an alloreactive T cell-distancing device comprising: (a) an extracellular membrane-distal domain comprising a binding domain capable of binding a member of a central supramolecular activation cluster (SMAC) of the immunological synapse or a member closely associated therewith; (b) an extracellular elongation domain comprising at least one rigid protein module; and (c) a transmembrane domain. In some embodiments, domains (a)-(c) are connected from N-terminus to C-terminus in the following order via one or more hinges: transmembrane domain, extracellular elongation domain, and extracellular membrane-distal domain. In some embodiments, domains (a)-(c) are connected from N-terminus to C-terminus in the following order via one or more hinges: transmembrane domain, extracellular elongation domain, and extracellular membrane-distal domain. In some embodiments, domains are connected or attached to other domains without hinges/hinge domains (e.g., in either orientation).

In some embodiments, an alloreactive T cell-distancing device further comprises an extracellular membrane-proximal domain. In some embodiments, an elongation domain and a membrane-proximal domain are a single domain. In some embodiments, a T cell-distancing device further encompasses an intracellular domain optionally capable of associating, or co-clustering with, MHC molecules. In some aspects, the present invention provides an alloreactive T cell-distancing device comprising: (a) an extracellular membrane-distal domain comprising a binding domain capable of binding a member of a central supramolecular activation cluster (SMAC) of the immunological synapse or a member closely associated therewith; (b) an extracellular elongation domain comprising at least one rigid protein module; (c) an extracellular membrane-proximal domain, optionally less than 5 nm in length and/or lacking a glycosylphosphatidylinositol (GPI) anchor; (d) a transmembrane domain; and optionally (e) an intracellular domain optionally capable of associating, or co-clustering with, MHC molecules. In some embodiments, domains (a)-(e) are connected from N-terminus to C-terminus in the following order via one or more hinges: intracellular domain, transmembrane domain, extracellular membrane-proximal domain, extracellular elongation domain, and extracellular membrane-distal domain. In some embodiments, domains (a)-(e) are connected from C-terminus to N-terminus in the following order via one or more hinges: intracellular domain, transmembrane domain, extracellular membrane-proximal domain, extracellular elongation domain, and extracellular membrane-distal domain. In some embodiments, domains are connected or attached to other domains without hinges/hinge domains.

In some embodiments, an alloreactive T cell-distancing device comprises (a) an extracellular membrane-distal domain comprising a binding domain capable of binding a member of a central supramolecular activation cluster (SMAC) of the immunological synapse or a member closely associated therewith; and (b) an elongation domain comprising at least one rigid protein module, wherein said membrane-distal domain is linked via a membrane-proximal domain and a transmembrane domain to an intracellular domain optionally capable of associating, or co-clustering, with, MHC molecules. In some embodiments, a device excludes a membrane-proximal domain, transmembrane domain, and/or intracellular domain of CD22.

In some aspects, the present disclosure provides a nucleic acid molecule comprising a nucleotide sequence encoding an alloreactive T cell-distancing device as described herein. In some aspects, provided herein is a nucleic acid molecule comprising a nucleotide encoding an alloreactive T cell-distancing device comprising (a) an extracellular membrane-distal domain comprising a binding domain capable of binding a member of a central supramolecular activation cluster (SMAC) of the immunological synapse or a member closely associated therewith; and (b) an elongation domain comprising at least one rigid protein module, wherein said membrane-distal domain is linked via a membrane-proximal domain and a transmembrane domain to an intracellular domain optionally capable of associating, or co-clustering, with, MHC molecules, excluding a membrane-proximal domain, transmembrane domain, and/or intracellular domain of CD22.

In some embodiments, a member of the central SMAC is selected from CD2, CD8, CD4, a signaling lymphocytic activation molecule (SLAM) and a CD28 family member. In some embodiments, the CD28 family member is selected from CD28, ICOS, BTLA, CTLA-4 and PD-1.

In some embodiments, a binding domain is a CD2-binding domain selected from an LFA-3 (CD58) CD2-binding domain and a synthetic anti-CD2 antibody.

In some embodiments, an at least one rigid protein module comprises an α-helix-forming peptide sequence, such as (EAAAK)n; or a proline-rich peptide sequence, such as (XP)n, with X designating any amino acid, e.g., Ala, Lys, or Glu. In some embodiments, an at least one rigid protein module is a fibronectin type III repeat or an Ig domain harboring the typical motifs of the Ig fold (Ig-like domain). In some embodiments, an elongation domain comprises at least two Ig-like domains and/or at least three fibronectin type III repeats. In some embodiments, a rigid elongation domain comprises the complete extracellular domain of LFA-3 (containing two Ig-like domains), CD22 (containing seven Ig-like domains), CD45 (comprising three fibronectin type III repeats), or CD148 (comprising five fibronectin type III repeats) or any combination of Ig-like domains and/or fibronectin type III domains. In some embodiments, a complete extracellular domain of CD45 is the complete extracellular domain of the CD45 isoform CD45RO, CD45RAB or CD45RABC.

In some embodiments, a membrane-proximal domain comprises an Ig-like domain (such as an LFA-3 Ig-like domain) or a fibronectin type III repeat. In some embodiments, a transmembrane domain and/or intracellular domain is the transmembrane domain and/or intracellular domain of LFA-3. In some embodiments, a member of the central SMAC is selected from CD2, CD8, CD4, a signaling lymphocytic activation molecule (SLAM), and a CD28 family member; the at least one rigid protein module comprises an α-helix-forming peptide sequence (such as (EAAAK)n), a proline-rich peptide sequence (such as (XP)n, with X designating any amino acid), a fibronectin type III repeat or an Ig domain harboring the typical motifs of the Ig fold (Ig-like domain); a membrane-proximal domain comprises an Ig-like domain (such as an LFA-3 Ig-like domain) or a fibronectin type III repeat; and a transmembrane domain and/or intracellular domain is the transmembrane domain and/or intracellular domain of LFA-3.

In some embodiments, a binding domain is a CD2-binding domain selected from an LFA-3 (CD58) CD2-binding domain or a synthetic anti-CD2 antibody; the CD28 family member is selected from CD28, ICOS, BTLA, CTLA-4 and PD-1; and an elongation domain comprises at least two Ig-like domains and/or at least three fibronectin type III repeats. In some embodiments, a rigid elongation domain comprises the complete extracellular domain of LFA-3 (containing two Ig-like domains), CD22 (containing seven Ig-like domains), CD45 (comprising three fibronectin type III repeats), or CD148 (comprising five fibronectin type III repeats) or any combination of Ig-like domains and/or fibronectin type III domains. In some embodiments, a complete extracellular domain of CD45 is the complete extracellular domain of the CD45 isoform CD45RO, CD45RAB or CD45RABC.

In some embodiments, an alloreactive T cell-distancing device as provided herein comprises an LFA-3 CD2-binding domain; a rigid elongation domain comprising at least two CD22 Ig-like domains and at least one LFA-3 Ig-like domain; or a complete extracellular CD45 domain and at least one LFA-3 Ig-like domain; an LFE-3 Ig-like membrane-proximal domain, and an LFE-3 transmembrane and intracellular domain. In some embodiments, a rigid elongation domain comprises a complete extracellular CD45 domain selected from that of CD45RO, CD45RAB and CD45RABC and one LFA-3 Ig-like domain, and a complete extracellular CD45 domain is located between the LFE-3 Ig-like membrane-proximal domain and the LFA-3 Ig-like rigid elongation domain.

In some aspects, the present disclosure provides a vector comprising the nucleic acid molecule of any one of the preceding embodiments. In some embodiments, the vector is a DNA vector, such as a plasmid or viral vector; or a non-viral vector, such as a polymer nanoparticle, lipid, calcium phosphate, DNA-coated microparticle or transposon.

In some aspects, the present disclosure provides a method for producing a donor-derived allogeneic cell, cell-line or stem cell-line expressing an alloreactive T cell-distancing device, said method comprising contacting a donor-derived allogeneic cell, cell-line or stem cell-line with any one of the nucleic acid molecules or vectors described herein, thereby reducing the destruction by allorejection of said donor-derived allogeneic cell, cell-line or stem cell-line is in adoptive cell therapy or stem cell transplantation. Similarly, a differentiated cell, organ or tissue derived from said stem cell-line is destroyed less from allorejection in cell, organ or tissue transplantation compared to a cell, organ or tissue not derived from a stem cell-line not contacted with any one of the nucleic acid molecules or vectors described herein. In some embodiments, a donor-derived allogeneic cell is an immune cell, such as a cytotoxic T cell, regulatory T cell (Treg), B cell or NK cell; or a hematopoietic stem cell. In some embodiments, an immune cell further expresses a chimeric antigen receptor (CAR). In some embodiments, a donor-derived allogeneic cell-line is an induced pluripotent stem cell-line. In some embodiments, a differentiated cell derived from an induced pluripotent stem cell-line is a retinal pigment epithelial cell, cardiac cell or neural cell.

In some aspects, the present disclosure provides a donor-derived allogeneic cell, cell-line or stem cell-line or a differentiated cell, organ or tissue derived from stem cells, expressing or comprising any one of the nucleic acid molecules or vectors described herein, thereby reducing the destruction of said donor-derived allogeneic cell, cell-line or stem cell-line by allorejection in adoptive cell therapy. Similarly, a differentiated cell, organ or tissue derived from said stem cell-line is destroyed less from allorejection in cell, organ or tissue transplantation compared to a cell, organ or tissue not derived from a stem cell-line not contacted with any one of the nucleic acid molecules or vectors described herein.

In some aspects, the present disclosure provides a method of transplantation therapy in a subject in need thereof, said method comprising administering to said subject in need any one of the donor-derived allogeneic cell, cell-line or stem cell-line or a differentiated cell, organ or tissue derived from stem cells described herein.

In some aspects, the present disclosure provides a method comprising administering to a subject any one of the donor-derived allogeneic cell described herein.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 depicts engineering strategies to generate hypoimmunogenic human pluripotent stem cells (taken from [19]).

FIGS. 2A-2B depict the KS model. FIG. 2A illustrates the mechanism by which the KS model ensures the exclusion or inclusion of cell surface molecule in the contact zone between cells (illustration and text copied from [22]). In a resting T cell (upper left panel), random protein interactions in the membrane lead to phosphorylation and dephosphorylation of molecules with tyrosine-phosphorylation motifs by Src kinases and tyrosine phosphatases. Triggering occurs as the local balance of those constitutive processes is altered by the formation of close-contact zones between the T cell and an antigen-presenting cell (APC) (lower left, upper right and lower right panels). This process is ‘nucleated’ by small proteins such as CD2 and results in the local size-dependent exclusion of large proteins such as CD45 from the close-contact zone. The ‘fates’ of the three TCRs here (1, 2, 3) are presented according to the KS model. In the absence of peptide-MHC contact, TCR 1 diffuses from the contact zone. If TCR 1 had been phosphorylated in the close-contact zone or before its formation, rapid dephosphorylation outside the close-contact zone would prevent signaling. TCR 2, phosphorylated before close contact zone formation, binds cognate MHC-peptide and is thereby ‘held’ in the close-contact zone long enough for ‘downstream’ events to occur. TCR 3, phosphorylated after close-contact zone formation, is phosphorylated by free Lck only, accounting for coreceptor-independent triggering. Relatively large amounts of phosphorylation of TCR 2 and TCR 3 lead to ‘downstream’ signaling after Zap70 recruitment. FIG. 2B shows the size-dependent sorting mechanism that operates at the contact zone between a T cell (top) and an antigen-presenting cell (bottom) (copied from: Aricescuet al. Curr. Opin. Cell Biol. 2007).

FIG. 3 provides a schematic illustration of the immunological synapse and spatial distribution of TCR/MHC and costimulatory molecules in the plasma membranes of a T cell (top) interfacing with an antigen-presenting cell (bottom) (copied from [24]). Illustration is for explanatory purposes and relative sizes of different molecules are not necessarily to scale. Regions include central supramolecular activation cluster (cSMAC), peripheral SMAC (pSMAC), CD2/LFA3 corolla and distal SMAC (dSMAC). CD2 is positioned in the T cell plasma membrane and locates to both the cSMAC and corolla. CD2 binds to lymphocyte-associated antigen 3 (LFA3) which is located in the plasma membrane of the antigen-presenting cell. Among other molecules, TCR/pMHC and CD28/CD80/86 complexes also locate to the cSMAC. LFA-1/ICAM-1 complexes predominantly locate to the pSMAC.

FIGS. 4A-4C provide a model explaining the contrasting effects of wild type and elongated CD48 on T cell antigen recognition (illustration and text copied from [25]). FIG. 4A shows a schematic representation of the various forms of CD48 used in [25]. Segments derived from mouse CD48 and segments inserted from human CD2 or mouse CD22 are depicted as heavy and light lines, respectively. The asterisk represents the CD22 mutation, R130A. FIG. 4B shows how CD2 molecules on T cells and CD48 molecules on I-EK1 CHO APCs interact to form contacts in which the intermembrane separation distance is determined by the dimensions of the CD2/CD48 complex. Wild type CD48 (left) enhances T cell antigen recognition because the separation distance (≈15 nm) is optimal for TCR engagement of peptide-MHC. Elongated CD48 (CD48-CD22, right) inhibits T cell antigen recognition by forming contacts in which the intermembrane distance (>20 nm) is too great for TCR to engage pMHC. FIG. 4C shows that elongated forms of CD48 inhibit T cell antigen recognition. Antigen recognition by 2B4.CD2 cells using as APCs I-E^{k1} CHO cells expressing no CD48 (CD48 neg CHO), wild type CD48 (CD48 CHO), CD48-CD2, or CD48-CD22. The arrow marks the response curves produced in the presence of the two elongated constructs.

FIGS. 5A-5L provide schematic representations of exemplary T cell-distancing device constructs described herein. FIG. 5A shows a non-limiting example of a generic structure of a T cell-distancing device expressed on the surface of a cell membrane, and comprising a membrane-distal domain (MD), an elongation domain (EL), a membrane-proximal domain (MP), a transmembrane domain (TM), and an intracellular domain (IC). FIG. 5B shows schematic representations of non-limiting, alternative designs of the elLFA-3 device. Shown in the left are two molecules harboring 5 Ig-like domains derived from LFA-3 and CD22, in analogy to the CD48-CD22 configuration, which exhibited the strongest inhibition in ([25], [36] and see FIG. 6). These two molecules differ in their membrane-proximal Ig-like domains and in their transmembrane and cytoplasmic portions: whereas ‘elLFA-3, CD22 anchor’ (left) precisely recapitulates CD48-CD22, ‘elLFA-3, LFA-3 anchor’ (2^{nd} right) preserves the native membrane-proximal Ig domain, and the transmembrane and cytoplasmic portion of LFA-3, as discussed in the text. Shown in the right are three molecules harboring as the backbone of the extracellular stalk the ectodomains of the three CD45 isoforms CD45RO, CD45RAB and CD45RABC. According to [45], the size of the CD45RO-derived portion is 22 nm and that of CDRABC is 40 nm, which add to the three Ig-like domains of LFA-3 incorporated into these constructs. All constructs will be provided with a peptide tag for easy detection. The CD45 part of these sketches was taken from www.bio-rad-antibodies.com/cd45-characterization-isoforms-structure-function-antibodies-minireview.html#. FIG. 5C shows schematic representations of alternative variations of the device designs shown in FIG. 5B. FIG. 5D also shows schematic representations of alternative designs of T cell-distancing devices, similar to the ones in FIG. 5B but lacking a membrane proximal LFA-3 domain. Human designs 1882, 1883 and 1884 comprise intracellular and membrane-distal domains of LFA-3, while mouse designs 1885, 1886 and 1887 comprise intracellular and membrane-distal domains of CD48. FIG. 5E shows mRNA constructs used for transfection of donor-derived allogeneic cells for expressing the devices represented in FIG. 5C. FIG. 5F shows schematic representations of additional designs of T cell-distancing devices for use in human and mouse. FIG. 5G shows mRNA constructs used for transfection of donor-derived allogeneic cells for expressing the devices represented in FIG. 5F. FIGS. 5H and 5I also show variations of the designs of T cell-distancing devices comprising CD58 ectodomains in the extracellular membrane-distal domains. The devices in FIG. 5H comprise one CD58 domain with the devices on the right side comprising a hinge (Li, e.g., SEQ ID NO: 105) between the extracellular membrane-distal domain and the elongation domain. FIG. 5I shows devices similar to the ones in FIG. 5H, but comprising two CD58 domains in the extracellular membrane-distal domain. FIG. 5J shows devices comprising anti-CD2 scFv fragments in the extracellular membrane-distal domain, connected to the elongation domain through a hinge (CD8a on the left, Li on the right). FIG. 5K shows mRNA constructs used for transfection of donor-derived allogeneic cells for expressing the devices represented in FIGS. 5H and 5I. FIG. 5L show mRNA constructs used for transfection of donor-derived allogeneic cells for expressing the devices represented in FIG. 5J.

FIG. 6 depicts the expected effect of the expression of elLFA-3 on the interaction with an alloreactive T cell. Left, normal T cell-target cell interactions. Right, the extended interface forced by elLFA-3. Here ‘Target cell’ (bottom) refers to the donor cell, which should be protected from attack by the alloreactive host T cell (‘T lymphocyte’, top). The arrows represent the anticipated associations between CD2 and the TCR in the T cell and LFA-3 and MHC in the target cell, which are expected to prevent segregation of elLFA-3 from the contact zone, thus guaranteeing the inhibitory effect. Based on www.microbiologybook.org/bowers/target.jpg.

FIG. 7 depicts the mechanism of action of a T cell-distancing device expressed on the surface of a transplanted engineered Treg. The Treg comprises a TCR or CAR construct directed to a target, which can be a cell or a non-cell target (e.g., a tumor antigen). The T cell-distancing devices are attached to the cell membrane via the LFA-3 transmembrane and intracellular domains, and are expected to be excluded from the contact zone, as predicted by the KS model, so as to not interfere with the intended Engineered Treg activity, as shown on the left side of the figure. The right side of the figure shows the role of the T cell-distancing device in inhibiting host immune cell reaction towards the Engineered Tregs; CD2 plays a key role in the formation and stabilization of the immunological synapse of T cells and NK cells. Multiple interactions of CD2 on potentially alloreactive anti-donor host T (or NK) cells with the T cell-distancing device on the donor-derived Engineered Tregs enforce a large distance between the two cells, decreasing both contact of the TCR with the donor MHC and the exclusion of phosphatases (mainly CD45) from the contact zone.

FIG. 8 shows the expected outcomes of a p-galactosidase assay on different experimental settings for the detection of T-cell activity on a target cell expressing a T cell-distancing device. The target cell is an antigen-presenting cell (APC) that presents a peptide recognized by a T-cell receptor (TCR). Upon binding of the TCR to the peptide (shown in experimental setting 1), p-galactosidase catalyzes the hydrolysis of the galactoside analog chlorophenol red-p-D-galactopyranoside (CPRG) which is converted to chlorophenol red (CPR). In the presence of a T cell-distancing device on the APC (as shown in experimental setting 2), T-cell activity is blocked and CPRG is not converted to CPR. The T cell-distancing device is expected to be excluded from the immune synapse, and therefore does not substantively prevent an engineered T cell (C) from responding to its target cell (experimental setting 3).

FIG. 9 shows the expected outcomes of a $-galactosidase assay on different experimental settings for the detection of CAR T-cell activity on target cell. When the CAR construct recognizes its target on an antigen-presenting cell (APC), the CAR T-cell is activated (experimental setting 1). In the presence of a device on the APC (as shown in experimental setting 2), T-cell activity is blocked and CPRG is not converted to CPR. The T cell-distancing device is expected to be excluded from the immune synapse, and therefore does not substantively prevent an engineered CAR T-cell expressing the T cell-distancing device (C) from acting on its target (experimental setting 3).

FIGS. 10A-10B show Pmel-TCR activation levels in CD8 T-cells exposed to RMA cells loaded with gp100 peptide. In FIG. 10A, Pmel-TCR activation was determined by expressing Pmel-TCR in CD8 T cells, which were then co-cultured with RMA cells loaded with different concentrations of gp100 peptide (0-1000 ng/ml). Supernatant was collected and analysed for INF-γ expression. In FIG. 10B, Pmel-TCR activation was determined by expressing Pmel-TCR in CD8 T cells, which were then co-cultured overnight with RMA cells loaded with different concentrations of gp100 peptide (0-5 ng/ml). Supernatant was collected and analysed for INF-γ expression.

### DETAILED DESCRIPTION OF THE INVENTION

Disclosed herein are compositions and methods designed to confer resistance to allorejection on all cell types used in ACT and/or regenerative medicine, independently of donor/host HLA disparity. Some embodiments of methods described herein involve the delivery of a single gene for expression of a cell-distancing device on therapeutic cells that decreases the therapeutic cell from being attacked by host immune cells such as T cells and NK cells. The cell-distancing devices which is expressed on the surface of a therapeutic cell engages with host immune cells (e.g., T cells and NK cells), but reduces the chance of them from attacking the therapeutic cells which comprise them. Delivery of a single gene can replace multiple gene editing steps that are currently explored and simplifies reprogramming protocol while preserving the designated therapeutic activity of the gene-modified allogeneic cells.

Provided herein are cell-distancing devices (e.g., a protein that is expressed on the surface of a therapeutic cell); nucleic acids encoding cell-distancing devises; methods of making therapeutic cells that comprise or express one or more cell-distancing devices; therapeutic cells that comprise or express one or more cell-distancing device; and methods of using such cells or administering such cells to a subject.

Herein, cells that are designed or prepared to be administered to a subject are referred to as therapeutic cells or donor cells. Therapeutic cells are any cells (allogeneic, autologous) that are designed and or prepared with the goal of administering them to a subject for any purpose such as for providing treatment. These cells may be one of many cells cultured under certain conditions, or part of an organ that is harvested, part of an organoid, or an organism. In some embodiments, a cell to be administered to a host/subject is engineered so that it expresses exogenous nucleic acid, proteins/peptides or in which the genome has been artificially manipulated. In some embodiments, a cell disclosed herein is a eukaryotic cell (derived from a eukaryotic organism). In some embodiments, a eukaryotic cell is derived from ectoderm, endoderm, or mesoderm. In some embodiments, therapeutic cells or donor cells may be immune cells (e.g., a T cell or B cell).

Regardless of the type of cell, a therapeutic cell, especially if is it allogeneic to the subject to which the cell is to be administered, needs to be protected from the host immune cells, e.g., from host T-cells and NK cells, so that it survives long enough to reach its target and effectuate its function.

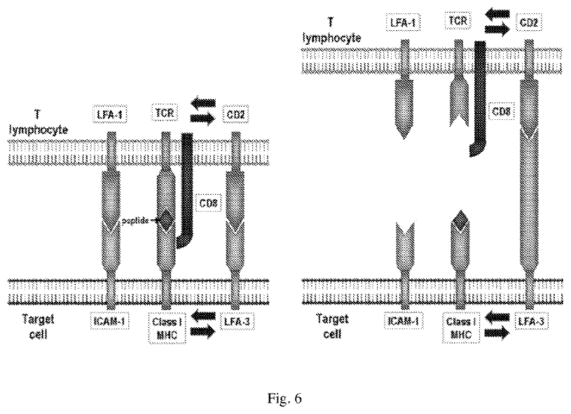

The cell-distancing device as provided herein is to be expressed on the surface of cells to be protected in a host (e.g., a subject into which a therapeutic cell is administered) so that the device engages with host immune cells but reduces activation of those cells, and thus destruction by those host immune cells of the therapeutic cell. In some embodiments, the cell-distancing device engages with an element involved in the synapse between immune cells and the donor or therapeutic cell. These elements may be members of a central supramolecular activation cluster (SMAC) of the immunological synapse or a member closely associated therewith (see e.g., FIG. 3). In some embodiments, the cell-distancing device presented herein engages with an element on the surface of host immune cells, and reduces the chance of engagement of other elements of the immune cell that are involved in attacking host cells. FIG. 6 provides an example of a cell-distancing device that engages host T cells, and more specifically, CD2 on the surface of host T cells, and increases the distance between host T cells and the therapeutic cell that expresses the cell-distancing device such that the TCR on the host T cells cannot engage with the MHC-presented antigen on the therapeutic cell.

### Cell Distancing Device

In some aspects, provided herein is a device comprising (a) an extracellular membrane-distal domain comprising a binding domain that is capable of binding to a member of a central supramolecular activation cluster (SMAC) of the immunological synapse or a member closely associated therewith; (b) an elongation domain comprising at least one rigid protein module; and (c) a transmembrane domain. In some embodiments, a cell-distancing device of the present disclosure further comprises a membrane-proximal domain that is present between an elongation domain and a transmembrane domain. In some embodiments, an elongation domain and a membrane-proximal region are considered to be a single domain that is present between an extracellular membrane-distal domain and a transmembrane domain. In some embodiments, a cell-distancing device of the present disclosure further comprises an intracellular domain. In some embodiments, an intracellular domain is capable of binding or binds to class I MHC. In some embodiments one or more domains of a cell-distancing device is connected to another domain vial a hinge domain. In some embodiments, an extracellular membrane-distal domain is connected to a hinge domain via its N and C termini. In some embodiments, an extracellular membrane-distal domain is connected to a hinge domain via its N termini. In some embodiments, an extracellular membrane-distal domain is connected to a hinge domain via its C termini. In some embodiments, a cell distancing device comprises a transmembrane domain, an extracellular elongation domain, and an extracellular membrane-distal domain that are connected from N-terminus to C-terminus in the following order (optionally via one or more hinges): transmembrane domain, extracellular elongation domain, and extracellular membrane-distal domain. In some embodiments, a cell distancing device comprises a transmembrane domain, an extracellular elongation domain, and an extracellular membrane-distal domain that are connected from C-terminus to N-terminus in the following order (optionally via one or more hinges): transmembrane domain, extracellular elongation domain, and extracellular membrane-distal domain. In some embodiments, a membrane-proximal domain connects an elongation domain with a transmembrane domain.

In some embodiments, a cell distancing device comprises an intracellular domain, a transmembrane domain, an extracellular elongation domain, and an extracellular membrane-distal domain that are connected from N-terminus to C-terminus in the following order (optionally via one or more hinges): intracellular domain, transmembrane domain, extracellular elongation domain, and extracellular membrane-distal domain. In some embodiments, a cell distancing device comprises a intracellular domain, transmembrane domain, an extracellular elongation domain, and an extracellular membrane-distal domain that are connected from C-terminus to N-terminus in the following order (optionally via one or more hinges): transmembrane domain, extracellular elongation domain, and extracellular membrane-distal domain. In some embodiments, a membrane-proximal domain connects an elongation domain with a transmembrane domain.

In some embodiments, the length between the N-terminus and C-terminus of a cell-distancing device is at least 10 nm (e.g., at least 10, at least 11, at least 12, at least 13, at least 14, at least 15, at least 16, at least 17, at least 18, at least 19, at least 20, at least 21, at least 22, at least 23, at least 24, or at least 25 or more nm). In some embodiments, the length between the N-terminus and C-terminus of a cell-distancing device is 5-40 nm (e.g., 1-40, 10-40, 10-30, 10-25, 12-24, 15-15, 15-30, 5-20, 15-20, or 25-30 nm). In some embodiments, the length between the farthest extracellular part of the devise from the cell membrane of the cell comprising the device and the cell membrane is at least 10 nm (e.g., at least 10, at least 11, at least 12, at least 13, at least 14, at least 15, at least 16, at least 17, at least 18, at least 19, at least 20, at least 21, at least 22, at least 23, at least 24, or at least 25 or more nm). In some embodiments, the length between the farthest extracellular part of the devise from the cell membrane of the cell comprising the device and the cell membrane is 5-40 nm (e.g., 1-40, 10-40, 10-30, 10-25, 12-24, 15-15, 15-30, 5-20, 15-20, or 25-30 nm).

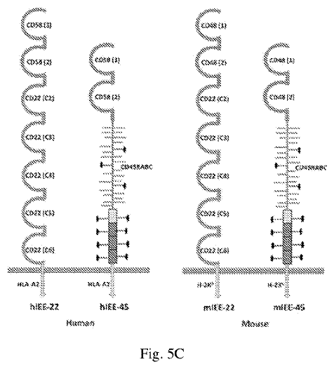

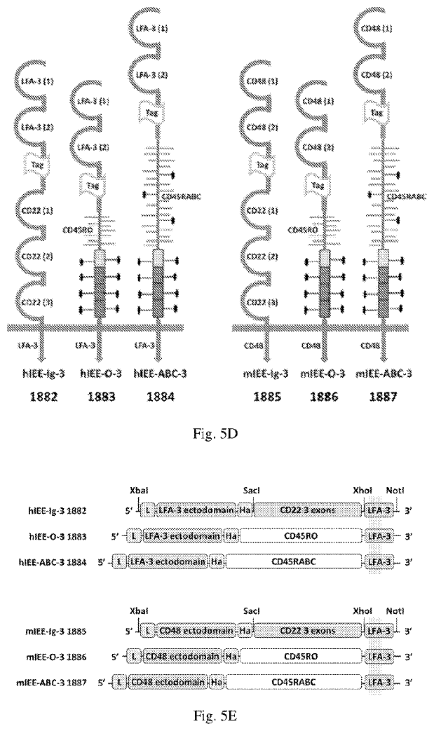

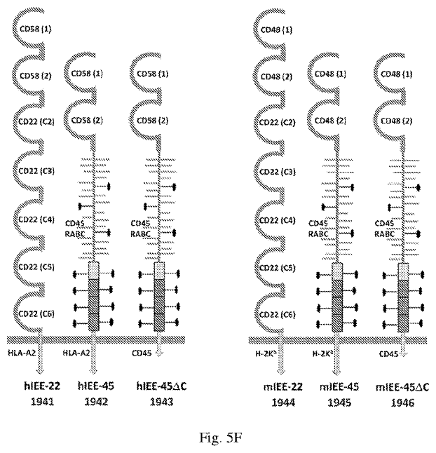

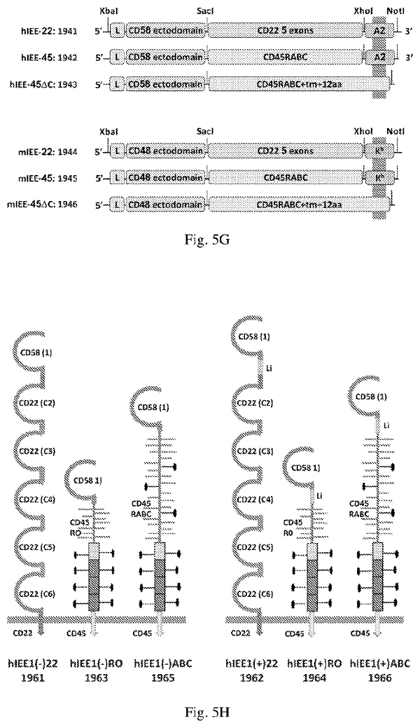

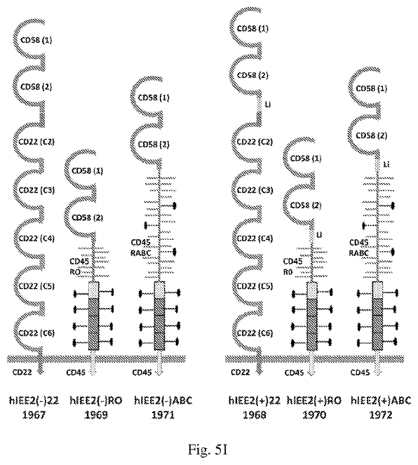

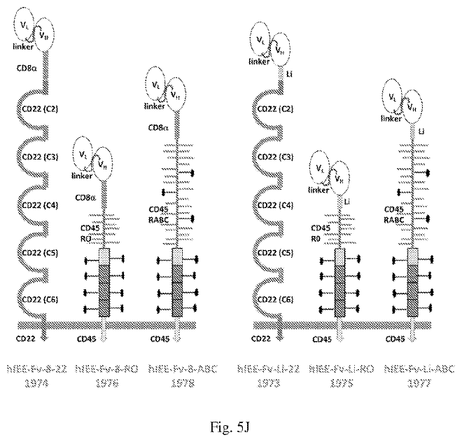

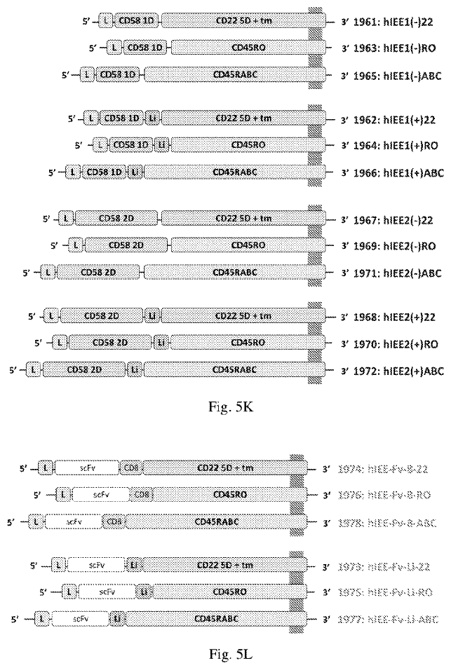

In some aspects, provided herein is a device comprising (a) an extracellular membrane-distal domain comprising a binding domain that is capable of binding to a member of a central supramolecular activation cluster (SMAC) of the immunological synapse or a member closely associated therewith; (b) an elongation domain comprising at least one rigid protein module; (c) a membrane-proximal domain; and (d) a transmembrane domain. In some embodiments, a membrane-distal domain is connected to the elongation domain by one or more hinges. In some embodiments, an elongation domain is connected to the membrane-proximal domain by one or more hinges. See for example FIG. 5A. In some embodiments, an elongation domain is connected to the membrane-distal domain by one or more hinges. In some embodiments, the device as provided herein further comprises an extracellular-membrane proximal domain. In some embodiments, an extracellular-membrane proximal domain and elongation domain are considered as a single domain. In some embodiments, the device as provided herein further comprises an intracellular domain. In some embodiments, the intracellular domain is connected to the transmembrane domain by one or more hinges. The cell-distancing device as provided herein may also comprise one or more tags that can be used as a market to identify the device or part/domain thereof.

### Extracellular Membrane-Distal Domain

In some embodiments, an “extracellular membrane-distal domain” refers to the extracellular domain of the cell-distancing device as provided herein that is farthest from the transmembrane domain. The extracellular membrane-distal domain provides a binding domain for the cell-distancing device to engage with a cell-surface protein (e.g., a member of a central supramolecular activation cluster (SMAC) of the immunological synapse or a member closely associated therewith) on a host immune cell (e.g., a T cell or NK cell). In some embodiments, an extracellular membrane-distal domain is attached to a hinge domain via its N-, C-, or both N and C-termini. That is in some embodiments, an extracellular membrane-distal domain is the second farthest from the transmembrane domain and has a hinge that is even farther than the extracellular membrane-distal domain relative to the transmembrane domain.

In some embodiments of the devices provided herein, the extracellular membrane-distal domain is capable of binding to a member of the SMAC of the immunological synapse. Binding of the most distal domain of a cell surface protein to a SMAC member acts to separate the cell expressing the device, such as an engineered cell, from the cell expressing the SMAC member, such as an NK cell or T cell (e.g., a CD8+ T cell). In some embodiments, the member of the central SMAC is selected from the group consisting of CD2, CD8, CD4, a signaling lymphocytic activation molecule (SLAM), and a CD28 family member. In some embodiments, the CD28 family member is selected from CD28, ICOS, BTLA, CTLA-4 and PD-1.

In some embodiments, the extracellular membrane-distal domain is a portion of a human protein.

In some embodiments, a binding domain comprises a natural binding domain or an antibody or fragment thereof that binds to a member of the SMAC of an immunological synapse.

In some embodiments, a binding domain is a natural binding domain of CD2, e.g., a binding domain in LFA-3 (CD58 or CD48) that binds to CD2. In some embodiments, a binding domain is an antibody or a fragment thereof (e.g., an antibody, scFV, Fab, or VH or VL) that binds to a member of the SMAC, e.g., CD2.

In some embodiments, a binding domain is a CD2-binding domain selected from a CD2-binding domain of LFA-3 (CD58 or CD48), and a synthetic anti-CD2 antibody or functional fragment thereof.

In some embodiments, an extracellular membrane-distal domain comprises multiple domains of CD48 or CD58. In some embodiments, an extracellular membrane-distal domain comprises CD58 domain A or a fragment thereof, CD58 domain B or a fragment thereof, or CD58 domain A and domain B or fragments thereof. In some embodiments, the extracellular membrane-distal domain comprises two domains of CD58. In some embodiments, the extracellular membrane-distal domain comprises two domains of CD48.

In some embodiments, the term “synthetic anti-CD2 antibody,” as used herein, refers to any extracellular binding domain excluding the naturally occurring CD2-binding domain of LFA-3, such as (i) an antibody, derivative or fragment thereof, such as a humanized antibody; a human antibody; a functional fragment of an antibody; a single-domain antibody, such as a Nanobody; a recombinant antibody; and/or a single chain variable fragment (ScFv); (ii) an antibody mimetic, such as an affibody molecule; an affilin; an affimer; an affitin; an alphabody; an anticalin; an avimer; a DARPin; a fynomer; a Kunitz domain peptide; and a monobody; or (iii) an aptamer. In some embodiments, the synthetic anti-CD2 antibody is an anti-CD2 ScFv.

In some embodiments, the SLAM is selected from SLAMF1 (CD150), SLAMF2 (CD48, FimH, 2B4), SLAMF3 (CD229, LY9), SLAMF4 (CD244), SLAMF5 (CD84), SLAMF6 (CD352), SLAMF7 (CD319, CRACC), SLAMF8 (CD353), and SLAMF9.

SEQ ID NOs: 1-14 and SEQ ID NOs: 56-69 provide examples of nucleic acid sequences that encode extracellular membrane-distal domains that can bind CD2 and amino acid sequences of extracellular membrane-distal domains that can bind CD2, respectively. Nucleic acid sequences in Table 4 correspond to amino acid sequences in Table 5. In some embodiments a device as provided herein has an extracellular membrane-distal domain comprising an amino acid sequence that is at least 50% (e.g., at least 50%, at least 60%, at least 70%, at least 80%, at least 85%, at least 90%, at least 95%, at least 97%, at least 99%) identical to any one of SEQ ID NOs: 56-69. In some embodiments a device as provided herein has an extracellular membrane-distal domain comprising an amino acid sequence that is identical to any one of SEQ ID NOs: 56-69. In some embodiments, an extracellular membrane-distal domain comprises one or more (e.g., two or three) domains included in any one of SEQ ID NOs: 56-69. In some embodiments, one or more domains in an extracellular membrane-distal domain comprises a sequence that is at least 50% (e.g., at least 50%, at least 60%, at least 70%, at least 80%, at least 85%, at least 90%, at least 95%, at least 97%, at least 99%) identical to the sequence of a domain in SEQ ID NOs: 56-69. In some embodiments, an extracellular membrane-distal domain comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one SEQ ID NOs: 56-69. In some embodiments, a first contiguous amino acid sequence region is at least 10 amino acid long (e.g., at least 10, at least 20, at least 30, at least 40, at least 50 amino acids, or at least 100 or more amino acids long).

In some embodiments, a membrane-distal domain binds to the SMAC member with a dissociation constant of at least 10⁻⁶ M (e.g., at least 10⁻⁶ M, at least 10⁻⁷ M; at least 10⁻⁸ M; at least 10⁻⁹ M; at least 10⁻¹⁰ M; at least 10⁻¹¹ M; at least 10⁻¹² M; or at least 10⁻¹³ M). Methods of measuring the K_{D} of a binding molecule with respect to an epitope or antigen are well known in the art (see, e.g., Pichler et al. J. Immunol. Methods. 1997; 201(2):189-206).

An extracellular membrane-distal domain may be located at the N-terminus or C-terminus of a protein. The membrane-distal domain may be separated from the transmembrane domain by one or more intervening domains, such as an elongation domain, membrane-proximal domain, hinge domain, and/or one or more linkers.

In some embodiments, a membrane-distal domain is at least 30 amino acids long (e.g., at least 30 amino acids, at least 40 amino acids, at least 50 amino acids, at least 60 amino acids, at least 70 amino acids, at least 80 amino acids, at least 90 amino acids, at least 100 amino acids, at least 110 amino acids, at least 120 amino acids long, at least 150 amino acids long, at least 200 amino acids long, at least 250 amino acids long, at least 300 amino acids long, at least 350 amino acids long, at least 400 amino acids long, at least 450 amino acids long, at least 500 amino acids long, or at least 600 amino acids long). In some embodiments, a membrane-distal domain is at most 5,000 amino acids long (e.g., at most 5,000 amino acids, at most 4,500 amino acids, at most 4,000 amino acids, at most 3,500 amino acids, at most 3,000 amino acids, at most 2,500 amino acids, at most 2,000 amino acids, at most 1,800 amino acids, at most 1,600 amino acids, at most 1,400 amino acids, at most 1,200 amino acids, at most 1,000 amino acids, at most 900 amino acids, at most 800 amino acids, at most 700 amino acids, at most 600 amino acids, at most 500 amino acids, at most 450 amino acids, at most 400 amino acids, at most 350 amino acids, at most 300 amino acids, at most 250 amino acids, or at most 200 amino acids long). In some embodiments, a membrane-distal domain is 10-5,000 amino acids long (e.g., 10-5,000, 20-4,800, 40-4,500, 100-4,000, 200-3,500, 400-3,000, 400-2,500, 400-2,000, 400-1,000, 500-800, 500-900, 500-950, 5-30, 10-20, 10-50, 50-200, 100-200, 100-400, 200-250, 250-300, 200-300, 200-500, or 500-5000 amino acids long).

In some embodiments, the binding domain of the membrane-distal domain is at least 5 nm (e.g., at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 or more nm) away from the membrane of the cell in which is it comprised. In some embodiments, the membrane-distal domain is provided such a distance from the membrane of the cell in which the device is comprises by the elongation domain. In some embodiments, the binding domain of the membrane-distal domain is at least 5-40 nm (e.g., 1-40, 10-40, 10-30, 10-25, 12-24, 15-15, 15-30, 5-20, 15-20, or 25-30 nm) away from the membrane of the cell in which is it comprised.

### Elongation Domain

In some embodiments, an “elongation domain” refers to a domain of the cell-distancing device as provided herein that increases the distance between a membrane-distal domain and a transmembrane domain of the device. In some embodiments, expression of a cell-distancing devices as described herein increases the distance between the cell surface of the cell expressing it and a host immune cell when the membrane-distal domain of the device is engaged with its partner on the host immune cell (e.g., engagement between CD-2 binding membrane-distal domain and CD2 on the host immune cell). In some embodiments, this distance is increased by at least 10% (e.g., by at least 10%, at least 20% at least 30%, at least 40%, at least 50%, at least 75%, at least 100%) relative to the distance of a therapeutic cell that does not express a cell-distancing device and a host immune cell. In some embodiments, this distance is increased by at least 1 nm (e.g., by at least 1 nm, at least 1.5 nm, at least 2 nm, at least 2.5 nm, at least 3 nm, at least 4, at least 5 or more nm) relative to the distance of a therapeutic cell that does not express a cell-distancing device and a host immune cell. In some embodiments, a cell distancing device comprised in a cell results in a distance between that cell and a host immune cell that is at least 10 nm (e.g., at least 10, at least 11, at least 12, at least 13, at least 14, at least 15, at least 16, at least 17, at least 18, at least 19, at least 20, at least 21, at least 22, at least 23, at least 24, or at least 25 or more nm). In some embodiments, a cell distancing device comprised in a cell results in a distance between that cell and a host immune cell that is at least 5-40 nm (e.g., 1-40, 10-40, 10-30, 10-25, 12-24, 15-20, 15-30, 5-20, 15-20, or 25-30 nm).

In some embodiments, an elongation domain as provided herein comprises at least one rigid protein module. In some embodiments, a “rigid protein module” or “rigid domain” refers to a protein or a fragment thereof, such as a protein domain or peptide, comprising a secondary or tertiary structure that is common to at least two different conformations of a protein comprising the rigid protein module. Binding of a protein to a ligand may induce a conformational change in the protein characterized by the movement of flexible domains, such as linkers and hinges, while rigid domains maintain the same structure. A rigid protein module that retains the same structure despite conformational changes in other parts of the protein is thus useful for maintaining a desired structure in a portion of the protein. An elongation domain positioned between a membrane-distal domain and a membrane-proximal domain of a cell-distancing device, for example, may maintain a certain physical distance between the membrane proximal-domain and the membrane-distal domain. In some embodiments, the elongation domain comprises 1, 2, 3, 4, 5, 6, 7, 8, 9, or 10 rigid protein modules. In some embodiments, the elongation domain comprises two or more rigid protein modules with the same amino acid sequence. In some embodiments, the elongation domain comprises two or more rigid protein modules with different amino acid sequences. In some embodiments, one or more rigid protein modules are derived from a human protein. In some embodiments, each of the rigid protein modules are derived from a human protein. In some embodiments, the entire elongation domain is human. The at least one rigid protein module may be based on any rigid motif commonly used as a spacer or a linker in protein engineering, such as alpha helix-forming linkers with the sequence of (EAAAK)n (SEQ ID NO: 171) according to [51]. The α-helical structure was shown to be rigid and stable, with intra-segment hydrogen bonds and a closely packed backbone. Therefore, stiff α-helical linkers may act as rigid spacers between protein domains. For example, an empirical rigid linker with the sequence of A(EAAAK)nA (n=2-5) (SEQ ID NO:171) was shown to be stabilized by the Glu− -Lys+ salt bridges within segments and analysis showed that helical linkers can separate functional domains more effectively than non-helical linkers.

Another type of rigid linker that can be used as a rigid protein domain in the cell-distancing devices disclosed herein has a Pro-rich sequence, (XP)n, with X designating any amino acid, e.g., Ala, Lys, or Glu. The presence of Pro in non-helical linkers can increase the stiffness, and allows for effective separation of the protein domains. The structure of proline-rich sequences was extensively investigated by several groups; For example, lH-NMR spectroscopy was conducted to elucidate the conformation of the (Ala-Pro)7 dipeptide repeat in the N-terminal alkali light chain of skeletal muscle and was shown to exhibit an extended and rigid conformation, probably due to the high frequency of Pro, which imposes strong conformational constrain. Another study of 33-residue peptides containing repeating -Glu-Pro- or -Lys-Pro- also suggested that the X-Pro backbone displayed a relatively elongated and stiff conformation.

Thus, rigid linkers exhibit relatively stiff structures, e.g., by adopting α-helical structures or by containing multiple Pro residues. The length of the linkers can be easily adjusted by changing the copy number to achieve an optimal distance between domains. The linkers are rigid enough to maintain distance, therefore their length is limited to preserve distancing via the rigid domain. In some embodiments, the linkers are less than 5 nm long (e.g., less than 5 nm, less than 4 nm, less than 3 nm, less than 2 nm, less than tnm, or less than 0.5 nm), and in some embodiments, as short as possible without impacting folding or function of the ligand or rigid protein module.

Thus, in some embodiments, the at least one rigid protein module comprises an α-helix-forming peptide sequence, such as (EAAAK)n (SEQ ID NO: 171); or a proline-rich peptide sequence, such as (XP)n, with X designating any amino acid, e.g., Ala, Lys, or Glu.

In some embodiments, the at least one rigid protein module is a fibronectin type III repeat or an Ig domain harboring the typical motifs of the Ig fold (Ig-like domain).

In some embodiments, the elongation domain comprises at least two Ig-like domains and/or at least three fibronectin type III repeats.

In some embodiments, the rigid elongation domain comprises the complete extracellular domain of LFA-3 (containing two Ig-like domains), CD22 (containing seven Ig-like domains), CD45 (comprising three fibronectin type III repeats), CD43, or CD148 (comprising five fibronectin type III repeats) or any combination of Ig-like domains and/or fibronectin type III domains of LFA-3, CD22, CD45, CD148, CD43, ICAM-1, or VCAM-1 or any other protein of the Ig and fibronectin type III superfamilies. In some embodiments, an elongation domain comprises 1-10 (e.g., 1, 2, 3, 4, 5, 6, 7, 8, 9, or 10) domains (e.g., Ig-like domains) from LFA-3, CD22, CD45, CD43, or CD148. In some embodiments, an elongation domain comprises two mor more copies of the same domain. In some embodiments, the domains in an elongation domain of a cell-distancing device are different. For example, an elongation domain may comprise an Ig-like domain from CD22 and an Ig-like domain from FLFA-3. In another example, an elongation domain may comprise an Ig-like domain from CD22, a fibronectin type II domain from CD45, and an Ig-like domain from FLFA-3

In some embodiments, the complete extracellular domain of CD45 is the complete extracellular domain of the CD45 isoform CD45RO, CD45RAB or CD45RABC. In some embodiments, the complete extracellular domain has a length greater than 150 Å (e.g., greater than 150 Å, greater than 200 Å, greater than 250 Å, or greater than 200 Å or more).

Six different human isoforms of CD45 mRNAs have been isolated, which contain all three exons (ABC isoform), two of the three exons (AB and BC isoform), only one exon (A isoform and B isoform), or no exons (0 isoform). All of the isoforms have the same eight amino acids at their amino-terminus, which are followed by the various combinations of A, B, and C peptides (66, 47, and 48 amino acids long, respectively). The remaining regions (the 383-amino-acid extracellular region, the 22-amino-acid transmembrane peptide, and the 707 amino-acid-cytoplasmic region) have the identical sequences in all isoforms. The suffix RA, RB, or RO indicates the requirement of the amino acid residues corresponding to exon A (RA), exon B (RB), or a lack of amino acid residues corresponding to exon A, B and C (RO) for the CD45 epitope expression, respectively (see FIGS. 5B-5L).

In some embodiments, the elongation domain comprises a domain from LFA-3 (CD58 or CD48), CD22 (e.g., one or more Ig-like domains of CD22) or CD45 (e.g., CD45RO, CD45RAB or CD45RABC).

In some embodiments, the native structure of the rigid protein module and/or rigid elongation domain is maintained from the extracellular domain down through the membrane-proximal domain and/or through the transmembrane domain to reduce floppiness between the extracellular membrane-distal domain and the transmembrane domain. In some embodiments, the “floppiness” or “rotational freedom” of a surface protein, such as a distancing device, refers to the maximum deviation from 90° of the angle formed by (1) a line tangent to the cell membrane and intersecting with the distancing device; and (2) a line connecting the transmembrane domain to the extracellular membrane-distal domain and intersecting with the line of (1) at the transmembrane domain. In some embodiments, the “floppiness” or “rotational freedom” of a domain of a molecule, such as the elongation domain of a distancing device, refers to the maximum deviation from 90° of the angle formed by (1) a line connecting a first terminal end and a second terminal end of the domain; and (2) a line intersecting with the line of (1) at the first terminal end of the domain and connecting to any point that the second terminal end may be located while the first terminal end is fixed. A molecule, such as a distancing device, that extends straight up from the cell membrane, and thus forms a 90° angle with the cell surface, has a rotational freedom of 0°, and thus minimal floppiness. The farther a molecule is capable of deviating from this upright angle, such as through conformational changes in one or more membrane-proximal domains, hinges, and/or membrane-distal domains, and thus the shallower the angle formed by this bending, the more floppiness, or rotational freedom, the molecule is said to have. A molecule that is capable of bending to form an angle as shallow as 600 with the cell membrane is said to deviate from this 90° by up to 30°, and has greater floppiness than a molecule that is capable of bending only far enough to form an angle as shallow as 75°, deviating up to 15°. In some embodiments, the distancing device is capable of deviating from an upright position 450 or less, 400 or less, 350 or less, 300 or less, 250 or less, 200 or less, 15° or less, 10° or less, or 5° or less. In some embodiments, the rotational freedom of the distancing device is 15° or less, 100 or less, 9° or less, 8° or less, 7° or less, 6° or less, 5° or less, 4° or less, 3° or less, 2° or less, or 1° or less. Methods of measuring the deviation of a transmembrane protein, such as any of the distancing devices provided herein, are known in the art. In some embodiments, sedimentation, gel filtration, and rotary shadow electron microscopy can be used to evaluate the size and shape of proteins. See, e.g., Erickson (Shulin Li (ed.), Biological Procedures Online, Volume 11, Number 1) and Chang et al. Nat Immunol. 2016. 17(5):574-582. In some embodiments, X-ray crystallography or NMR spectroscopy or cryo-electron microscopy or cryo-tomo election microscopy is used to measure shape, size and/or dimensions of a protein. In some embodiments, rigidity is measured by calculating the rotational freedom between each domain pair in a protein. Further, variable-angle total internal reflection fluorescence microscopy (VA-TIRFM) can be used to measure how upright a protein is relative to the cell surface. In some embodiments, the rotational freedom of elongation domains present in the cell-distancing device as provided herein is 15° or less, 10° or less, 9° or less, 8° or less, 7° or less, 6° or less, 5° or less, 4° or less, 3° or less, 2° or less, or 1° or less. In some embodiments, rigidity of elongation domains present in the cell-distancing device as provided herein is 15° or less, 10° or less, 9° or less, 8° or less, 7° or less, 6° or less, 5° or less, 4° or less, 3° or less, 2° or less, or 1° or less.

An elongation domain may be located immediately adjacent to the membrane-distal domain. In some embodiments, the membrane-distal domain and the elongation domain are connected with a hinge.

SEQ ID NOs: 15-24 and SEQ ID NOs: 70-79 provide examples of nucleic acid sequences that encode elongation domains and amino acid sequences of elongation domains, respectively. Nucleic acid sequences in Table 4 correspond to amino acid sequences in Table 5. In some embodiments, a device as provided herein has an elongation domain comprising an amino acid sequence that is at least 50% (e.g., at least 50%, at least 60%, at least 70%, at least 80%, at least 85%, at least 90%, at least 95%, at least 97%, at least 99%) identical to any one of SEQ ID NOs: 70-79. In some embodiments, a device as provided herein has an elongation domain comprising an amino acid sequence that is identical to any one of SEQ ID NOs: 70-79. In some embodiments, an elongation domain comprises one or more (e.g., two or three) domains included in any one of SEQ ID NOs: 70-79. In some embodiments, one or more domains in an elongation domain comprises a sequence that is at least 50% (e.g., at least 50%, at least 60%, at least 70%, at least 80%, at least 85%, at least 90%, at least 95%, at least 97%, at least 99%) identical to the sequence of a domain in any one of SEQ ID NOs: 70-79. In some embodiments, an elongation domain comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one of SEQ ID NOs: 70-79. In some embodiments, an elongation domain comprises at least a first contiguous amino acid sequence region that is identical to any one of SEQ ID NOs: 70-79. In some embodiments, a first contiguous amino acid sequence region is at least 10 amino acid long (e.g., at least 10, at least 20, at least 30, at least 40, at least 50 amino acids, or at least 100 or more amino acids long).

In some embodiments, an elongation domain is at least 30 amino acids long (e.g., at least 30 amino acids, at least 40 amino acids, at least 50 amino acids, at least 60 amino acids, at least 70 amino acids, at least 80 amino acids, at least 90 amino acids, at least 100 amino acids, at least 110 amino acids, at least 120 amino acids long, at least 150 amino acids long, at least 200 amino acids long, at least 250 amino acids long, at least 300 amino acids long, at least 350 amino acids long, at least 400 amino acids long, at least 450 amino acids long, at least 500 amino acids long, at least 525 amino acids long, at least 550 amino acids long, at least 575 amino acids long, at least 600 amino acids long, or at least 650 amino acids long). In some embodiments, a membrane-distal domain is at most 5,000 amino acids long (e.g., at most 5,000 amino acids, at most 4,500 amino acids, at most 4,000 amino acids, at most 3,500 amino acids, at most 3,000 amino acids, at most 2,500 amino acids, at most 2,000 amino acids, at most 1,800 amino acids, at most 1,600 amino acids, at most 1,400 amino acids, at most 1,200 amino acids, at most 1,000 amino acids, at most 900 amino acids, at most 800 amino acids, at most 700 amino acids, at most 600 amino acids, at most 500 amino acids long, at most 450 amino acids long, at most 400 amino acids long, at most 300 amino acids long, at most 200 amino acids long, or at most 100 amino acids long). In some embodiments, elongation domain is 10-5,000 amino acids long (e.g., 10-5,000, 20-4,800, 40-4,500, 100-4,000, 200-3,500, 400-3,000, 400-2,500, 400-2,000, 400-1,000, 450-500, 500-520, 500-550, 520-550, 500-600, 525-575, 550-600, 575-600, 500-800, 500-900, 500-950, 600-100, 600-700, 700-800, 800-900, or 500-1,0000 amino acids long). In some embodiments, an elongation domain is 200-800 amino acids long (e.g., 200-800, 200-600, 250-550, 300-500, 350-500, 300-400, 400-500, 400-600, 300-800, 400-800, 400-600, or 300-700 amino acids long)

In some embodiments, an elongation domain is at least 100 Å, at least 120 Å, at least 150 A, at least 175 Å, at least 200 Å, at least 250 Å, at least 300 Å, at least 350 Å, at least 400 Å, at least 450 Å, at least 500 Å, at least 550 Å, at least 600 Å, at least 650 Å, at least 700 Å, at least 750 Å, at least 800 Å, at least 850 Å, at least 900 Å, at least 950 Å, or up to 1000 Å in length. In some embodiments, each of the one or more rigid protein modules is at least 10 Å, at least 20 Å, at least 30 Å, at least 40 Å, at least 50 Å, at least 60 Å, at least 70 Å, at least 80 Å, at least 90 Å, at least 100 Å, at least 110 Å, at least 120 Å, at least 130 Å, at least 140 Å, at least 150 Å, at least 160 Å, at least 170 Å, at least 180 Å, at least 190 Å, or up to 200 Å in length.

In some embodiments, the elongation domain does not comprise of domain/s of CD22, CD45, CD48, CD58, or CD2.

### Extracellular Membrane-Proximal Domain

In some embodiments, an “extracellular membrane-proximal domain” refers to the extracellular domain of the cell distancing devise that is closest to the transmembrane domain. In some embodiments, a cell-distancing device does not comprise a separate membrane-proximal domain, but rather the membrane-proximal region of the elongation domain is directly attached to a transmembrane domain without an intervening membrane-proximal domain.

In some embodiments, the membrane-proximal domain comprises an Ig-like domain (such as an LFA-3 Ig-like domain) or a fibronectin type III repeat. In some embodiments, the extracellular membrane-proximal domain is a portion or entirety of a human protein. In some embodiments, the extracellular membrane-proximal domain is from a protein selected from LFA-3 (CD58 or CD48), CD45 (e.g, CD45RO, CD45RAB or CD45RABC), CD22, HLA-A2 or H-2K(b). In some embodiments, the extracellular membrane-proximal domain is not a membrane-proximal domain of CD22, CD45, CD48, CD58, or CD2.

SEQ ID NOs: 32-36 or SEQ ID NOs: 87-91 provide examples of nucleic acid sequences encoding extracellular membrane-proximal domains and amino acid sequences of extracellular membrane-proximal domains, respectively. Nucleic acid sequences in Table 4 correspond to amino acid sequences in Table 5. In some embodiments a device as provided herein has an extracellular membrane-proximal domain comprising an amino acid sequence that is at least 50% (e.g., at least 50%, at least 60%, at least 70%, at least 80%, at least 85%, at least 90%, at least 95%, at least 97%, at least 99%) identical to any one of SEQ ID NOs: 87-91. In some embodiments a device as provided herein has an extracellular membrane-proximal domain comprising an amino acid sequence that is identical to any one of SEQ ID NOs: 87-91. In some embodiments, an extracellular membrane-proximal domain comprises one or more (e.g., two or three) domains included in any one of SEQ ID NOs: 87-91. In some embodiments, one or more domains in an extracellular membrane-proximal domain comprises a sequence that is at least 50% (e.g., at least 50%, at least 60%, at least 70%, at least 80%, at least 85%, at least 90%, at least 95%, at least 97%, at least 99%) identical to the sequence of a domain in any one of SEQ ID NOs: 87-91. In some embodiments, an extracellular membrane-proximal domain comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one of SEQ ID NOs: 87-91. In some embodiments, an extracellular membrane-proximal domain comprises at least a first contiguous amino acid sequence region that is identical to any one of SEQ ID NOs: 87-91. In some embodiments, a first contiguous amino acid sequence region is at least 3 amino acid long (e.g., at least 3, at least 4, at least 5, at least 6, at least 7, at least 8, at least 9, at least 10, at least 20, at least 30, at least 40, at least 50 amino acids, or at least 100 or more amino acids long).

In some embodiments, a cell-distancing device does not comprise a separate membrane-proximal domain, but rather the membrane-proximal region of the elongation domain is directly attached to a transmembrane domain without an intervening membrane-proximal domain. Examples of nucleic acids encoding such elongation domains are provided in nucleic acid sequences of any one of SEQ ID NOs: 15-24. Corresponding examples of amino acid sequences of such domains are provided in SEQ ID NOs: 70-79. In some embodiments, a device does not comprise a membrane-proximal domain. In some embodiments, elongation domain with a proximal region that is attached to a transmembrane domain (or in some embodiments, via a hinge) comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one of SEQ ID NOs: 70-79. In some embodiments, elongation domain with a proximal region that is attached to a transmembrane domain (or in some embodiments, via a hinge) comprises at least a first contiguous amino acid sequence region that is identical to any one of SEQ ID NOs: 70-79. In some embodiments, a first contiguous amino acid sequence region is at least 10 amino acid long (e.g., at least 10, at least 20, at least 30, at least 40, at least 50 amino acids, or at least 100 or more amino acids long).

SEQ ID NOs: 25-31 and amino acid sequences of SEQ ID NOs: 80-86 provide examples of sequences comprising or encoding an elongation domain, transmembrane domain and intracellular domain. In some embodiments, a cell-distancing device comprises an elongation domain, transmembrane domain and/or intracellular domain, comprising a sequence that is at least 50% (e.g., at least 50%, at least 60%, at least 70%, at least 80%, at least 85%, at least 90%, at least 95%, at least 97%, at least 99%) identical to any one of SEQ ID NOs: 80-86. In some embodiments, a cell-distancing device comprises an elongation domain, transmembrane domain and/or intracellular domain, comprising a sequence that is identical to any one of SEQ ID NOs: 80-86.

In some embodiments, an extracellular membrane-proximal domain is at least 3 amino acids long (e.g., at least 3, at least 4, at least 5, at least 6, at least 7, at least 8, at least 9, at least 10, at least 12, at least 15, at least 20, at least 30 amino acids, at least 40 amino acids, at least 50 amino acids, at least 60 amino acids, at least 70 amino acids, at least 80 amino acids, at least 90 amino acids, at least 100 amino acids, at least 110 amino acids, at least 120 amino acids long, at least 150 amino acids long, at least 200 amino acids long, at least 250 amino acids long, at least 300 amino acids long, at least 350 amino acids long, at least 400 amino acids long, at least 450 amino acids long, or at least 500 amino acids long). In some embodiments, a membrane-proximal domain is at most 5,000 amino acids long (e.g., at most 5,000 amino acids, at most 4,500 amino acids, at most 4,000 amino acids, at most 3,500 amino acids, at most 3,000 amino acids, at most 2,500 amino acids, at most 2,000 amino acids, at most 1,800 amino acids, at most 1,600 amino acids, at most 1,400 amino acids, at most 1,200 amino acids, at most 1,000 amino acids, at most 900 amino acids, at most 800 amino acids, at most 700 amino acids, at most 600 amino acids, or at most 500 amino acids long). In some embodiments, a membrane-proximal domain is 10-5,000 amino acids long (e.g., 10-5,000, 20-4,800, 40-4,500, 100-4,000, 200-3,500, 400-3,000, 400-2,500, 400-2,000, 400-1,000, 500-800, 500-900, 500-950, or 500-1,0000 amino acids long). In some embodiments, an extracellular membrane-proximal domain is 1-15 amino acids long (e.g., 1, 2, 3, 4, 5, 6, 7, 8, 9, 10, 11, 12, 13, 14, or 15 amino acids long). In some embodiments, an extracellular membrane-proximal domain is 1-10 (e.g., 1-3, 1-4, 1-5, 1-6, 1-7, 1-8, 1-9, or 1-10) amino acids long.

In some embodiments, a membrane-proximal domain is less than 10 nm (e.g., less than 10, less than 9, less than 8, less than 7, less than 6, less than 5, less than 4, less than 3, less than 2, less than 1, less than 1, less than 0.5, less than 0.1 nm, or less than 0.01 nm) long (e.g., from N-terminus to C-terminus).

### Transmembrane Domain

As used herein, a “transmembrane domain” refers to a domain of a cell-distancing device that is embedded in the phospholipid bilayer of a cell comprising the device. In some embodiments, the transmembrane domain is the transmembrane domain of LFA-3 (CD48 or CD58). In some embodiments, the transmembrane domain is a transmembrane domain of CD45 (e.g, CD45RO, CD45RAB or CD45RABC), CD22, HLA-A2 or H-2K(b).

In some embodiments, the transmembrane domain and the membrane-proximal domain are derived from the same protein. In some embodiments, having a transmembrane domain and membrane-proximal domain derived from the same protein reduces floppiness of the device. In some embodiments, the transmembrane domain, the extracellular membrane-proximal domain and the elongation domain are derived from the same protein. In some embodiments, the transmembrane domain is a portion of a human protein. In some embodiments, the transmembrane domain is not a transmembrane domain of CD22. In some embodiments, the transmembrane domain is not, or does not comprise, a transmembrane domain of CD22, CD45, CD48, CD58, or CD2.

SEQ ID NOs: 43-48 and amino acid sequences of SEQ ID NOs: 98-103 provide examples of transmembrane domains. In some embodiments a device as provided herein has a transmembrane domain comprising an amino acid sequence that is at least 50% (e.g., at least 50%, at least 60%, at least 70%, at least 80%, at least 85%, at least 90%, at least 95%, at least 97%, at least 99%) identical to any one of SEQ ID NOs: 98-103. In some embodiments a device as provided herein has a transmembrane domain comprising an amino acid sequence that is identical to any one of SEQ ID NOs: 98-103. In some embodiments, a transmembrane domain comprises one or more (e.g., two or three) domains included in any one of SEQ ID NOs: 98-103. In some embodiments, one or more domains in a transmembrane domain comprises a sequence that is at least 50% (e.g., at least 50%, at least 60%, at least 70%, at least 80%, at least 85%, at least 90%, at least 95%, at least 97%, at least 99%) identical to the sequence of a domain in any one of SEQ ID NOs: 98-103. In some embodiments, one or more domains in a transmembrane domain comprises a sequence that is identical to the sequence of a domain in any one of SEQ ID NOs: 98-103

SEQ ID NOs: 43-48 and SEQ ID NOs: 98-103 provide nucleic acid sequences encoding transmembrane domains and amino acid sequences of transmembrane domains, respectively. In some embodiments, a transmembrane domain comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one of SEQ ID NOs: 98-103. In some embodiments, a first contiguous amino acid sequence region is at least 10 amino acid long (e.g., at least 10, at least 20, at least 30, at least 40, at least 50 amino acids, or at least 100 or more amino acids long). In some embodiments, a transmembrane domain comprises at least a first contiguous amino acid sequence region that is identical to any one of SEQ ID NOs: 98-103.

SEQ ID NOs: 37-42 and SEQ ID NOs: 92-97 provide examples of nucleic acid sequences encoding and amino acid sequences comprising transmembrane domains and intracellular domains, wherein the transmembrane domain and the intracellular domain are from the same protein (e.g., LFA-3 (CD48 or CD58), CD45 (e.g, CD45RO, CD45RAB or CD45RABC), CD22, HLA-A2 or H-2K(b)), respectively. In some embodiments, a device as provided herein has a transmembrane domain and an intracellular domain comprising an amino acid sequence that is at least 50% (e.g., at least 50%, at least 60%, at least 70%, at least 80%, at least 85%, at least 90%, at least 95%, at least 97%, at least 99%) identical to any one of SEQ ID NOs: 92-97. In some embodiments, a device as provided herein has a transmembrane domain and an intracellular domain comprising an amino acid sequence that is identical to any one of SEQ ID NOs: 92-97. In some embodiments, a transmembrane domain and an intracellular domain comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one of SEQ ID NOs: 92-97. In some embodiments, a transmembrane domain and an intracellular domain comprises at least a first contiguous amino acid sequence region that is identical to any one of SEQ ID NOs: 92-97. In some embodiments, a first contiguous amino acid sequence region is at least 10 amino acid long (e.g., at least 10, at least 20, at least 30, at least 40, at least 50 amino acids, or at least 100 or more amino acids long).

In some embodiments, transmembrane domain is at least 10 amino acids long (e.g., at least 10, at least 12, at least 15, at least 20, at least 25, at least 30 amino acids, at least 40 amino acids, at least 50 amino acids, at least 60 amino acids, at least 70 amino acids, at least 80 amino acids, at least 90 amino acids, at least 100 amino acids, at least 110 amino acids, at least 120 amino acids long, at least 150 amino acids long, at least 200 amino acids long, at least 250 amino acids long, at least 300 amino acids long, at least 350 amino acids long, at least 400 amino acids long, at least 450 amino acids long, or at least 500 amino acids long). In some embodiments, a membrane-distal domain is at most 5,000 amino acids long (e.g., at most 5,000 amino acids, at most 4,500 amino acids, at most 4,000 amino acids, at most 3,500 amino acids, at most 3,000 amino acids, at most 2,500 amino acids, at most 2,000 amino acids, at most 1,800 amino acids, at most 1,600 amino acids, at most 1,400 amino acids, at most 1,200 amino acids, at most 1,000 amino acids, at most 900 amino acids, at most 800 amino acids, at most 700 amino acids, at most 600 amino acids, or at most 500 amino acids long). In some embodiments, a membrane-distal domain is 10-5,000 amino acids long (e.g., 10-5,000, 20-4,800, 40-4,500, 100-4,000, 200-3,500, 400-3,000, 400-2,500, 400-2,000, 400-1,000, 500-800, 500-900, 500-950, or 500-1,0000 amino acids long).

In some embodiments, a transmembrane domain is 0.5-100 nm (e.g., 0.5-100, 1-50, 2-40, 3-30, 4-20, 5-15, 5-10, or 7.5-12.5 nm) long. In some embodiments, a transmembrane domain is 5-10 nm long.

### Hinge

As used herein, a “hinge” refers to a peptide and/or amino acid sequence that serves to connect two domains or that is adjacent to a domain of the cell-distancing device as disclosed herein. In some embodiments, a hinge comprises 1, 2, 3, 4, 5, 6, 7, 8, 9 or 10 amino acids, such as glycines, or a number of amino acids, such as glycine, within a range defined by any two of the aforementioned numbers. In some embodiments, a glycine spacer comprises at least 3 glycines. In some embodiments, the glycine spacer comprises an amino acid sequence set forth in SEQ ID NO: 105, SEQ ID NO: 169 or SEQ ID NO: 170. In some embodiments, one or more hinges comprises a hinge domain of CD8 provided as SEQ ID NO: 104. In some embodiments, one or more hinges comprises a hinge domain of human CD8. In some embodiments, one or more hinges comprises a sequence as set forth in any one of SEQ ID NOs: 104-108 (e.g., encoded by nucleic acid sequences of SEQ ID NOs: 49-53, respectively). In some embodiments, one or more hinges comprises a sequence at least 70% (at least 80%, at least 85%, at least 90%, at least 95%, at least 97%, at least 99%) identical to any one of SEQ ID NOs: 104-108, or SEQ ID NOs: 169-170. In some embodiments, a hinge comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one of SEQ ID NOs: 104-108. In some embodiments, a hinge comprises at least a first contiguous amino acid sequence region that is identical to any one of SEQ ID NOs: 104-108, or SEQ ID NOs: 169-170. In some embodiments, a first contiguous amino acid sequence region is at least 10 amino acid long (e.g., at least 10, at least 20, at least 30, at least 40, at least 50 amino acids, or at least 100 or more amino acids long).

In some embodiments, the portion of the device comprising the extracellular membrane-distal domain, the elongation domain, the membrane-proximal domain, and any hinges between and/or adjacent to these domains is at least 150 Å (e.g., at least 150 Å, at least 175 Å, at least 200 Å, at least 250 Å, at least 300 Å, at least 350 Å, at least 400 Å, at least 450 Å, at least 500 Å, at least 550 Å, at least 600 Å, at least 650 Å, at least 700 Å, at least 750 Å, at least 800 Å, at least 850 Å, at least 900 Å, at least 950 Å, or up to 1000 Å) in length.

In some embodiments, the portion of the device comprising the extracellular membrane-distal domain, the elongation domain, the membrane-proximal domain, and any hinges between and/or adjacent to these domains is at least 10 nm (e.g., at least 10, at least 12, at least 15 nm, at least 17.5 nm, at least 20 nm, at least 25 nm, at least 30 nm, at least 35 nm, at least 40 nm, at least 45 nm, at least 50 nm, at least 55 nm, at least 60 nm, at least 65 nm, at least 70 nm, at least 75 nm, at least 80 nm, at least 85 nm, at least 90 nm, at least 95 nm, or up to 100 nm) in length.

### Tags

In some embodiments, cell-distancing devices comprise one or more tags. In some embodiments, a tag is a peptide, protein, or small molecule that serves as a marker to identify the cell-distancing device or the cells that comprise it. Some non-limiting examples of tags include peptide tags such as HA-tag, myc tag, or His6 tag, and small molecules such as radiolabels, immunoluminescent tags and fluorophores. SEQ ID NO: 109 (e.g., encoded by nucleic acid sequence of SEQ ID NO: 54) provides an example sequence of a HA-tag.

### Intracellular Domain

In some embodiments, the cell-distancing device of the present disclosure comprises an intracellular domain that is connected to the transmembrane domain. As used herein, “intracellular domain” refers to a domain of the device that is present in the cytoplasm of the cell in which it is expressed or comprised. In some embodiments, the intracellular domain is connected to the transmembrane domain by one or more hinges. In some embodiments, the intracellular domain is capable of binding to an intracellular domain of an MHC molecule of the cell that expresses or comprises the device. In some embodiments, the MHC molecule is an MHC-I or MHC-II molecule. In some embodiments, the MHC molecule is a human leukocyte antigen (HLA) molecule. Binding of an MHC molecule to the T cell-distancing device on the surface of the same cell causes the MHC molecule to co-cluster with the T cell-distancing device. Because the T cell-distancing device maintains a physical distance between the expressing cell and a potentially alloreactive T cell or NK cell that is greater than the distance formed by the SMAC of the immunological synapse, the co-clustered MHC is has a reduced chance of interacting with a T cell receptor on the T cell or other receptor on an NK cell. Furthermore, this co-clustering reduces the ability of MHC molecules to interact with other potentially alloreactive T cells or NK cells at another region of the cell surface, thus providing a general dampening of T cell or NK cell activity.

In some embodiments, an intracellular domain of a cell-distancing device comprises one or more intracellular domains of LFA-3 (including CD48 or CD58). In some embodiments, an intracellular domain of a cell-distancing device comprises one or more intracellular domains of CD45 (e.g, CD45RO, CD45RAB or CD45RABC), CD22, and HLA (e.g., HLA-A2 or H-2K(b)). In some embodiments, an intracellular domain is not, or does not comprise, an intracellular domain of CD22, CD45, CD48, CD58, or CD2. In some embodiments, the intracellular domain and transmembrane domain are from the same protein. In some embodiments, the intracellular domain, transmembrane domain and extracellular membrane-proximal domain are from the same protein. In some embodiments, the intracellular domain, transmembrane domain, extracellular membrane-proximal domain and elongation domain are from the same protein. In some embodiments, a device as provided herein has a transmembrane domain and an intracellular domain such as the amino acid sequence of the transmembrane domain and the intracellular domain is at least 50% (e.g., at least 50%, at least 60%, at least 70%, at least 80%, at least 85%, at least 90%, at least 95%, at least 97%, at least 99%) identical to any one of SEQ ID NOs: 92-97. In some embodiments, a device as provided herein has a transmembrane domain and an intracellular domain such as the amino acid sequence of the transmembrane domain and intracellular domain is identical to any one of SEQ ID NOs: 92-97. Examples of nucleic acid sequences encoding a transmembrane domain and intracellular domain are provided in SEQ ID NOs: 37-42. In some embodiments, a combined sequence of transmembrane domain and intracellular domain comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one of SEQ ID NOs: 37-42. In some embodiments, a combined sequence of transmembrane and intracellular domain comprises at least a first contiguous amino acid sequence region that is identical to any one of SEQ ID NOs: 37-42. In some embodiments, a first contiguous amino acid sequence region is at least 10 amino acid long (e.g., at least 10, at least 20, at least 30, at least 40, at least 50 amino acids, or at least 100 or more amino acids long).

In some embodiments, a cell-distancing device comprises an intracellular domain and a transmembrane domain directly attached to, or combined with, an elongation domain. SEQ ID NOs: 25-31 and amino acid sequences of SEQ ID NOs: 80-86 provide examples of elongation domains directly attached to, or combined with, an intracellular domain through a transmembrane domain. In some embodiments, a cell-distancing device comprises a sequence that is at least 50% (e.g., at least 50%, at least 60%, at least 70%, at least 80%, at least 85%, at least 90%, at least 95%, at least 97%, at least 99%) identical to any one of SEQ ID NOs: 80-86. In some embodiments, a cell-distancing device comprises a sequence that is identical to any one of SEQ ID NOs: 80-86. In some embodiments, a combined transmembrane and elongation domain comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one of SEQ ID NOs: 80-86. In some embodiments, a combined transmembrane and elongation domain comprises at least a first contiguous amino acid sequence region that is identical to any one of SEQ ID NOs: 80-86. In some embodiments, a first contiguous amino acid sequence region is at least 10 amino acid long (e.g., at least 10, at least 20, at least 30, at least 40, at least 50 amino acids, at least 100, or at least 200 or more amino acids long).

It is to be understood that any configuration of a particular domain of the device described herein can be combined with any configuration of other domains of the device. For example, a device may contain a LFA-3 sequence in its extracellular membrane-distal domain and CD22 domains in its elongation and/or membrane-proximal domains. In other embodiments, a device may contain a CD2-binding antibody fragment in its extracellular membrane-distal domain and CD22 domains in its elongation and/or membrane-proximal domains. In yet another example, a device may contain a CD2-finding antibody fragment in its extracellular membrane-distal domain and LFA-domains in its elongation and/or membrane-proximal domains.

In some embodiments, multiple domains of the cell-distancing device comprise domains from the same protein. For example, both the elongation domain and the membrane-proximal domain may comprise CD45 domains/sequences. In some embodiments, both the elongation domain and the membrane-proximal domain may comprise LFA-3 (including CD58 or CD48) domains/sequence. In some embodiments, both the membrane-proximal domain and transmembrane domain may comprise LFA-3 (including CD58 or CD48) domains/sequence.

Six different human isoforms of CD45 mRNAs have been isolated, which contain all three exons (ABC isoform), two of the three exons (AB and BC isoform), only one exon (A isoform and B isoform), or no exons (O isoform). All of the isoforms have the same eight amino acids at their amino-terminus, which are followed by the various combinations of A, B, and C peptides (66, 47, and 48 amino acids long, respectively). The remaining regions (the 383-amino-acid extracellular region, the 22-amino-acid transmembrane peptide, and the 707 amino-acid-cytoplasmic region) have the identical sequences in all isoforms. The suffix RA, RB, or RO indicates the requirement of the amino acid residues corresponding to exon A (RA), exon B (RB), or a lack of amino acid residues corresponding to exon A, B and C (RO) for the CD45 epitope expression, respectively (see FIG. 5B-5L).

In some embodiments, the membrane-proximal domain comprises an Ig-like domain (such as an LFA-3 Ig-like domain) or a fibronectin type III repeat.

In some embodiments, the transmembrane domain and/or intracellular domain is the transmembrane domain and/or intracellular domain of LFA-3.

In particular embodiments, the member of the central SMAC is selected from CD2, CD8, CD4, a signaling lymphocytic activation molecule (SLAM), and a CD28 family member; the at least one rigid protein module comprises an α-helix-forming peptide sequence (such as (EAAAK)n), a proline-rich peptide sequence (such as (XP)n, with X designating any amino acid), a fibronectin type III repeat or an Ig domain harboring the typical motifs of the Ig fold (Ig-like domain); the membrane-proximal domain comprises an Ig-like domain (such as an LFA-3 Ig-like domain) or a fibronectin type III repeat; and the transmembrane domain and/or intracellular domain is the transmembrane domain and/or intracellular domain of LFA-3.

In particular embodiments, the binding domain is a CD2-binding domain selected from an LFA-3 (CD58) CD2-binding domain or a synthetic anti-CD2 antibody; the CD28 family member is selected from CD28, ICOS, BTLA, CTLA-4 and PD-1; and the elongation domain comprises at least two Ig-like domains and/or at least three fibronectin type III repeats.

In particular embodiments, the rigid elongation domain comprises the complete extracellular domain of LFA-3 (containing two Ig-like domains), CD22 (containing seven Ig-like domains), CD45 (comprising three fibronectin type III repeats), or CD148 (comprising five fibronectin type III repeats) or any combination of Ig-like domains and/or fibronectin type III domains.

In particular embodiments, the complete extracellular domain of CD45 is the complete extracellular domain of the CD45 isoform CD45RO, CD45RAB or CD45RABC.

In particular embodiments, the alloreactive T cell-distancing device comprises an LFA-3 CD2-binding domain; a rigid elongation domain comprising at least two CD22 Ig-like domains and at least one LFA-3 Ig-like domain; or a complete extracellular CD45 domain and at least one LFA-3 Ig-like domain; an LFE-3 Ig-like membrane-proximal domain, and an LFE-3 transmembrane and intracellular domain.

In particular embodiments, the rigid elongation domain comprises a complete extracellular CD45 domain selected from that of CD45RO, CD45RAB and CD45RABC and one LFA-3 Ig-like domain, and the complete extracellular CD45 domain is located between the LFE-3 Ig-like membrane-proximal domain and the LFA-3 Ig-like rigid elongation domain.

In some embodiments, a domain comprised of an elongation domain, extracellular membrane-proximal domain, transmembrane domain, and intracellular domains is encoded by a nucleic acid sequence that is at least 50% (e.g., at least 50%, at least 60%, at least 70%, at least 80%, at least 85%, at least 90%, at least 95%, at least 97%, at least 99%) identical to any one of SEQ ID NOs: 25-31. In some embodiments, a domain comprised of an elongation domain, extracellular membrane-proximal domain, transmembrane domain, and intracellular domains is encoded by a nucleic acid sequence that is identical to any one of SEQ ID NOs: 25-31. In some embodiments, a domain comprised of an elongation domain, extracellular membrane-proximal domain, transmembrane domain, and intracellular domains comprises an amino acid sequence that is at least 50% (e.g., at least 50%, at least 60%, at least 70%, at least 80%, at least 85%, at least 90%, at least 95%, at least 97%, at least 99%) identical to any one of SEQ ID NOs: 80-86. In some embodiments, a domain comprised of an elongation domain, extracellular membrane-proximal domain, transmembrane domain, and intracellular domains comprises an amino acid sequence that is identical to any one of SEQ ID NOs: 80-86.

In some embodiments, a cell-distancing device has an amino acid sequence that is at least 50% (e.g., at least 50%, at least 60%, at least 70%, at least 80%, at least 85%, at least 90%, at least 95%, at least 97%, at least 99%) identical to any one of SEQ ID NOs: 55 or 110-168. In some embodiments, the cell-distancing device has an amino acid sequence that is identical to any one of SEQ ID NOs: 55 or 110-168. In some embodiments, a device comprises at least a first contiguous amino acid sequence region that is at least 50% (e.g., at least 50%, at least 60%, at least 70%, at least 80%, at least 85%, at least 90%, at least 95%, at least 97%, at least 99%) identical to any one of SEQ ID NOs: 55 or 110-168. In some embodiments, a device comprises at least a first contiguous amino acid sequence region that is identical to any one of SEQ ID NOs: 55 or 110-168. In some embodiments, a first contiguous amino acid sequence region is at least 10 amino acid long (e.g., at least 10, at least 20, at least 30, at least 40, at least 50 amino acids, at least 100, at least 200, at least 300, at least 400, or at least 500 or more amino acids long).

It is to be understood that any of the domains and sequences presented in Tables 4 and 5 can be combined to encompass a cell-distancing device.

### Nucleic Acid Molecules Comprising a Nucleotide Sequence Encoding Cell-Distancing Device

In some aspects, the present disclosure provides a nucleic acid molecule comprising a nucleotide sequence encoding any one of the cell-distancing devices (e.g., a T-cell distancing device) disclosed herein. In some embodiments, a nucleic acid molecule comprising a nucleotide sequence encodes a cell-distancing device comprising (a) an extracellular membrane-distal domain comprising a binding domain that is capable of binding to a member of a central supramolecular activation cluster (SMAC) of the immunological synapse or a member closely associated therewith; (b) an elongation domain comprising at least one rigid protein module; (c) a membrane-proximal domain; and (d) a transmembrane domain; and optionally (e) an intracellular domain. In some embodiments, a device does not comprise a membrane-proximal domain.

In some embodiments, a nucleic acid molecule comprising a nucleotide sequence encodes a cell-distancing device comprising (a) an extracellular membrane-distal domain comprising a binding domain capable of binding a member of a central supramolecular activation cluster (SMAC) of the immunological synapse or a member closely associated therewith; and (b) an elongation domain comprising at least one rigid protein module, wherein said membrane-distal domain is linked via a membrane-proximal domain and a transmembrane domain to an intracellular domain optionally capable of associating, or co-clustering, with, MHC molecules. In some embodiments, the membrane-proximal domain, transmembrane domain, and/or intracellular domain is not that of CD22.

Nucleic acid molecules comprising a nucleotide sequence encoding a cell-distancing device may be comprised on a vector (e.g., a viral vector or non-viral vector such as plasmid).

Matuskova and Durinikova [52] teach that there are two systems for the delivery of transgenes into a cell—viral and non-viral. The non-viral approaches are represented by polymer nanoparticles, lipids, calcium phosphate, electroporation/nucleofection or biolistic delivery of DNA-coated microparticles or mRNA. The non-viral approach also provides transposon systems, such as the transposon system commonly known as “Sleeping Beauty” (for protocols using Sleeping Beauty transposons see for example [53].

The viral approach provides two main types of vectors that can be used in accordance with the present invention depending on whether the DNA is integrated into chromatin of the host cell or not. Retroviral vectors such as those derived from gammaretroviruses or lentiviruses persist in the nucleus as integrated provirus and reproduce with cell division. Other types of vectors (e.g. those derived from herpesviruses or adenoviruses) remain in the cell in the episomal form.

In some embodiments, the vector is a DNA vector, such as a plasmid or viral vector; or a non-viral vector, such as a polymer nanoparticle, lipid, calcium phosphate, DNA-coated microparticle or transposon.

In some embodiments, the DNA vector is a viral vector selected from a modified virus derived from a virus selected from the group consisting of a retrovirus, lentivirus, gammavirus, adenovirus, adeno-associated virus, poxvirus, alphavirus, and herpes virus.

In some embodiments, a nucleic encoding a cell-distancing device are comprised in a viral vector (e.g., a retrovirus, adenovirus, adeno-associated virus, or herpes simplex virus), non-viral vector, can be injected using methods such as electroporation, sonoporation or magnetiofection, or can be encompassed in formulations comprising liposomes or dendrimers. Any known gene delivery method can be used to deliver the nucleic acids disclosed herein to a cell to be protected from host immunity.

### Methods for Producing Cells Expressing a Cell-Distancing Device

In some aspects, the present disclosure provides methods for producing a therapeutic cell (e.g., donor-derived allogeneic cell, cell-line or stem cell-line) expressing any one of the cell-distancing devices disclosed herein. In some embodiments, a method of making such cells or cell-line comprises contacting cell, cell-line or stem cell-line (for example a donor-derived T cell, or iPSC) with any one of the nucleic acid molecules comprising a nucleotide sequence encoding an alloreactive T cell-distancing device as described herein. In some embodiments, a method of making such cells or cell-line comprises delivering any one of the nucleic acid molecules comprising a nucleotide sequence encoding an alloreactive T cell-distancing device as described herein to a cell, cell-line or stem cell-line (for example a donor-derived T cell, or iPSC) to be protected.

In some embodiments, the nucleic acid comprises a nucleotide sequence encoding an alloreactive T cell-distancing device comprising an extracellular membrane-distal domain, an elongation domain, and a transmembrane domain. In some embodiments, the nucleic acid comprises a nucleotide sequence encoding an alloreactive T cell-distancing device comprising an extracellular membrane-distal domain, an elongation domain, an extracellular membrane-proximal domain, and a transmembrane domain. In some embodiments, the nucleic acid comprises a nucleotide sequence encoding an alloreactive T cell-distancing device comprising an extracellular membrane-distal domain, an elongation domain, an extracellular membrane-proximal domain, a transmembrane domain, and intracellular domain. In some embodiments, the nucleic acid comprises a nucleotide sequence encoding an alloreactive T cell-distancing device comprising an extracellular membrane-distal domain, an elongation domain, an extracellular membrane-proximal domain, a transmembrane domain, an intracellular domain, and one or more hinges or one or more tags.

In some embodiments, the nucleic acid comprises a nucleotide sequence encoding an alloreactive T cell-distancing device comprising (a) an extracellular membrane-distal domain comprising a binding domain capable of binding a member of a central supramolecular activation cluster (SMAC) of the immunological synapse or a member closely associated therewith; and (b) an elongation domain comprising at least one rigid protein module, wherein said membrane-distal domain is linked via a membrane-proximal domain and a transmembrane domain to an intracellular domain optionally capable of associating, or co-clustering, with, MHC molecules; or a vector comprising said nucleic acid molecule, wherein said donor-derived allogeneic cell, cell-line or stem cell-line expressing the alloreactive T cell-distancing device is protected from allorejection in adoptive cell therapy or stem cell transplantation, and a differentiated cell, organ or tissue derived from said stem cell-line is protected from allorejection in cell, organ or tissue transplantation.

Cells to be protected using the compositions and methods provided herein may be allogeneic or autologous.

Any method can be used to introduce any one of the nucleic acid molecules described herein into a cell, cell-line or stem cell-line. In some embodiments, a physical method such as electroporation, direct micro injection, biolistic particle delivery, or laser-based transfection is used. In some embodiments, a biological method such as virus-mediated transfer (e.g., using herpes simplex virus, adeno virus, adeno-associated virus, vaccinia virus, or Sindbis virus) is used. In some embodiments, a chemical agent such as a cationic polymer, calcium phosphate or a cationic lipid is used. See e.g., Kim and Eberwine (Alal Bioanal Chem. 2010; 397(8): 3173-3178); Chong et al. (PeerJ. 2021 9: e11165); www.promega.com/resources/guides/cell-biology/transfection/; and www.thermofisher.com/us/en/home/references/gibco-cell-culture-basics/transfection-basics/transfection-methods.html, each of which is incorporated herein by reference in its entirety. In some embodiments, transfection of cell with nucleic acid is transient. In some embodiments, transfection of cell with nucleic acid is stable.

In some embodiments, a nucleic acid molecule is single-stranded (e.g., RNA). In some embodiments, a nucleic acid molecule to engineer a cell as provided herein (e.g., comprising nucleic acid encoding a cell-distancing device or any other protein) is double-stranded (e.g., a DNA).

In some embodiments, a donor-derived allogeneic cell, cell-line or stem cell-line may be transfected with the appropriate nucleic acid molecule described herein by e.g. RNA transfection or by incorporation in a plasmid fit for replication and/or transcription in a eukaryotic cell or a viral vector.

In some embodiments, the vector is a DNA vector, such as a plasmid or viral vector; or a non-viral vector, such as a polymer nanoparticle, lipid, calcium phosphate, DNA-coated microparticle or transposon.

In some embodiments, the vector is a viral vector selected from a modified virus derived from a virus selected from the group consisting of a retrovirus, lentivirus, gammavirus, adenovirus, adeno-associated virus, pox virus, alphavirus, and herpes virus.

Combinations of retroviral vector and an appropriate packaging line can also be used, where the capsid proteins will be functional for infecting human cells. Several amphotropic virus-producing cell-lines are known, including PA12 [54], PA317 [55] and CRIP [56]. Alternatively, non-amphotropic particles can be used, such as, particles pseudotyped with VSVG, RD 114 or GAL V envelope. Cells can further be transduced by direct co-culture with producer cells, e.g., by the method of Bregni, et ai. [57], or culturing with viral supernatant alone or concentrated vector stocks, e.g., by the method of Xu, et al. [58] and Hughes, et at [59].

### Cells Comprising Cell-Distancing Devices

In some aspects, the present disclosure provides a therapeutic cell or donor-derived cell to be protected from a host immune response. In some embodiments, a cell to be protected from host immunity is a donor-derived allogeneic cell, cell-line or stem cell-line or a differentiated cell, organ or tissue derived from stem cells, expressing a nucleotide sequence encoding an alloreactive T cell-distancing device comprising an extracellular membrane-distal domain, an elongation domain, and a transmembrane domain. In some embodiments, a cell as provided herein comprises a cell-distancing device comprising an extracellular membrane-distal domain, an elongation domain, an extracellular membrane-proximal domain, and a transmembrane domain. In some embodiments, a cell as provided herein comprises a cell-distancing device comprising an extracellular membrane-distal domain, an elongation domain, an extracellular membrane-proximal domain, a transmembrane domain, and intracellular domain. In some embodiments, a cell as provided herein comprises a cell-distancing device comprising an extracellular membrane-distal domain, an elongation domain, an extracellular membrane-proximal domain, a transmembrane domain, an intracellular domain, and one or more hinges or one or more tags.

In some embodiments, a cell to be protected is a donor-derived allogeneic cell, cell-line or stem cell-line or a differentiated cell, organ or tissue derived from stem cells, expressing a nucleotide sequence encoding an alloreactive T cell-distancing device comprising (a) an extracellular membrane-distal domain comprising a binding domain capable of binding a member of a central supramolecular activation cluster (SMAC) of the immunological synapse or a member closely associated therewith; and (b) an elongation domain comprising at least one rigid protein module, wherein said membrane-distal domain is linked via a membrane-proximal domain and a transmembrane domain to an intracellular domain optionally capable of associating, or co-clustering, with, MHC molecules; or a DNA vector comprising said nucleic acid molecule, and displaying the alloreactive T cell-distancing device of the present invention on the cell, organ or tissue surface, wherein said donor-derived allogeneic cell, cell-line or stem cell-line is protected from allorejection in adoptive cell therapy or stem cell transplantation, and a differentiated cell, organ or tissue derived from said stem cell-line is protected from allorejection in cell, organ or tissue transplantation.

It should be clear that any one of the above embodiments defining the cell distancing devices disclosed herein (e.g., an alloreactive T cell-distancing device), and the nucleic acid molecule and vector encoding it define them also when employed in methods for producing a donor-derived allogeneic cell, cell-line or stem cell-line expressing an alloreactive T cell-distancing device and when expressed in the donor-derived allogeneic cell, cell-line or stem cell-line expressing an alloreactive T cell-distancing device per se.

In some embodiments, the presently described donor-derived allogeneic cells comprising or encoding any one of the cell-distancing devices described herein, made by the introduction of a nucleic acid encoding one or more of the T cell-distancing devices as described herein, are allogeneic cells from a mammal (e.g., humans, non-human primates (e.g., chimpanzees, macaques, gorillas, etc.), rodents (e.g., mice, rats, etc.), lagomorphs (e.g., rabbits, hares, pikas, etc.), ungulates (e.g., cattle, horses, pigs, sheep, etc.), or other mammals). In some embodiments, allogenic cells are immune cells. In some embodiments allogeneic cells are T cells (e.g., human T cells). In some embodiments, a cell as provided herein is a human cell.

In some embodiments, a cell to be protected is a stem cell. A stem cell to be protected may be an embryonic stem cell, tissue-specific stem cell, mesenchymal stem cell, or an induced pluripotent stem cell (iPSC).

In some embodiments, a cell to be protected is an immune cell. Non-limited examples of an immune cells include granulocytes, mast cells, monocytes, neutraphils, dendritic cells, NK cells, or adaptive cells like B cells and T cells. T cells may be ctytotoxic T cells, helper T cells or regulatory T cells. In some embodiments, a cell is a lymphocyte (e.g., a NK1.1+, CD3+, CD4+, or CD8+ cell). In some embodiments, allogenic cell is a T cell, a precursor T cell, or a hematopoietic stem cell. In some embodiments, the cell is a CD4+ T cell (e.g., a FOXP3-CD4+ T cell or a FOXP3+CD4+ T cell) or a CD8+ T cell(e.g., a FOXP3-CD8+ T cell or a FOXP3+CD8+ T cell). In some embodiments, the cell is an NK-T cell (e.g., a FOXP3—NK-T cell or a FOXP3+NK-T cell). In some embodiments, the cell is a regulatory B (Breg) cell (e.g., a FOXP3—B cell or a FOXP3+B cell). In some embodiments, the cell is a CD25− T cell. In some embodiments, the cell is a regulatory T (Treg) cell. Non-limiting examples of Treg cells are Tr1, Th3, CD8+CD28−, and Qa-1 restricted T cells. In some embodiments, the Treg cell is a FOXP3+ Treg cell. In some embodiments, the Treg cell expresses CTLA-4, LAG-3, CD25, CD39, neuropilin-1, galectin-1, and/or IL-2Ra on its surface. In some embodiments, the cell is ex vivo. In some embodiments, a cell is in vivo. In some embodiments, a cell as provided herein is an engineered cell. In some embodiments, an engineered cell is a cell in which one or more genes/loci are manipulated or edited (e.g., to express one or more exogenous genes). In some embodiments, the cell is a human cell. In some embodiments, a cell as described herein is isolated from a biological sample. A biological sample may be a sample from a subject (e.g., a human subject) or a composition produced in a lab (e.g., a culture of cells). A biological sample obtained from a subject make be a liquid sample (e.g., blood or a fraction thereof, a bronchial lavage, cerebrospinal fluid, or urine), or a solid sample (e.g., a piece of tissue) In some embodiments, the cell is obtained from peripheral blood. In some embodiments, the cell is obtained from umbilical cord blood.

In some embodiments, allogenic cells in which a cell-distancing device is inserted is isolated from a donor, e.g., using antibodies. In some embodiments, an isolated donor cell is an immune cell, e.g., from the blood or from a particular organ such as the thymus. In some embodiments, immune cells isolated from a donor are T cells such as Treg cells (e.g., CD3+, CD4+, and/or CD8+ cells). In some embodiments, isolation to a donor cell such as a T cell comprises contacting a composition comprising cells to be isolated with a particular binding agent, e.g., an antibody specific to a protein expressed by the cells to be isolated (e.g., an anti-CD3, anti-CD4, or anti-CD8 antibody). In some embodiments, isolation to a donor cell such as a T cell comprises use of flow cytometry.

In some embodiments, a cell is isolated from a donor and then engineered into a particular type of cell. For example, bulk T cells may be isolated from a donor's blood and engineered to stably express FOXP3 by manipulating the Foxp3 gene locus in the cell's genome. See e.g., Honaker et al. (Sci Transl Med 2020 Jun. 3; 12(546):eaay6422), methods described in which are incorporated herein by reference. Another non-limited example of engineering a donor cell into a regulatory type T cell is provided in WO2019180724, which describes incorporation of a membrane-bound IL-10 on cells and which is incorporated herein by reference in its entirety.

In some embodiments, an isolated cell from a donor, e.g., a T cell isolated from the blood of a donor, is not engineered besides incorporating a cell-distancing device.

A T cell or T lymphocyte is an immune system cell that matures in the thymus and produces a T cell receptor (TCR), e.g., an antigen-specific heterodimeric cell surface receptor typically comprised of an alpha-beta heterodimer or a gamma-delta heterodimer. T cells of a given clonality typically express only a single TCR clonotype that recognizes a specific antigenic epitope presented by a syngeneic antigen-presenting cell in the context of a major histocompatibility complex-encoded determinant. T cells can be naïve (“TN”; not exposed to antigen; increased expression of CD62L, CCR7, CD28, CD3, CD127, and CD45RA, and decreased or no expression of CD45RO as compared to TCM (described herein)), memory T cells (TM) (antigen experienced and long-lived), including stem cell memory T cells, and effector cells (antigen-experienced, cytotoxic). TM can be further divided into subsets of central memory T cells (TCM, expresses CD62L, CCR7, CD28, CD95, CD45RO, and CD127) and effector memory T cells (TEM, express CD45RO, decreased expression of CD62L, CCR7, CD28, and CD45RA). Effector T cells (TE) refers to antigen-experienced CD8+ cytotoxic T lymphocytes that express CD45RA, have decreased expression of CD62L, CCR7, and CD28 as compared to TCM, and are positive for granzyme and perforin. Helper T cells (TH) are CD4+ cells that influence the activity of other immune cells by releasing cytokines. CD4+ T cells can activate and suppress an adaptive immune response, and which of those two functions is induced will depend on the presence of other cells and signals. T cells can be collected using known techniques, and the various subpopulations or combinations thereof can be enriched or depleted by known techniques, for example, using antibodies that specifically recognize one or more T cell surface phenotypic markers, by affinity binding to antibodies, flow cytometry, fluorescence activated cell sorting (FACS), or immunomagnetic bead selection. Other exemplary T cells include regulatory T cells (Treg, also known as suppressor T cells), such as CD4+CD25+(Foxp3+) regulatory T cells and Treg17 cells, as well as Tr1, Th3, CD8+CD28−, or Qa-1 restricted T cells. In some embodiments, the donor-derived allogencic cell expressing the T cell-distancing device is a T cell that is capable of binding to peptide:MHC on an antigen-presenting cell with at least 70%, at least 80%, at least 90%, or at least 100% affinity, relative to a control T cell comprising the same TCR that does not express the T cell-distancing device. Methods of measuring the affinity of a T cell to an antigen-presenting cell or a peptide:MHC complex, such as micropipette assays, are known in the art. See, e.g., Huang et al. J Immunol. 2007. 179(11):7633-7662.

In some embodiments of the methods and cells provided herein, the donor-derived allogeneic cell comprises at least 50, at least 100, at least 200, at least 300, at least 400, at least 500, at least 600, at least 700, at least 800, at least 900, at least 1000, at least 1200, at least 1400, at least 1600, at least 1800, at least 2000, at least 2500, at least 3000, at least 3500, at least 4000, at least 4500, at least 5000, at least 6000, at least 7000, at least 8000, at least 9000, at least 10⁵, or at least 10⁶ cell-distancing device molecules. T cells comprise, on average, about 10s T cell receptors, though it is estimated that engagement of about 300-400 T cell receptors on the surface of a T cell can facilitate T cell activation and/or killing of a target cell. Thus, a greater number of T cell-distancing device molecules on the surface of a donor-derived allogeneic cell promotes sequestration of more synaptic molecules (e.g., CD2 molecules) away from T cell receptors, thereby reducing the probability that the allogeneic cell will be killed by a T cell.

### Device Effectiveness

### Expression of the Device

In some embodiments, the expression of a cell-distancing device on the surface the cytoplasm of a cell engineered to express the T cell-distancing device can be evaluated using one or more experimental assays. Non-limiting examples of experimental assays to measure the expression of a T cell-distancing device include antibody-based assays such as Western Blots, and flow cytometry assays.

### Protection of Therapeutic Cell

In some embodiments, the inhibitory effect of the cell-distancing device on the activation of a host immune cells (e.g., T cells or NK cells) can be evaluated using one or more experimental assays. In some embodiments, activity of the host T cells is measured, e.g., by measuring the amount of a particular cytokine expressed by it. In some embodiments, protection conferred by a cell-distancing device on the cells which expresses or comprises it is measured by measuring the viability or lysis of the cells in the presence of host T-cells (either in vitro or in vivo).

Non-limiting examples of experimental assays to measure the inhibitory effect of a T cell-distancing device on T cell activation of host T cells include functional assays (e.g., that measure cytokine (like IFN-γ) production or expression by T cells), structural assays (e.g., using tetramers), and measurement of viability or lysis of the cell expressing the device, or the effect that such cells would have, e.g., on a target cell. See e.g., Expert Rev. Vaccines 9(6), 595-600 (2010); and Clin Diagn Lab Immunol. 2000 November; 7(6): 859-864.

In some embodiments, a therapeutic cell that expresses a cell-distancing device induces at least 10% (e.g., at least 10%, at least 20%, at least 30%, at least 40%, at least 50%, at least 60%, at least 70%, at least 80%, at least 90%, at least 95%, at least 99%) less cytokine production (e.g., IFN production) in host immune cells (e.g., host T cells) compared to a cell that does not express a cell-distancing device (e.g., a cell that is of the same time as the cell comprising the cell distancing device). In some embodiments, a therapeutic cell that expresses a cell-distancing device induces at least 1.5 times (e.g., at least 1.5 times, at least 2 times, at least 3 times, at least 5 times, at least 10 times, at least 20 times, at least 30 times, at least 50 times, at least 100 times, at least 200 times, at least 500 times) less cytokine production (e.g., IFN production) in host immune cells (e.g., host T cells) compared to a cell that does not express a cell-distancing device (e.g., a cell that is of the same time as the cell comprising the cell distancing device). In some embodiments, a therapeutic cell that expresses a cell-distancing device induces at least an order of magnitude less cytokine production (e.g., IFN production) in host immune cells (e.g., host T cells) compared to a cell that does not express a cell-distancing device.

In some embodiments, a therapeutic cell that expresses a cell-distancing device induces at least 10% (e.g., at least 10%, at least 20%, at least 30%, at least 40%, at least 50%, at least 60%, at least 70%, at least 80%, at least 90%, at least 95%, at least 99%) less proliferation in host immune cells (e.g., host T cells) compared to a cell that does not express a cell-distancing device. In some embodiments, a therapeutic cell that expresses a cell-distancing device induces at least 1.5 times (e.g., at least 1.5 times, at least 2 times, at least 3 times, at least 5 times, at least 10 times, at least 20 times, at least 30 times, at least 50 times, at least 100 times, at least 200 times, at least 500 times) less proliferation in host immune cells (e.g., host T cells) compared to a cell that does not express a cell-distancing device. In some embodiments, a therapeutic cell that expresses a cell-distancing device induces at least an order of magnitude less proliferation in host immune cells (e.g., host T cells) compared to a cell that does not express a cell-distancing device.

In some embodiments, a therapeutic cell that expresses a cell-distancing device has at least 10% (e.g., at least 10%, at least 20%, at least 30%, at least 40%, at least 50%, at least 60%, at least 70%, at least 80%, at least 90%, at least 95%, at least 99%) more viability or proliferation in the presence of host immune cells (e.g., host T cells) compared to a cell of the same type that does not express a cell-distancing device under the same conditions. In some embodiments, a therapeutic cell that expresses a cell-distancing device has at least 1.5 times (e.g., at least 1.5 times, at least 2 times, at least 3 times, at least 5 times, at least 10 times, at least 20 times, at least 30 times, at least 50 times, at least 100 times, at least 200 times, at least 500 times) more viability or proliferation in the presence of host immune cells (e.g., host T cells) compared to a cell that does not express a cell-distancing device under the same conditions. In some embodiments, a therapeutic cell that expresses a cell-distancing device has at least an order of magnitude more viability or proliferation in the presence of host immune cells (e.g., host T cells) compared to a cell that does not express a cell-distancing device under the same conditions.

### Effect on Function of Cells Expressing a Cell-Distancing Device

In some embodiments, a therapeutic cell or donor-derived allogeneic cell is an immune cell, such as a cytotoxic T cell, regulatory T cell (Treg), B cell or NK cell; or a hematopoietic stem cell. In some embodiments, the effect of a T cell-distancing device on the function of a T Cell Receptor (TCR or CAR) expressed on the same therapeutic cells or donor-derived allogeneic cell can be measured using one or more experimental assays as described herein. In some embodiments, the T cell-distancing device expressed on a donor-derived allogeneic cell does not disturb (e.g., impede) the function (e.g., of a TCR or CAR expressed) by that allogeneic cell. In some embodiments, the disruption of function (e.g., TCR or CAR function) of a donor-derived allogeneic cell by the expression of a cell-distancing device on the donor-derived allogeneic cell is less than 50% (e.g., less than 50%, less than 40%, less than 30%, less than 20%, less than 10%, less than 5%, or less than 3%) of the donor-derived allogeneic cell function.

In some embodiments, the immune cell is further expressing a chimeric antigen receptor (CAR).

In some embodiments, the effect activity of a T cell-distancing device on the function of a CAR expressed by the same donor-derived allogeneic cell can be measured using one or more experimental assays as described herein. In some embodiments, the T cell-distancing device does not disturb (e.g., impede) the function of a CAR expressed by that allogeneic cell. In some embodiments, the disruption of a CAR function of a donor-derived allogeneic cell by the expression of a T cell-distancing device on the donor-derived allogeneic cell is less than 50% (e.g., less than 50%, less than 40%, less than 30%, less than 20%, less than 10%, less than 5%, or less than 3%) of the donor-derived allogeneic cell function.

In some embodiments, the donor-derived allogeneic cell-line is an induced pluripotent stem cell-line.

In some embodiments, the differentiated cell derived from an induced pluripotent stem cell-line is a retinal pigment epithelial cell, cardiac cell or neural cell. In some aspects, the present disclosure provides a method of transplantation therapy in a subject in need thereof, said method comprising administering to said subject in need a donor-derived allogeneic cell, cell-line or stem cell-line or a differentiated cell, organ or tissue derived from stem cells, of any one of the above embodiments.

### Methods of Administering Cells Comprising Cell-Distancing Device

In some aspects, the present disclosure provides a method comprising administering to a subject any one of the cells described herein to be protected and comprising any one of the cell-distancing devices described herein. In some embodiments, a method comprising administering to a subject a donor-derived allogeneic cell that comprises or expresses any one of the cell-distancing devices disclosed herein. In some aspects, the present disclosure provides a method comprising administering to a subject a composition comprising donor-derived allogeneic cells that comprises or expresses any one of the cell-distancing devices disclosed herein. In some embodiments, compositions comprising cells as disclosed herein also comprise a pharmaceutically acceptable carrier.

A cell administered to a subject can be any type of cell, e.g., an isolated cell isolated from a biological sample as described above, or an isolated cell that is then engineered to express a protein, e.g., to express stable FOXP3 or IL-10. In some embodiments, a cell administered to a subject is an immune cells. Non-limited examples of an immune cells include granulocytes, mast cells, monocytes, neutraphils, dendritic cells, NK cells, or adaptive cells like B cells and T cells. T cells may be ctytotoxic T cells, helper T cells or regulatory T cells.

In some embodiments, the subject is a human. In some embodiments, the subject has or is at risk of developing an autoimmune condition, an allergic condition, and/or an inflammatory condition. In some embodiments, the subject has or is at risk of developing an autoimmune condition selected from the group consisting of type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, bullous pemphigoid, pemphigus vulgaris, autoimmune hepatitis, psoriasis, Sjogren's syndrome, or celiac disease. In some embodiments, the allergic condition is selected from the group consisting of allergic asthma, atopic dermatitis, pollen allergy, food allergy, drug hypersensitivity, or contact dermatitis. In some embodiments, the inflammatory condition is selected from the group consisting of pancreatic islet cell transplantation, asthma, steroid-resistant asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GVHD), tolerance induction for transplantation, transplant rejection, or sepsis. In some embodiments, the subject has or is at risk of developing type 1 diabetes mellitus. In some embodiments, the subject has or is at risk of developing inflammatory bowel disease. In some embodiments, the subject has or is at risk of developing acute respiratory distress syndrome (ARDS).

In some embodiments, a T cell-distancing device expressed by a donor-derived allogeneic cell administered to a subject confers protection to the donor-derived allogeneic cells from the subject's immune cells. In some embodiments, a donor-derived allogeneic cell administered to a subject and expressing a T cell-distancing device is at least 1.5 times (e.g., at least 1.5, 2, 3, 4, 5, 6, 7, 8, 9, 10, 20, 50, or 100 or more than 100 times) better at evading the subject's immune system than the same donor-derived allogeneic cell not expressing or comprising the T cell-distancing device.

In some embodiments, following transplantation, methods provided herein prevent, attenuates or confers resistance to allorejection of said donor-derived allogeneic cell, cell-line or stem cell-line or differentiated cell, organ or tissue derived from stem cells by alloreactive host lymphocytes, as compared with methods of transplantation therapy using allogeneic cells, cell-lines or stem cell-lines or differentiated cells, organs or tissue derived from stem cells that do not express the alloreactive T cell-distancing device of the present invention.

In some embodiments, methods provided herein prevent, attenuate or confer resistance to rejection (e.g., allorejection) of said donor-derived allogeneic cells, cell-lines, tissue or organs by alloreactive host lymphocytes selected from CD8 and CD4 T cells and NK cells.

In some embodiments, the transplantation therapy includes adoptive immune cell therapy, stem cell transplantation or transplantation of organ or tissue derived from stem cells.

### Definitions

The term “allogeneic” as used herein refers to tissues, organs or cells that are genetically dissimilar from, and hence immunologically incompatible with, a host receiving them, although from individuals of the same species. The phrase “donor-derived” as used herein refers to tissues, organs or cells extracted from an individual's organism (e.g., a donor) and intended to be received by a host which may or may not be the same, or of the same species, as the donor.

As used herein, the terms “subject” or “individual” or “animal” or “patient” or “mammal,” refers to any subject, particularly a mammalian subject, for whom diagnosis, prognosis, or therapy is desired, for example, a human.

The term “treating” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local. In some embodiments, the pharmaceutical composition is adapted for oral administration.

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

The term “therapeutically effective amount” as used herein means an amount of the nucleic acid sequence/molecule or vector that will elicit the biological or medical response of a tissue, system, animal or human that is being sought, i.e. treatment of a disease associated with or caused by a cell state, such as cancer. The amount must be effective to achieve the desired therapeutic effect as described above, depending inter alia on the type and severity of the condition to be treated and the treatment regime. The therapeutically effective amount is typically determined in appropriately designed clinical trials (dose range studies) and the person skilled in the art will know how to properly conduct such trials to determine the effective amount. As generally known, an effective amount depends on a variety of factors including the affinity of the ligand to the receptor, its distribution profile within the body, a variety of pharmacological parameters such as half-life in the body, on undesired side effects, if any, and on factors such as age and gender, etc.

The transition phrase “consisting essentially of” or “essentially consisting of”, when referring to an amino acid or nucleic acid sequence, refers to a sequence that includes the listed sequence and is open to present or absent unlisted sequences that do not materially affect the basic and novel properties of the protein itself or the protein encoded by the nucleic acid sequence.

Unless otherwise indicated, all numbers used in this specification are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

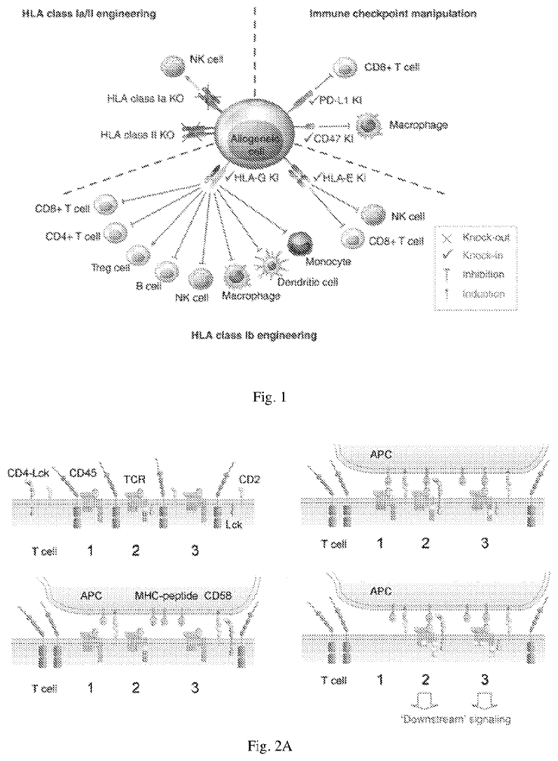

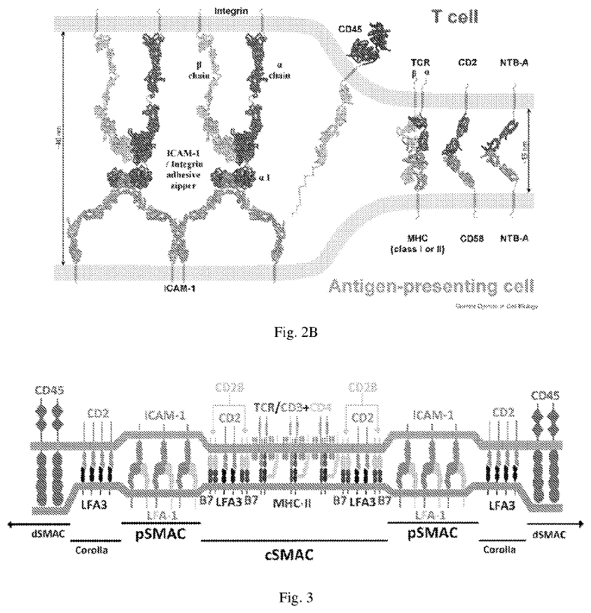

The new molecular device disclosed herein is based, at least in part, on the kinetic segregation (KS) model for T cell activation by an antigen-presenting cell or a target cell (APC/T) ([20-23], see FIGS. 2A and 2B). Ligation of the TCR by p/MHC complexes on APC/T triggers T cell activation signaling. One of the earliest events in this process is the phosphorylation of tyrosine residues in the immune-receptor-tyrosine-based-activation-motifs (ITAMs) of the TCR CD3 γ, δ, ε and ξ subunits, mainly by the Src family nonreceptor tyrosine kinase Lck, which is noncovalently associated with the CD4 and CD8 coreceptors. This step, in turn, activates the ZAP70 protein tyrosine kinase, leading to phosphorylation of downstream adapter proteins and enzymes and, eventually, to the transmission of the integrated signals into the T cell nucleus. CD45 is an abundant cell surface protein tyrosine phosphatase with exceptionally high catalytic activity, which plays a critical role in the regulation of T cell activation. Prior to encountering antigen, CD45 dephosphorylates a C-terminal negative regulatory tyrosine on Lck, allowing the latter to phosphorylate CD3 ITAMs upon TCR ligation.

A higher resolution illustration of the immunological synapse, with emphasis on the important role of adhesion molecules in its stabilization is presented in FIG. 3 (taken from [24]).

The repeated demonstrations that global phosphatase inhibitors and kinase activators can induce spontaneous T cell activation in the absence of antigen have prompted the notion that CD45 serves as a safeguard, reducing non-specific T cell activation by maintaining a sub-threshold level of phosphorylated ITAMs. This scenario immediately raised the question of how CD45 activity is reduced considering the high rate of ITAM phosphorylation which follows TCR ligation, as CD45 cannot discriminate between ‘legitimate’ and ‘prohibited’ phosphotyrosines. The KS model posits the forced segregation of CD45 from the contact zone, providing a mechanistic explanation for the regulation of TCR signaling by CD45. This model has received ample experimental support since first introduced (e.g., [25-30]).

Following are distinct and pertinent features of the KS model as they relate to some of the cell-distancing devices as provided herein:

First, the close contact zone that initially forms between the two cells is primarily occupied with compact ‘binding’ cell surface molecules (see [30]), including the TCR, CD4/CD8, CD28, CD2 and SLAMF6 on the T cell and p/MHCI, B7, LFA-3 (lymphocyte functional antigen-3, the CD2 ligand, CD58 in humans and CD48 in mice) and SLAMF6 on the APC/T, creating an interface of ≈15 nm. To allow these interactions, bulky T cell surface molecules, including CD45, CD148, CD43 and LFA-1, some spanning 40 nm and more, are excluded from the contact zone.

Second, in the periphery of the contact zone, T cell-APC/T interactions are stabilized by the formation of zipper-like complexes between T cell integrins (e.g. LFA-1) and cell adhesion molecules (such as ICAM-1) on the interacting cells. Sorting of large integrins to these designated areas is governed by the actin cytoskeleton [31] so that separation between narrow antigen-specific interfaces and wide non-specific ones guarantees that T cell signaling is not sterically hindered.

Third, the exclusion of CD45 and CD148 from the contact zone is critical for TCR signaling. An important structural component of the CD45 ectodomain which confers the rigidity necessary for exclusion comprises three fibronectin type III repeats [32]. The expression of truncated forms of these phosphatases prevented their exclusion and resulted in strong inhibition of T cell activation [26,29].

The exclusion of elongated Lck from the contact zone prevented T cell activation [23], corroborating the importance of molecular dimensions and size-based sorting for T cell signaling.

Elongation of the TCR-p/MHC axis through incremental extensions of the p/MHC ectodomain almost completely abolished TCR triggering without affecting TCR-p/MHC ligation, an effect that was ascribed to increased retention of CD45 at the contact zone [27].

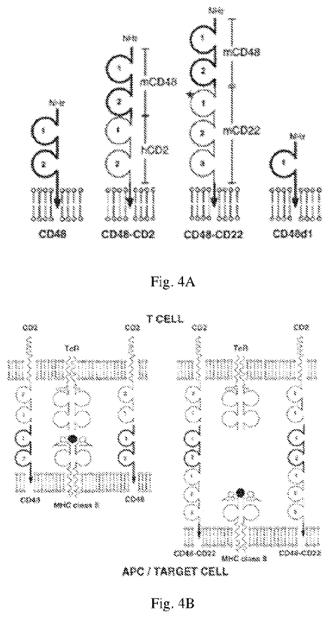

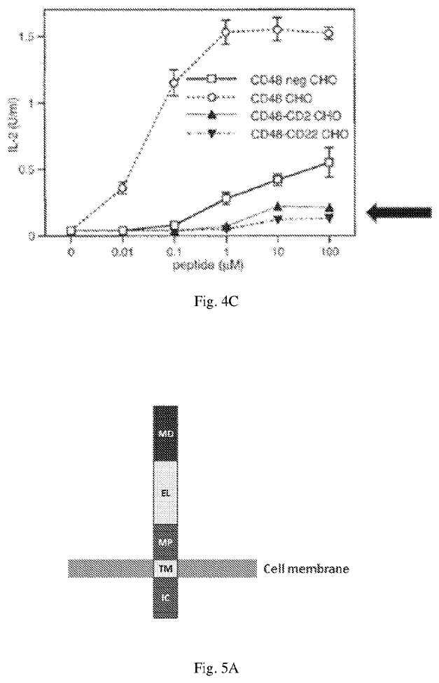

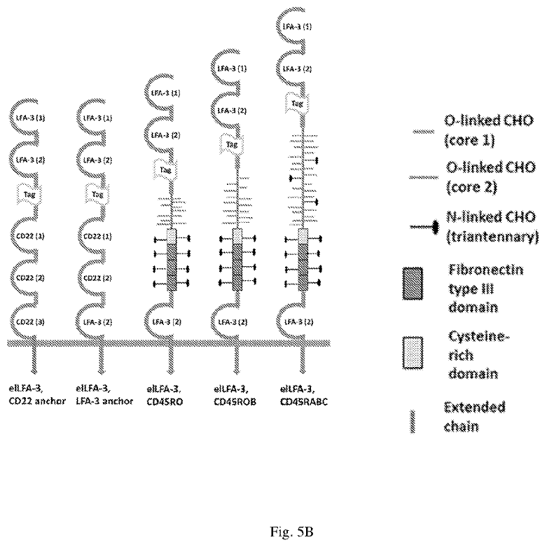

The CD2 adhesion and costimulatory molecule is normally expressed by T cells and NK cells and binds its natural ligand LFA-3 (CD58 in FIG. 2D, FIG. 3), which is mainly expressed on APCs (see [24] for a recent review on CD2 immunobiology). CD2 has been shown to physically associate with the TCR-CD3 complex at the T cell surface [33], playing a major role in cytoskeletal polarization at the contact zone [34,35]. Artificially elongated derivatives of the CD2-LFA-3 axis (achieved via genetic engineering of CD48, the mouse CD2 ligand) prevented TCR-mediated signaling in a T cell hybridoma [25] and severely reduced proliferation of primary T cells in response to TCR stimulation [36]. The authors attributed this inhibitory effect to the increased intermembrane spacing, which was too wide to accommodate TCR-p/MHC interactions ([25], see FIG. 4). Reexamining these findings with high resolution technologies, the later study [36] proposed that reorganization of the immunological synapse enforced by the extended CD48 ectodomain sequestered the TCR in a location where it could no longer interact with p/MHC, dramatically reducing T cell sensitivity. This study [36] provided solid evidence that even nanoscale increases in the intermembrane spacing forced by the CD2-CD48 axis resulted in a significant reduction in the magnitude of primary T cell response to p/MHC antigen on APCs.

Although the composition of activating and inhibitory receptors forming the immunological synapse of NK cells differs from that of T cells, the same principles govern synapse organization in these two cell types [37-40]. Indeed, similarly to T cells, ligand dimensions have also been shown to be important in controlling NK cell responses [41]. In this study, the expression by target cells of elongated forms of different sizes of H60a, a ligand for the mouse NK activation receptor NKG2D, resulted in size-dependent inhibition of target cell lysis. Similarly, the expression on target cells of an elongated, single-chain H-2K^{b}, which is a target MHC-I antigen of the NK inhibitory receptor Ly49C, resulted in decreased inhibition compared to the expression of wild type H-2K^{b} [41].

CD2 has also been assigned a central role in the organization of the NK immunological synapse, similarly to its role in the T cell synapse [42].

These reports, and especially [25] and [36] strongly favor the notion of stable, actin-mediated association between CD2 and the TCR-CD3 complex on T cells or NK antigen/ligand receptors in NK cells.

With the KS model and the potent inhibitory capacity of elongated LFA-3 (elLFA-3) serving as guidelines, it is provided herein to exploit elLFA-3 as a means to protect allogeneic cells (e.g., T cells) employed in ACT and allogeneic cells used for tissue or organ regeneration from alloreactive host T and NK cells. Examples of some elLFA-3 configurations as provided herein can be found in FIGS. 5A-5D. FIG. 6 explains the anticipated outcome of the use of the cell-distancing device as provided herein.

In their original study on elongated CD48 [25], the investigators created CD48-CD2 and CD48-CD22 by replacing the CD48 transmembrane domain with that of either human CD2 or mouse CD22, respectively, preserving the two Ig-like extracellular domains of CD48 at the N-terminus of the polypeptide, free to engage the T cell CD2 (FIG. 4A). In parallel to activation-induced association of CD2 with the TCR-CD3 complex and stabilization of the T cell face of the contact zone, the reciprocal association of LFA-3 with HLA molecules at the APC/T face is predicted to further stabilize these intercellular interactions. Indeed, evidence for such an association has been reported [43] [44]. Embodiments of the cell-distancing devise as provided herein provide a stabilization effect, and at the same time, prevent or attenuate the segregation of the CD2-engated elLFA-3 from the contact zone. See e.g., the LFA-3 anchor incorporated into the four right hand side constructs in FIG. 5.

One concern associated with the expression of elLFA-3 is that this artificially extended protein may exert a negative effect on on-target T cell activity in ACT due to size-enforced hindrance of antigen binding by the TCR or CAR. Yet, this is an unlikely scenario, as no association between elLFA-3 and the TCR-CD3 complex is expected so that this molecule is prone to be excluded from the contact zone similarly to all other over-sized membrane proteins, including CD45.

Most approaches for preventing allorejection in any clinical scenario (see below) attempt to reduce, or even completely abolish, disparity between donor and recipient HLA, usually by meticulous selection of donor, or, more recently, by gene editing. Other strategies that permit such disparity may face the problem of anti-donor HLA antibodies, which has been associated with allograft rejection in solid organ transplantation [46]. The effect, if any, of host humoral response against donor HLA is hard to predict at this stage.

The rapid progress made in recent years in induced pluripotent stem cells (iPSC) technologies offer a broad spectrum of potential clinical applications. While autologous iPSC lines can be generated, they are unlikely to serve as a workable source for a large number of patients in the clinical setting, owing mainly to time, labor and cost required for achieving the precise differentiation state and, if necessary, genetic reprogramming, while adhering to strict GMP guidelines. As an alternative, great efforts are made to establish universal libraries of iPSC lines as a source, for example, for T cell engineering towards adoptive cell therapy, [47] as well as all other promising therapeutic applications [48-50].

The cell-distancing device as disclosed herein is efficacious in conferring similar protection on any allogeneic cell-line. This is because engineering therapeutic cells (e.g., iPSC lines) to express any of the cell-distancing devices described herein (e.g., elLFA-3) can suffice to protect the fully differentiated tissue or organ to be transplanted from allorejection by recipient T cells, turning elLFA-3 into a universal genetic tool of immense therapeutic potential.

If fully functional, any cell manipulated to express elLFA-3 would inevitably evade T cell recognition or be recognized to a lesser degree, thus acquiring an immune-privileged status. Such an outcome may prevent or attenuate T cell-mediated elimination of these cells (or tissues and organs originating from gene-modified iPSCs or ES cells) in the event of infection or cellular transformation. Having raised this concern, one should bear in mind that a similar risk is posed by all protocols employing iPSCs or ES cells manipulated to prevent or attenuate allorejection, which are mentioned above. A counteracting strategy that would not eliminate the entire cell population or a whole tissue (say, by a suicide gene) should be worked out.

### EXAMPLES

In some embodiments, the expression and/or activity of a T cell-distancing device can be evaluated using one or more assays. The following paragraphs provide non-limiting examples of assays that can be used to evaluate T cell-distancing device expression and/or activity.

### Example 1: Evaluation of Surface Expression of T Cell-Distancing Constructs Following mRNA Transfection In Vitro

Expression of T cell-distancing devices was tested in mouse RMA cells and in human K562, HEK293 and PBMC-derived T cells. Cells were transfected by electroporation with constructs as shown in FIGS. 5E, 5G, 5K and 5L to express the T cell-distancing devices as shown in FIGS. 5D, 5F, 5H, 5I and 5JC. A pGEM4Z mRNA synthesis vector was used, that contained a T7 promoter, a strong Kozak sequence, vendor-proprietary 5′ and 3′ UTRs, an ORF sequence from ATG start codon to the stop codon (TAA, TAG, TGA), CleanCap 5′ capping, and a 120-nucleotide polyA tail. The pT7 vectors containing the genes were restricted with XbaI and NotI enzymes, extracted from agarose gel and ligated into the pGEM4Z vector. Each T cell-distancing device comprised an extracellular membrane-distal LFA-3 domain, an extracellular elongation (extender) domain and a transmembrane domain as indicated in Table 1, as well as a Human influenza hemagglutinin (HA) tag. Evaluation of cell surface expression of the T cell-distancing devices was done by flow cytometry with an antibody against LFA-3. Functional expression of the constructs expressing the anti-CD2 scfv was determined via flow cytometry with a fluorescently tagged ectodomain of CD2. The expression levels of the different constructs are indicated in Table 1. Functional binding of the CD2 ectodomain to the devices expressing the anti-CD2 scfv is thereby confirmed using this approach as well.

TABLE 1
Expression of T cell-distancing devices on the surface of cells transfected with plasmids
encoding T cell-distancing devices. Each construct used comprises sequences coding
for an extracellular membrane-distal LFA-3 domain, an extracellular elongation (extender)
domain and a transmembrane domain as indicated in the corresponding row. The presence
or absence of a GPI anchor and/or linker is also indicated for each construct.

| | | | LFA-3 | GPI | TM | | | Expression |
|---|---|---|---|---|---|---|---|---|
| Version | Name | Species | domains | anchor | Domain | Extender | Linker | assessment |
| v1.0 | 1882 | Hs | A | (+) | CD58 | CD22 (4D) | (−) | no |
| v1.0 | 1883 | Hs | A | (+) | CD58 | CD45RO | (−) | no |
| v1.0 | 1884 | Hs | A | (+) | CD58 | CD45RABC | (−) | no |
| v1.0 | 1882L | Mm | A | (+) | CD48 | CD22 (4D) | GGGS | no |
| | | | | | | | (SEQ ID | |
| | | | | | | | NO: 105) | |
| v1.0 | 1883L | Mm | A | (+) | CD48 | CD45RO | GGGS | no |
| | | | | | | | (SEQ ID | |
| | | | | | | | NO: 105) | |
| v1.0 | 1884L | Mm | A | (+) | CD48 | CD45RABC | GGGS | no |
| | | | | | | | (SEQ ID | |
| | | | | | | | NO: 105) | |
| v1.1 | New hIEE-22 | Hs | A | (+) | CD22 | CD22 (5D) | (−) | no |
| v1.1 | New hIEE-45 | Hs | A | (+) | CD45 | CD45RO | (−) | no |
| v1.1 | New hIEE-45deltaC | Hs | A | (+) | CD45 | CD45RABC | (−) | no |
| v2.0 | hIEE1(−)22 | Hs | A | (−) | CD22 | CD22 (5D) | (−) | ++ |
| v2.0 | hIEE1(+)22 | Hs | A | (−) | CD22 | CD22 (5D) | GGGS | ++ |
| | | | | | | | (SEQ ID | |
| | | | | | | | NO: 105) | |
| v2.0 | hIEE1(−)RO | Hs | A | (−) | CD45 | CD45RO | (−) | ++ |
| v2.0 | hIEE1(+)RO | Hs | A | (−) | CD45 | CD45RO | GGGS | ++ |
| | | | | | | | (SEQ ID | |
| | | | | | | | NO: 105) | |
| v2.0 | hIEE1(−)ABC | Hs | A | (−) | CD45 | CD45RABC | (−) | ++ |
| v2.0 | hIEE1(+)ABC | Hs | A | (−) | CD45 | CD45RABC | GGGS | ++ |
| | | | | | | | (SEQ ID | |
| | | | | | | | NO: 105) | |
| v2.0 | hIEE2(−)22 | Hs | AB | (−) | CD22 | CD22 (5D) | (−) | ++ |
| v2.0 | hIEE2(+)22 | Hs | AB | (−) | CD22 | CD22 (5D) | GGGS | ++ |
| | | | | | | | (SEQ ID | |
| | | | | | | | NO: 105) | |
| V2.0 | hIEE2(−)RO | Hs | AB | (−) | CD45 | CD45RO | (−) | ++ |
| v2.0 | hIEE2(+)RO | Hs | AB | (−) | CD45 | CD45RO | GGGS | ++ |
| | | | | | | | (SEQ ID | |
| | | | | | | | NO: 105) | |
| v2.0 | hIEE2(−)ABC | Hs | AB | (−) | CD45 | CD45RABC | (−) | + |
| v2.0 | hIEE2(+)ABC | Hs | AB | (−) | CD45 | CD45RABC | GGGS | + |
| | | | | | | | (SEQ ID | |
| | | | | | | | NO: 105) | |
| v2.0 | hIEE-Fv(+)22 | Hs | aCD2 scFV | (−) | CD22 | CD22 (5D) | GGGS | ++ |
| | | | | | | | (SEQ ID | |
| | | | | | | | NO: 105) | |
| v2.0 | hIEE-Fv(8)22 | Hs | aCD2 scFV | (−) | CD22 | CD22 (5D) | CD8 | ++ |
| | | | | | | | Hinge | |
| v2.0 | hIEE-Fv(+)RO | Hs | aCD2 scFV | (−) | CD45 | CD45RO | GGGS | ++ |
| | | | | | | | (SEQ ID | |
| | | | | | | | NO 105) | |
| v2.0 | hIEE-Fv(8)RO | Hs | aCD2 scFV | (−) | CD45 | CD45RO | CD8 | ++ |
| | | | | | | | Hinge | |
| v2.0 | hIEE-Fv(+)ABC | Hs | aCD2 scFV | (−) | CD45 | CD45RABC | GGGS | + |
| | | | | | | | (SEQ ID | |
| | | | | | | | NO: 105) | |
| v2.0 | hIEE-Fv(8)ABC | Hs | aCD2 scFV | (−) | CD45 | CD45RABC | CD8 | + |
| | | | | | | | Hinge | |

Hs: human;
Mm: mouse

### Example 2: Assessment of Protection of Allogeneic Cells by T Cell-Distancing Device Against Antigen-Specific T-Cells

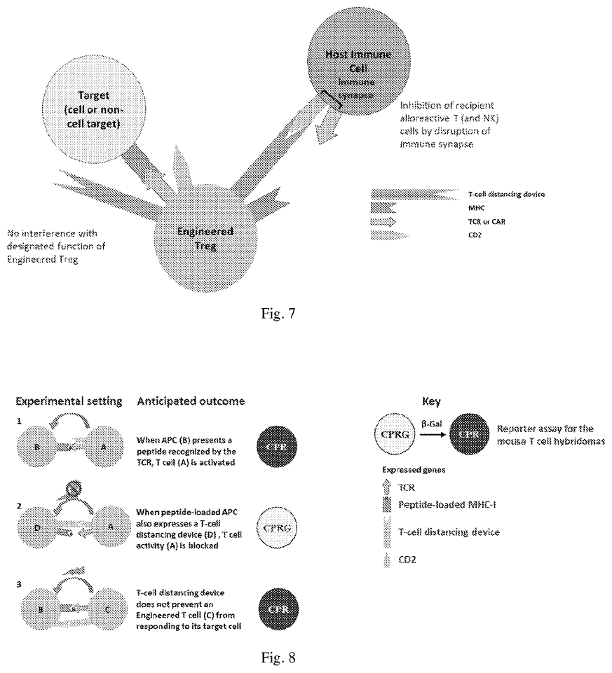

A T cell-distancing device expressed on the surface of a donor-derived cell protects the donor-derived cell from being attacked by the host immune cells, while preserving the function of the donor-derived cell as illustrated in FIG. 7.

Experimental settings 1 and 2 as shown in FIG. 8 describe the assay for determining activation levels of T-cells on donor-derived cells in the presence of a T cell-distancing device expressed on the donor-derived cells.

Protection Assay of Gp100-Presenting RMA Cells Co-Cultured with BUSA14

RMA cells are electroporated with mRNA constructs coding for (i) a control sequence, or (ii) a T cell-distancing device, and incubated for 6-8 hours. Following incubation, the cells are loaded with 300 ng/ml of gp100 peptide, and then co-cultured in a 1:1 ratio in a 96-well plate with BUSA14 cells transfected with a β-galactosidase expression construct. Co-cultured cells are lysed and analyzed by a CPRG assay. T cell activation is significantly higher when RMA cells are transfected with the construct coding for the T cell-distancing device compared to when RMA cells are transfected with the control sequence.

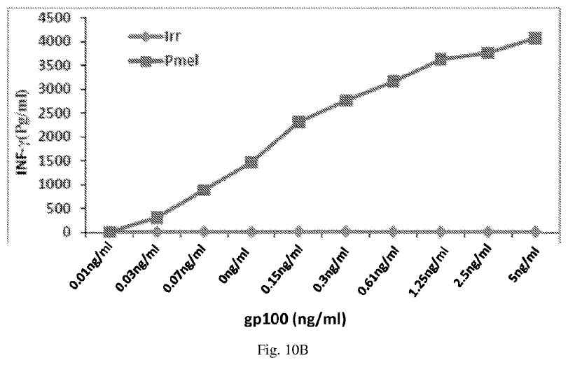

To measure the effect of T-cell distancing devices comprised in allogeneic cells on T-cell activation, CD8 T cells were transfected with a pGEM4Z vector, comprising: (i) a control sequence, (ii) a Pmel-TCR construct, (iii) a Pmel-TCR construct and a control sequence, or (iv) a Pmel-TCR construct and a T cell-distancing device construct. See constructs in Table 2. First, expression of Pmel-TCR on the surface of CD8 T cells was confirmed. The CD8 T cells were incubated for 6 hours. Following incubation, the cells were co-cultured overnight with RMA cells transfected with (i) a control sequence, or (ii) a T cell-distancing device construct, and loaded with gp100 peptide (0-1,000 ng/ml (FIG. 10A) and 0-5 ng/ml (FIG. 10B)). Table 3 below shows the different experimental conditions of the co-culture. The supernatant from the co-culture was collected and analyzed for INF-γ expression. Results are shown in FIGS. 10A and 10B.

TABLE 2
Pmel TCR constructs.

| # | Clone number | Description |
|---|---|---|
| 1 | 1979 | Pmel VαCα In pGEM4Z vector |
| 3 | 1980 | Pmel VβCβ In pGEM4Z vector |

TABLE 3
Experimental plan for evaluating the ability of the T
cell-distancing device (or, Immune
Evasive Engineering, IEE) constructs to inhibit
activation of CD8 T cells in the presence of
RMA cells expressing gp100.

| | |
|---|---|
| 1 | CD8(Irr) + RMA(Irr) |
| 2 | CD8(Irr) + RMA(Irr) + gp100 |
| 3 | CD8(Irr) + RMA(IEE) |
| 4 | CD8(Irr) + RMA(IEE) + gp100 |
| 5 | . CD8(Pmel) + RMA(Irr) |
| 6 | CD8(Pmel)- + RMA(Irr) + gp100 |
| 7 | CD8(Pmel) + RMA(IEE) |
| 8 | CD8(Pmel) + RMA(IEE) + gp100 |
| 9 | OKT3 (positive control) |
| 10 | CD8(Pmel + Irr) + RMA |
| 11 | CD8(Pmel + Irr) + RMA + gp100 |
| 12 | CD8(Pmel + IEE) + RMA |
| 13 | CD8(Pmel + IEE) + RMA + gp100 |
| 14 | CD8(Pmel) + RMA(Irr) + gp100 1:3 |
| 15 | CD8(Pmel) + RMA(IEE) + gp100 1:3 |
| 16 | CD8(Pmel + Irr) + RMA + gp100 1:3 |
| 17 | CDS(Pmel + IEE) + RMA + gp100 1:3 |

### Example 3: Effect of T-Rea Expressed T Cell-Distancing Device on T-Reg Function

An experiment is set up as shown in Experimental setting 3 in FIG. 8. Tregs expressing a T cell-distancing device are incubated with target cells of Tregs. T cell-distancing device is expected to be excluded from the immune synapse, based on the KS model, and therefore does not inhibit the function of the engineered Treg.

### Example 4: Effect of T Cell-Distancing Device Expressed by CAR T-Cell on CAR T-Cell Function

### Experimental Set-Up

Reporter Jurkat cells are divided in six experimental groups. Groups A to E are transfected with a construct coding for the following:
Group A: an anti-A2 chimeric antigen receptor (CAR) only,
Group B: an A2 protein only,
Group C: anti-A2 CAR and a T cell-distancing device,
Group D: A2 and a T cell-distancing device,
Group E: a T cell-distancing device.
Group F comprises non-transfected Jurkat cells. Cells from different groups are co-cultured overnight at a 1:1 ratio, following incubation. T cell activation is measured by quantifying luciferase activity.

### Results

Group A+Group F result in low level of T cell activation

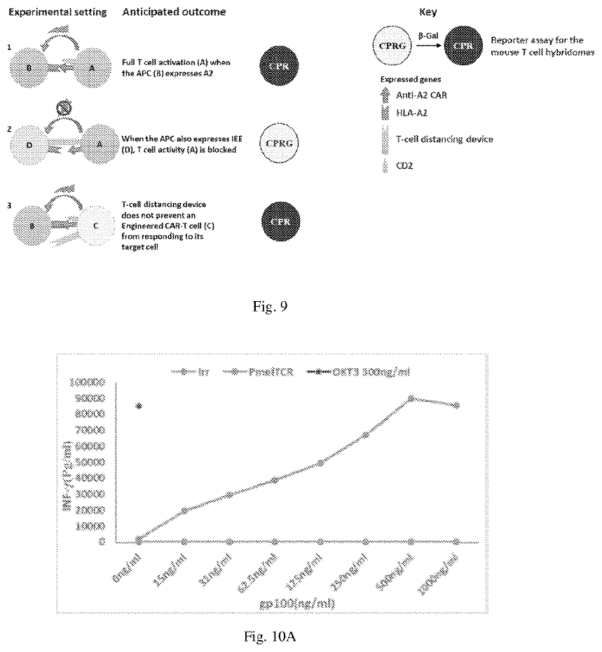

Group A+Group B result in high level of T cell activation (experimental setting 1 in FIG. 9)

Group A+Group D result in low level of T cell activation owing to blocking by the T cell-distancing device (experimental setting 2 in FIG. 9).

Group C+Group B result in T cell activation level similar to ‘2’ (Group A+Group B) (experimental setting 3 in FIG. 9).

Group E only result in low level of T cell activation

The same experimental setting is applied using mRNA-transfected B3Z cells to answer the same questions in mouse T cells, with similar results.

### Example 5: Evaluation of T Cell-Distancing Device Function in Mixed Lymphocyte Reaction (MLR)

Two one-way MLR experiments are performed, using human PBMCs obtained from two unrelated healthy individuals (one individual is referred to as the “Donor” and the other as the “Recipient”). Prior to the MLR coculture assay, Recipient T cells are first pre-stimulated by Donor monocyte-derived dendritic cells (DCs) for 5-7 days to allow activation and proliferation of Recipient anti-Donor T cells. In parallel, Donor T cells are similarly stimulated by Recipient DCs to enrich for activated Donor anti-Recipient T cells.

In the Recipient anti-Donor one-way MLR, pre-stimulated Recipient T cells are stained with CFSE and cocultured with non-stimulated Donor T cells transfected with a T cell distancing device or irrelevant mRNA. Activation of CFSE-stained Recipient T cells are monitored by CFSE dilution and intracellular staining for IFN-7.

In the Donor anti-Recipient MLR, CFSE-stained pre-stimulated Donor anti-Recipient T cells transfected with a T cell distancing device or irrelevant mRNA are cocultured with non-stimulated Recipient T cells (or PBMCs) and their activation is similarly monitored.

The experiments show that:

Donor T cells expressing human T cell distancing device mRNA reduce Recipient anti-Donor T cell response compared to the same Donor T cells transfected with irrelevant mRNA.
The expression of a T cell distancing device by Donor T cells does not impair their allo-reactivity against Recipient T cells (or Recipient PBMCs) compared to the same cells transfected with irrelevant mRNA.

### Example 6: Assessment of elLFA-3 Constructs in Protecting Cancer Cells

For assessing the ability of the human elLFA-3 constructs of the present invention to protect human cells from T cell attack different experimental systems are evaluated in parallel:
1. Matching pairs of human melanomas transfected with an elLFA-3 construct and autologous tumor-infiltrating lymphocytes (TILs).
2. In a study on Tregs in celiac disease (CeD) genes encoding the pairs of a and 3 chains comprising two distinct TCRs, each specific to a different gliadin α-derived peptide (known as glia-α1a and glia-α2) bound to the CeD-associated HLA-II molecule DQ2.5, were cloned. Co-expression of each of these two TCR α and β chain pair in mRNA-transfected human Jurkat CD4 T cells was confirmed. In parallel, α and β chain of DQ2.5: DQA1*05:01:01:01 and DQB1*02:01:01, respectively, were also cloned. An identical DQ2.5 αchain allele and a closely-related DQ2.5 β chain allele (DQB1*02:01:01) encode the HLA-DQ2.5 product expressed by the human B cell lymphoma, Raji, which efficiently presents both glia-ala and glia-α2 (51).

Using this experimental system, protection of elLFA-3-expressing mRNA electroporated Raji cell pre-loaded with the respective peptide from recognition by NFAT-Luciferase reporter Jurkat cells expressing the matching TCR is evaluated. Alternatively, other human cell lines (e.g., the lymphoblastoid B cell line 721.221 (52) and the B myeloma cell line AF10, a subclone of the IgE-producing U266 myeloma (53)) are co-transfected with mRNAs encoding the two DQ2.5 and the el-LFA-3 constructs under study.

### In-Vivo Assessment

For assessing the ability of elLFA-3 to confer protection from allorejection, the H-2^{b}transplantable melanoma cell line B16, stably transfected with a mouse elLFA-3 construct selected through ex-vivo experiments is exploited. These cells are introduced subcutaneously to one flank of recipient allogenic BALB/c mice (H-2^{d}) while wild type B16 cells are similarly introduced to the other flank. The elLFA-3-expressing B16 cells exhibit higher persistence and proliferative capacity in the recipient mice compared with their wild type, non-protected counterparts.

TABLE 4
Example Nucleic Acid Sequences

| | | | |
|---|---|---|---|
| SEQ | | | |
| ID | Construct | | |
| NO | Number | Description | Sequence |

Extracellular membrane-distal domains (denoted by underlining). Some examples below
include a hinge denoted by bold lettering. Some examples comprise an indication of
restriction sites, denoted by bold and underlined lettering.

| | | | |
|---|---|---|---|
| 1 | 1882, | LFA-3 | GTTGCTGGGAGCGACGCGGGGCGGGCCCTGGGGGTCCTCAGC |
| | 1883, | ectodomain | GTGGTCTGCCTGCTGCACTGCTTTGGTTTCATCAGCTGTTTTTCC |
| | 1884 | | CAACAAATATATGGTGTTGTGTATGGGAATGTAACTTTCCATGT |
| | | | ACCAAGCAATGTGCCTTTAAAAGAGGTCCTATGGAAAAAACAA |
| | | | AAGGATAAAGTTGCAGAACTGGAAAATTCTGAGTTCAGAGCTT |
| | | | TCTCATCTTTTAAAAATAGGGTTTATTTAGACACTGTGTCAGGTA |
| | | | GCCTCACTATCTACAACTTAACATCATCAGATGAAGATGAGTAT |
| | | | GAAATGGAATCGCCAAATATTACTGATACCATGAAGTTCTTTCT |
| | | | TTATGTGCTTGAGTCTCTTCCATCTCCCACACTAACTTGTGCATT |
| | | | GACTAATGGAAGCATTGAAGTCCAATGCATGATACCAGAGCAT |
| | | | TACAACAGCCATCGAGGACTTATAATGTACTCATGGGATTGTCC |
| | | | TATGGAGCAATGTAAACGTAACTCAACCAGTATATATTTTAAGA |
| | | | TGGAAAATGATCTTCCACAAAAAATACAGTGTACTCTTAGCAAT |
| | | | CCATTATTTAATACAACATCATCAATCATTTTGACAACCTGTATC |
| | | | CCAAGCAGCGGTCATTCAAGACACAGA |
| 2 | 1882, | LFA-3 | TTTTCCCAACAAATATATGGTGTTGTGTATGGGAATGTAACTTTC |
| | 1883, | ectodomain | CATGTACCAAGCAATGTGCCTTTAAAAGAGGTCCTATGGAAAA |
| | 1884 | | AACAAAAGGATAAAGTTGCAGAACTGGAAAATTCTGAGTTCAG |
| | | | AGCTTTCTCATCTTTTAAAAATAGGGTTTATTTAGACACTGTGTC |
| | | | AGGTAGCCTCACTATCTACAACTTAACATCATCAGATGAAGATG |
| | | | AGTATGAAATGGAATCGCCAAATATTACTGATACCATGAAGTTC |
| | | | TTTCTTTATGTGCTTGAGTCTCTTCCATCTCCCACACTAACTTGTG |
| | | | CATTGACTAATGGAAGCATTGAAGTCCAATGCATGATACCAGA |
| | | | GCATTACAACAGCCATCGAGGACTTATAATGTACTCATGGGATT |
| | | | GTCCTATGGAGCAATGTAAACGTAACTCAACCAGTATATATTTT |
| | | | AAGATGGAAAATGATCTTCCACAAAAAATACAGTGTACTCTTAG |
| | | | CAATCCATTATTTAATACAACATCATCAATCATTTTGACAACCTG |
| | | | TATCCCAAGCAGCGGTCATTCAAGACACAGA |
| 3 | | N-terminal | TTTTCCCAACAAATATATGGTGTTGTGTATGGGAATGTAACTTTC |
| | | Ig-like | CATGTACCAAGCAATGTGCCTTTAAAAGAGGTCCTATGGAAAA |
| | | domain of | AACAAAAGGATAAAGTTGCAGAACTGGAAAATTCTGAGTTCAG |
| | | LFA-3 | AGCTTTCTCATCTTTTAAAAATAGGGTTTATTTAGACACTGTGTC |
| | | (CD58) | AGGTAGCCTCACTATCTACAACTTAACATCATCAGATGAAGATG |
| | | | AGTATGAAATGGAATCGCCAAATATTACTGATACCATGAAGTTC |
| | | | TTTCTTTATGTG |
| 4 | | two N- | TTTTCCCAACAAATATATGGTGTTGTGTATGGGAATGTAACTTTC |
| | | terminal Ig- | CATGTACCAAGCAATGTGCCTTTAAAAGAGGTCCTATGGAAAA |
| | | like | AACAAAAGGATAAAGTTGCAGAACTGGAAAATTCTGAGTTCAG |
| | | domains of | AGCTTTCTCATCTTTTAAAAATAGGGTTTATTTAGACACTGTGTC |
| | | LFA-3 | AGGTAGCCTCACTATCTACAACTTAACATCATCAGATGAAGATG |
| | | (CD58) | AGTATGAAATGGAATCGCCAAATATTACTGATACCATGAAGTTC |
| | | | TTTCTTTATGTGCTTGAGTCTCTTCCATCTCCCACACTAACTTGTG |
| | | | CATTGACTAATGGAAGCATTGAAGTCCAATGCATGATACCAGA |
| | | | GCATTACAACAGCCATCGAGGACTTATAATGTACTCATGGGATT |
| | | | GTCCTATGGAGCAATGTAAACGTAACTCAACCAGTATATATTTT |
| | | | AAGATGGAAAATGATCTTCCACAAAAAATACAGTGTACTCTTAG |
| | | | CAATCCATTATTTAATACAACATCATCAATCATTTTGACAACCTG |
| | | | TATCCCAAGC |
| 5 | 1885, | CD48 | TGCTTCATAAAACAGGGATGGTGTCTGGTCCTGGAACTGCTAC |
| | 1886, | ectodomain | TGCTGCCCTTGGGAACTGGATTTCAAGGTCATTCAATACCAGAT |
| | 1887, | | ATAAATGCCACCACCGGCAGCAATGTAACCCTGAAAATCCATAA |
| | 1944, | | GGACCCACTTGGACCATATAAACGTATCACCTGGCTTCATACTA |
| | 1945, | | AAAATCAGAAGATTTTAGAGTACAACTATAATAGTACAAAGAC |
| | 1946 | | AATCTTCGAGTCTGAATTTAAAGGCAGGGTTTATCTTGAAGAAA |
| | | | ACAATGGTGCACTTCATATCTCTAATGTCCGGAAAGAGGACAA |
| | | | AGGTACCTACTACATGAGAGTGCTGCGTGAAACTGAGAACGAG |
| | | | TTGAAGATAACCCTGGAAGTATTTGATCCTGTGCCCAAGCCTTC |
| | | | CATAGAAATCAATAAGACTGAAGCCTCCACTGATTCCTGTCACC |
| | | | TGAGGCTATCGTGTGAGGTAAAGGACCAGCATGTTGACTATAC |
| | | | TTGGTATGAGAGCAGCGGACCTTTCCCCAAAAAGAGTCCAGGA |
| | | | TATGTGCTCGATCTCATCGTCACACCACAGAACAAGTCTACATTT |
| | | | TACACCTGCCAAGTCAGCAATCCTGTAAGCAGCAAGAACGACA |
| | | | CAGTGTACTTCACTCTACCTTGTGATCT |
| 6 | 1885, | CD48 | TTTCAAGGTCATTCAATACCAGATATAAATGCCACCACCGGCAG |
| | 1886, | ectodomain | CAATGTAACCCTGAAAATCCATAAGGACCCACTTGGACCATATA |
| | 1887, | | AACGTATCACCTGGCTTCATACTAAAAATCAGAAGATTTTAGAG |
| | 1944, | | TACAACTATAATAGTACAAAGACAATCTTCGAGTCTGAATTTAA |
| | 1945, | | AGGCAGGGTTTATCTTGAAGAAAACAATGGTGCACTTCATATCT |
| | 1946 | | CTAATGTCCGGAAAGAGGACAAAGGTACCTACTACATGAGAGT |
| | | | GCTGCGTGAAACTGAGAACGAGTTGAAGATAACCCTGGAAGTA |
| | | | TTTGATCCTGTGCCCAAGCCTTCCATAGAAATCAATAAGACTGA |
| | | | AGCCTCCACTGATTCCTGTCACCTGAGGCTATCGTGTGAGGTAA |
| | | | AGGACCAGCATGTTGACTATACTTGGTATGAGAGCAGCGGACC |
| | | | TTTCCCCAAAAAGAGTCCAGGATATGTGCTCGATCTCATCGTCA |
| | | | CACCACAGAACAAGTCTACATTTTACACCTGCCAAGTCAGCAAT |
| | | | CCTGTAAGCAGCAAGAACGACACAGTGTACTTCACTCTACCTTG |
| | | | TGATCT |
| 7 | 1941, | CD58 | GTTGCTGGGAGCGACGCGGGGCGGGCCCTGGGGGTCCTCAGC |
| | 1942, | ectodomain | GTGGTCTGCCTGCTGCACTGCTTTGGTTTCATCAGCTGTTTTTCC |
| | 1943 | | CAACAAATATATGGTGTTGTGTATGGGAATGTAACTTTCCATGT |
| | | | ACCAAGCAATGTGCCTTTAAAAGAGGTCCTATGGAAAAAACAA |
| | | | AAGGATAAAGTTGCAGAACTGGAAAATTCTGAGTTCAGAGCTT |
| | | | TCTCATCTTTTAAAAATAGGGTTTATTTAGACACTGTGTCAGGTA |
| | | | GCCTCACTATCTACAACTTAACATCATCAGATGAAGATGAGTAT |
| | | | GAAATGGAATCGCCAAATATTACTGATACCATGAAGTTCTTTCT |
| | | | TTATGTGCTTGAGTCTCTTCCATCTCCCACACTAACTTGTGCATT |
| | | | GACTAATGGAAGCATTGAAGTCCAATGCATGATACCAGAGCAT |
| | | | TACAACAGCCATCGAGGACTTATAATGTACTCATGGGATTGTCC |
| | | | TATGGAGCAATGTAAACGTAACTCAACCAGTATATATTTTAAGA |
| | | | TGGAAAATGATCTTCCACAAAAAATACAGTGTACTCTTAGCAAT |
| | | | CCATTATTTAATACAACATCATCAATCATTTTGACAACCTGTATC |
| | | | CCAAGCAGCGGTCATTCAAGACACAGA |
| 8 | 1941, | CD58 | TTTTCCCAACAAATATATGGTGTTGTGTATGGGAATGTAACTTTC |
| | 1942, | ectodomain | CATGTACCAAGCAATGTGCCTTTAAAAGAGGTCCTATGGAAAA |
| | 1943 | | AACAAAAGGATAAAGTTGCAGAACTGGAAAATTCTGAGTTCAG |
| | | | AGCTTTCTCATCTTTTAAAAATAGGGTTTATTTAGACACTGTGTC |
| | | | AGGTAGCCTCACTATCTACAACTTAACATCATCAGATGAAGATG |
| | | | AGTATGAAATGGAATCGCCAAATATTACTGATACCATGAAGTTC |
| | | | TTTCTTTATGTGCTTGAGTCTCTTCCATCTCCCACACTAACTTGTG |
| | | | CATTGACTAATGGAAGCATTGAAGTCCAATGCATGATACCAGA |
| | | | GCATTACAACAGCCATCGAGGACTTATAATGTACTCATGGGATT |
| | | | GTCCTATGGAGCAATGTAAACGTAACTCAACCAGTATATATTTT |
| | | | AAGATGGAAAATGATCTTCCACAAAAAATACAGTGTACTCTTAG |
| | | | CAATCCATTATTTAATACAACATCATCAATCATTTTGACAACCTG |
| | | | TATCCCAAGCAGCGGTCATTCAAGACACAGA |
| 9 | 1961, | CD58 1D | GTTGCTGGGAGCGACGCGGGGCGGGCCCTGGGGGTCCTCAGC |
| | 1962, | | GTGGTCTGCCTGCTGCACTGCTTTGGTTTCATCAGCTGTTTTTCC |
| | 1963, | | CAACAAATATATGGTGTTGTGTATGGGAATGTAACTTTCCATGT |
| | 1965, | | ACCAAGCAATGTGCCTTTAAAAGAGGTCCTATGGAAAAAACAA |
| | 1964, | | AAGGATAAAGTTGCAGAACTGGAAAATTCTGAGTTCAGAGCTT |
| | 1966 | | TCTCATCTTTTAAAAATAGGGTTTATTTAGACACTGTGTCAGGTA |
| | | | GCCTCACTATCTACAACTTAACATCATCAGATGAAGATGAGTAT |
| | | | GAAATGGAATCGCCAAATATTACTGATACCATGAAGTTCTTTCT |
| | | | TTATGTG |
| 10 | 1961, | CD58 1D | TTTTCCCAACAAATATATGGTGTTGTGTATGGGAATGTAACTTTC |
| | 1962, | | CATGTACCAAGCAATGTGCCTTTAAAAGAGGTCCTATGGAAAA |
| | 1963, | | AACAAAAGGATAAAGTTGCAGAACTGGAAAATTCTGAGTTCAG |
| | 1965, | | AGCTTTCTCATCTTTTAAAAATAGGGTTTATTTAGACACTGTGTC |
| | 1964, | | AGGTAGCCTCACTATCTACAACTTAACATCATCAGATGAAGATG |
| | 1966 | | AGTATGAAATGGAATCGCCAAATATTACTGATACCATGAAGTTC |
| | | | TTTCTTTATGTG |
| 11 | 1967, | CD58 2D | GTTGCTGGGAGCGACGCGGGGGGGGCCCTGGGGGTCCTCAGC |
| | 1968, | | GTGGTCTGCCTGCTGCACTGCTTTGGTTTCATCAGCTGTTTTTCC |
| | 1969, | | CAACAAATATATGGTGTTGTGTATGGGAATGTAACTTTCCATGT |
| | 1970, | | ACCAAGCAATGTGCCTTTAAAAGAGGTCCTATGGAAAAAACAA |
| | 1971, | | AAGGATAAAGTTGCAGAACTGGAAAATTCTGAGTTCAGAGCTT |
| | 1972 | | TCTCATCTTTTAAAAATAGGGTTTATTTAGACACTGTGTCAGGTA |
| | | | GCCTCACTATCTACAACTTAACATCATCAGATGAAGATGAGTAT |
| | | | GAAATGGAATCGCCAAATATTACTGATACCATGAAGTTCTTTCT |
| | | | TTATGTGCTTGAGTCTCTTCCATCTCCCACACTAACTTGTGCATT |
| | | | GACTAATGGAAGCATTGAAGTCCAATGCATGATACCAGAGCAT |
| | | | TACAACAGCCATCGAGGACTTATAATGTACTCATGGGATTGTCC |
| | | | TATGGAGCAATGTAAACGTAACTCAACCAGTATATATTTTAAGA |
| | | | TGGAAAATGATCTTCCACAAAAAATACAGTGTACTCTTAGCAAT |
| | | | CCATTATTTAATACAACATCATCAATCATTTTGACAACCTGTATC |
| | | | CCAAGC |
| 12 | 1967, | CD58 2D | TTTTCCCAACAAATATATGGTGTTGTGTATGGGAATGTAACTTTC |
| | 1968, | | CATGTACCAAGCAATGTGCCTTTAAAAGAGGTCCTATGGAAAA |
| | 1969, | | AACAAAAGGATAAAGTTGCAGAACTGGAAAATTCTGAGTTCAG |
| | 1970, | | AGCTTTCTCATCTTTTAAAAATAGGGTTTATTTAGACACTGTGTC |
| | 1971, | | AGGTAGCCTCACTATCTACAACTTAACATCATCAGATGAAGATG |
| | 1972 | | AGTATGAAATGGAATCGCCAAATATTACTGATACCATGAAGTTC |
| | | | TTTCTTTATGTGCTTGAGTCTCTTCCATCTCCCACACTAACTTGTG |
| | | | CATTGACTAATGGAAGCATTGAAGTCCAATGCATGATACCAGA |
| | | | GCATTACAACAGCCATCGAGGACTTATAATGTACTCATGGGATT |
| | | | GTCCTATGGAGCAATGTAAACGTAACTCAACCAGTATATATTTT |
| | | | AAGATGGAAAATGATCTTCCACAAAAAATACAGTGTACTCTTAG |
| | | | CAATCCATTATTTAATACAACATCATCAATCATTTTGACAACCTG |
| | | | TATCCCAAGC |
| 13 | 1973, | CD2 scFv | GCCTTACCAGTGACCGCCTTGCTCCTGCCGCTGGCCTTGCTGCT |
| | 1974, | | CCACGCCGCCAGGCCGGACGTGGTGATGACCCAGAGCCCCCCC |
| | 1975, | | AGCCTGCTGGTGACCCTGGGCCAGCCCGCCAGCATCAGCTGCA |
| | 1976, | | GAAGCAGCCAGAGCCTGCTGCACAGCAGCGGCAACACCTACCT |
| | 1977, | | GAACTGGCTGCTGCAGAGACCCGGCCAGAGCCCCCAGCCCCTG |
| | 1978 | | ATCTACCTGGTGAGCAAGCTGGAGAGCGGCGTGCCCGACAGAT |
| | | | TCAGCGGCAGCGGCAGCGGCACCGACTTCACCCTGAAGATCAG |
| | | | CGGCGTGGAGGCCGAGGACGTGGGCGTGTACTACTGCATGCA |
| | | | GTTCACCCACTACCCCTACACCTTCGGCCAGGGCACCAAGCTGG |
| | | | AGATCAAGGGCGGCGGCGGCAGCGGCGGCGGCGGCAGCGGC |
| | | | GGCGGCGGCAGCCAGGTGCAGCTGGTGCAGAGCGGCGCCGAG |
| | | | GTGAAGAAGCCCGGCGCCAGCGTGAAGGTGAGCTGCAAGGCC |
| | | | AGCGGCTACACCTTCACCGAGTACTACATGTACTGGGTGAGAC |
| | | | AGGCCCCCGGCCAGGGCCTGGAGCTGATGGGCAGAATCGACC |
| | | | CCGAGGACGGCAGCATCGACTACGTGGAGAAGTTCAAGAAGA |
| | | | AGGTGACCCTGACCGCCGACACCAGCAGCAGCACCGCCTACAT |
| | | | GGAGCTGAGCAGCCTGACCAGCGACGACACCGCCGTGTACTAC |
| | | | TGCGCCAGAGGCAAGTTCAACTACAGATTCGCCTACTGGGGCC |
| | | | AGGGCACCCTGGTGACCGTGAGCAGC |
| 14 | 1973, | CD2 scFv | GACGTGGTGATGACCCAGAGCCCCCCCAGCCTGCTGGTGACCC |
| | 1974, | with a | TGGGCCAGCCCGCCAGCATCAGCTGCAGAAGCAGCCAGAGCCT |
| | 1975, | (Gly₄Ser)₃ | GCTGCACAGCAGCGGCAACACCTACCTGAACTGGCTGCTGCAG |
| | 1976, | between V_{L} | AGACCCGGCCAGAGCCCCCAGCCCCTGATCTACCTGGTGAGCA |
| | 1977, | and V_{H} | AGCTGGAGAGCGGCGTGCCCGACAGATTCAGCGGCAGCGGCA |
| | 1978 | (denoted by | GCGGCACCGACTTCACCCTGAAGATCAGCGGCGTGGAGGCCGA |
| | | bold and | GGACGTGGGCGTGTACTACTGCATGCAGTTCACCCACTACCCCT |
| | | underlined | ACACCTTCGGCCAGGGCACCAAGCTGGAGATCAAGGGCGGCG |
| | | lettering) | GCGGCAGCGGCGGCGGCGGCAGCGGCGGCGGCGGCAGCCAG |
| | | | GTGCAGCTGGTGCAGAGCGGCGCCGAGGTGAAGAAGCCCGGC |
| | | | GCCAGCGTGAAGGTGAGCTGCAAGGCCAGCGGCTACACCTTCA |
| | | | CCGAGTACTACATGTACTGGGTGAGACAGGCCCCCGGCCAGGG |
| | | | CCTGGAGCTGATGGGCAGAATCGACCCCGAGGACGGCAGCAT |
| | | | CGACTACGTGGAGAAGTTCAAGAAGAAGGTGACCCTGACCGCC |
| | | | GACACCAGCAGCAGCACCGCCTACATGGAGCTGAGCAGCCTGA |
| | | | CCAGCGACGACACCGCCGTGTACTACTGCGCCAGAGGCAAGTT |
| | | | CAACTACAGATTCGCCTACTGGGGCCAGGGCACCCTGGTGACC |
| | | | GTGAGCAGC |

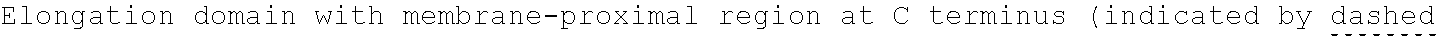

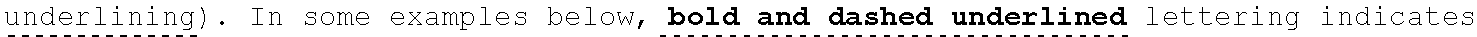

restriction sites.

| | | | |
|---|---|---|---|
| 15 | 1882, | CD22 3 | 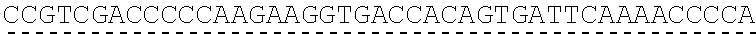 |
| | 1885 | exons | 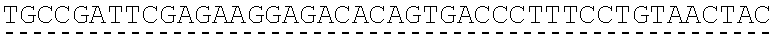 |
| | | | 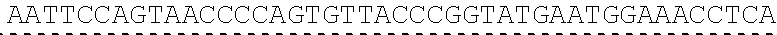 |
| | | | 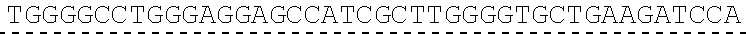 |
| | | | 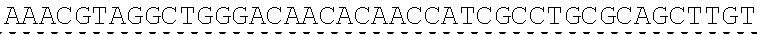 |
| | | | 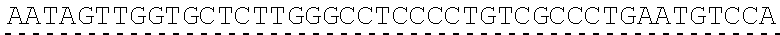 |
| | | | 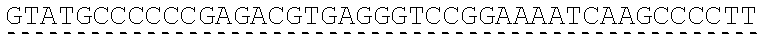 |
| | | | 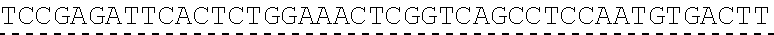 |
| | | | 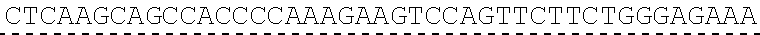 |
| | | | 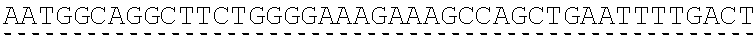 |
| | | | 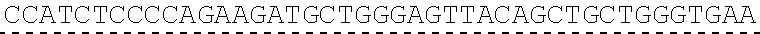 |
| | | | 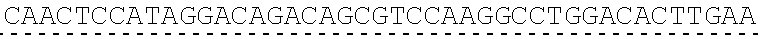 |
| | | | 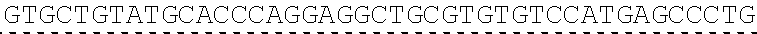 |
| | | | 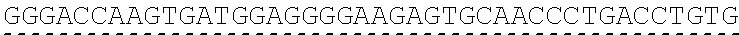 |
| | | | 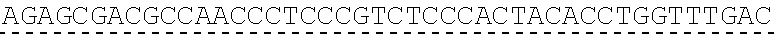 |
| | | | 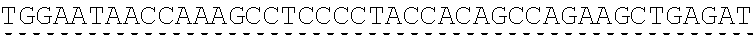 |
| | | | 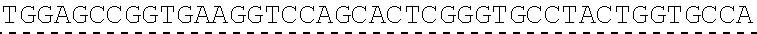 |
| | | | 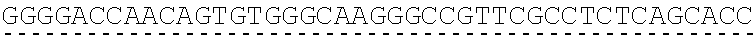 |
| | | | 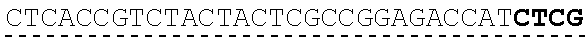 |
| 16 | 1883, | CD45RO | 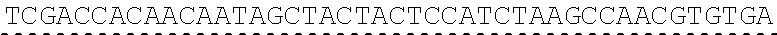 |
| | 1886 | | 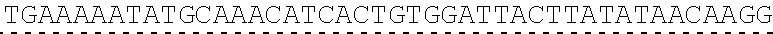 |
| | | | 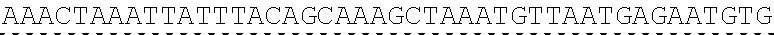 |
| | | | 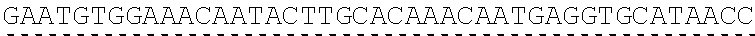 |
| | | | 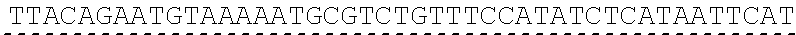 |
| | | | 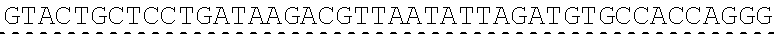 |
| | | | 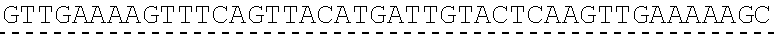 |
| | | | 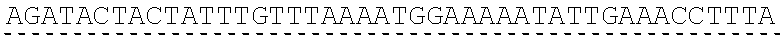 |
| | | | 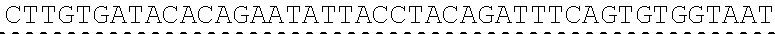 |
| | | | 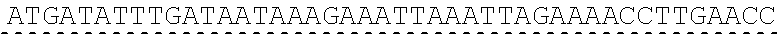 |
| | | | 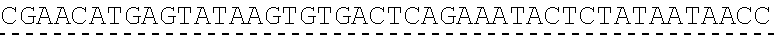 |
| | | | 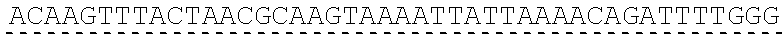 |
| | | | 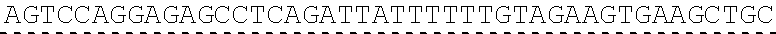 |
| | | | 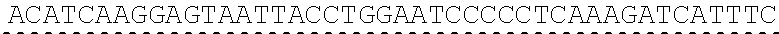 |
| | | | 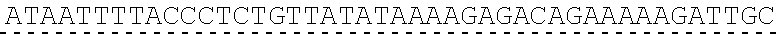 |
| | | | 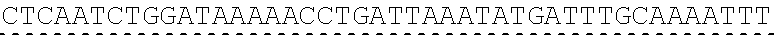 |
| | | | 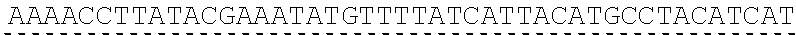 |
| | | | 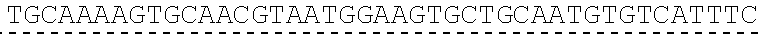 |
| | | | 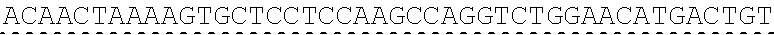 |
| | | | 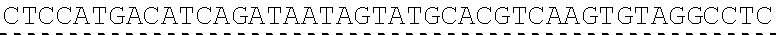 |
| | | | 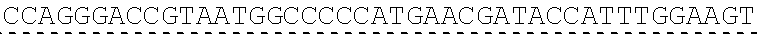 |
| | | | 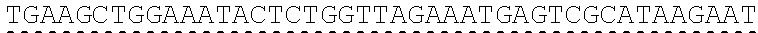 |
| | | | 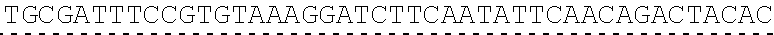 |
| | | | 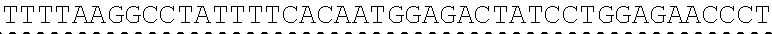 |
| | | | 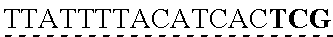 |
| 17 | | CD45RO | 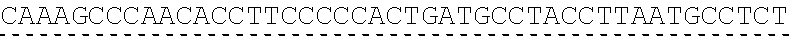 |
| | | | 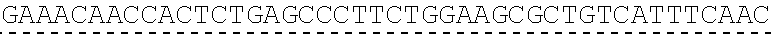 |
| | | | 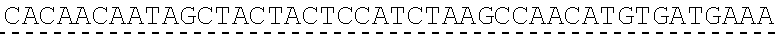 |
| | | | 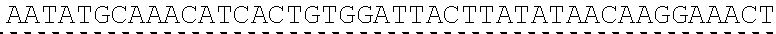 |
| | | | 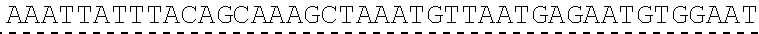 |
| | | | 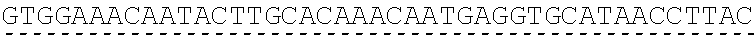 |
| | | | 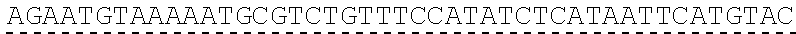 |
| | | | 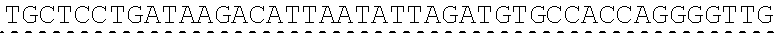 |
| | | | 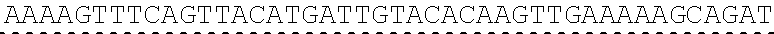 |
| | | | 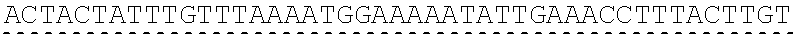 |
| | | | 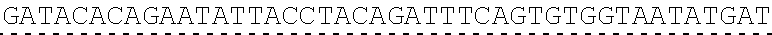 |
| | | | 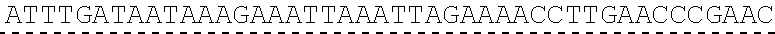 |
| | | | 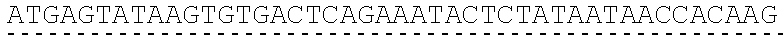 |
| | | | 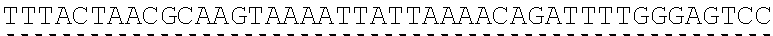 |
| | | | 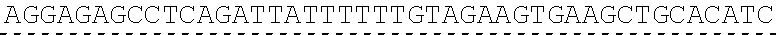 |
| | | | 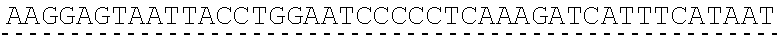 |
| | | | 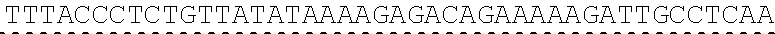 |
| | | | 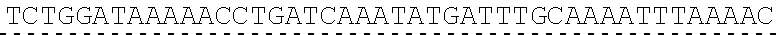 |
| | | | 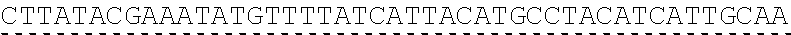 |
| | | | 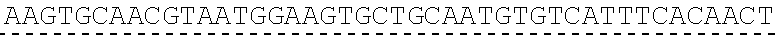 |
| | | | 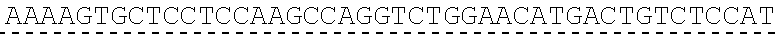 |
| | | | 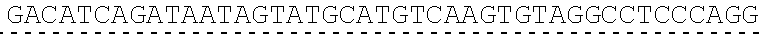 |
| | | | 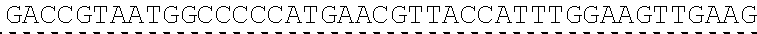 |
| | | | 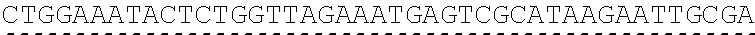 |
| | | | 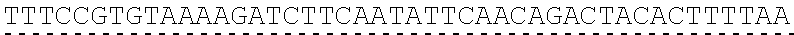 |
| | | | 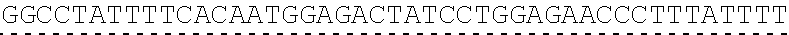 |
| | | | 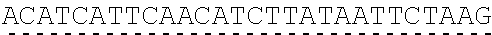 |
| 18 | 1884, | CD45RABC | 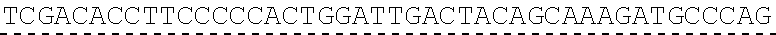 |
| | 1886 | | 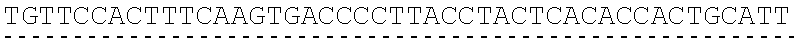 |
| | | | 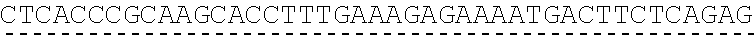 |
| | | | 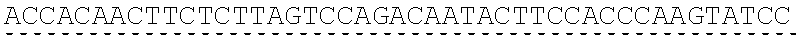 |
| | | | 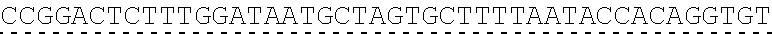 |
| | | | 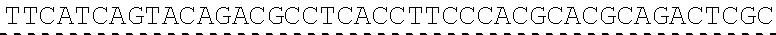 |
| | | | 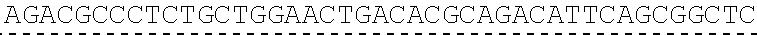 |
| | | | 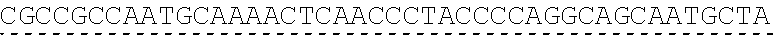 |
| | | | 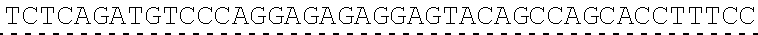 |
| | | | 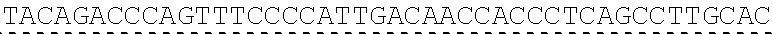 |
| | | | 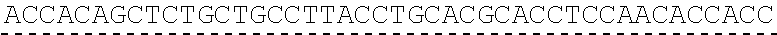 |
| | | | 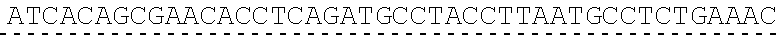 |
| | | | 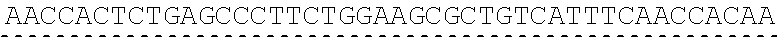 |
| | | | 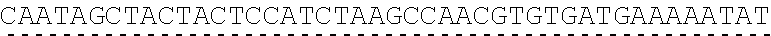 |
| | | | 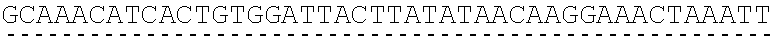 |
| | | | 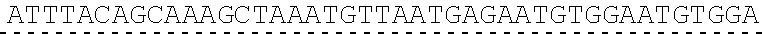 |
| | | | 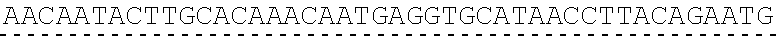 |
| | | | 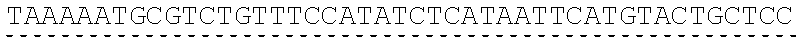 |
| | | | 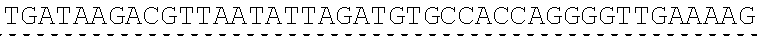 |
| | | | 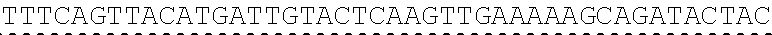 |
| | | | 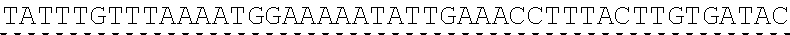 |
| | | | 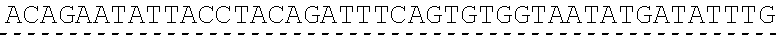 |
| | | | 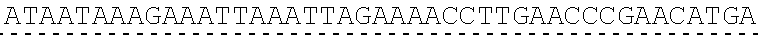 |
| | | | 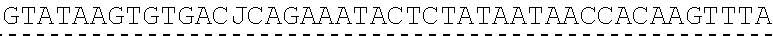 |
| | | | 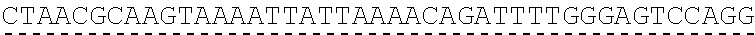 |
| | | | 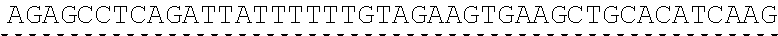 |
| | | | 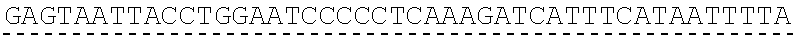 |
| | | | 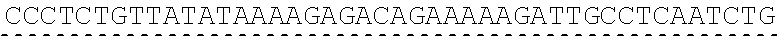 |
| | | | 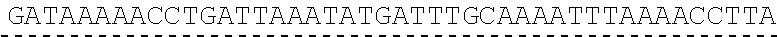 |
| | | | 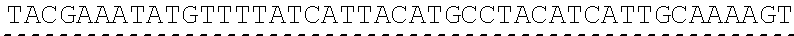 |
| | | | 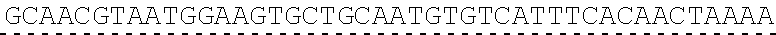 |
| | | | 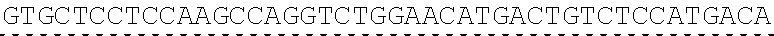 |
| | | | 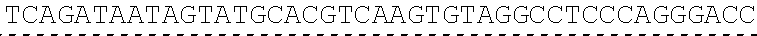 |
| | | | 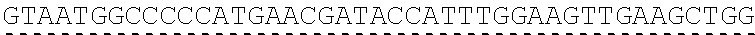 |
| | | | 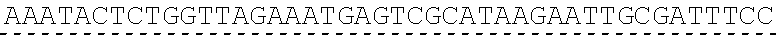 |
| | | | 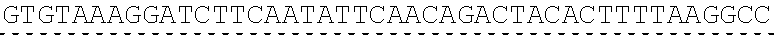 |
| | | | 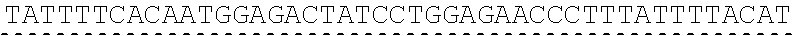 |
| | | | 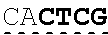 |
| 19 | 1941 | CD22 5 | 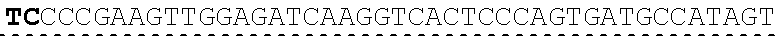 |
| | | exons | 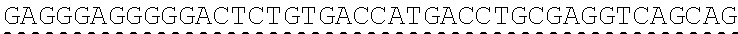 |
| | | | 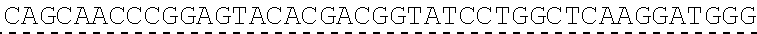 |
| | | | 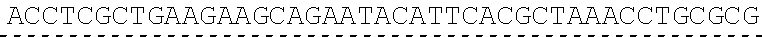 |
| | | | 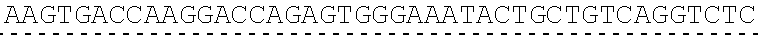 |
| | | | 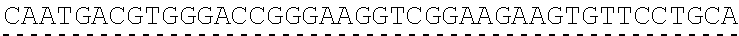 |
| | | | 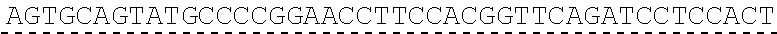 |
| | | | 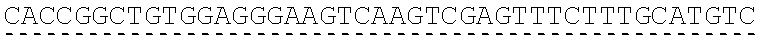 |
| | | | 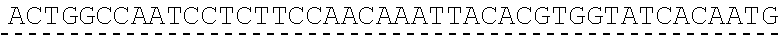 |
| | | | 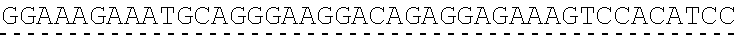 |
| | | | 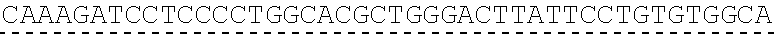 |
| | | | 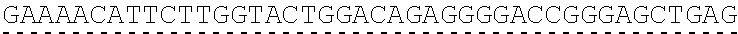 |
| | | | 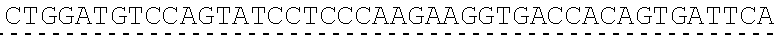 |
| | | | 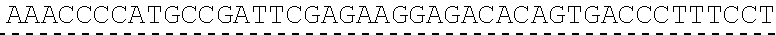 |
| | | | 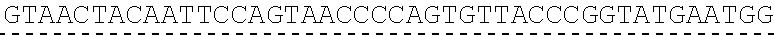 |
| | | | 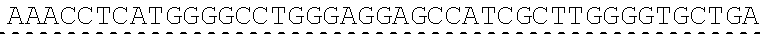 |
| | | | 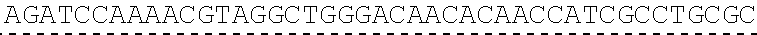 |
| | | | 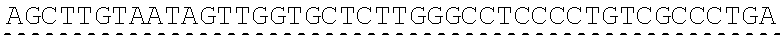 |
| | | | 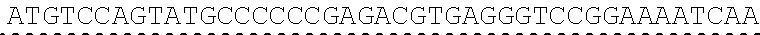 |
| | | | 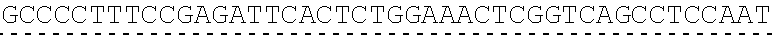 |
| | | | 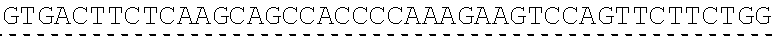 |
| | | | 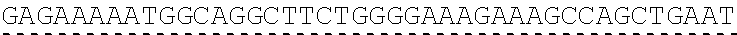 |
| | | | 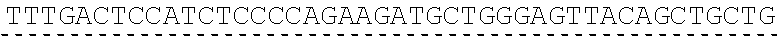 |
| | | | 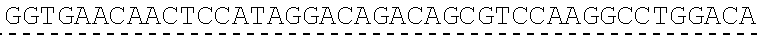 |
| | | | 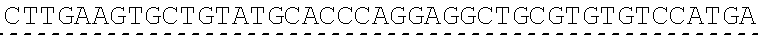 |
| | | | 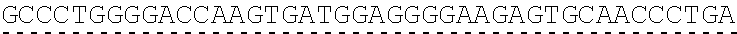 |
| | | | 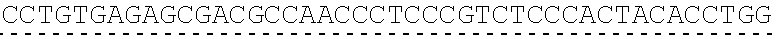 |
| | | | 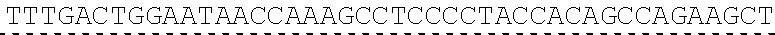 |
| | | | 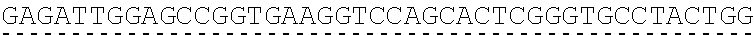 |
| | | | 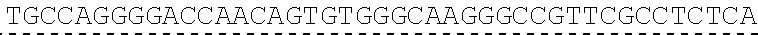 |
| | | | 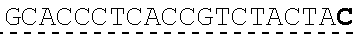 |
| 20 | 1944 | CD22 5 | 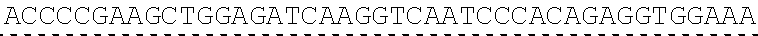 |
| | | exons | 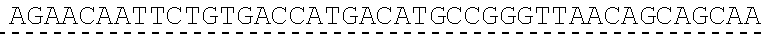 |
| | | | 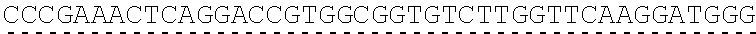 |
| | | | 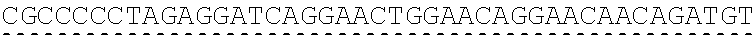 |
| | | | 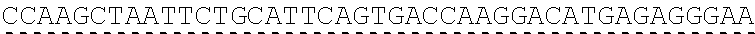 |
| | | | 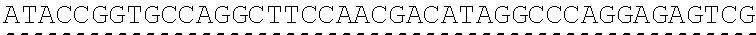 |
| | | | 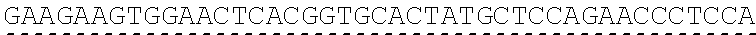 |
| | | | 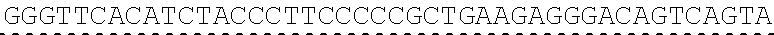 |
| | | | 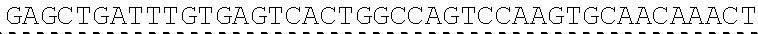 |
| | | | 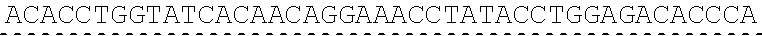 |
| | | | 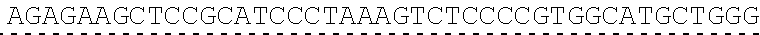 |
| | | | 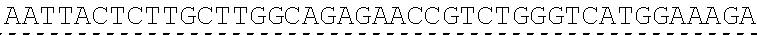 |
| | | | 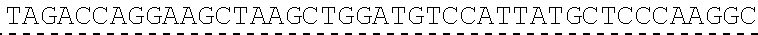 |
| | | | 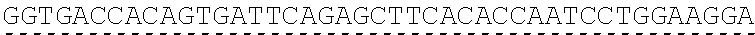 |
| | | | 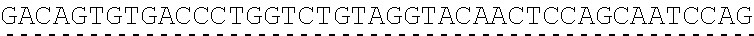 |
| | | | 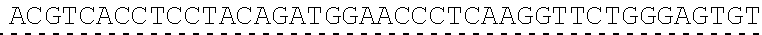 |
| | | | 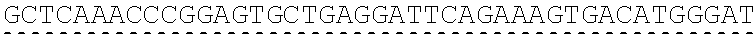 |
| | | | 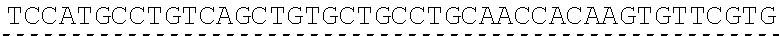 |
| | | | 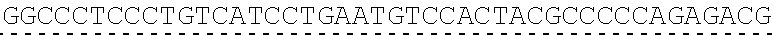 |
| | | | 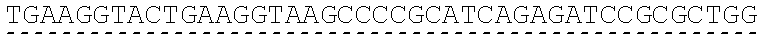 |
| | | | 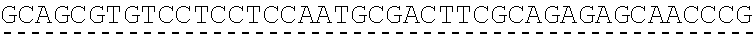 |
| | | | 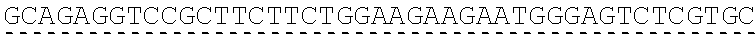 |
| | | | 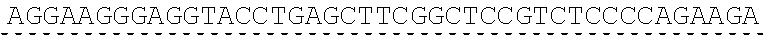 |
| | | | 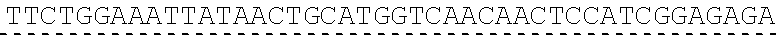 |
| | | | 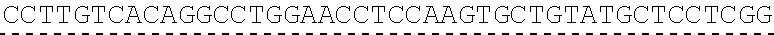 |
| | | | 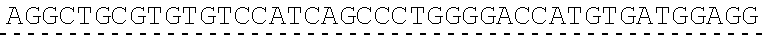 |
| | | | 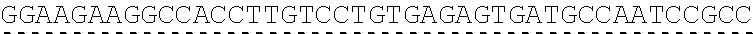 |
| | | | 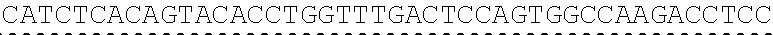 |
| | | | 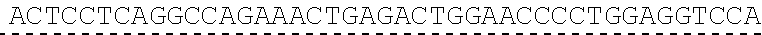 |
| | | | 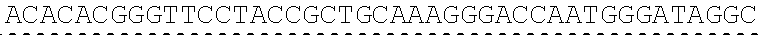 |
| | | | 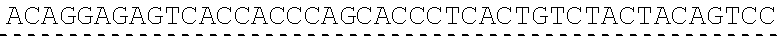 |
| | | | 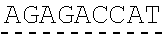 |
| 21 | | CD22 (five | 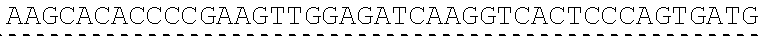 |
| | | Ig-like, | 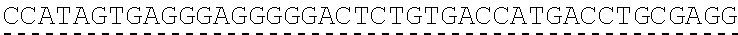 |
| | | membrane- | 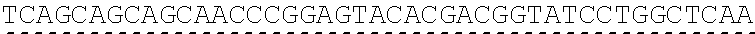 |
| | | proximal | 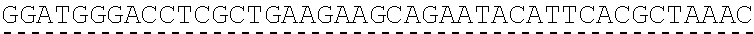 |
| | | extracellular | 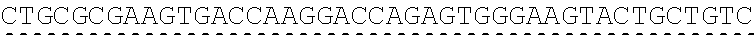 |
| | | domains) | 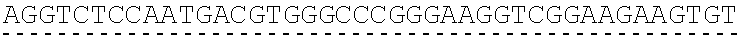 |
| | | | 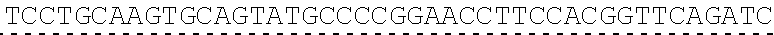 |
| | | | 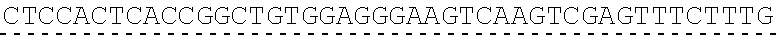 |
| | | | 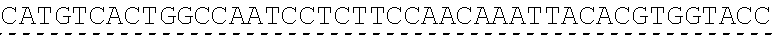 |
| | | | 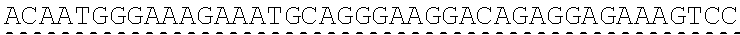 |
| | | | 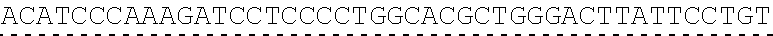 |
| | | | 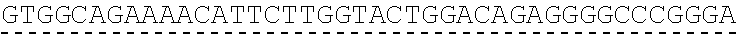 |
| | | | 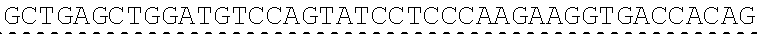 |
| | | | 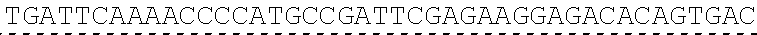 |
| | | | 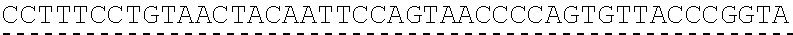 |
| | | | 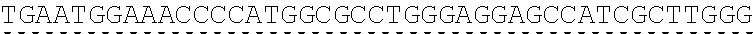 |
| | | | 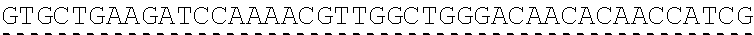 |
| | | | 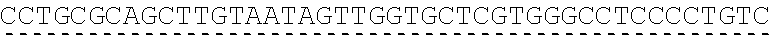 |
| | | | 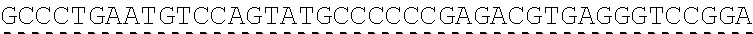 |
| | | | 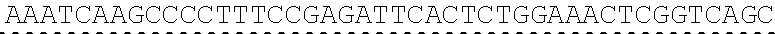 |
| | | | 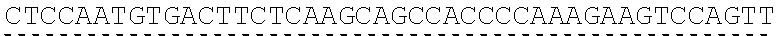 |
| | | | 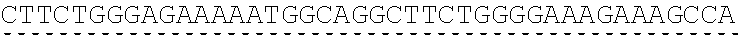 |
| | | | 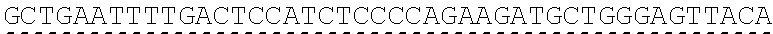 |
| | | | 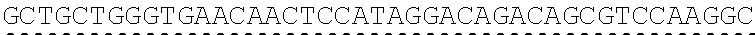 |
| | | | 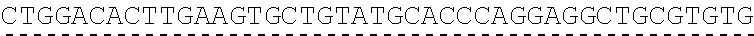 |
| | | | 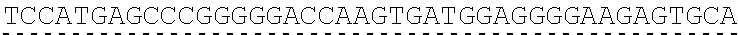 |
| | | | 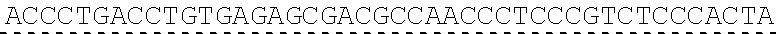 |
| | | | 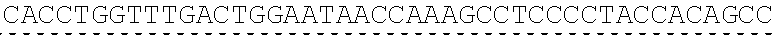 |
| | | | 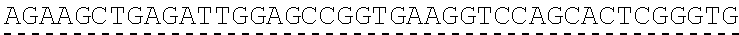 |
| | | | 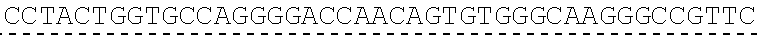 |
| | | | 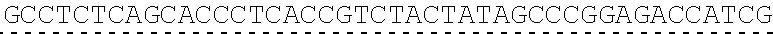 |
| | | | 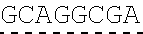 |
| 22 | 1942 | CD45RABC | 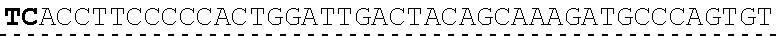 |
| | | | 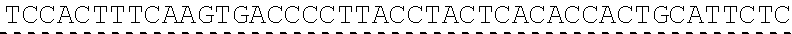 |
| | | | 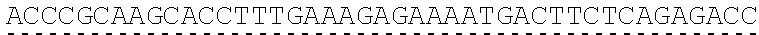 |
| | | | 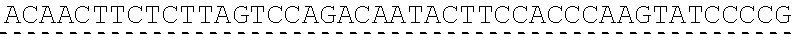 |
| | | | 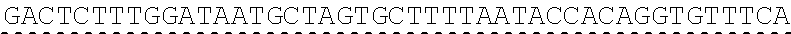 |
| | | | 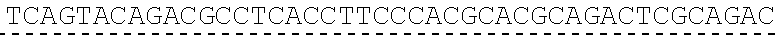 |
| | | | 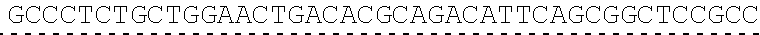 |
| | | | 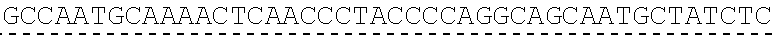 |
| | | | 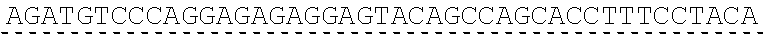 |
| | | | 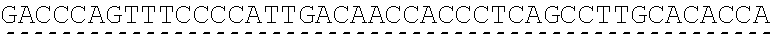 |
| | | | 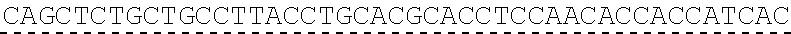 |
| | | | 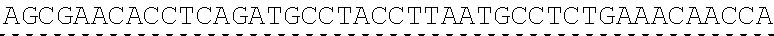 |
| | | | 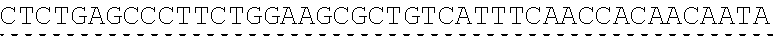 |
| | | | 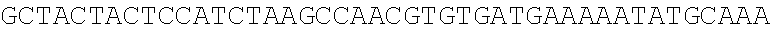 |
| | | | 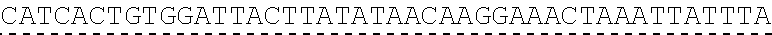 |
| | | | 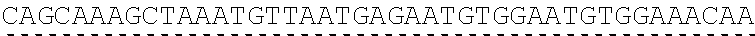 |
| | | | 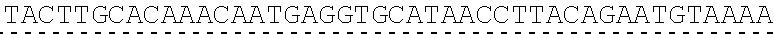 |
| | | | 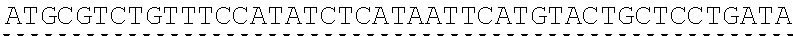 |
| | | | 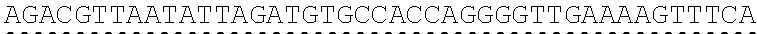 |
| | | | 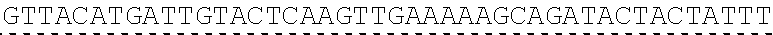 |
| | | | 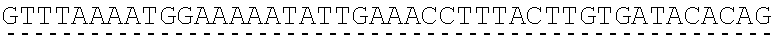 |
| | | | 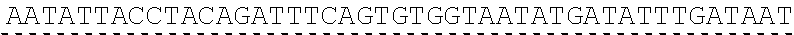 |
| | | | 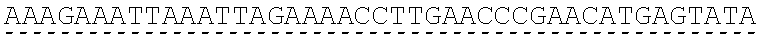 |
| | | | 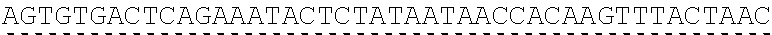 |
| | | | 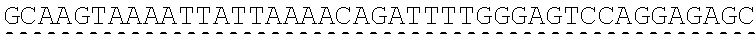 |
| | | | 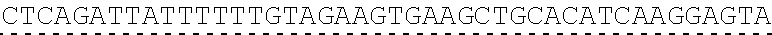 |
| | | | 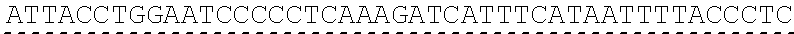 |
| | | | 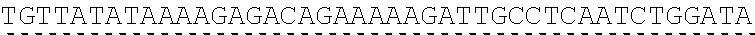 |
| | | | 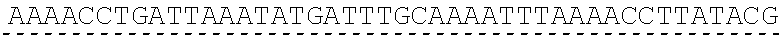 |
| | | | 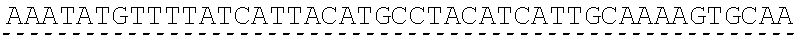 |
| | | | 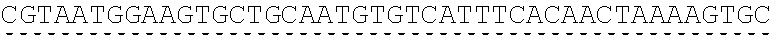 |
| | | | 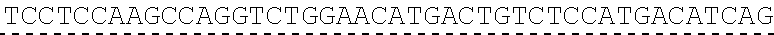 |
| | | | 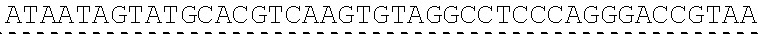 |
| | | | 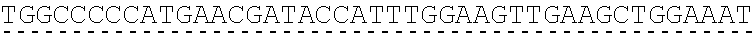 |
| | | | 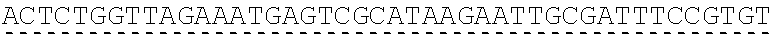 |
| | | | 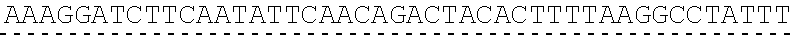 |
| | | | 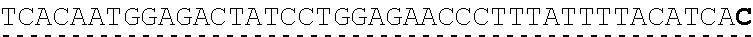 |
| 23 | 1945 | CD45RABC | 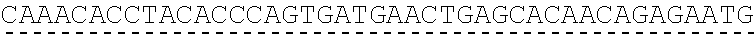 |
| | | | 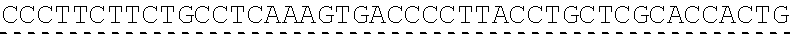 |
| | | | 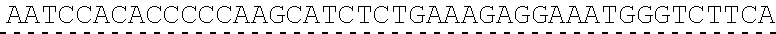 |
| | | | 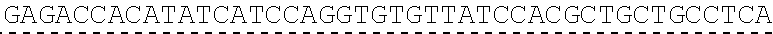 |
| | | | 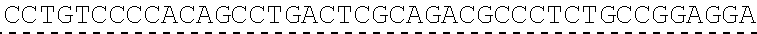 |
| | | | 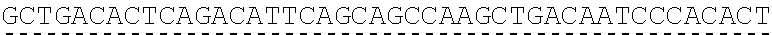 |
| | | | 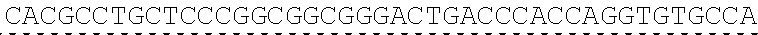 |
| | | | 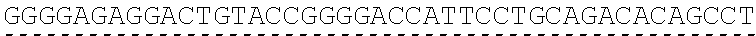 |
| | | | 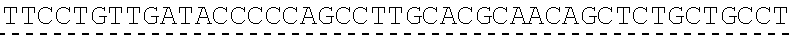 |
| | | | 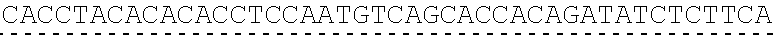 |
| | | | 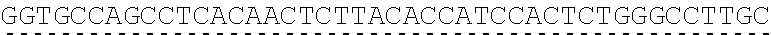 |
| | | | 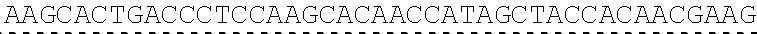 |
| | | | 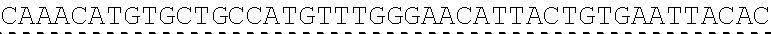 |
| | | | 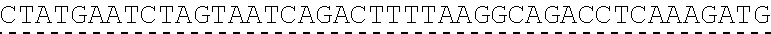 |
| | | | 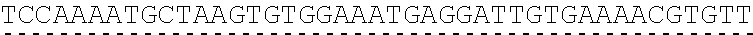 |
| | | | 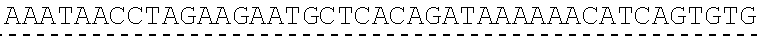 |
| | | | 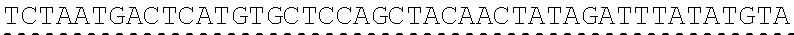 |
| | | | 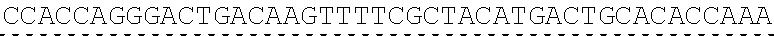 |
| | | | 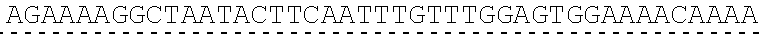 |
| | | | 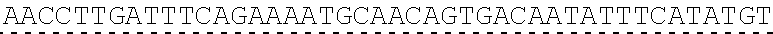 |
| | | | 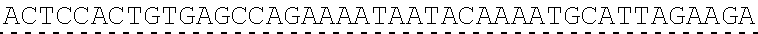 |
| | | | 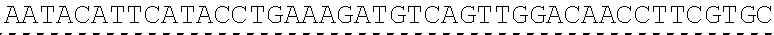 |
| | | | 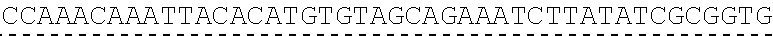 |
| | | | 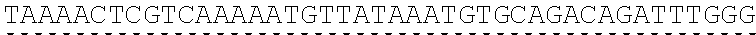 |
| | | | 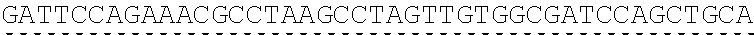 |
| | | | 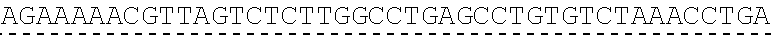 |
| | | | 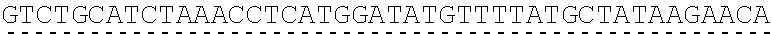 |
| | | | 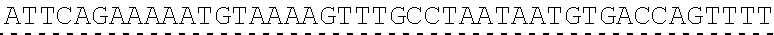 |
| | | | 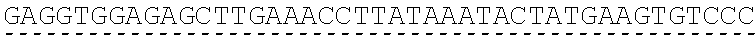 |
| | | | 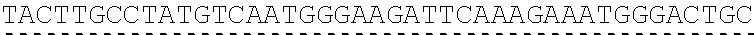 |
| | | | 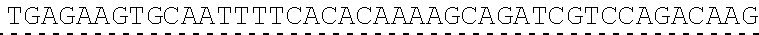 |
| | | | 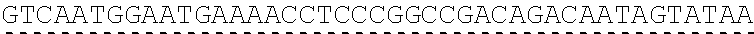 |
| | | | 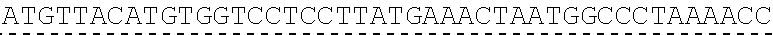 |
| | | | 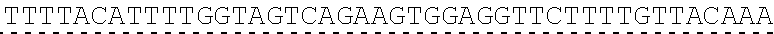 |
| | | | 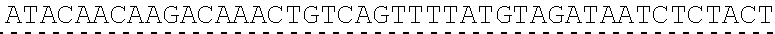 |
| | | | 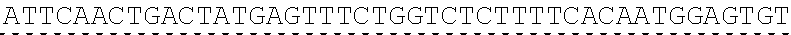 |
| | | | 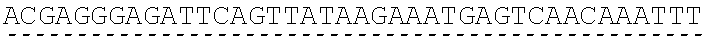 |
| 24 | | CD45RABC | 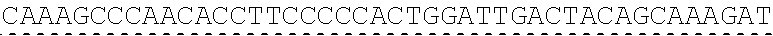 |
| | | | 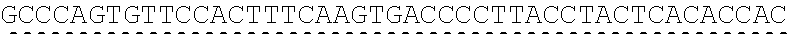 |
| | | | 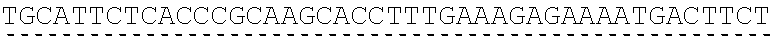 |
| | | | 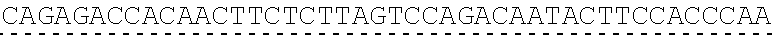 |
| | | | 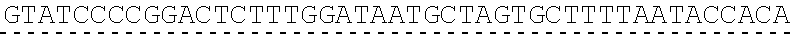 |
| | | | 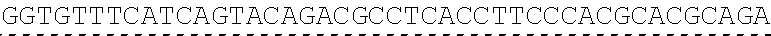 |
| | | | 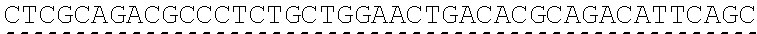 |
| | | | 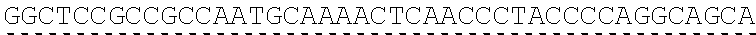 |
| | | | 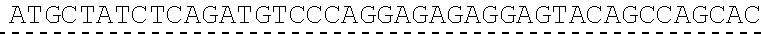 |
| | | | 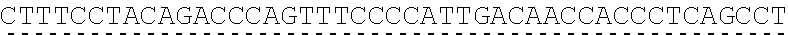 |
| | | | 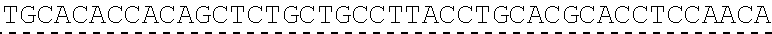 |
| | | | 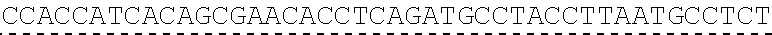 |
| | | | 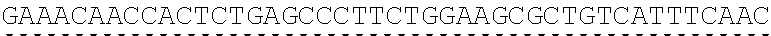 |
| | | | 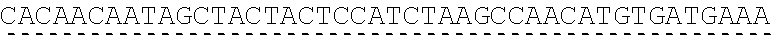 |
| | | | 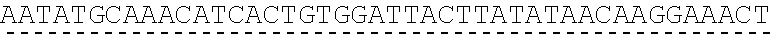 |
| | | | 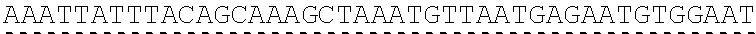 |
| | | | 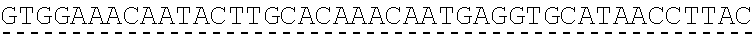 |
| | | | 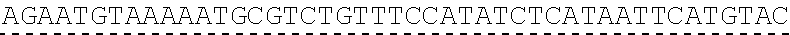 |
| | | | 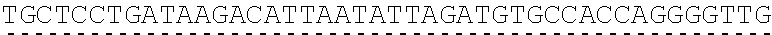 |
| | | | 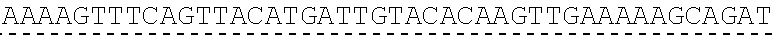 |
| | | | 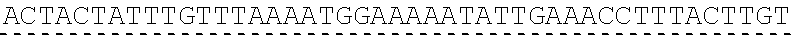 |
| | | | 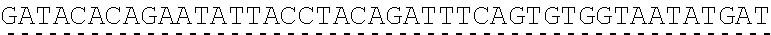 |
| | | | 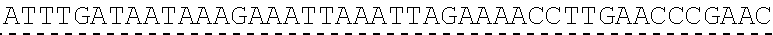 |
| | | | 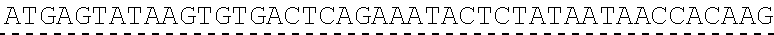 |
| | | | 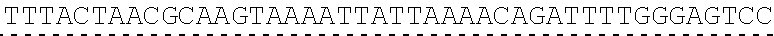 |
| | | | 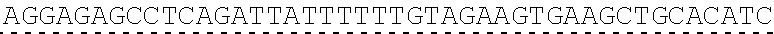 |
| | | | 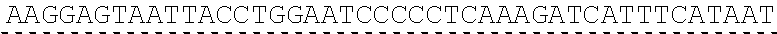 |
| | | | 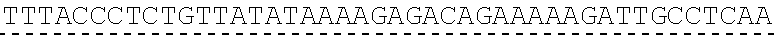 |
| | | | 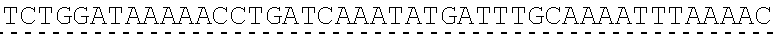 |
| | | | 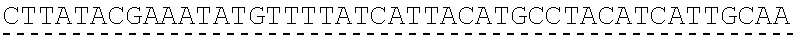 |
| | | | 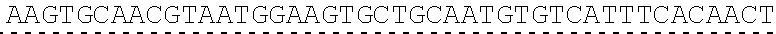 |
| | | | 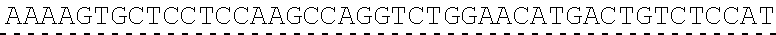 |
| | | | 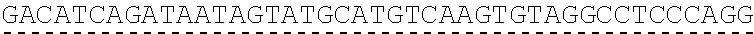 |
| | | | 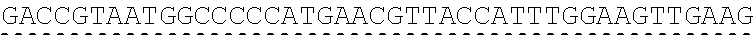 |
| | | | 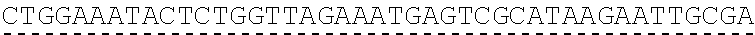 |
| | | | 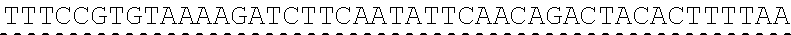 |
| | | | 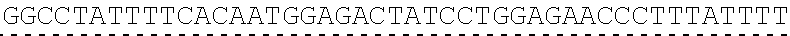 |
| | | | 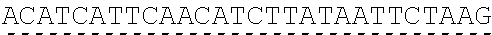 |

Elongation/(optional)extracellular membrane-proximal/transmembrane/intracellular

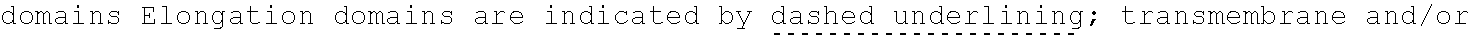

intracellular domains are indicated by double underlined lettering with the transmembrane

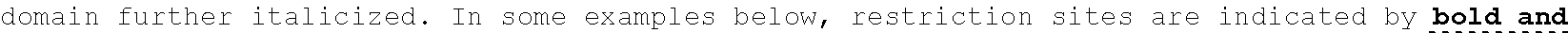

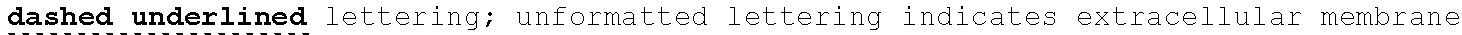

proximal domains.

| | | | |
|---|---|---|---|
| 25 | 1943 | CD45RABC + | 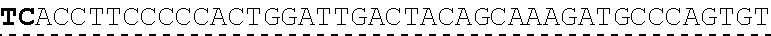 |
| | | tm + 12aa | 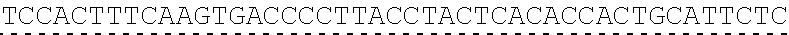 |
| | | | 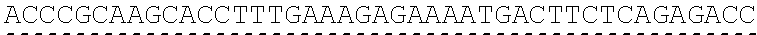 |
| | | | 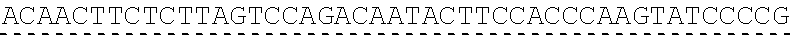 |
| | | | 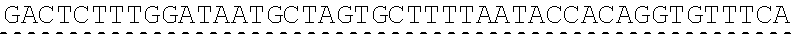 |
| | | | 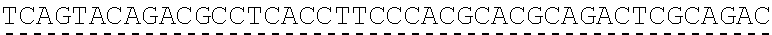 |
| | | | 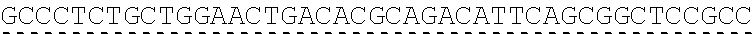 |
| | | | 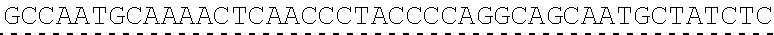 |
| | | | 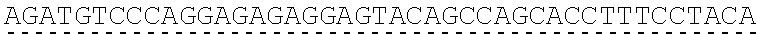 |
| | | | 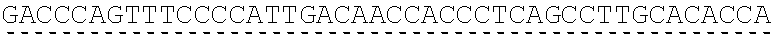 |
| | | | 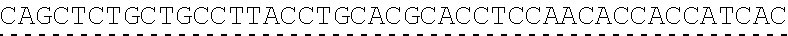 |
| | | | 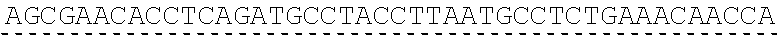 |
| | | | 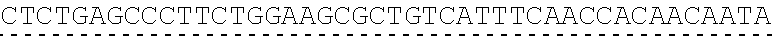 |
| | | | 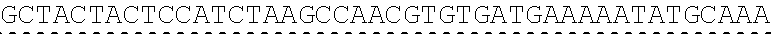 |
| | | | 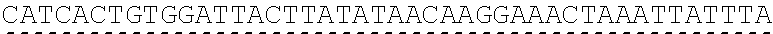 |
| | | | 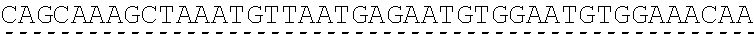 |
| | | | 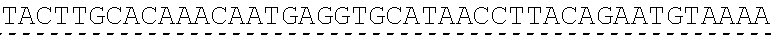 |
| | | | 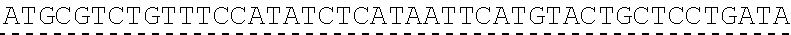 |
| | | | 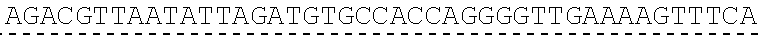 |
| | | | 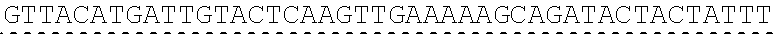 |
| | | | 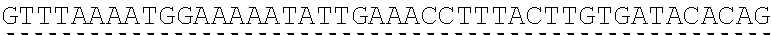 |
| | | | 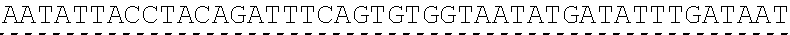 |
| | | | 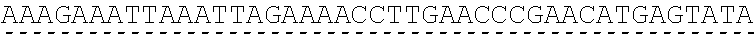 |
| | | | 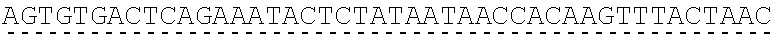 |
| | | | 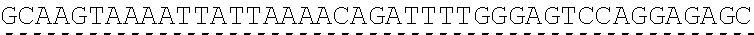 |
| | | | 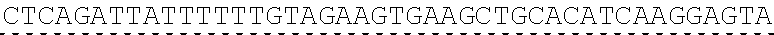 |
| | | | 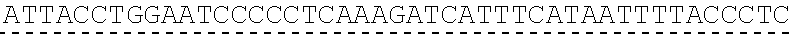 |
| | | | 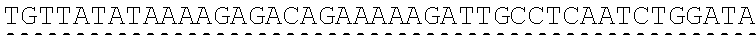 |
| | | | 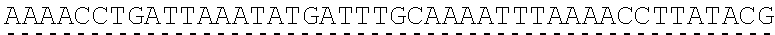 |
| | | | 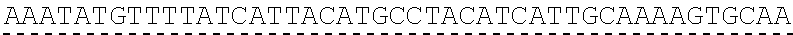 |
| | | | 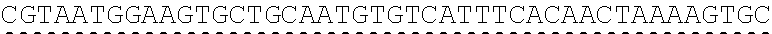 |
| | | | 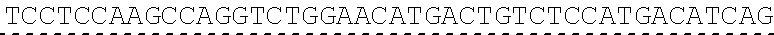 |
| | | | 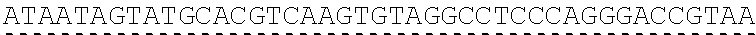 |
| | | | 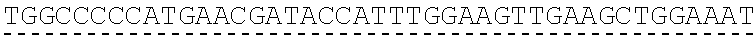 |
| | | | 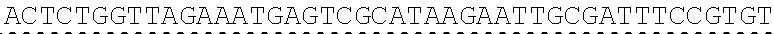 |
| | | | 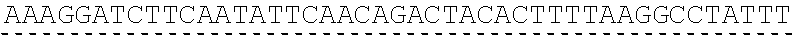 |
| | | | 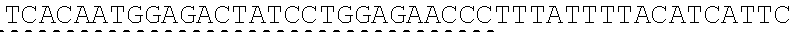 |
| | | | AACATCTTATAATTCTAAGGCACTGATAGCATTTCTGGCATTTCT |
| | | | GATTATTGTGACATCAATAGCCCTGCTTGTTGTTCTCTACAAAAT |
| | | | CTATGATCTACATAAGAAAAGATCCTGCAAT |
| 26 | 1946 | CD45RABC + | 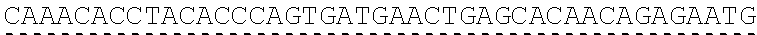 |
| | | tm +12 aa | 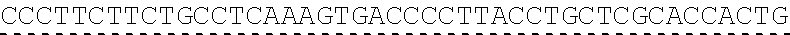 |
| | | | 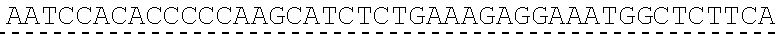 |
| | | | 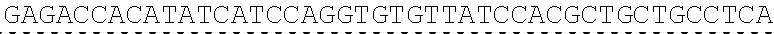 |
| | | | 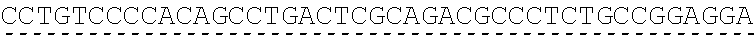 |
| | | | 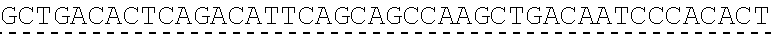 |
| | | | 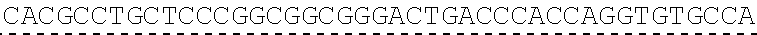 |
| | | | 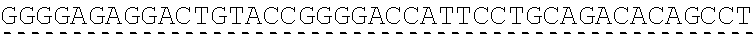 |
| | | | 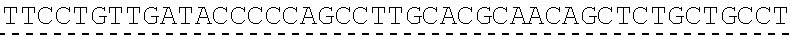 |
| | | | 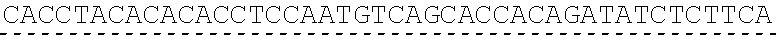 |
| | | | 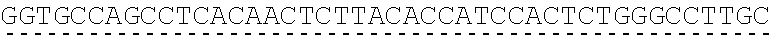 |
| | | | 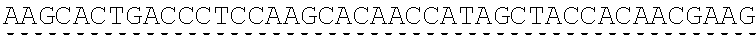 |
| | | | 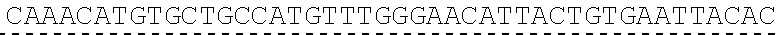 |
| | | | 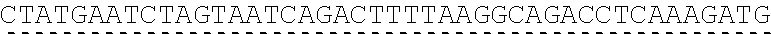 |
| | | | 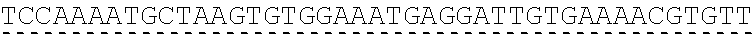 |
| | | | 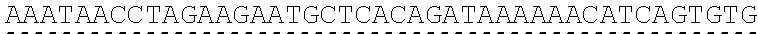 |
| | | | 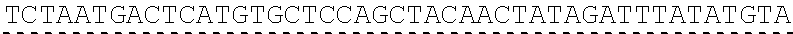 |
| | | | 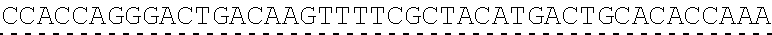 |
| | | | 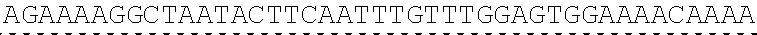 |
| | | | 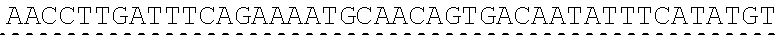 |
| | | | 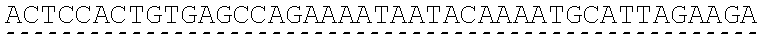 |
| | | | 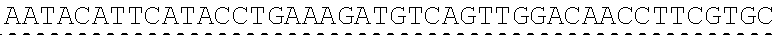 |
| | | | 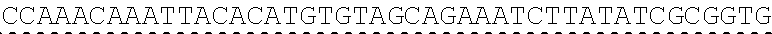 |
| | | | 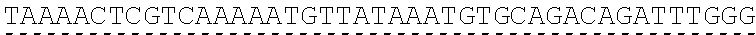 |
| | | | 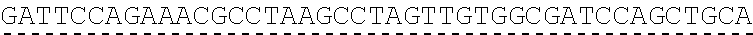 |
| | | | 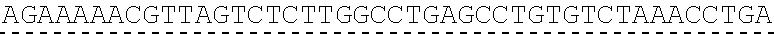 |
| | | | 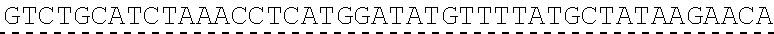 |
| | | | 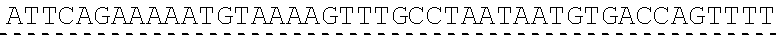 |
| | | | 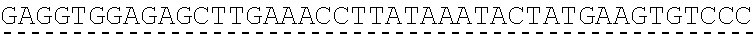 |
| | | | 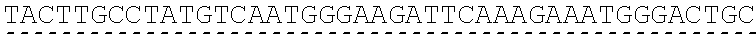 |
| | | | 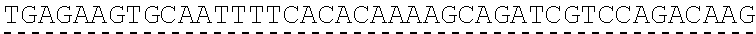 |
| | | | 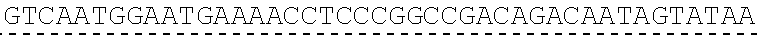 |
| | | | 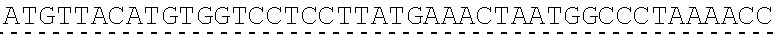 |
| | | | 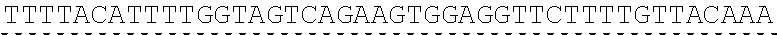 |
| | | | 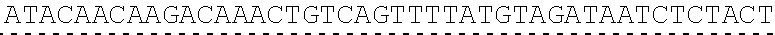 |
| | | | 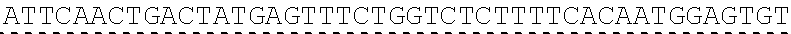 |
| | | | 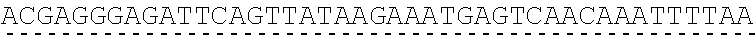 |
| | | | 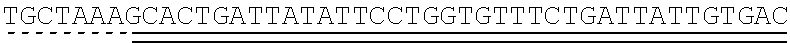 |
| | | | ATCAATAGCCTTGCTTGTTGTTTTGTATAAAATCTATGATCTGCG |
| | | | CAAGAAAAGATCCAGCAAT |
| 27 | 1961, | CD22 5D + | 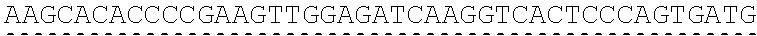 |
| | 1962, | tm | 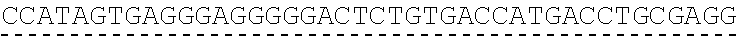 |
| | 1967, | | 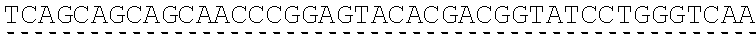 |
| | 1968, | | 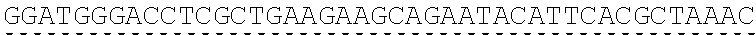 |
| | 1973, | | 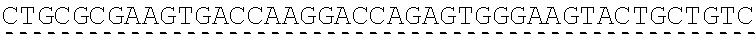 |
| | 1974, | | 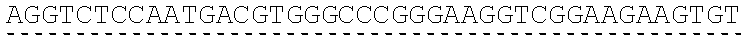 |
| | | | 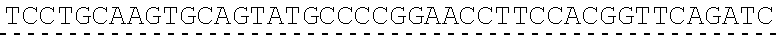 |
| | | | 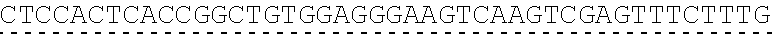 |
| | | | 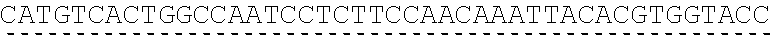 |
| | | | 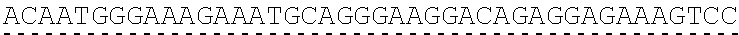 |
| | | | 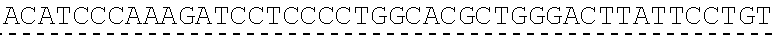 |
| | | | 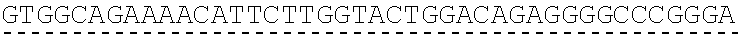 |
| | | | 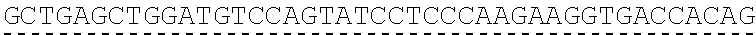 |
| | | | 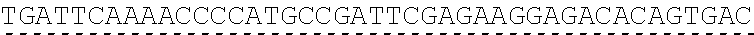 |
| | | | 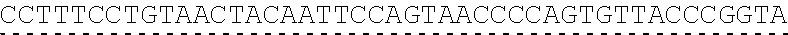 |
| | | | 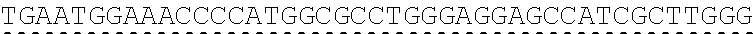 |
| | | | 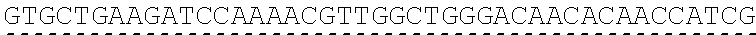 |
| | | | 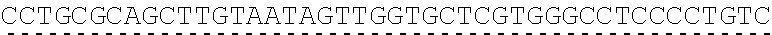 |
| | | | 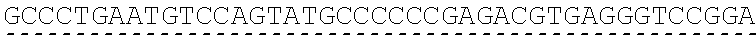 |
| | | | 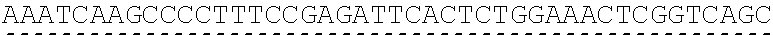 |
| | | | 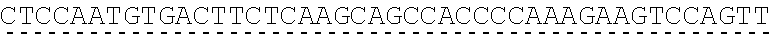 |
| | | | 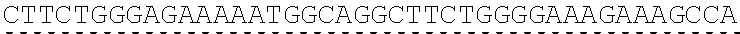 |
| | | | 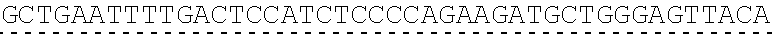 |
| | | | 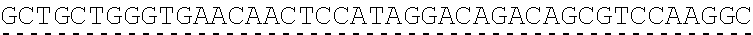 |
| | | | 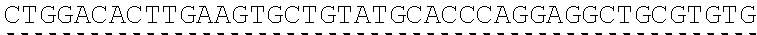 |
| | | | 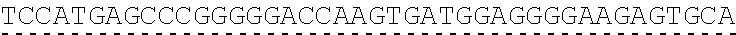 |
| | | | 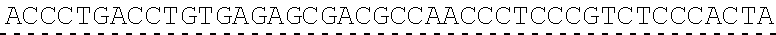 |
| | | | 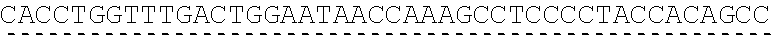 |
| | | | 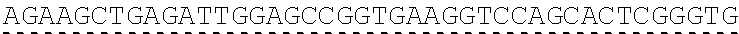 |
| | | | 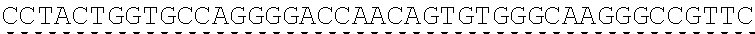 |
| | | | 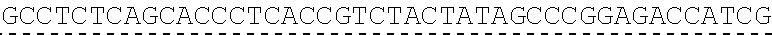 |
| | | | 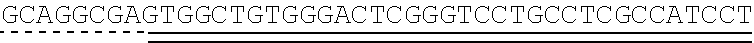 |
| | | | CATCCTGGCAATCTGTGGGCTCAAGCTCCAGCGACGTTGGAAG |
| | | | AGGACACAGAGCCAGCAGGGG |
| 28 | 1963, | CD45RO | 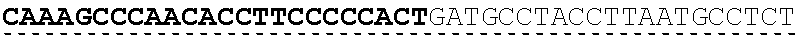 |
| | 1975, | | 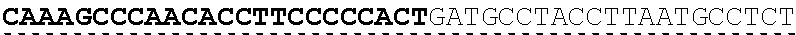 |
| | 1964, | | 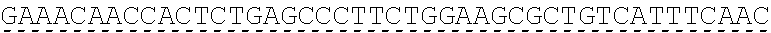 |
| | 1969, | | 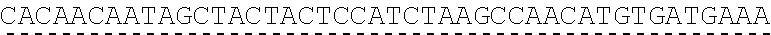 |
| | 1970, | | 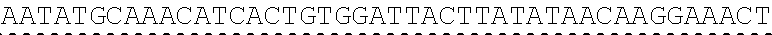 |
| | 1976 | | 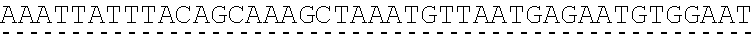 |
| | | | 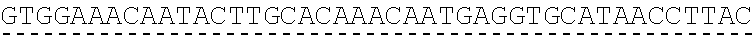 |
| | | | 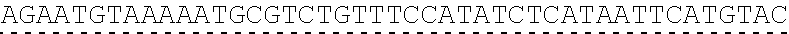 |
| | | | 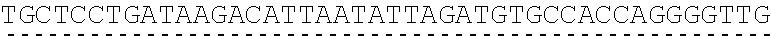 |
| | | | 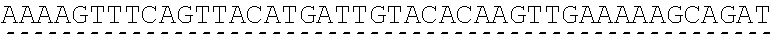 |
| | | | 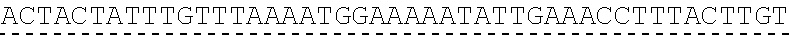 |
| | | | 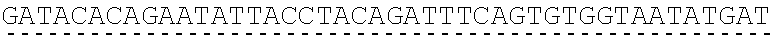 |
| | | | 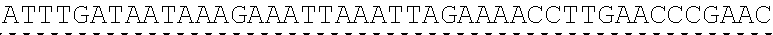 |
| | | | 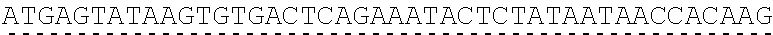 |
| | | | 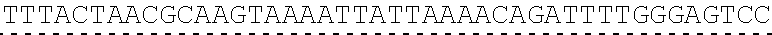 |
| | | | 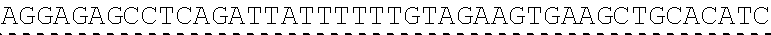 |
| | | | 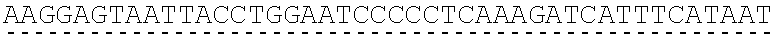 |
| | | | 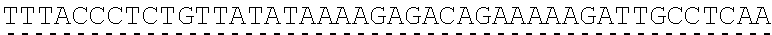 |
| | | | 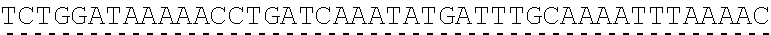 |
| | | | 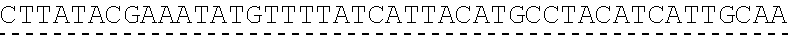 |
| | | | 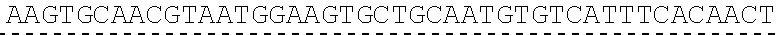 |
| | | | 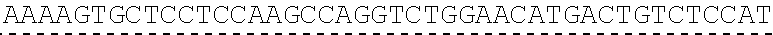 |
| | | | 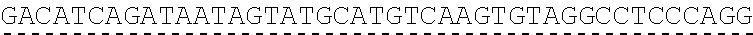 |
| | | | 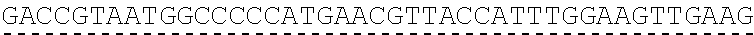 |
| | | | 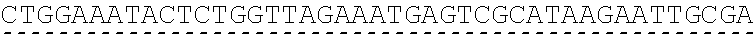 |
| | | | 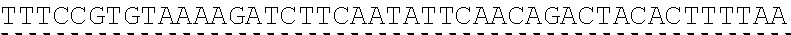 |
| | | | 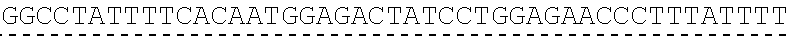 |
| | | | 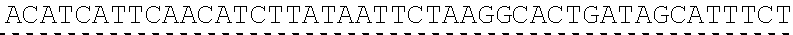 |
| | | | GGCATTTCTGATTATTGTGACATCAATAGCCCTGCTTGTTGTTCT |
| | | | CTACAAAATCTATGATCTACATAAGAAAAGATCCTGCAAT |
| 29 | 1963, | CD45RO | 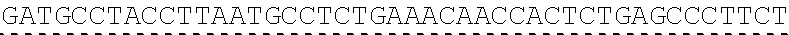 |
| | 1964, | | 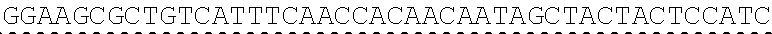 |
| | 1969, | | 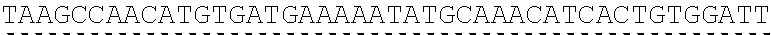 |
| | 1970, | | 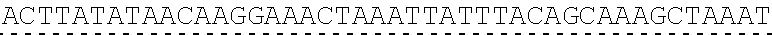 |
| | 1975, | | 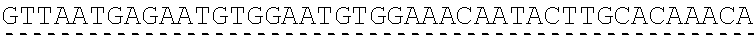 |
| | 1976 | | 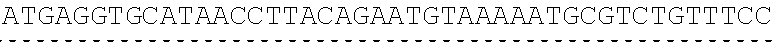 |
| | | | 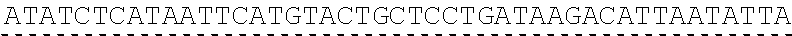 |
| | | | 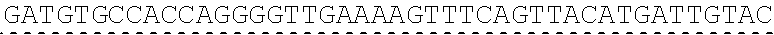 |
| | | | 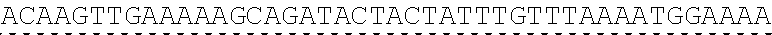 |
| | | | 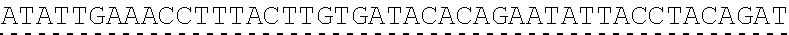 |
| | | | 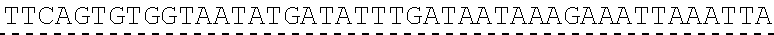 |
| | | | 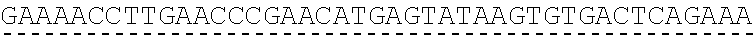 |
| | | | 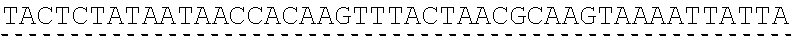 |
| | | | 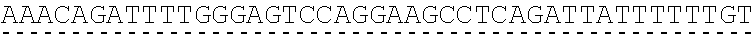 |
| | | | 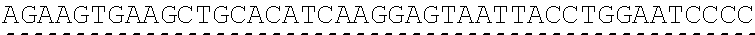 |
| | | | 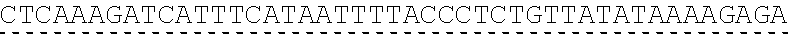 |
| | | | 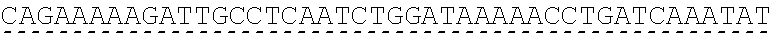 |
| | | | 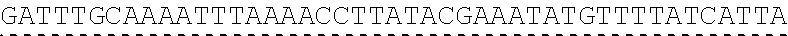 |
| | | | 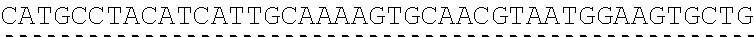 |
| | | | 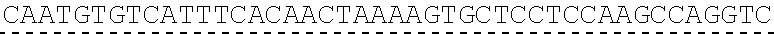 |
| | | | 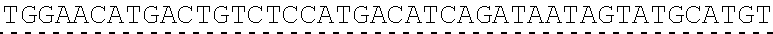 |
| | | | 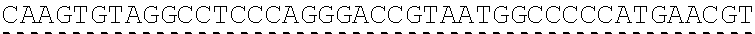 |
| | | | 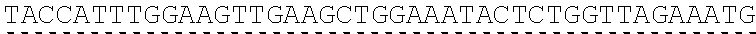 |
| | | | 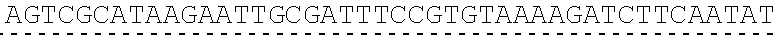 |
| | | | 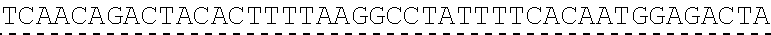 |
| | | | 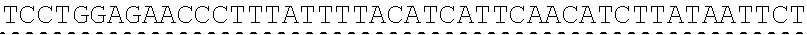 |
| | | | 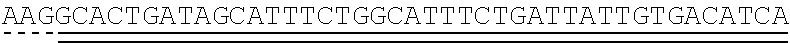 |
| | | | ATAGCCCTGCTTGTTGTTCTCTACAAAATCTATGATCTACATAAG |
| | | | AAAAGATCCTGCAAT |
| 30 | 1965, | CD45RABC | 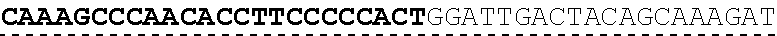 |
| | 1966, | | 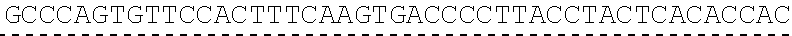 |
| | 1971, | | 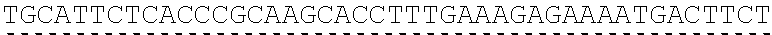 |
| | 1972, | | 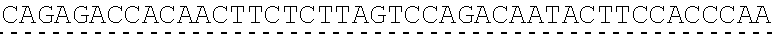 |
| | 1977, | | 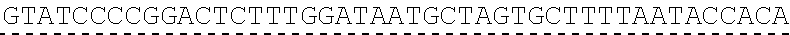 |
| | 1978 | | 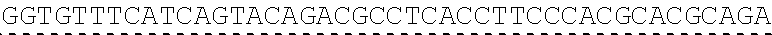 |
| | | | 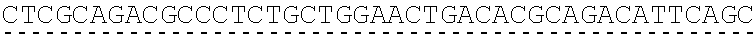 |
| | | | 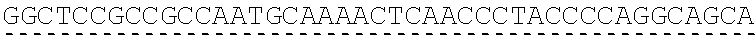 |
| | | | 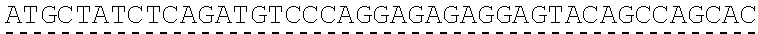 |
| | | | 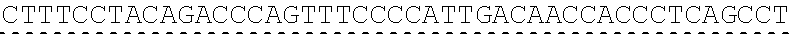 |
| | | | 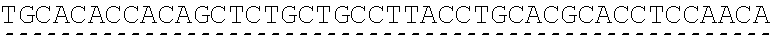 |
| | | | 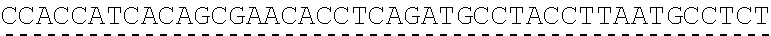 |
| | | | 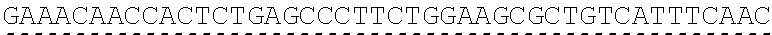 |
| | | | 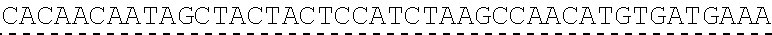 |
| | | | 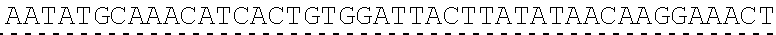 |
| | | | 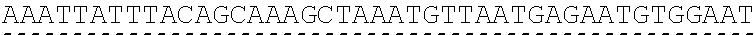 |
| | | | 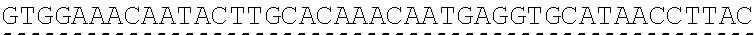 |
| | | | 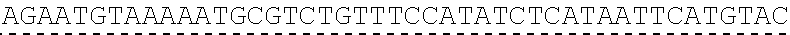 |
| | | | 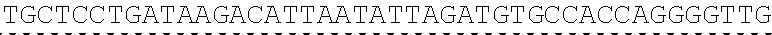 |
| | | | 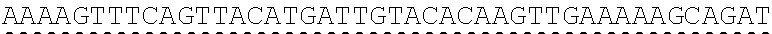 |
| | | | 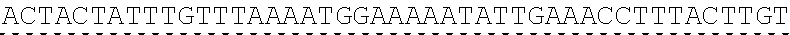 |
| | | | 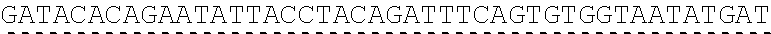 |
| | | | 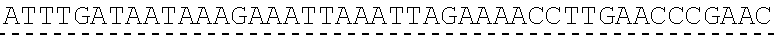 |
| | | | 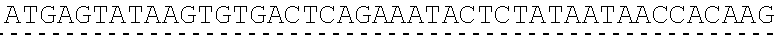 |
| | | | 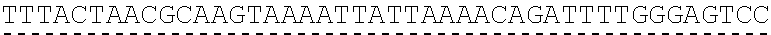 |
| | | | 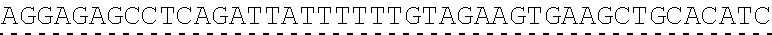 |
| | | | 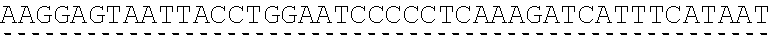 |
| | | | 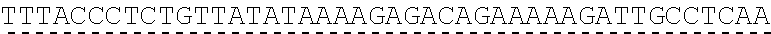 |
| | | | 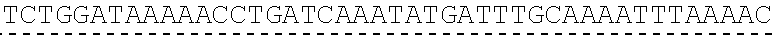 |
| | | | 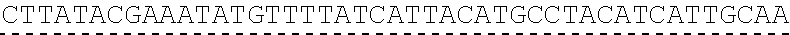 |
| | | | 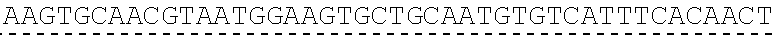 |
| | | | 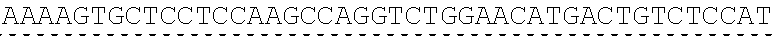 |
| | | | 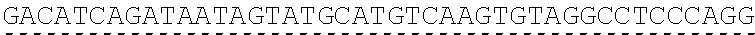 |
| | | | 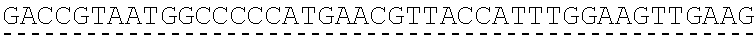 |
| | | | 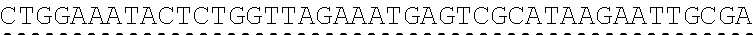 |
| | | | 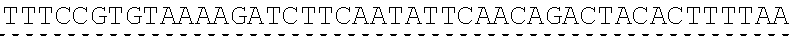 |
| | | | 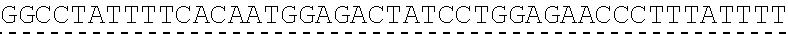 |
| | | | 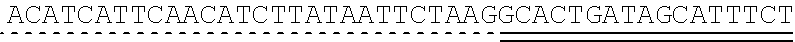 |
| | | | GGCATTTCTGATTATTGTGACATCAATAGCCCTGCTTGTTGTTCT |
| | | | CTACAAAATCTATGATCTACATAAGAAAAGATCCTGCAAT |
| 31 | 1965, | CD45RABC | 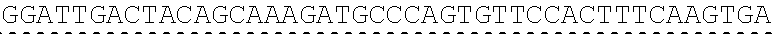 |
| | 1972, | | 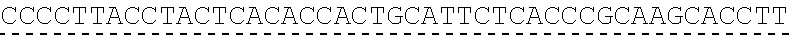 |
| | 1966, | | 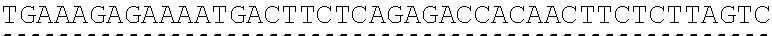 |
| | 1971, | | 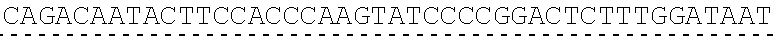 |
| | 1977, | | 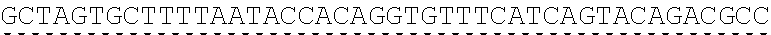 |
| | 1978 | | 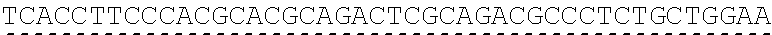 |
| | | | 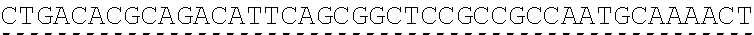 |
| | | | 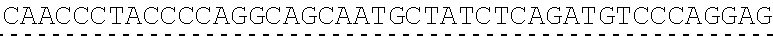 |
| | | | 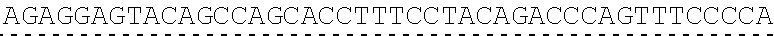 |
| | | | 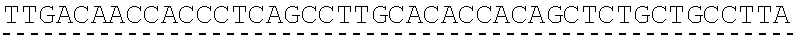 |
| | | | 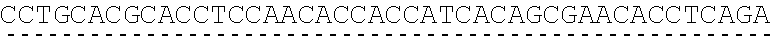 |
| | | | 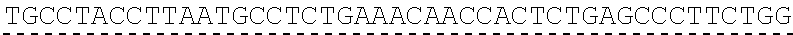 |
| | | | 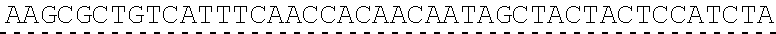 |
| | | | 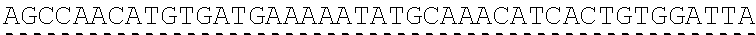 |
| | | | 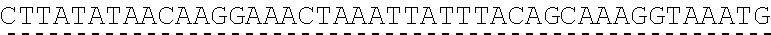 |
| | | | 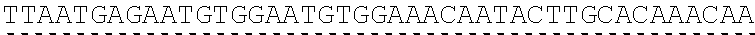 |
| | | | 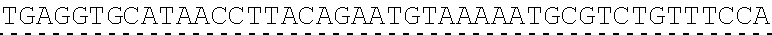 |
| | | | 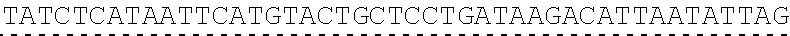 |
| | | | 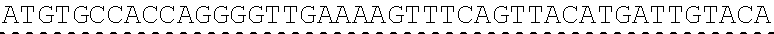 |
| | | | 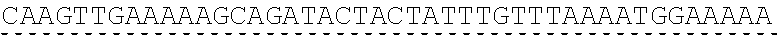 |
| | | | 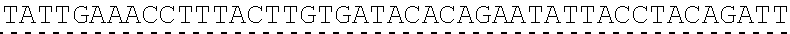 |
| | | | 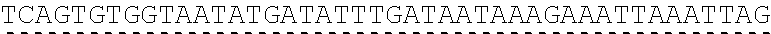 |
| | | | 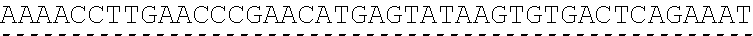 |
| | | | 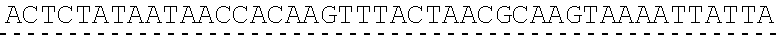 |
| | | | 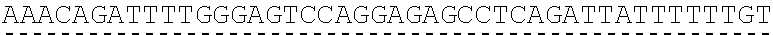 |
| | | | 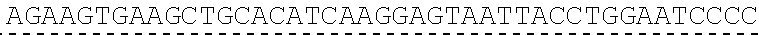 |
| | | | 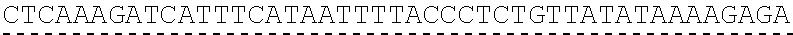 |
| | | | 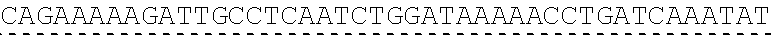 |
| | | | 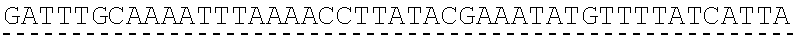 |
| | | | 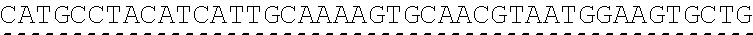 |
| | | | 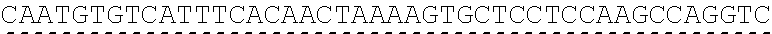 |
| | | | 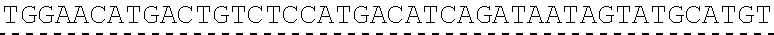 |
| | | | 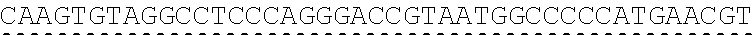 |
| | | | 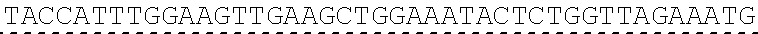 |
| | | | 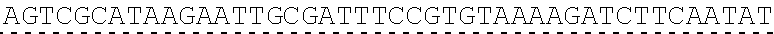 |
| | | | 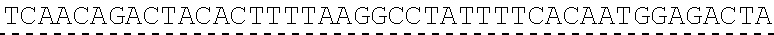 |
| | | | 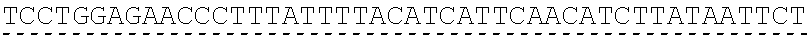 |
| | | | 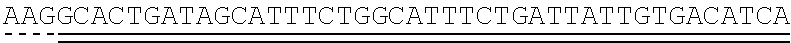 |
| | | | ATAGCCCTGCTTGTTGTTCTCTACAAAATCTATGATCTACATAAG |
| | | | AAAAGATCCTGCAAT |

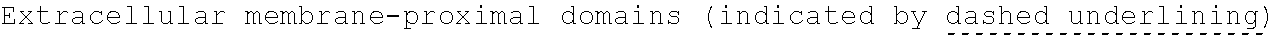

| | | | |
|---|---|---|---|
| 32 | 1941, | A2 | 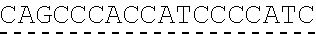 |
| | 1942 | | |
| 33 | 1943 | CD45RABC | 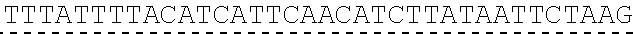 |
| | | +tm+12aa | |
| 34 | 1944, | Kb | 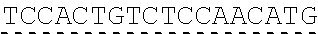 |
| | 1945 | | |
| 35 | | CD58 | 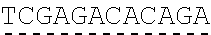 |
| | | extracellular | |
| | | proximal | |
| | | domain | |
| 36 | | HLA-A2 | 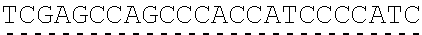 |

Transmembrane/intracellular domains (indicated by double underlined lettering). In some
examples below, double underlined and italicized lettering corresponds to the transmembrane
domain; membrane-proximal domains are indicated by unformatted lettering; restriction sites
are indicated by bold underlined lettering.

| | | | |
|---|---|---|---|
| 37 | 1882, | LFA-3 | AGACACAGATATGCACTTATACCCATACCATTAGCAGTAATTAC |
| | 1883, | | AACATGTATTGTGCTGTATATGAATGGTATTCTGAAATGTGACA |
| | 1884, | | GAAAACCAGACAGAACCAACTCCAAT |
| | 1885, | | |
| | 1886, | | |
| | 1887 | | |
| 38 | 1941, | A2 | CAGCCCACCATCCCCATCGTGGGCATCATTGCTGGCCTGGTTCT |
| | 1942 | | CTTTGGAGCTGTGATCACTGGAGCTGTGGTCGCTGCTGTGATGT |
| | | | GGAGGAGGAAAAGCTCAGATAGAAAAGGAGGGAGCTACTCTC |
| | | | AGGCTGCAAGCAGTGACAGTGCCCAGGGCTCTGATGTGTCTCT |
| | | | CACAGCTTGTAAAGTG |
| 39 | 1941, | A2 | GTGGGCATCATTGCTGGCCTGGTTCTCTTTGGAGCTGTGATCAC |
| | 1942 | | TGGAGCTGTGGTCGCTGCTGTGATGTGGAGGAGGAAAAGCTCA |
| | | | GATAGAAAAGGAGGGAGCTACTCTCAGGCTGCAAGCAGTGAC |
| | | | AGTGCCCAGGGCTCTGATGTGTCTCTCACAGCTTGTAAAGTG |
| 40 | 1944, | Kb | TCCACTGTCTCCAACATGGCGACCGTTGCTGTTCTGGTTGTCCTT |
| | 1945 | | GGAGCTGCAATAGTCACTGGAGCTGTGGTGGCTTTTGTGATGA |
| | | | AGATGAGAAGGAGAAACACAGGTGGAAAAGGAGGGGACTAT |
| | | | GCTCTGGCTCCAGGCTCCCAGACCTCTGATCTGTCTCTCCCAGAT |
| | | | TGTAAAGTGATGGTTCATGACCCTCATTCTCTAGCG |
| 41 | 1944, | Kb | GCGACCGTTGCTGTTCTGGTTGTCCTTGGAGCTGCAATAGTCAC |
| | 1945 | | TGGAGCTGTGGTGGCTTTTGTGATGAAGATGAGAAGGAGAAA |
| | | | CACAGGTGGAAAAGGAGGGGACTATGCTCTGGCTCCAGGCTCC |
| | | | CAGACCTCTGATCTGTCTCTCCCAGATTGTAAAGTGATGGTTCA |
| | | | TGACCCTCATTCTCTAGCG |
| 42 | 1943 | CD45RABC + | TTTATTTTACATCATTCAACATCTTATAATTCTAAGGCACTGATA |
| | | tm + 12 aa | GCATTTCTGGCATTTCTGATTATTGTGACATCAATAGCCCTGCTT |
| | | | GTTGTTCTCTACAAAATCTATGATCTACATAAGAAAAGATCCTG |
| | | | CAAT |

Transmembrane domains (indicated by double underlined and italicized lettering)

| | | | |
|---|---|---|---|
| 43 | 1941, | A2 | GTGGGCATCATTGCTGGCCTGGTTCTCTTTGGAGCTGTGATCAC |
| | 1942 | | TGGAGCTGTGGTCGCTGCTGTGATGTGG |
| 44 | 1944, | Kb | GCGACCGTTGCTGTTCTGGTTGTCCTTGGAGCTGCAATAGTCAC |
| | 1945 | | TGGAGCTGTGGTGGCTTTTGTGATG |
| 45 | 1963, | CD45RO | GCACTGATAGCATTTCTGGCATTTCTGATTATTGTGACATCAATA |
| | 1964, | | GCCCTGCTTGTTGTTCTCTAC |
| | 1969, | | |
| | 1970, | | |
| | 1975, | | |
| | 1976 | | |
| 46 | 1944 | CD45RABC | GCACTGATAGCATTTCTGGCATTTCTGATTATTGTGACATCAATA |
| | | | GCCCTGCTTGTTGTTCTC |
| 47 | 1961 | CD22 5D + | GTGGCTGTGGGACTCGGGTCCTGCCTCGCCATCCTCATCCTGGC |
| | | tm | AATCTGTGGGCTC |
| 48 | 1965, | CD45RABC | GCACTGATAGCATTTCTGGCATTTCTGATTATTGTGACATCAATA |
| | 1966, | | GCCCTGCTTGTTGTTCTCTAC |
| | 1971, | | |
| | 1972, | | |
| | 1978, | | |
| | 1977 | | |

Hinge (indicated by bold lettering)

| 49 | 1974, | CD8 | ACCACTACCCCAGCACCGAGGCCACCCACCCCGGCTCCTACCAT |
|---|---|---|---|
| | 1976, | | CGCCTCCCAGCCTCTGTCCCTGCGTCCGGAGGCATGTAGACCC |
| | 1978 | | GCAGCTGGTGGGGCCGTGCATACCCGGGGTCTTGACTTCGCCT |
| | | | GCGAT |
| 50 | 1962, | Li | GGCGGAGGCAGC |
| | 1964, | | |
| | 1966, | | |
| | 1968, | | |
| | 1970, | | |
| | 1972, | | |
| | 1973, | | |
| | 1975, | | |
| | 1977 | | |
| 51 | 1882, | L | GTTGCTGGGAGCGACGCGGGGCGGGCCCTGGGGGTCCTCAGC |
| | 1883, | | GTGGTCTGCCTGCTGCACTGCTTTGGTTTCATCAGCTGT |
| | 1884, | | |
| | 1941, | | |
| | 1942, | | |
| | 1943, | | |
| | 1961, | | |
| | 1962, | | |
| | 1963, | | |
| | 1964, | | |
| | 1965, | | |
| | 1966, | | |
| | 1967, | | |
| | 1968, | | |
| | 1969 | | |
| | 1970, | | |
| | 1971, | | |
| | 1972, | | |
| 52 | 1885, | L | TGCTTCATAAAACAGGGATGGTGTCTGGTCCTGGAACTGCTAC |
| | 1886, | | TGCTGCCCTTGGGAACTGGA |
| | 1887, | | |
| | 1944, | | |
| | 1945, | | |
| | 1946 | | |
| 53 | 1973, | L | GCCTTACCAGTGACCGCCTTGCTCCTGCCGCTGGCCTTGCTGCT |
| | 1974, | | CCACGCCGCCAGGCCG |
| | 1975, | | |
| | 1976, | | |
| | 1977, | | |
| | 1978 | | |

Tag (indicated by italicized lettering)

| | | | |
|---|---|---|---|
| 54 | 1882, | Ha | GGGTCCTACCCCTACGACGTTCCCGACTACGCTGGGAGCTCG |
| | 1883, | | |
| | 1884, | | |
| | 1885, | | |
| | 1886, | | |
| | 1887 | | |

Example cell-distancing devices with extracellular membrane-distal domain, elongation
domain, (optional) extracellular membrane-proximal domain, transmembrane domain,
and/or intracellular domain

| | | | |
|---|---|---|---|
| 55 | 1882 | hlEE-Ig-3 | TCTAGACGCCGCCACCATGGTTGCTGGGAGCGACGCGGGGCG |
| | | | GGCCCTGGGGGTCCTCAGCGTGGTCTGCCTGCTGCACTGCTTTG |
| | | | GTTTCATCAGCTGTTTTTCCCAACAAATATATGGTGTTGTGTATG |
| | | | GGAATGTAACTTTCCATGTACCAAGCAATGTGCCTTTAAAAGAG |
| | | | GTCCTATGGAAAAAACAAAAGGATAAAGTTGCAGAACTGGAAA |
| | | | ATTCTGAGTTCAGAGCTTTCTCATCTTTTAAAAATAGGGTTTATT |
| | | | TAGACACTGTGTCAGGTAGCCTCACTATCTACAACTTAACATCA |
| | | | TCAGATGAAGATGAGTATGAAATGGAATCGCCAAATATTACTG |
| | | | ATACCATGAAGTTCTTTCTTTATGTGCTTGAGTCTCTTCCATCTCC |
| | | | CACACTAACTTGTGCATTGACTAATGGAAGCATTGAAGTCCAAT |
| | | | GCATGATACCAGAGCATTACAACAGCCATCGAGGACTTATAAT |
| | | | GTACTCATGGGATTGTCCTATGGAGCAATGTAAACGTAACTCAA |
| | | | CCAGTATATATTTTAAGATGGAAAATGATCTTCCACAAAAAATA |
| | | | CAGTGTACTCTTAGCAATCCATTATTTAATACAACATCATCAATC |
| | | | ATTTTGACAACCTGTATCCCAAGCAGCGGTCATTCAAGACACAG |
| | | | AGGGTCCTACCCCTACGACGTTCCCGACTACGCTGGGAGCTCGC |
| | | | CGTCGACCCCCAAGAAGGTGACCACAGTGATTCAAAACCCCAT |
| | | | GCCGATTCGAGAAGGAGACACAGTGACCCTTTCCTGTAACTAC |
| | | | AATTCCAGTAACCCCAGTGTTACCCGGTATGAATGGAAACCTCA |
| | | | TGGGGCCTGGGAGGAGCCATCGCTTGGGGTGCTGAAGATCCA |
| | | | AAACGTAGGCTGGGACAACACAACCATCGCCTGCGCAGCTTGT |
| | | | AATAGTTGGTGCTCTTGGGCCTCCCCTGTCGCCCTGAATGTCCA |
| | | | GTATGCCCCCCGAGACGTGAGGGTCCGGAAAATCAAGCCCCTT |
| | | | TCCGAGATTCACTCTGGAAACTCGGTCAGCCTCCAATGTGACTT |
| | | | CTCAAGCAGCCACCCCAAAGAAGTCCAGTTCTTCTGGGAGAAA |
| | | | AATGGCAGGCTTCTGGGGAAAGAAAGCCAGCTGAATTTTGACT |
| | | | CCATCTCCCCAGAAGATGCTGGGAGTTACAGCTGCTGGGTGAA |
| | | | CAACTCCATAGGACAGACAGCGTCCAAGGCCTGGACACTTGAA |
| | | | GTGCTGTATGCACCCAGGAGGCTGCGTGTGTCCATGAGCCCTG |
| | | | GGGACCAAGTGATGGAGGGGAAGAGTGCAACCCTGACCTGTG |
| | | | AGAGCGACGCCAACCCTCCCGTCTCCCACTACACCTGGTTTGAC |
| | | | TGGAATAACCAAAGCCTCCCCTACCACAGCCAGAAGCTGAGAT |
| | | | TGGAGCCGGTGAAGGTCCAGCACTCGGGTGCCTACTGGTGCCA |
| | | | GGGGACCAACAGTGTGGGCAAGGGCCGTTCGCCTCTCAGCACC |
| | | | CTCACCGTCTACTACTCGCCGGAGACCATCTCGAGACACAGATA |
| | | | TGCACTTATACCCATACCATTAGCAGTAATTACAACATGTATTGT |
| | | | GCTGTATATGAATGGTATTCTGAAATGTGACAGAAAACCAGAC |
| | | | AGAACCAACTCCAATTGAGCGGCCGC |

TABLE 5
Example Amino Acid Sequences

| | | | |
|---|---|---|---|
| SEQ | | | |
| ID | Construct | | |
| NO | Number | Description | Sequence |

Extracellular membrane-distal domains (denoted by underlining). Some examples
include a hinge denoted by bold lettering.

| | | | |
|---|---|---|---|
| 56 | 1882, | LFA-3 | MVAGSDAGRALGVLSVVCLLHCFGFISCFSQQIYGVVYG |
| | 1883, | ectodomain | NVTFHVPSNVPLKEVLWKKQKDKVAELENSEFRAFSSFKN |
| | 1884 | | RVYLDTVSGSLTIYNLTSSDEDEYEMESPNITDTMKFFLYVL |
| | | | ESLPSPTLTCALTNGSIEVQCMIPEHYNSHRGLIMYSWDC |
| | | | PMEQCKRNSTSIYFKMENDLPQKIQCTLSNPLFNTTSSIILT |
| | | | TCIPSSGHSRHR |
| 57 | 1882, | LFA-3 | FSQQIYGVVYGNVTFHVPSNVPLKEVLWKKQKDKVAELE |
| | 1883, | ectodomain | NSEFRAFSSFKNRVYLDTVSGSLTIYNLTSSDEDEYEMESP |
| | 1884 | | NITDTMKFFLYVLESLPSPTLTCALTNGSIEVQCMIPEHYNS |
| | | | HRGLIMYSWDCPMEQCKRNSTSIYFKMENDLPQKIQCTL |
| | | | SNPLFNTTSSIILTTCIPSSGHSRHR |
| 58 | | N-terminal Ig- | FSQQIYGVVYGNVTFHVPSNVPLKEVLWKKQKDKVAELE |
| | | like domain of | NSEFRAFSSFKNRVYLDTVSGSLTIYNLTSSDEDEYEMESP |
| | | LFA-3 (CD58) | NITDTMKFFLYV |
| 59 | | two N- | FSQQIYGVVYGNVTFHVPSNVPLKEVLWKKQKDKVAELE |
| | | terminal Ig- | NSEFRAFSSFKNRVYLDTVSGSLTIYNLTSSDEDEYEMESP |
| | | like domains | NITDTMKFFLYVLESLPSPTLTCALTNGSIEVQCMIPEHYNS |
| | | of LFA-3 | HRGLIMYSWDCPMEQCKRNSTSIYFKMENDLPQKIQCTL |
| | | (CD58) | SNPLFNTTSSIILTTCIPS |
| 60 | 1885, | CD48 | MCFIKQGWCLVLELLLLPLGTGFQGHSIPDINATTGSNVT |
| | 1886, | ectodomain | LKIHKDPLGPYKRITWLHTKNQKILEYNYNSTKTIFESEFKG |
| | 1887 | | RVYLEENNGALHISNVRKEDKGTYYMRVLRETENELKITLE |
| | 1944, | | VFDPVPKPSIEINKTEASTDSCHLRLSCEVKDQHVDYTWYE |
| | 1945, | | SSGPFPKKSPGYVLDLIVTPQNKSTFYTCQVSNPVSSKNDT |
| | 1946 | | VYFTLPCDLARS |
| 61 | 1885, | CD48 | FQGHSIPDINATTGSNVTLKIHKDPLGPYKRITWLHTKNQK |
| | 1886, | ectodomain | ILEYNYNSTKTIFESEFKGRVYLEENNGALHISNVRKEDKGT |
| | 1887, | | YYMRVLRETENELKITLEVFDPVPKPSIEINKTEASTDSCHL |
| | 1944, | | RLSCEVKDQHVDYTWYESSGPFPKKSPGYVLDLIVTPQNK |
| | 1945, | | STFYTCQVSNPVSSKNDTVYFTLPCDL |
| | 1946 | | |
| 62 | 1941, | CD58 | MVAGSDAGRALGVLSVVCLLHCFGFISCFSQQIYGVVYG |
| | 1942, | ectodomain | NVTFHVPSNVPLKEVLWKKQKDKVAELENSEFRAFSSFKN |
| | 1943 | | RVYLDTVSGSLTIYNLTSSDEDEYEMESPNITDTMKFFLYVL |
| | | | ESLPSPTLTCALTNGSIEVQCMIPEHYNSHRGLIMYSWDC |
| | | | PMEQCKRNSTSIYFKMENDLPQKIQCTLSNPLFNTTSSIILT |
| | | | TCIPSSGHSRHR |
| 63 | 1941, | CD58 | FSQQIYGVVYGNVTFHVPSNVPLKEVLWKKQKDKVAELE |
| | 1942, | ectodomain | NSEFRAFSSFKNRVYLDTVSGSLTIYNLTSSDEDEYEMESP |
| | 1943 | | NITDTMKFFLYVLESLPSPTLTCALTNGSIEVQCMIPEHYNS |
| | | | HRGLIMYSWDCPMEQCKRNSTSIYFKMENDLPQKIQCTL |
| | | | SNPLFNTTSSIILTTCIPSSGHSRHR |
| 64 | 1961, | CD58 1D | MVAGSDAGRALGVLSVVCLLHCFGFISC |
| | 1962, | | FSQQIYGVVYGNVTFHVPSNVPLKEVLWKKQKDKVAELE |
| | 1963, | | NSEFRAFSSFKNRVYLDTVSGSLTIYNLTSSDEDEYEMESP |
| | 1965, | | NITDTMKFFLYV |
| | 1964, | | |
| | 1966 | | |
| 65 | 1961, | CD58 1D | FSQQIYGVVYGNVTFHVPSNVPLKEVLWKKQKDKVAELE |
| | 1962, | | NSEFRAFSSFKNRVYLDTVSGSLTIYNLTSSDEDEYEMESP |
| | 1963, | | NITDTMKFFLYV |
| | 1965, | | |
| | 1964, | | |
| | 1966 | | |
| 66 | 1967, | CD58 2D | MVAGSDAGRALGVLSVVCLLHCFGFISC |
| | 1969, | | FSQQIYGVVYGNVTFHVPSNVPLKEVLWKKQKDKVAELE |
| | 1968, | | NSEFRAFSSFKNRVYLDTVSGSLTIYNLTSSDEDEYEMESP |
| | 1970, | | NITDTMKFFLYVLESLPSPTLTCALTNGSIEVQCMIPEHYNS |
| | 1971, | | HRGLIMYSWDCPMEQCKRNSTSIYFKMENDLPQKIQCTL |
| | 1972 | | SNPLFNTTSSIILTTCIPS |
| 67 | 1967, | CD58 2D | FSQQIYGVVYGNVTFHVPSNVPLKEVLWKKQKDKVAELE |
| | 1968, | | NSEFRAFSSFKNRVYLDTVSGSLTIYNLTSSDEDEYEMESP |
| | 1969, | | NITDTMKFFLYVLESLPSPTLTCALTNGSIEVQCMIPEHYNS |
| | 1970, | | HRGLIMYSWDCPMEQCKRNSTSIYFKMENDLPQKIQCTL |
| | 1971, | | SNPLFNTTSSIILTTCIPS |
| | 1972 | | |
| 68 | 1973, | CD2 scFv | MALPVTALLLPLALLLHAARPDVVMTQSPPSLLVTLGQPA |
| | 1974, | | SISCRSSQSLLHSSGNTYLNWLLQRPGQSPQPLIYLVSKLES |
| | 1975, | | GVPDRFSGSGSGTDFTLKISGVEAEDVGVYYCMQFTHYPY |
| | 1976, | | TFGQGTKLEIKGGGGSGGGGSGGGGSQVQLVQSGAEVK |
| | 1977, | | KPGASVKVSCKASGYTFTEYYMYWVRQAPGQGLELMGRI |
| | 1978 | | DPEDGSIDYVEKFKKKVTLTADTSSSTAYMELSSLTSDDTA |
| | | | VYYCARGKFNYRFAYWGQGTLVTVSS |
| 69 | 1973, | CD2 scFv with | DVVMTQSPPSLLVTLGQPASISCRSSQSLLHSSGNTYLNW |
| | 1974, | a (Gly₄Ser)₃ | LLQRPGQSPQPLIYLVSKLESGVPDRFSGSGSGTDFTLKISG |
| | 1975, | between V_{L} | VEAEDVGVYYCMQFTHYPYTFGQGTKLEIKGGGGSGGG |
| | 1976, | and V_{H} | GSGGGGSQVQLVQSGAEVKKPGASVKVSCKASGYTFTEY |
| | 1977, | (denoted by | YMYWVRQAPGQGLELMGRIDPEDGSIDYVEKFKKKVTLT |
| | 1978 | bold lettering) | ADTSSSTAYMELSSLTSDDTAVYYCARGKFNYRFAYWGQ |
| | | | GTLVTVSS |

Elongation domain with membrane-proximal region at C terminus (indicated by dashed
underlining).

| | | | |
|---|---|---|---|
| 70 | 1882, | CD22 3 exons | 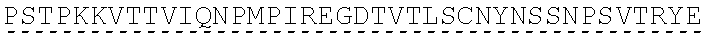 |
| | 1885 | | 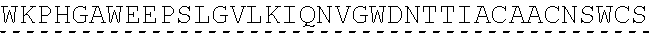 |
| | | | 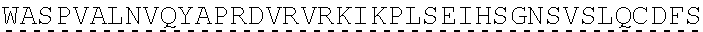 |
| | | | 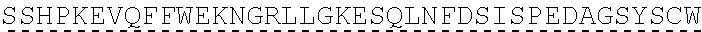 |
| | | | 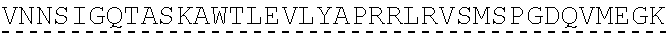 |
| | | | 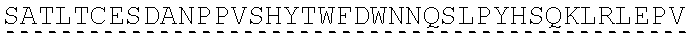 |
| | | | 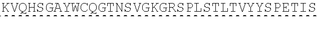 |
| 71 | | CD22 (five Ig- | 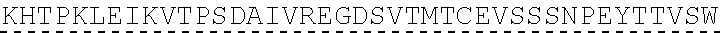 |
| | | like, | 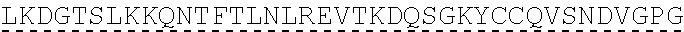 |
| | | membrane- | 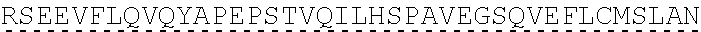 |
| | | proximal | 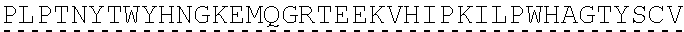 |
| | | extracellular | 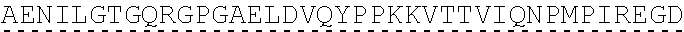 |
| | | domains) | 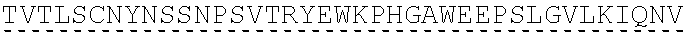 |
| | | | 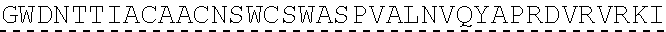 |
| | | | 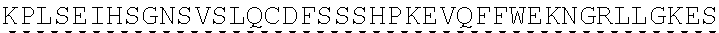 |
| | | | 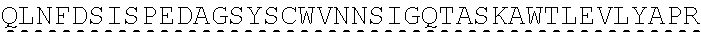 |
| | | | 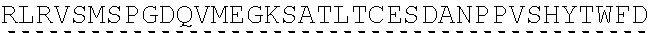 |
| | | | 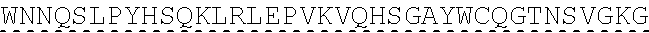 |
| | | | 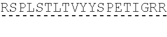 |
| 72 | 1883, | CD45RO | 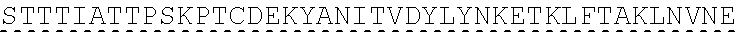 |
| | 1886 | | 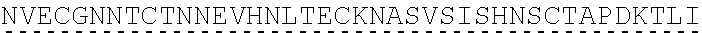 |
| | | | 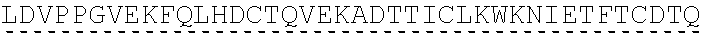 |
| | | | 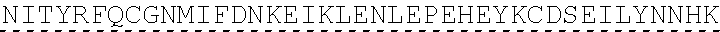 |
| | | | 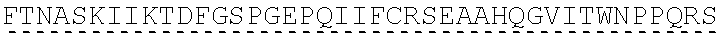 |
| | | | 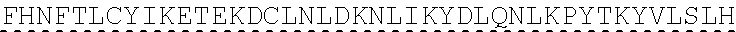 |
| | | | 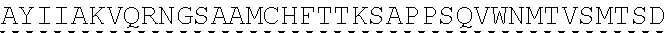 |
| | | | 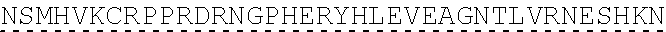 |
| | | | 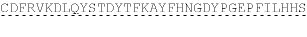 |
| 73 | 1884, | CD45RABC | 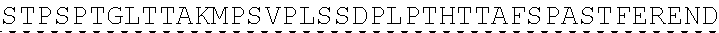 |
| | 1886 | | 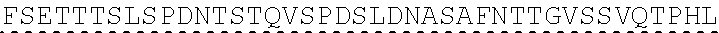 |
| | | | 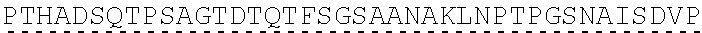 |
| | | | 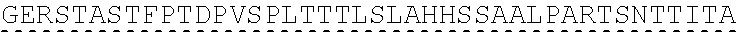 |
| | | | 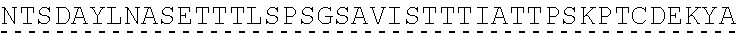 |
| | | | 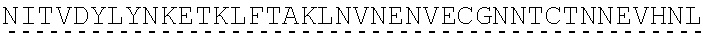 |
| | | | 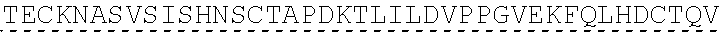 |
| | | | 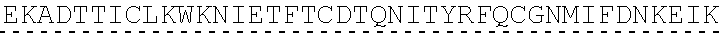 |
| | | | 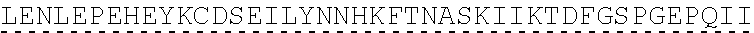 |
| | | | 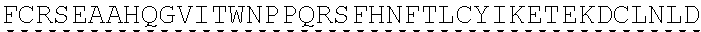 |
| | | | 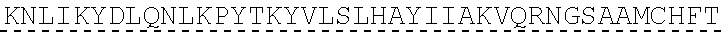 |
| | | | 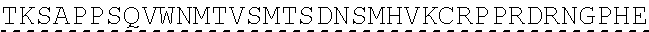 |
| | | | 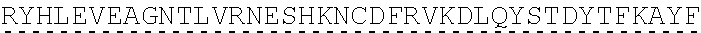 |
| | | | 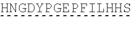 |
| 74 | 1941 | CD22 5 exons | 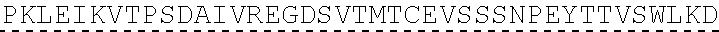 |
| | | | 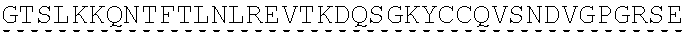 |
| | | | 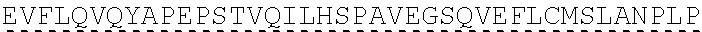 |
| | | | 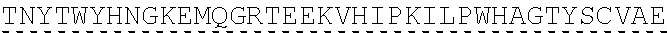 |
| | | | 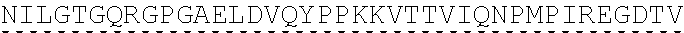 |
| | | | 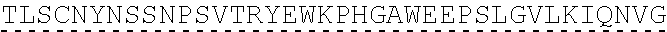 |
| | | | 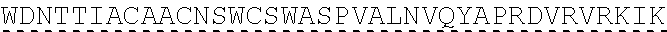 |
| | | | 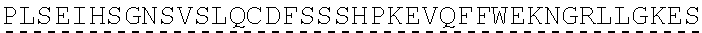 |
| | | | 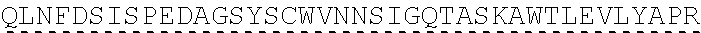 |
| | | | 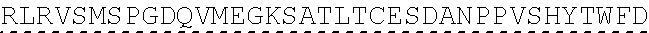 |
| | | | 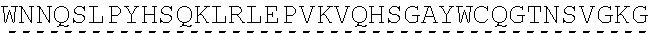 |
| | | | 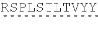 |
| 75 | 1944 | CD22 5 exons | 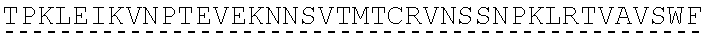 |
| | | | 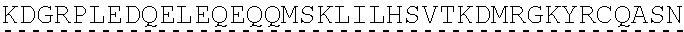 |
| | | | 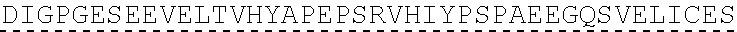 |
| | | | 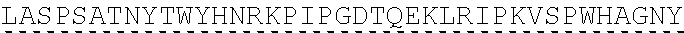 |
| | | | 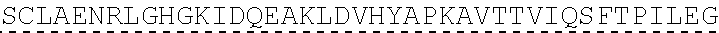 |
| | | | 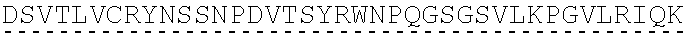 |
| | | | 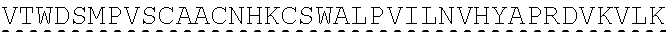 |
| | | | 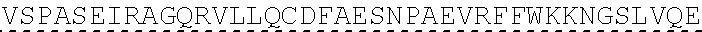 |
| | | | 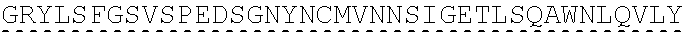 |
| | | | 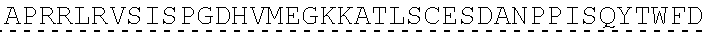 |
| | | | 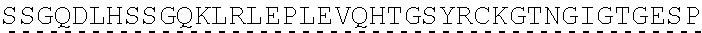 |
| | | | 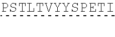 |
| 76 | 1942 | CD45RABC | 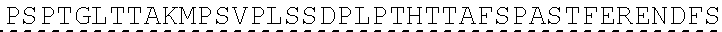 |
| | | | 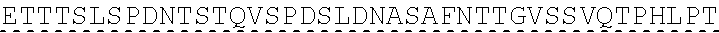 |
| | | | 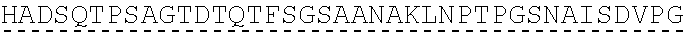 |
| | | | 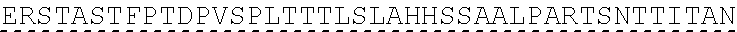 |
| | | | 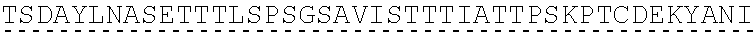 |
| | | | 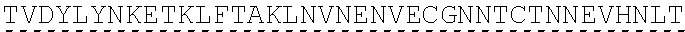 |
| | | | 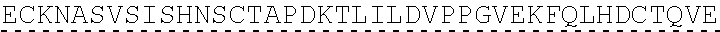 |
| | | | 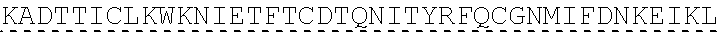 |
| | | | 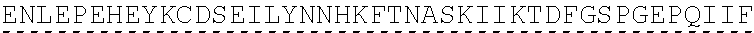 |
| | | | 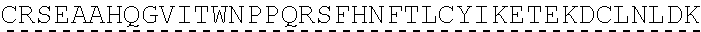 |
| | | | 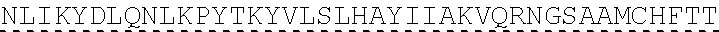 |
| | | | 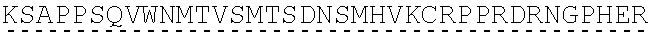 |
| | | | 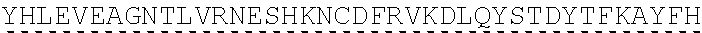 |
| | | | 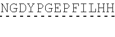 |
| 77 | 1945 | CD45RABC | 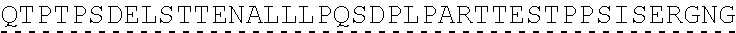 |
| | | | 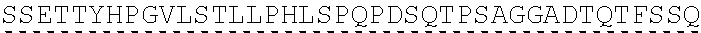 |
| | | | 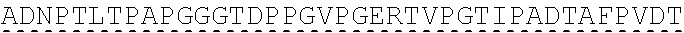 |
| | | | 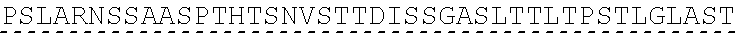 |
| | | | 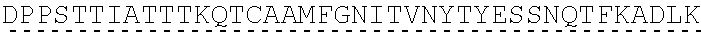 |
| | | | 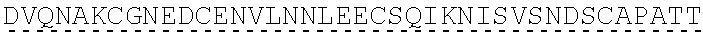 |
| | | | 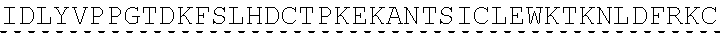 |
| | | | 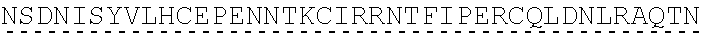 |
| | | | 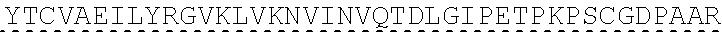 |
| | | | 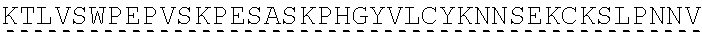 |
| | | | 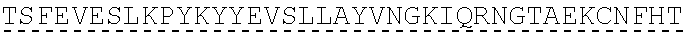 |
| | | | 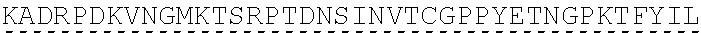 |
| | | | 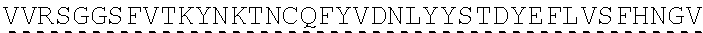 |
| | | | 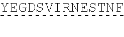 |
| 78 | | CD45RABC | 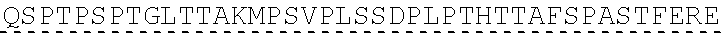 |
| | | | 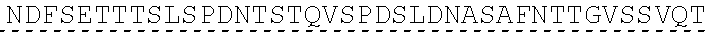 |
| | | | 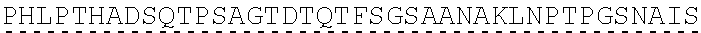 |
| | | | 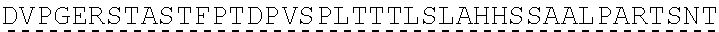 |
| | | | 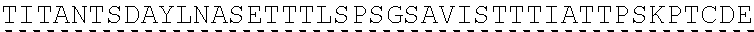 |
| | | | 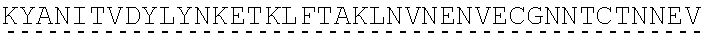 |
| | | | 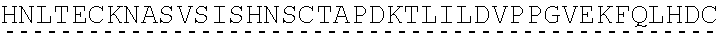 |
| | | | 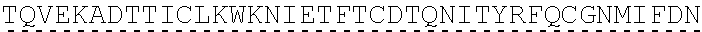 |
| | | | 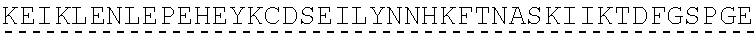 |
| | | | 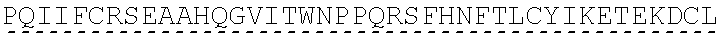 |
| | | | 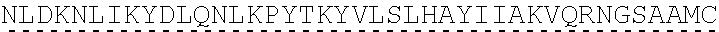 |
| | | | 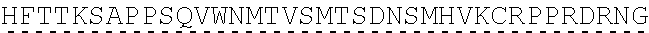 |
| | | | 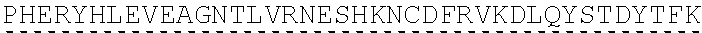 |
| | | | 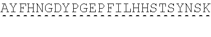 |
| 79 | | CD45RO | 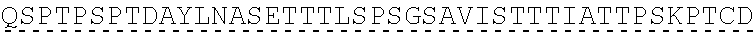 |
| | | | 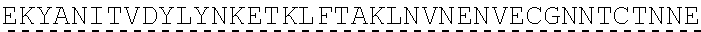 |
| | | | 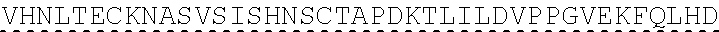 |
| | | | 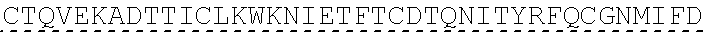 |
| | | | 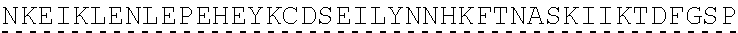 |
| | | | 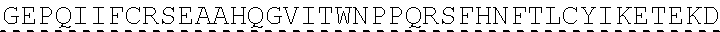 |
| | | | 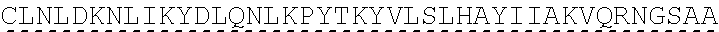 |
| | | | 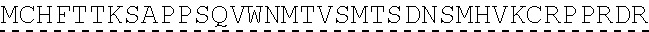 |
| | | | 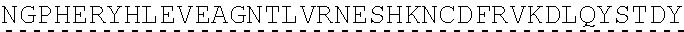 |
| | | | 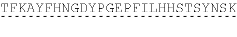 |

Elongation/(optional) extracellular membrane-proximal/transmembrane/intracellular
domains Elongation domains are indicated by dashed underlining; transmembrane and/or
intracellular domains are indicated by double underlined lettering with the
transmembrane domain further italicized. In some examples below, restriction sites
are indicated by bold and dashed underlined lettering; unformatted lettering
indicates an extracellular membrane-proximal domain.

| | | | |
|---|---|---|---|
| 80 | 1943 | CD45RABC + | 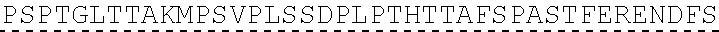 |
| | | tm + 12aa | 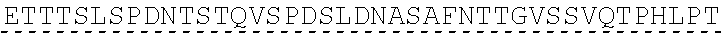 |
| | | | 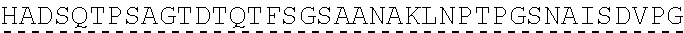 |
| | | | 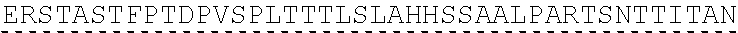 |
| | | | 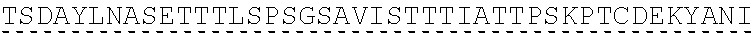 |
| | | | 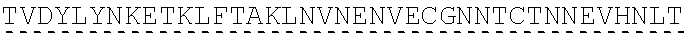 |
| | | | 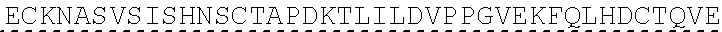 |
| | | | 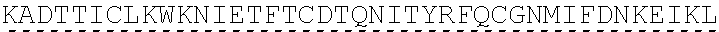 |
| | | | 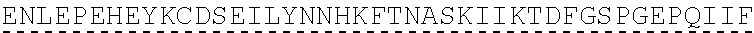 |
| | | | 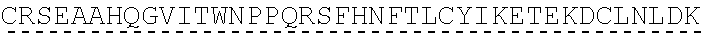 |
| | | | 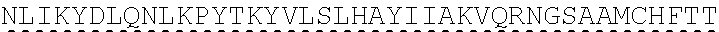 |
| | | | 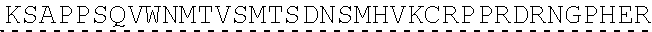 |
| | | | 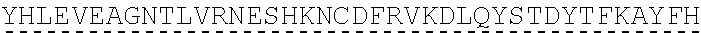 |
| | | | 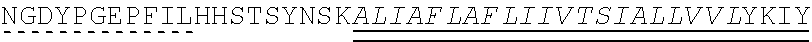 |
| | | | DLHKKRSCN |
| 81 | 1946 | CD45RABC + | 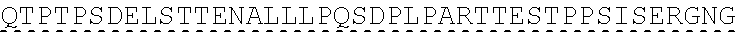 |
| | | tm + 12aa | 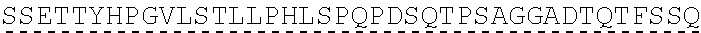 |
| | | | 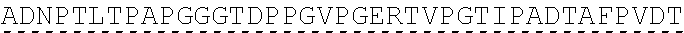 |
| | | | 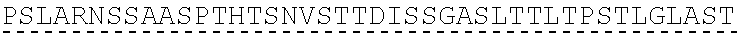 |
| | | | 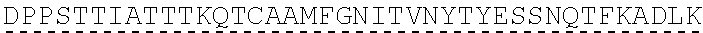 |
| | | | 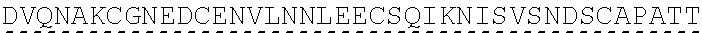 |
| | | | 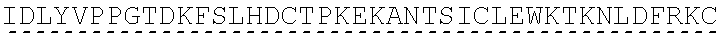 |
| | | | 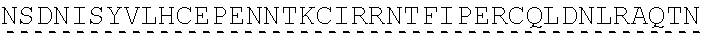 |
| | | | 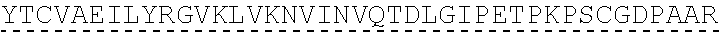 |
| | | | 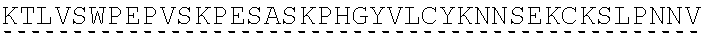 |
| | | | 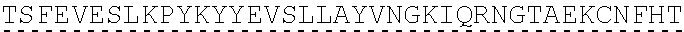 |
| | | | 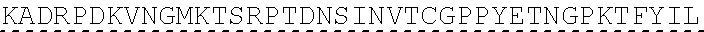 |
| | | | 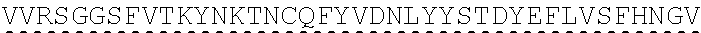 |
| | | | 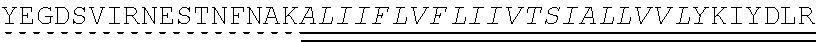 |
| | | | KKRSSN |
| 82 | 1961, | CD22 5D + tm | 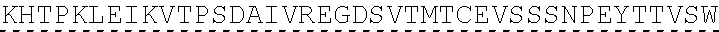 |
| | 1962, | | 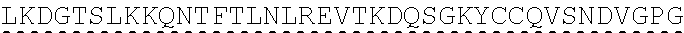 |
| | 1967, | | 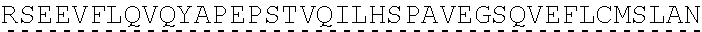 |
| | 1968, | | 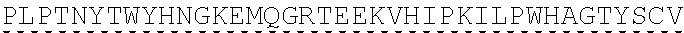 |
| | 1973, | | 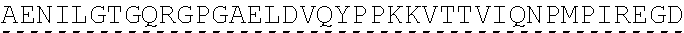 |
| | 1974, | | 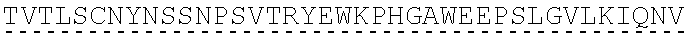 |
| | | | 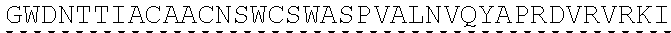 |
| | | | 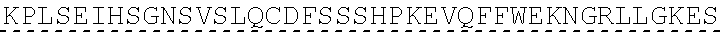 |
| | | | 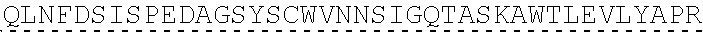 |
| | | | 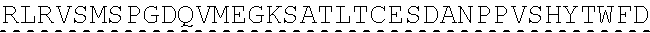 |
| | | | 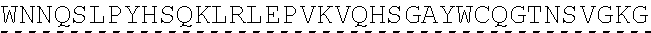 |
| | | | 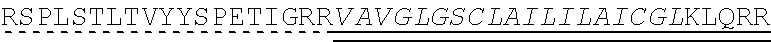 |
| | | | WKRTQSQQG |
| 83 | 1963, | CD45RO | 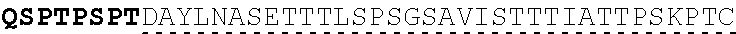 |
| | 1964, | | 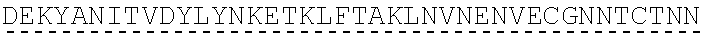 |
| | 1969, | | 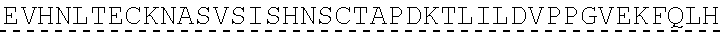 |
| | 1970, | | 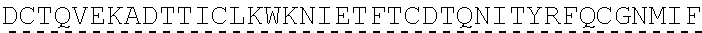 |
| | 1975, | | 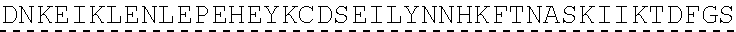 |
| | 1976 | | 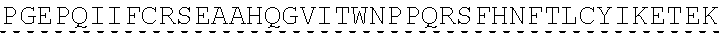 |
| | | | 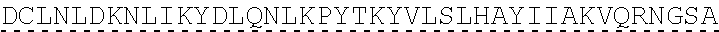 |
| | | | 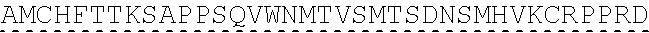 |
| | | | 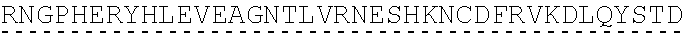 |
| | | | 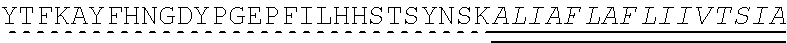 |
| | | | [symbol] |
| 84 | 1963, | CD45RO | 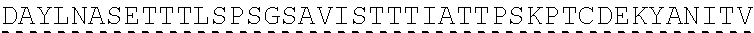 |
| | 1964, | | 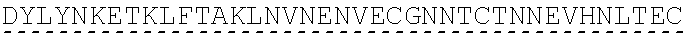 |
| | 1969, | | 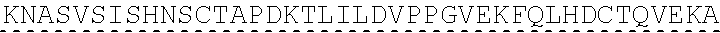 |
| | 1970, | | 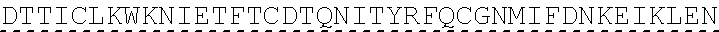 |
| | 1975, | | 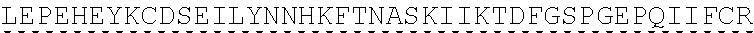 |
| | 1976 | | 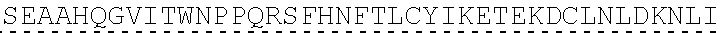 |
| | | | 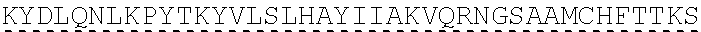 |
| | | | 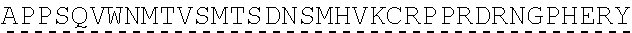 |
| | | | 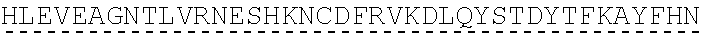 |
| | | | 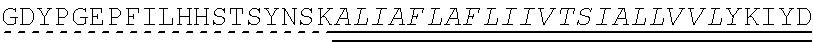 |
| | | | LHKKRSCN |
| 85 | 1965, | CD45RABC | 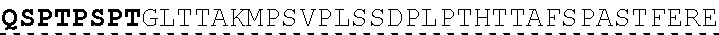 |
| | 1966, | | 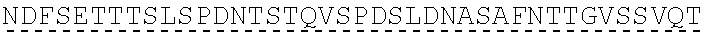 |
| | 1971, | | 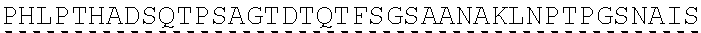 |
| | 1972, | | 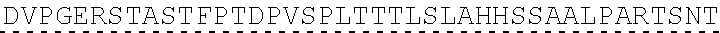 |
| | 1977, | | 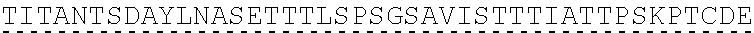 |
| | 1978 | | 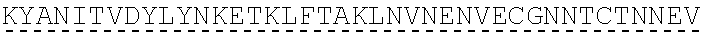 |
| | | | 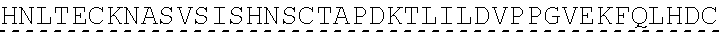 |
| | | | 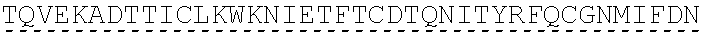 |
| | | | 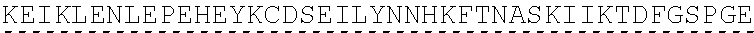 |
| | | | 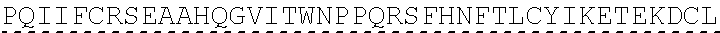 |
| | | | 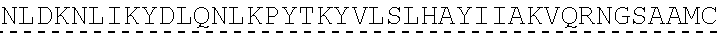 |
| | | | 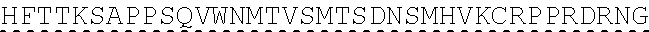 |
| | | | 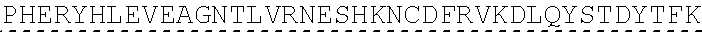 |
| | | | 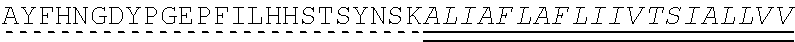 |
| | | | [symbol] |
| 86 | 1965, | CD45RABC | 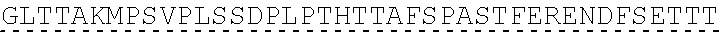 |
| | 1966, | | 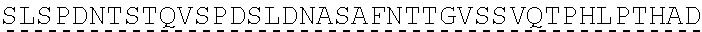 |
| | 1971, | | 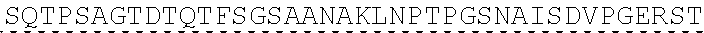 |
| | 1972, | | 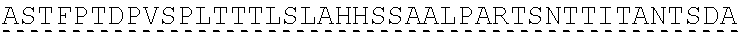 |
| | 1977, | | 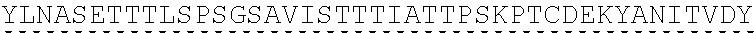 |
| | 1978 | | 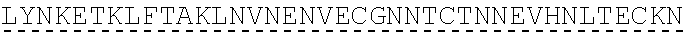 |
| | | | 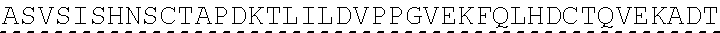 |
| | | | 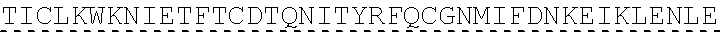 |
| | | | 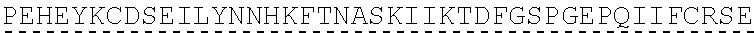 |
| | | | 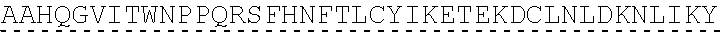 |
| | | | 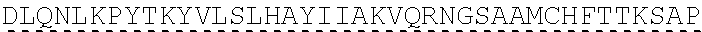 |
| | | | 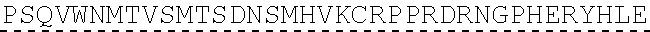 |
| | | | 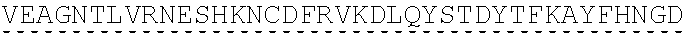 |
| | | | 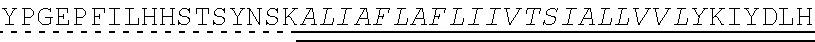 |
| | | | KKRSCN |

Extracellular membrane-proximal domains (indicated by dashed underlining)

| | | | |
|---|---|---|---|
| 87 | 1941, | A2 | 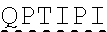 |
| | 1942 | | |
| 88 | 1943 | CD45RABC + | 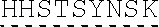 |
| | | tm + 12aa | |
| 89 | 1944, | Kb | 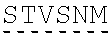 |
| | 1945 | | |
| 90 | | CD58 | 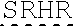 |
| | | (inventor's | |
| | | addition)- | |
| | | extracellular | |
| | | proximal | |
| | | domains | |
| 91 | | HLA-A2 (from | 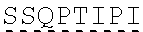 |
| | | inventor's | |
| | | addition) | |

Transmembrane/intracellular domains (indicated by double underlined lettering).
Double underlined and italicized lettering corresponds to the transmembrane
domain. In some examples below, unformatted lettering corresponds to a
membrane-proximal domain.

| | | | |
|---|---|---|---|
| 92 | 1882, | LFA-3 | RHRYALIPIPLAVITTCIVLYMNGILKCDRKPDRTNSN |
| | 1883, | | |
| | 1884, | | |
| | 1885, | | |
| | 1886, | | |
| | 1887 | | |
| 93 | 1941, | A2 | QPTIPIVGIIAGLVLFGAVITGAVVAAVMWRRKSSDRKGG |
| | 1942 | | SYSQAASSDSAQGSDVSLTACKV |
| 94 | 1941, | A2 | VGIIAGLVLFGAVITGAVVAAVMWRRKSSDRKGGSYSQA |
| | 1942 | | ASSDSAQGSDVSLTACKV |
| 95 | 1944, | Kb | STVSNMATVAVLVVLGAAIVTGAVVAFVMKMRRRNTGG |
| | 1945 | | KGGDYALAPGSQTSDLSLPDCKVMVHDPHSLA |
| 96 | 1944, | Kb | ATVAVLVVLGAAIVTGAVVAFVMKMRRRNTGGKGGDYA |
| | 1945 | | [symbol] [symbol] |
| 97 | 1943 | CD45RABC + | HHSTSYNSK [symbol] [symbol] |
| | | tm + 12 aa | |

Transmembrane domains (indicated by double underlined and italicized lettering)

| | | | |
|---|---|---|---|
| 98 | 1941, | A2 | [symbol] |
| | 1942 | | |
| 99 | 1944, | Kb | [symbol] |
| | 1945 | | |
| 100 | 1963, | CD45RO | [symbol] |
| | 1964, | | |
| | 1969, | | |
| | 1970, | | |
| | 1975, | | |
| | 1976 | | |
| 101 | 1944 | CD45RABC | [symbol] |
| 102 | 1961 | CD22 5D + tm | [symbol] |
| 103 | 1965, | CD45RABC | [symbol] |
| | 1966, | | |
| | 1971, | | |
| | 1972, | | |
| | 1977, | | |
| | 1978 | | |

Hinge (indicated by bold lettering)

| | | | |
|---|---|---|---|
| 104 | 1974, | CD8 Hinge | TTTPAPRPPTPAPTIASQPLSLRPEACRPAAGGAVHTRGL |
| | 1976, | | DFACD |
| | 1978 | | |
| 105 | 1962, | Li | GGGS |
| | 1964, | | |
| | 1966, | | |
| | 1968, | | |
| | 1970, | | |
| | 1972, | | |
| | 1973, | | |
| | 1975, | | |
| | 1977 | | |
| 106 | 1882, | L | MVAGSDAGRALGVLSVVCLLHCFGFISC |
| | 1883, | | |
| | 1884, | | |
| | 1941, | | |
| | 1942, | | |
| | 1943, | | |
| | 1961, | | |
| | 1962, | | |
| | 1963, | | |
| | 1964, | | |
| | 1965, | | |
| | 1966, | | |
| | 1967, | | |
| | 1968, | | |
| | 1969, | | |
| | 1970, | | |
| | 1971, | | |
| | 1972, | | |
| 107 | 1885, | L | MCFIKQGWCLVLELLLLPLGTG |
| | 1886, | | |
| | 1887, | | |
| | 1944, | | |
| | 1945, | | |
| | 1946 | | |
| 108 | 1973, | L | MALPVTALLLPLALLLHAARP |
| | 1974, | | |
| | 1975, | | |
| | 1976, | | |
| | 1977, | | |
| | 1978 | | |
| 169 | | | GGG |
| 170 | | | GGGSGGG |

Tag (indicated by italicized lettering)

| | | | |
|---|---|---|---|
| 109 | 1882, | Ha | GSYPYDVPDYAGSS |
| | 1883, | | |
| | 1884, | | |
| | 1885, | | |
| | 1886, | | |
| | 1887 | | |

Example cell-distancing devices with extracellular membrane-distal domain,
(optional) elongation domain, extracellular membrane-proximal domain,
transmembrane domain, and/or intracellular domain

| | | | |
|---|---|---|---|
| 110 | 1882 | hIEE-Ig-3 | MVAGSDAGRALGVLSVVCLLHCFGFISCFSQQIYGVVYGN |
| | | | VTFHVPSNVPLKEVLWKKQKDKVAELENSEFRAFSSFKNR |
| | | | VYLDTVSGSLTIYNLTSSDEDEYEMESPNITDTMKFFLYVLE |
| | | | SLPSPTLTCALTNGSIEVQCMIPEHYNSHRGLIMYSWDCP |
| | | | MEQCKRNSTSIYFKMENDLPQKIQCTLSNPLFNTTSSIILTT |
| | | | CIPSSGHSRHRGSYPYDVPDYAGSSPSTPKKVTTVIQNPM |
| | | | PIREGDTVTLSCNYNSSNPSVTRYEWKPHGAWEEPSLGVL |
| | | | KIQNVGWDNTTIACAACNSWCSWASPVALNVQYAPRD |
| | | | VRVRKIKPLSEIHSGNSVSLQCDFSSSHPKEVQFFWEKNGR |
| | | | LLGKESQLNFDSISPEDAGSYSCWVNNSIGQTASKAWTLE |
| | | | VLYAPRRLRVSMSPGDQVMEGKSATLTCESDANPPVSHY |
| | | | TWFDWNNQSLPYHSQKLRLEPVKVQHSGAYWCQGTNS |
| | | | VGKGRSPLSTLTVYYSPETISRHRYALIPIPLAVITTCIVLYM |
| | | | NGILKCDRKPDRTNSN |

### Examples of Sequences of Cell-Distancing Devices;

In the following examples:
Unbolded continuous underlining indicates an extracellular membrane-distal domain,
Dashed underlining indicates an elongation domain,
Double underlining indicates a combination of any of an extracellular membrane-proximal domain, a transmembrane domain and/or an intracellular domain
Italicized double underlining indicates a transmembrane domain.
Bold underlined lettering indicates restriction sites. Kozak sequences are boxed. Hinges are indicated by bold lettering and tags are represented in italics.

1882: hIEE-Ig-3 (FIG. 5E)
(SEQ ID NO: 55)

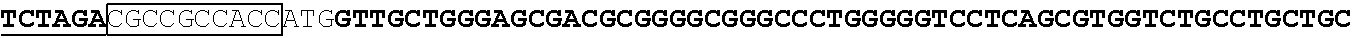

ACTGCTTTGGTTTCATCAGCTGTTTTTCCCAACAAATATATGGTGTTGTGTATGGGAATGTAACTTTCCATGTACCA
AGCAATGTGCCTTTAAAAGAGGTCCTATGGAAAAAACAAAAGGATAAAGTTGCAGAACTGGAAAATTCTGAGTTCAG
AGCTTTCTCATCTTTTAAAAATAGGGTTTATTTAGACACTGTGTCAGGTAGCCTCACTATCTACAACTTAACATCAT
CAGATGAAGATGAGTATGAAATGGAATCGCCAAATATTACTGATACCATGAAGTTCTTTCTTTATGTGCTTGAGTCT
CTTCCATCTCCCACACTAACTTGTGCATTGACTAATGGAAGCATTGAAGTCCAATGCATGATACCAGAGCATTACAA
CAGCCATCGAGGACTTATAATGTACTCATGGGATTGTCCTATGGAGCAATGTAAACGTAACTCAACCAGTATATATT
TTAAGATGGAAAATGATCTTCCACAAAAAATACAGTGTACTCTTAGCAATCCATTATTTAATACAACATCATCAATC
ATTTTGACAACCTGTATCCCAAGCAGCGGTCATTCAAGACACAGAGGGTCCTACCCCTACGACGTTCCCGACTACGC

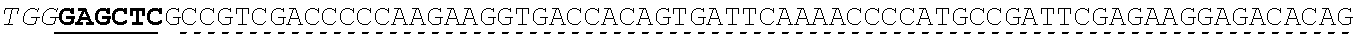

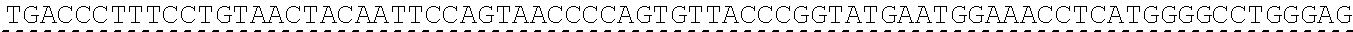

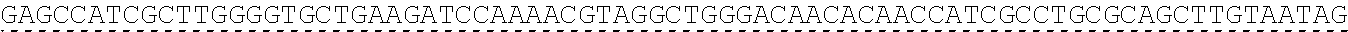

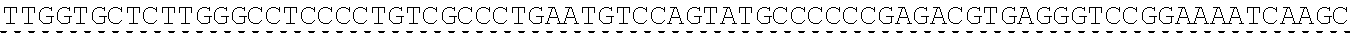

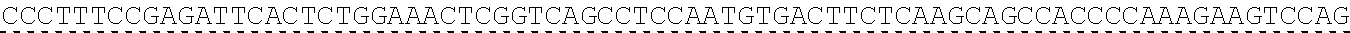

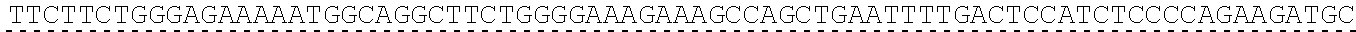

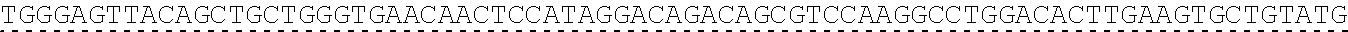

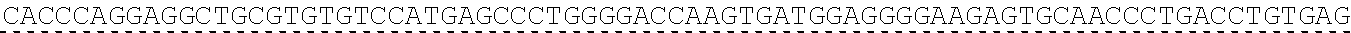

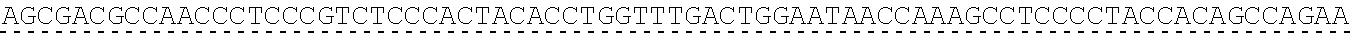

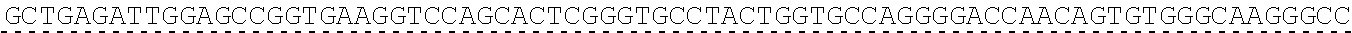

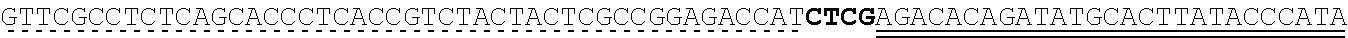

CCATTAGCAGTAATTACAACATGTATTGTGCTGTATATGAATGGTATTCTGAAATGTGACAGAAAACCAGACAGAAC
CAACTCCAATTGAGCGGCCGC

(SEQ ID NO: 110)

MVAGSDAGRALGVLSVVCLLHCFGFISCFSQQIYGVVYGNVTFHVPSNVPLKEVLWKKQKDKVAELENSEFRAFSSF
KNRVYLDTVSGSLTIYNLTSSDEDEYEMESPNITDTMKFFLYVLESLPSPTLTCALINGSIEVQCMIPEHYNSHRGL
IMYSWDCPMEQCKRNSTSIYFKMENDLPQKIQCTLSNPLFNTTSSIILTTCIPSSGHSRHRGSYPYDVPDYAGSSPS
TPKKVTTVIQNPMPIREGDTVTLSCNYNSSNPSVTRYEWKPHGAWEEPSLGVLKIQNVGWDNTTIACAACNSWCSWA
SPVALNVQYAPRDVRVRKIKPLSEIHSGNSVSLQCDFSSSHPKEVOFFWEKNGRLLGKESQLNFDSISPEDAGSYSC
WVNNSIGQTASKAWTLEVLYAPRRLRVSMSPGDQVMEGKSATLTCESDANPPVSHYTWFDWNNQSLPYHSQKLRLEP
VKVQHSGAYWCQGTNSVGKGRSPLSTLTVYYSPETISRHRYALIPIPLAVITTCIVLYMNGILKCDRKPDRTNSN

1883: hIEE-0-3 (FIG. 5E)
(SEQ ID NO: 111)

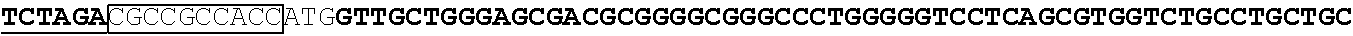

ACTGCTTTGGTTTCATCAGCTGTTTTTCCCAACAAATATATGGTGTTGTGTATGGGAATGTAACTTTCCATGTACCA
AGCAATGTGCCTTTAAAAGAGGTCCTATGGAAAAAACAAAAGGATAAAGTTGCAGAACTGGAAAATTCTGAGTTCAG
AGCTTTCTCATCTTTTAAAAATAGGGTTTATTTAGACACTGTGTCAGGTAGCCTCACTATCTACAACTTAACATCAT
CAGATGAAGATGAGTATGAAATGGAATCGCCAAATATTACTGATACCATGAAGTTCTTTCTTTATGTGCTTGAGTCT
CTTCCATCTCCCACACTAACTTGTGCATTGACTAATGGAAGCATTGAAGTCCAATGCATGATACCAGAGCATTACAA
CAGCCATCGAGGACTTATAATGTACTCATGGGATTGTCCTATGGAGCAATGTAAACGTAACTCAACCAGTATATATT
TTAAGATGGAAAATGATCTTCCACAAAAAATACAGTGTACTCTTAGCAATCCATTATTTAATACAACATCATCAATC
ATTTTGACAACCTGTATCCCAAGCAGCGGTCATTCAAGACACAGAGGGTCCTACCCCTACGACGTTCCCGACTACGC

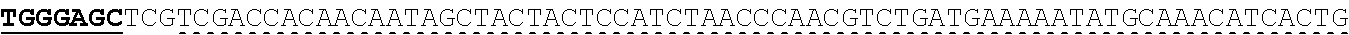

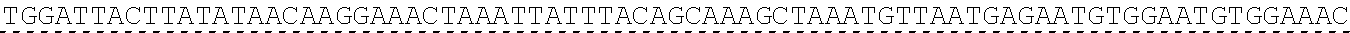

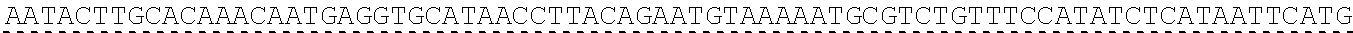

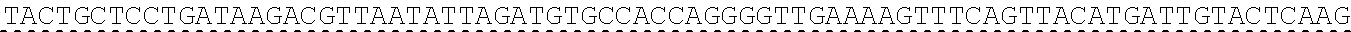

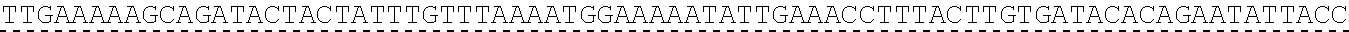

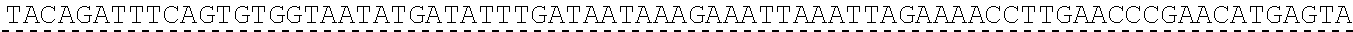

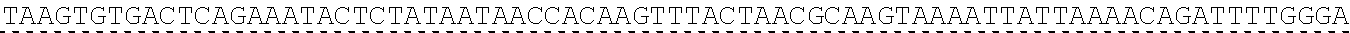

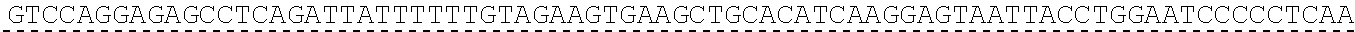

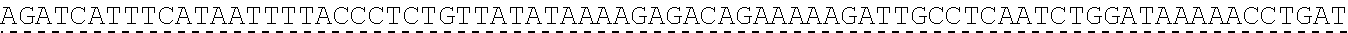

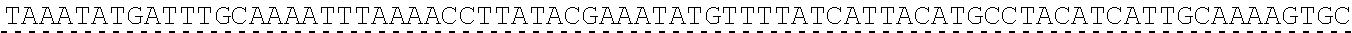

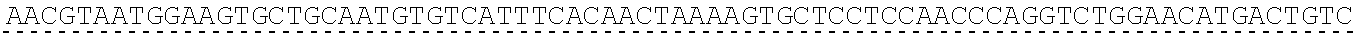

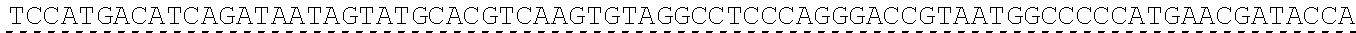

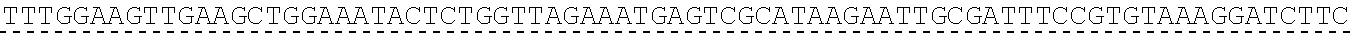

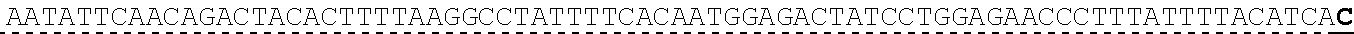

TCGAGACACAGATATGCACTTATACCCATACCATTAGCAGTAATTACAACATGTATTGTGCTGTATATGAATGGTAT
TCTGAAATGTGACAGAAAACCAGACAGAACCAACTCCAATTGAGCGGCCGC

(SEQ ID NO: 112)

MVAGSDAGRALGVLSVVCLLHCFGFISCFSQQIYGVVYGNVTFHVPSNVPLKEVLWKKQKDKVAELENSEFRAFSSF
KNRVYLDTVSGSLTIYNLTSSDEDEYEMESPNITDTMKFFLYVLESLPSPTLTCALINGSIEVQCMIPEHYNSHRGL
IMYSWDCPMEQCKRNSTSIYFKMENDLPQKIQCTLSNPLFNTTSSIILTTCIPSSGHSRHRGSYPYDVPDYAGSSST

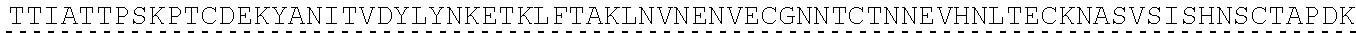

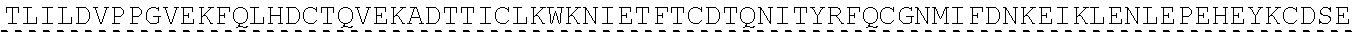

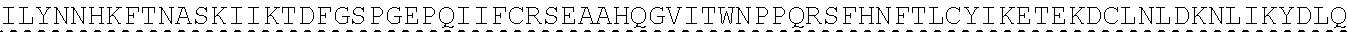

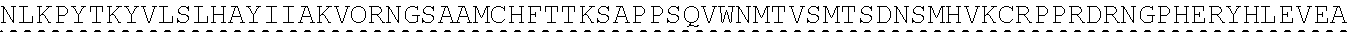

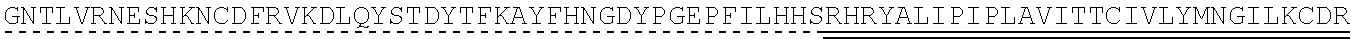

KPDRINSN

1884: hIEE-ABC-3 (FIG. 5E)
(SEQ ID NO: 113)

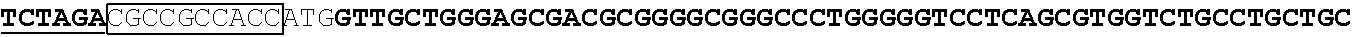

ACTGCTTTGGTTTCATCAGCTGTTTTTCCCAACAAATATATGGTGTTGTGTATGGGAATGTAACTTTCCATGTACCA
AGCAATGTGCCTTTAAAAGAGGTCCTATGGAAAAAACAAAAGGATAAAGTTGCAGAACTGGAAAATTCTGAGTTCAG
AGCTTTCTCATCTTTTAAAAATAGGGTTTATTTAGACACTGTGTCAGGTAGCCTCACTATCTACAACTTAACATCAT
CAGATGAAGATGAGTATGAAATGGAATCGCCAAATATTACTGATACCATGAAGTTCTTTCTTTATGTGCTTGAGTCT
CTTCCATCTCCCACACTAACTTGTGCATTGACTAATGGAAGCATTGAAGTCCAATGCATGATACCAGAGCATTACAA
CAGCCATCGAGGACTTATAATGTACTCATGGGATTGTCCTATGGAGCAATGTAAACGTAACTCAACCAGTATATATT
TTAAGATGGAAAATGATCTTCCACAAAAAATACAGTGTACTCTTAGCAATCCATTATTTAATACAACATCATCAATC
ATTTTGACAACCTGTATCCCAAGCAGCGGTCATTCAAGACACAGAGGGTCCTACCCCTACGACGTTCCCGACTACGC

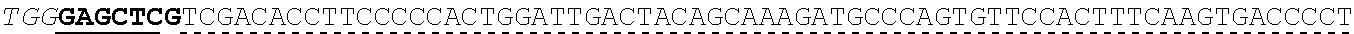

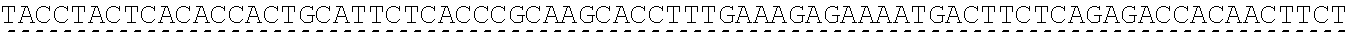

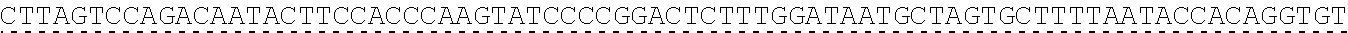

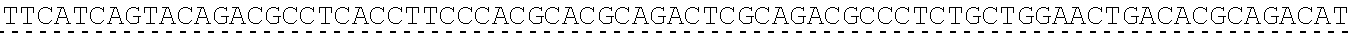

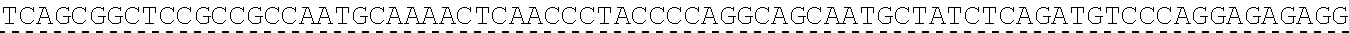

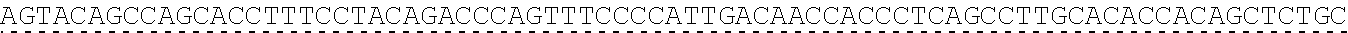

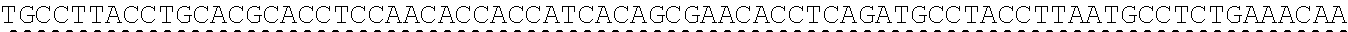

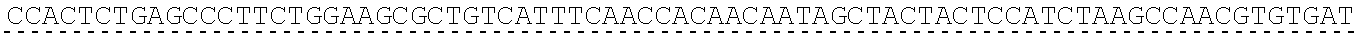

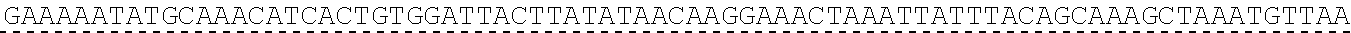

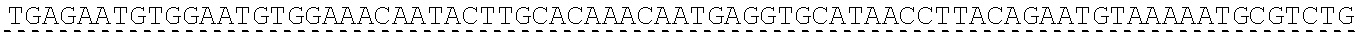

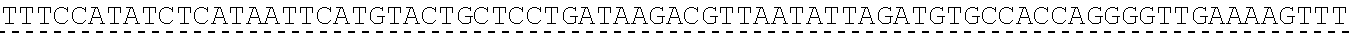

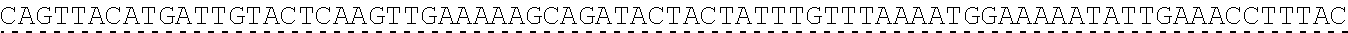

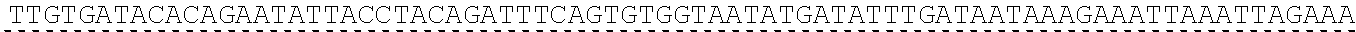

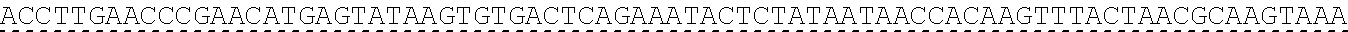

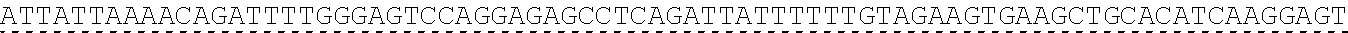

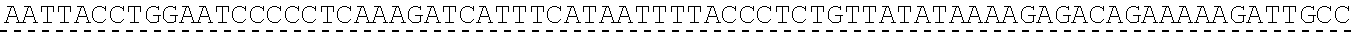

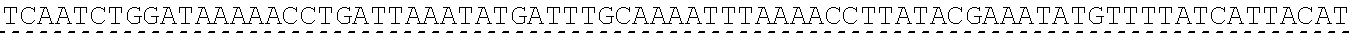

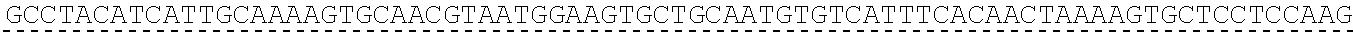

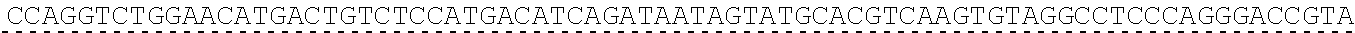

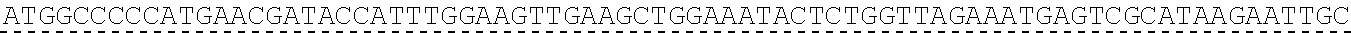

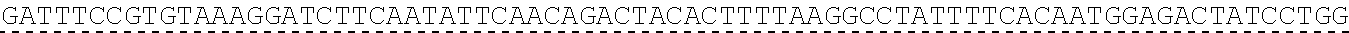

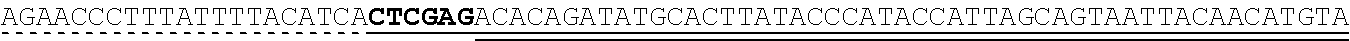

TTGTGCTGTATATGAATGGTATTCTGAAATGTGACAGAAAACCAGACAGAACCAACTCCAATTGAGCGGCCGC

(SEQ ID NO: 114)

MVAGSDAGRALGVLSVVCLLHCFGFISCFSQQIYGVVYGNVTFHVPSNVPLKEVLWKKQKDKVAELENSEFRAFSSF
KNRVYLDTVSGSLTIYNLTSSDEDEYEMESPNITDTMKFFLYVLESLPSPTLTCALINGSIEVQCMIPEHYNSHRGL
IMYSWDCPMEQCKRNSTSIYFKMENDLPQKIQCTLSNPLFNTTSSIILTTCIPSSGHSRHRGSYPYDVPDYAGSSST

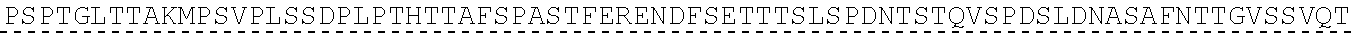

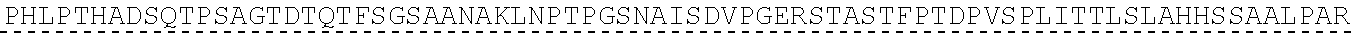

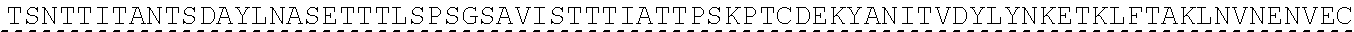

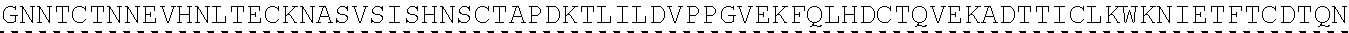

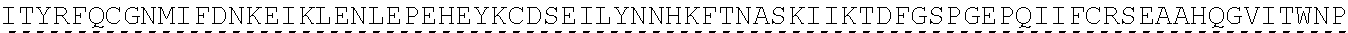

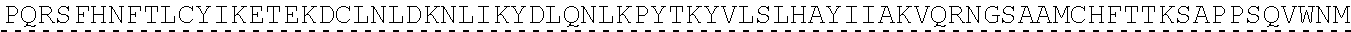

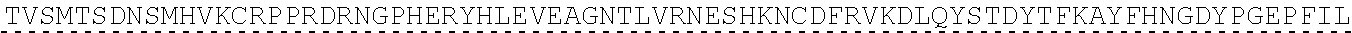

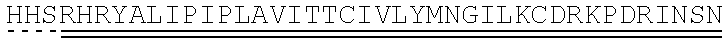

1885: mIEE-Ig-3 (FIG. 5E)
(SEQ ID NO: 115)

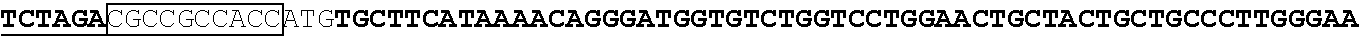

CTGGATTTCAAGGTCATTCAATACCAGATATAAATGCCACCACCGGCAGCAATGTAACCCTGAAAATCCATAAGGAC
CCACTTGGACCATATAAACGTATCACCTGGCTTCATACTAAAAATCAGAAGATTTTAGAGTACAACTATAATAGTAC
AAAGACAATCTTCGAGTCTGAATTTAAAGGCAGGGTTTATCTTGAAGAAAACAATGGTGCACTTCATATCTCTAATG
TCCGGAAAGAGGACAAAGGTACCTACTACATGAGAGTGCTGCGTGAAACTGAGAACGAGTTGAAGATAACCCTGGAA
GTATTTGATCCTGTGCCCAAGCCTTCCATAGAAATCAATAAGACTGAAGCCTCGACTGATTCCTGTCACCTGAGGCT
ATCGTGTGAGGTAAAGGACCAGCATGTTGACTATACTTGGTATGAGAGCAGCGGACCTTTCCCCAAAAAGAGTCCAG
GATATGTGCTCGATCTCATCGTCACACCACAGAACAAGTCTACATTTTACACCTGCCAAGTCAGCAATCCTGTAAGC
AGCAAGAACGACACAGTGTACTTCACTCTACCTTGTGATCTAGCCAGATCTGGGTCCTACCCCTACGACGTTCCCGA

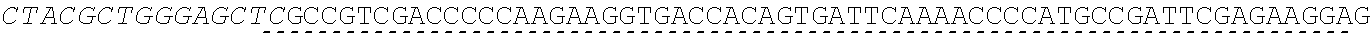

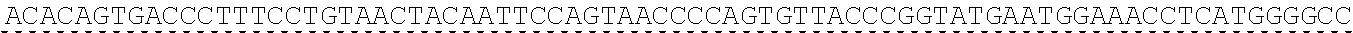

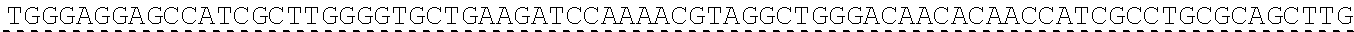

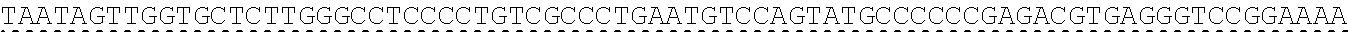

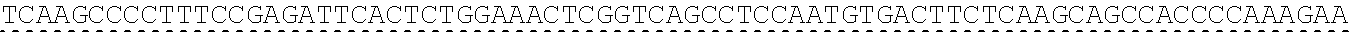

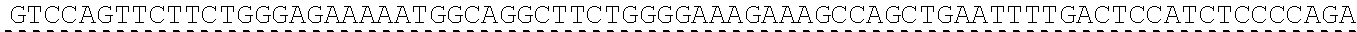

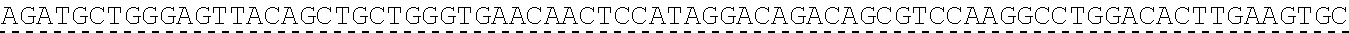

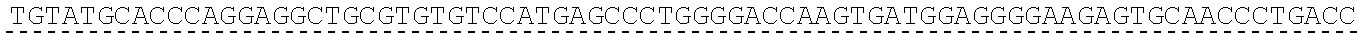

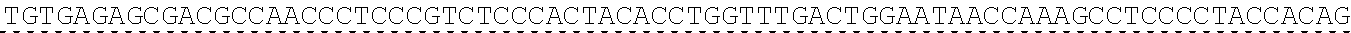

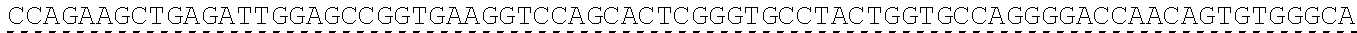

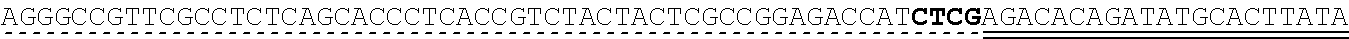

CCCATACCATTAGCAGTAATTACAACATGTATTGTGCTGTATATGAATGGTATTCTGAAATGTGACAGAAAACCAGA
CAGAACCAACTCCAATTGAGCGGCCGC

(SEQ ID NO: 116)

MCFIKQGWCLVLELLLLPLGTGFQGHSIPDINATTGSNVTLKIHKDPLGPYKRITWLHTKNQKILEYNYNSTKTIFE
SEFKGRVYLEENNGALHISNVRKEDKGTYYMRVLRETENELKITLEVFDPVPKPSIEINKTEASTDSCHLRLSCEVK
DQHVDYTWYESSGPFPKKSPGYVLDLIVTPQNKSTFYTCQVSNPVSSKNDTVYFTLPCDLARSGSYPYDVPDYAGSS

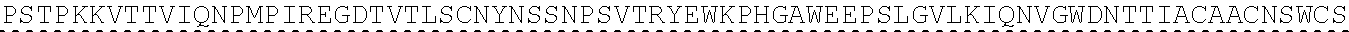

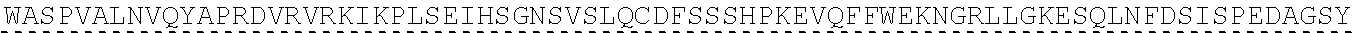

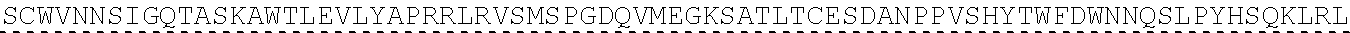

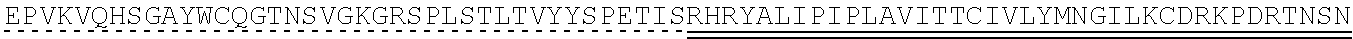

1886: mIEE-O-3 (FIG. 5E)
(SEQ ID NO: 117)

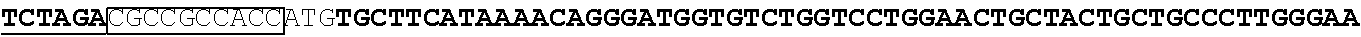

CTGGATTTCAAGGTCATTCAATACCAGATATAAATGCCACCACCGGCAGCAATGTAACCCTGAAAATCCATAAGGAC
CCACTTGGACCATATAAACGTATCACCTGGCTTCATACTAAAAATCAGAAGATTTTAGAGTACAACTATAATAGTAC
AAAGACAATCTTCGAGTCTGAATTTAAAGGCAGGGTTTATCTTGAAGAAAACAATGGTGCACTTCATATCTCTAATG
TCCGGAAAGAGGACAAAGGTACCTACTACATGAGAGTGCTGCGTGAAACTGAGAACGAGTTGAAGATAACCCTGGAA
GTATTTGATCCTGTGCCCAAGCCTTCCATAGAAATCAATAAGACTGAAGCCTCGACTGATTCCTGTCACCTGAGGCT
ATCGTGTGAGGTAAAGGACCAGCATGTTGACTATACTTGGTATGAGAGCAGCGGACCTTTCCCCAAAAAGAGTCCAG
GATATGTGCTCGATCTCATCGTCACACCACAGAACAAGTCTACATTTTACACCTGCCAAGTCAGCAATCCTGTAAGC
AGCAAGAACGACACAGTGTACTTCACTCTACCTTGTGATCTAGCCAGATCTGGGTCCTACCCCTACGACGTTCCCGA

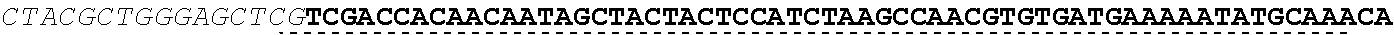

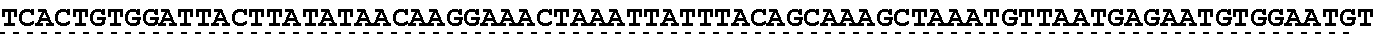

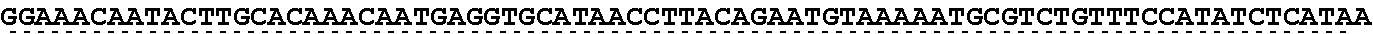

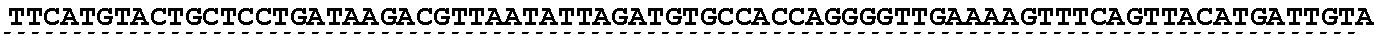

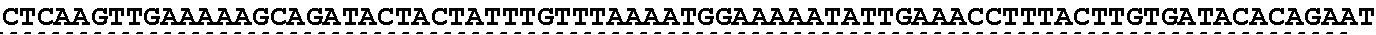

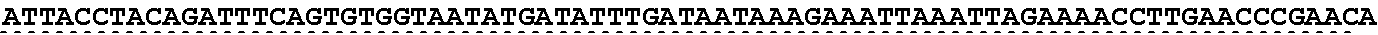

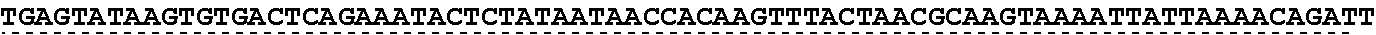

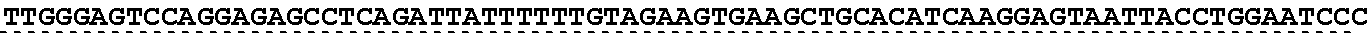

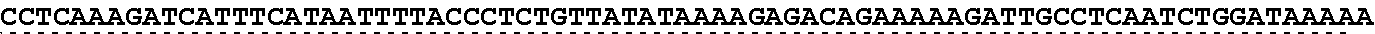

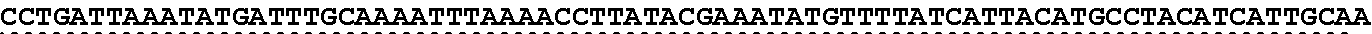

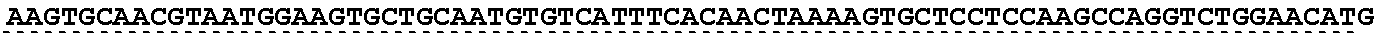

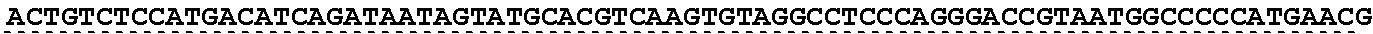

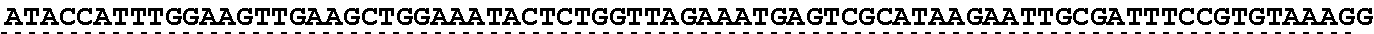

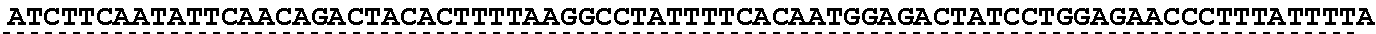

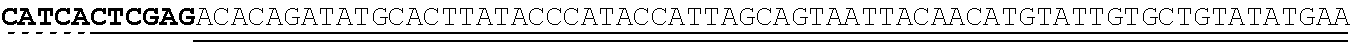

TGGTATTCTGAAATGTGACAGAAAACCAGACAGAACCAACTCCAATTGAGCGGCCGC

(SEQ ID NO: 118)

MCFIKQGWCLVLELLLLPLGTGFQGHSIPDINATTGSNVTLKIHKDPLGPYKRITWLHTKNQKILEYNYNSTKTIFE
SEFKGRVYLEENNGALHISNVRKEDKGTYYMRVLRETENELKITLEVFDPVPKPSIEINKTEASTDSCHLRLSCEVK
DQHVDYTWYESSGPFPKKSPGYVLDLIVTPQNKSTFYTCQVSNPVSSKNDTVYFTLPCDLARSGSYPYDVPDYAGSS

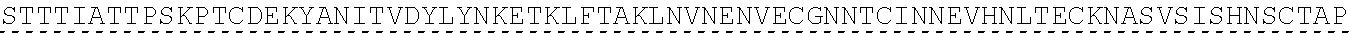

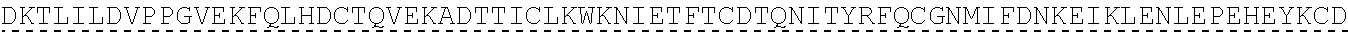

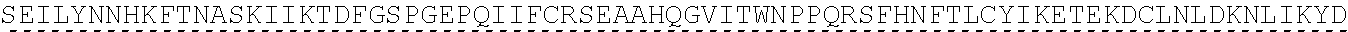

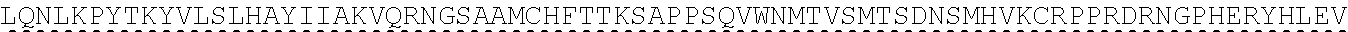

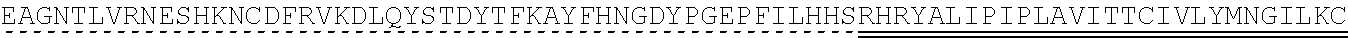

DRKPDRTNSN

1887: mIEE-ABC-3 (FIG. 5E)

(SEQ ID NO: 119)

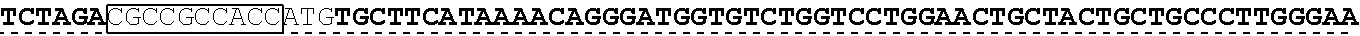

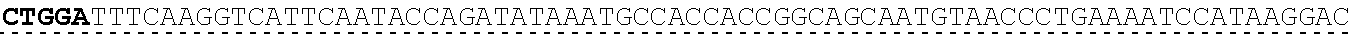

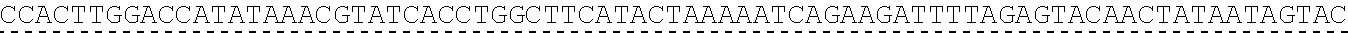

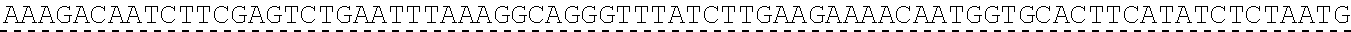

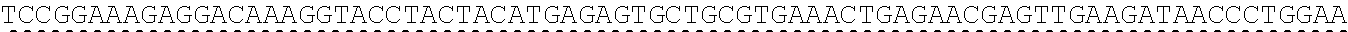

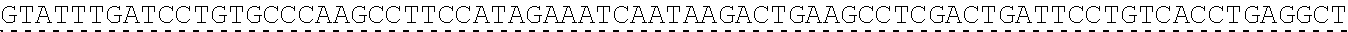

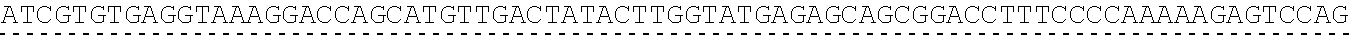

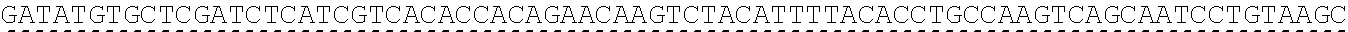

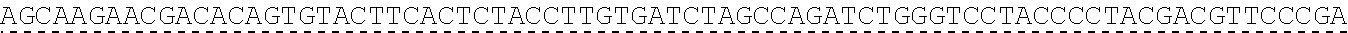

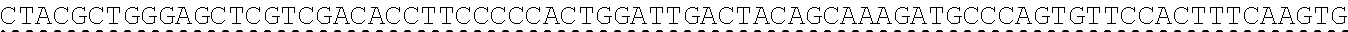

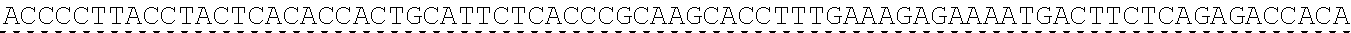

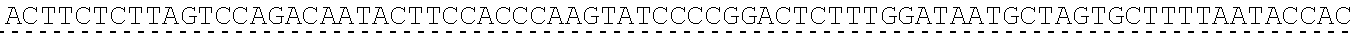

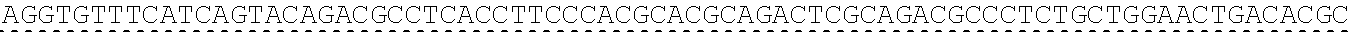

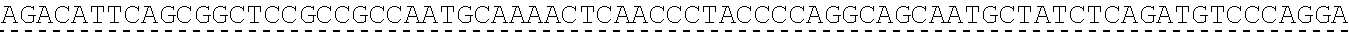

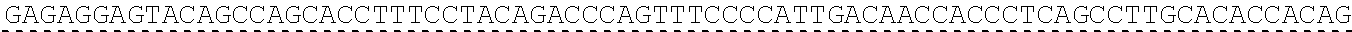

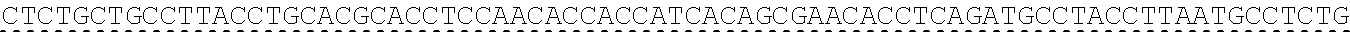

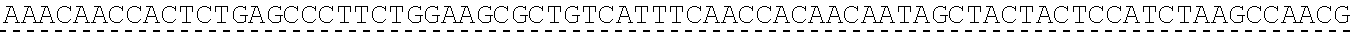

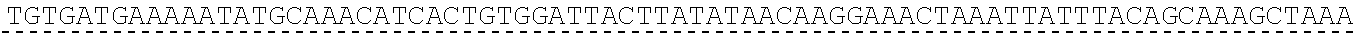

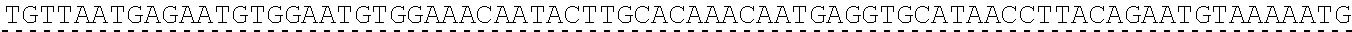

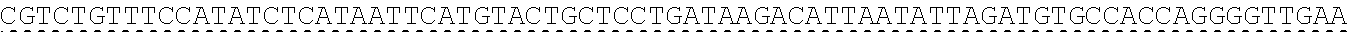

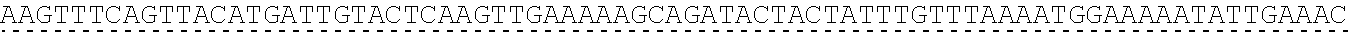

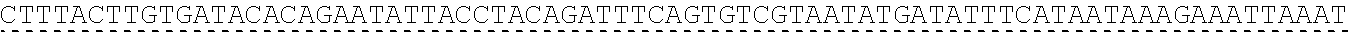

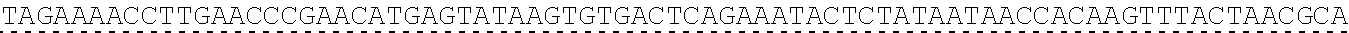

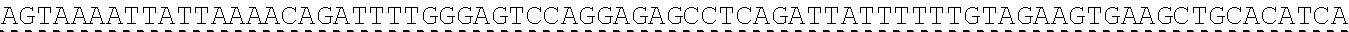

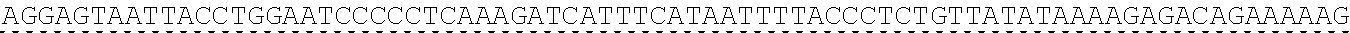

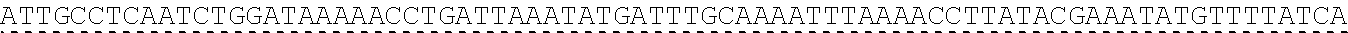

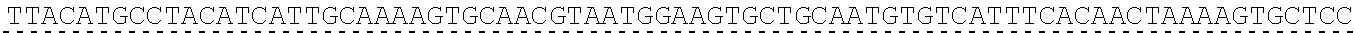

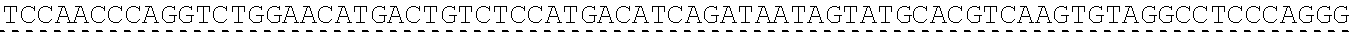

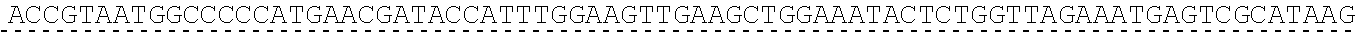

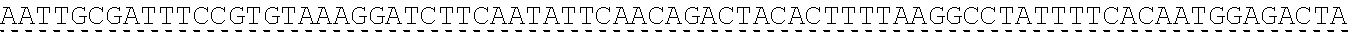

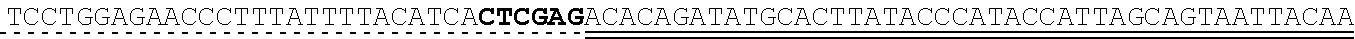

CATGTATTGTGCTGTATATGAATGGTATTCTGAAATGTGACAGAAAACCAGACAGAACCAACTCCAATTGAGCGGCC

(SEQ ID NO: 120)

MCFIKOGWCLVLELLLLPLGTGFQGHSIPDINATTGSNVTLKIHKDPLGPYKRITWLHTKNQKILEYNYNSTKTIFE
SEFKGRVYLEENNGALHISNVRKEDKGTYYMRVLRETENELKITLEVFDPVPKPSIEINKTEASTDSCHLRLSCEVK
DQHVDYTWYESSGPFPKKSPGYVLDLIVTPQNKSTFYTCQVSNPVSSKNDTVYFTLPCDLARSGSYPYDVPDYAGSS

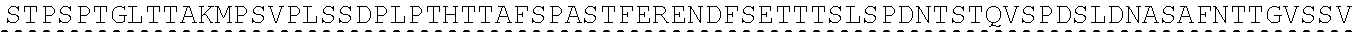

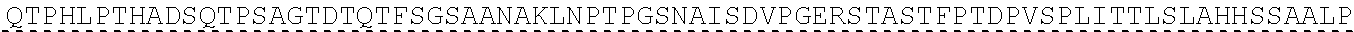

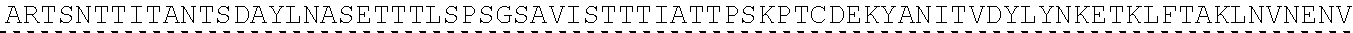

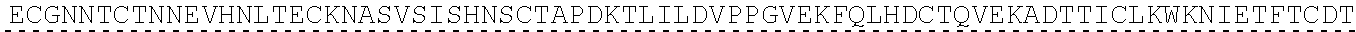

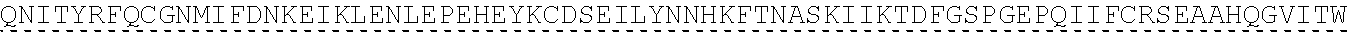

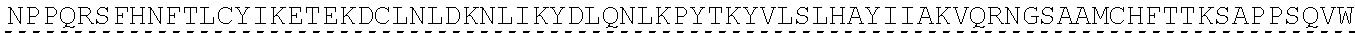

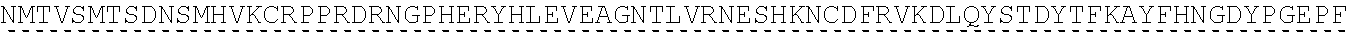

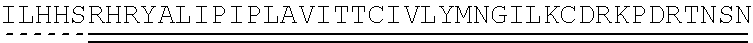

1941: hIEE-22 (FIG. 5G)
(SEQ ID NO: 121)

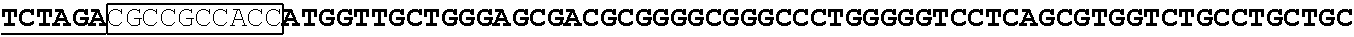

ACTGCTTTGGTTTCATCAGCTGTTTTTCCCAACAAATATATGGTGTTGTGTATGGGAATGTAACTTTCCATGTACCA
AGCAATGTGCCTTTAAAAGAGGTCCTATGGAAAAAACAAAAGGATAAAGTTGCAGAACTGGAAAATTCTGAGTTCAG
AGCTTTCTCATCTTTTAAAAATAGGGTTTATTTAGACACTGTGTCAGGTAGCCTCACTATCTACAACTTAACATCAT
CAGATGAAGATGAGTATGAAATGGAATCGCCAAATATTACTGATACCATGAAGTTCTTTCTTTATGTGCTTGAGTCT
CTTCCATCTCCCACACTAACTTGTGCATTGACTAATGGAAGCATTGAAGTCCAATGCATGATACCAGAGCATTACAA
CAGCCATCGAGGACTTATAATGTACTCATGGGATTGTCCTATGGAGCAATGTAAACGTAACTCAACCAGTATATATT
TTAAGATGGAAAATGATCTTCCACAAAAAATACAGTGTACTCTTAGCAATCCATTATTTAATACAACATCATCAATC

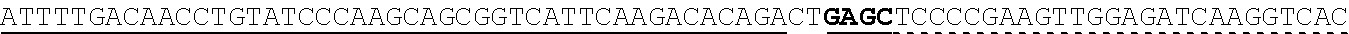

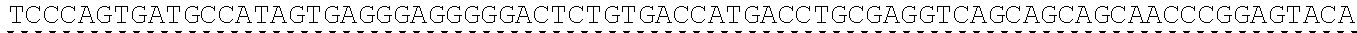

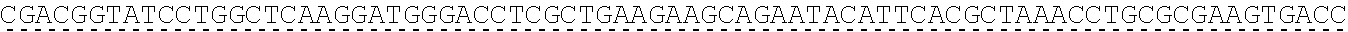

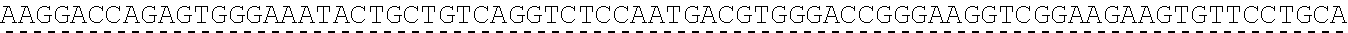

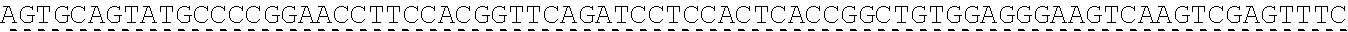

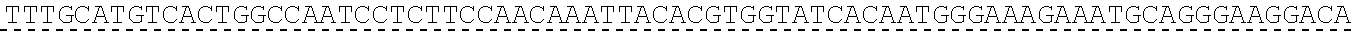

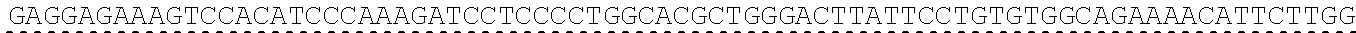

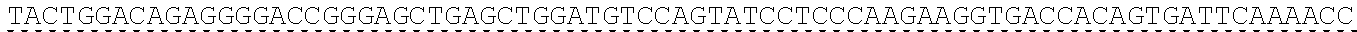

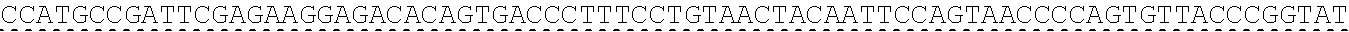

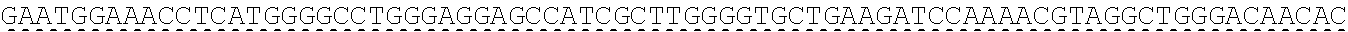

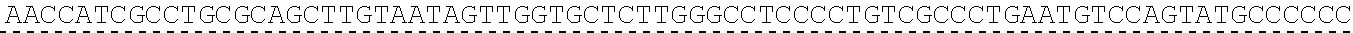

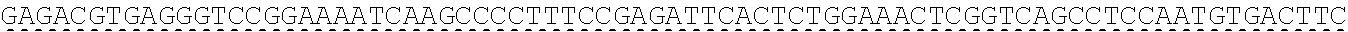

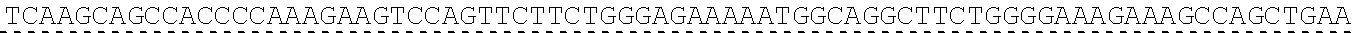

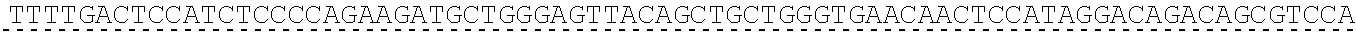

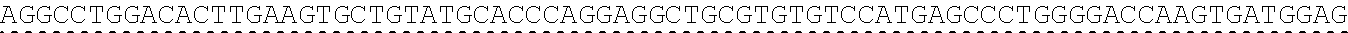

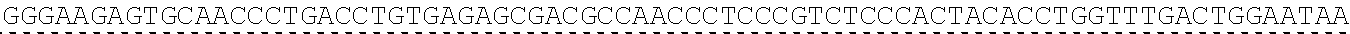

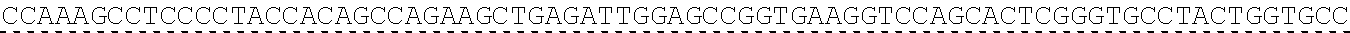

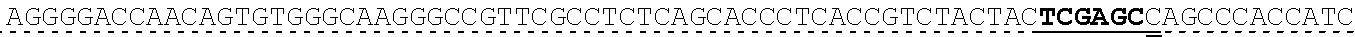

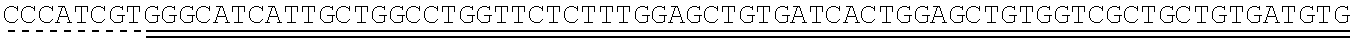

GAGGAGGAAAAGCTCAGATAGAAAAGGAGGGAGCTACTCTCAGGCTGCAAGCAGTGACAGTGCCCAGGGCTCTGATG
TGTCTCTCACAGCTTGTAAAGTGTGAGCGGCCGC

(SEQ ID NO: 122)

| | |
|---|---|
| MVAGSDAGRALGVLSVVCLLHCFGFISCFSQQIYGVVYGNVTFHVPSNVPLKEVLWKKQK | 60 |
| DKVAELENSEFRAFSSFKNRVYLDTVSGSLTIYNLTSSDEDEYEMESPNITDTMKFFLYV | 120 |
| LESLPSPTLTCALINGSIEVQCMIPEHYNSHRGLIMYSWDCPMEQCKRNSTSIYFKMEND | 180 |
| 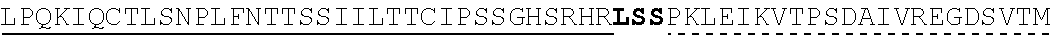 | 240 |
| 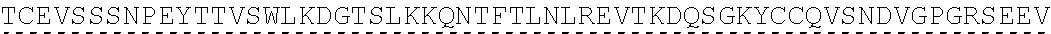 | 300 |
| 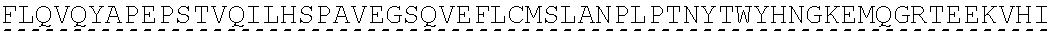 | 360 |
| 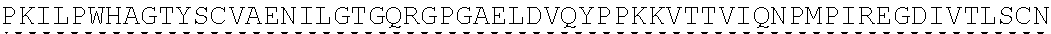 | 420 |
| 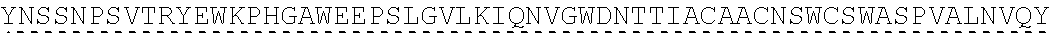 | 480 |
| 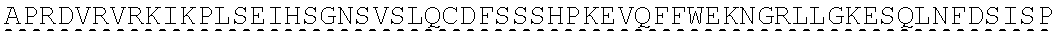 | 540 |
| 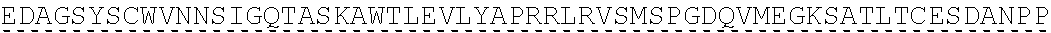 | 600 |
| 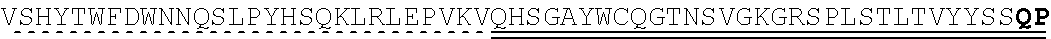 | 660 |
| 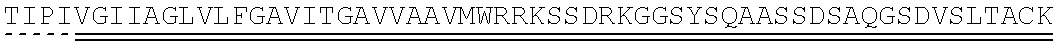 | 720 |
| V | 721 |

1942: hIEE-45 (FIG. 5G)
(SEQ ID NO: 123)

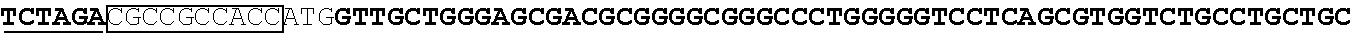

ACTGCTTTGGTTTCATCAGCTGTTTTTCCCAACAAATATATGGTGTTGTGTATGGGAATGTAACTTTCCATGTACCA
AGCAATGTGCCTTTAAAAGAGGTCCTATGGAAAAAACAAAAGGATAAAGTTGCAGAACTGGAAAATTCTGAGTTCAG
AGCTTTCTCATCTTTTAAAAATAGGGTTTATTTAGACACTGTGTCAGGTAGCCTCACTATCTACAACTTAACATCAT
CAGATGAAGATGAGTATGAAATGGAATCGCCAAATATTACTGATACCATGAAGTTCTTTCTTTATGTGCTTGAGTCT
CTTCCATCTCCCACACTAACTTGTGCATTGACTAATGGAAGCATTGAAGTCCAATGCATGATACCAGAGCATTACAA
CAGCCATCGAGGACTTATAATGTACTCATGGGATTGTCCTATGGAGCAATGTAAACGTAACTCAACCAGTATATATT
TTAAGATGGAAAATGATCTTCCACAAAAAATACAGTGTACTCTTAGCAATCCATTATTTAATACAACATCATCAATC

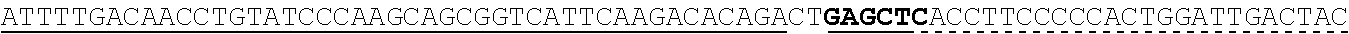

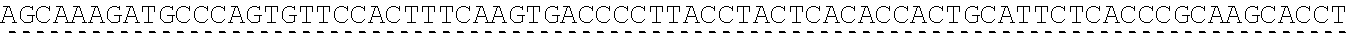

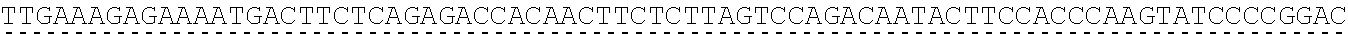

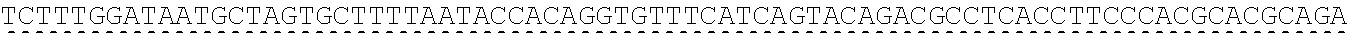

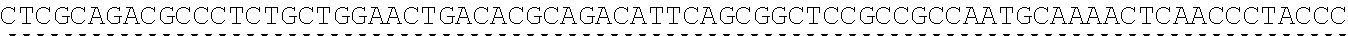

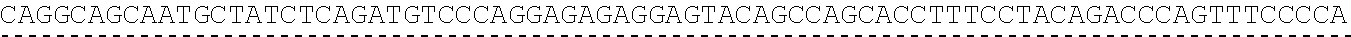

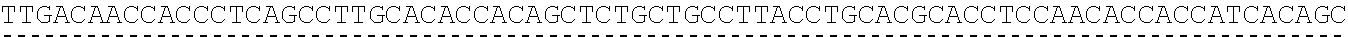

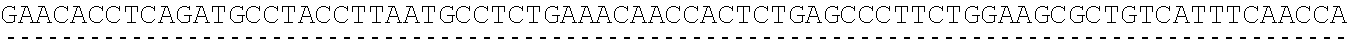

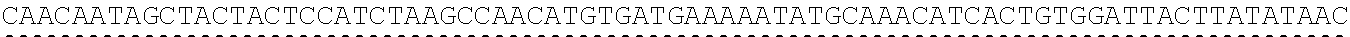

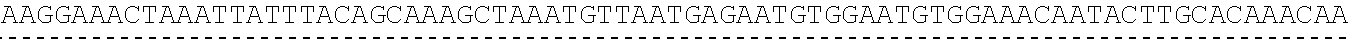

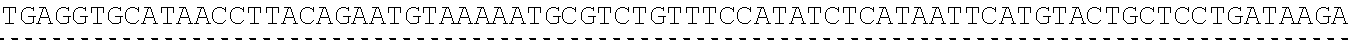

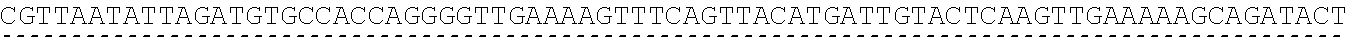

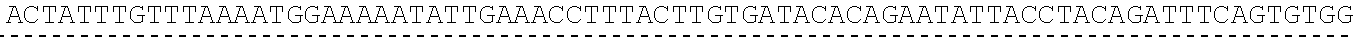

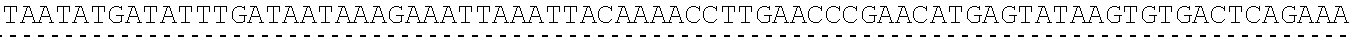

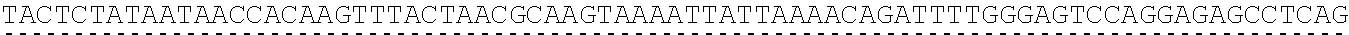

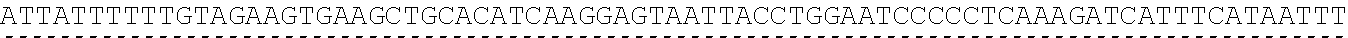

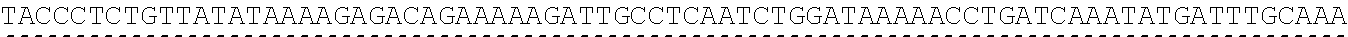

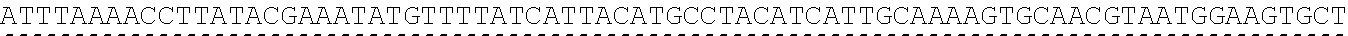

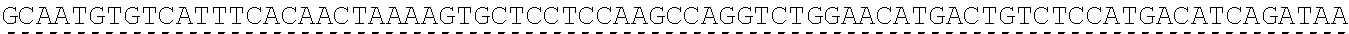

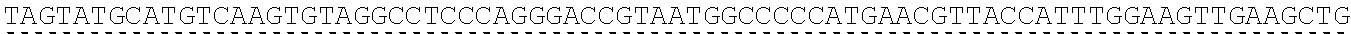

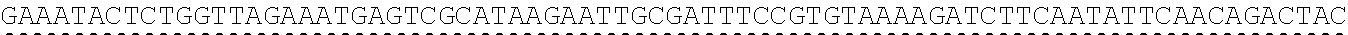

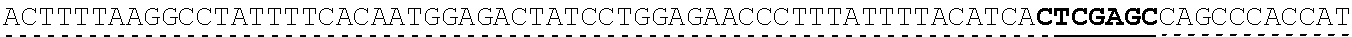

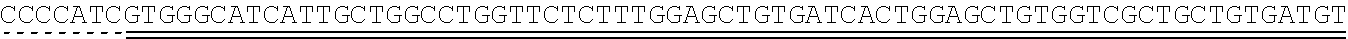

GGAGGAGGAAAAGCTCAGATAGAAAAGGAGGGAGCTACTCTCAGGCTGCAAGCAGTGACAGTGCCCAGGGCTCTGAT
GTGTCTCTCACAGCTTGTAAAGTGTGAGCGGCCGC

(SEQ ID NO: 124)

| | |
|---|---|
| MVAGSDAGRALGVLSVVCLLHCFGFISCFSQQIYGVVYGNVTFHVPSNVPLKEVLWKKQK | 60 |
| DKVAELENSEFRAFSSFKNRVYLDTVSGSLTIYNLTSSDEDEYEMESPNITDTMKFFLYV | 120 |
| LESLPSPTLICALINGSIEVQCMIPEHYNSHRGLIMYSWDCPMEQCKRNSTSIYFKMEND | 180 |
| 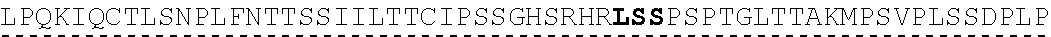 | 240 |
| 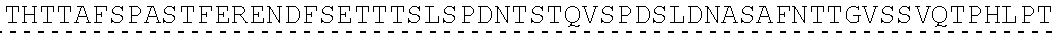 | 300 |
| 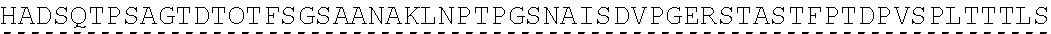 | 360 |
| 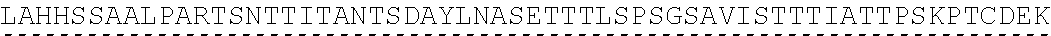 | 420 |
| 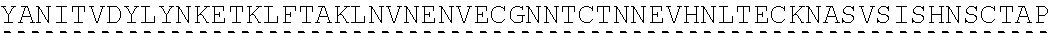 | 480 |
| 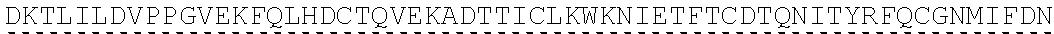 | 540 |
| 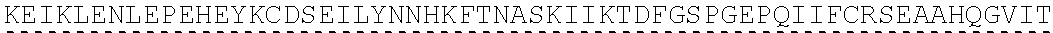 | 600 |
| 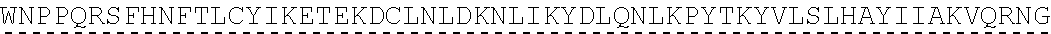 | 660 |
| 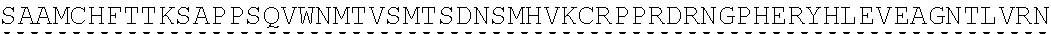 | 720 |
| 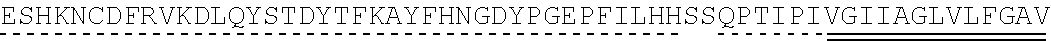 | 780 |
| ITGAVVAAVMWRRKSSDRKGGSYSQAASSDSAQGSDVSLTACKV | |

1943: hIEE-45 (FIG. 5G)
(SEQ ID NO: 125)

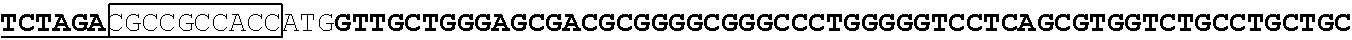

ACTGCTTTGGTTTCATCAGCTGTTTTTCCCAACAAATATATGGTGTTGTGTATGGGAATGTAACTTTCCATGTACCA
AGCAATGTGCCTTTAAAAGAGGTCCTATGGAAAAAACAAAAGGATAAAGTTGCAGAACTGGAAAATTCTGAGTTCAG
AGCTTTCTCATCTTTTAAAAATAGGGTTTATTTAGACACTGTGTCAGGTAGCCTCACTATCTACAACTTAACATCAT
CAGATGAAGATGAGTATGAAATGGAATCGCCAAATATTACTGATACCATGAAGTTCTTTCTTTATGTGCTTGAGTCT
CTTCCATCTCCCACACTAACTTGTGCATTGACTAATGGAAGCATTGAAGTCCAATGCATGATACCAGAGCATTACAA
CAGCCATCGAGGACTTATAATGTACTCATGGGATTGTCCTATGGAGCAATGTAAACGTAACTCAACCAGTATATATT
TTAAGATGGAAAATGATCTTCCACAAAAAATACAGTGTACTCTTAGCAATCCATTATTTAATACAACATCATCAATC

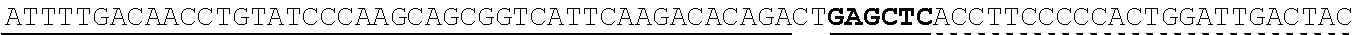

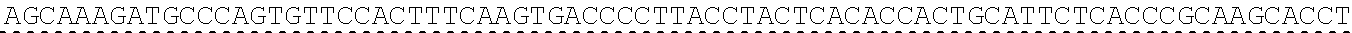

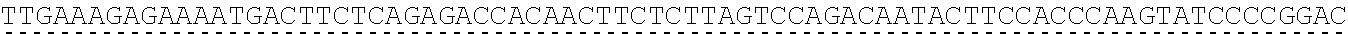

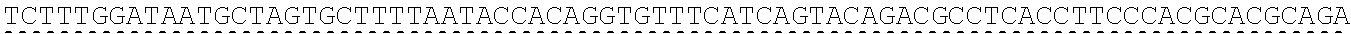

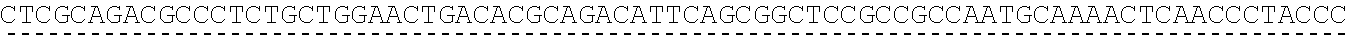

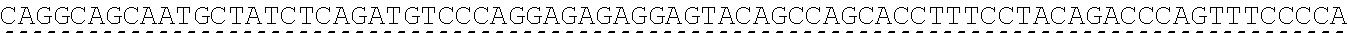

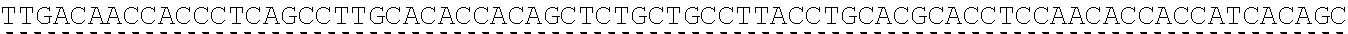

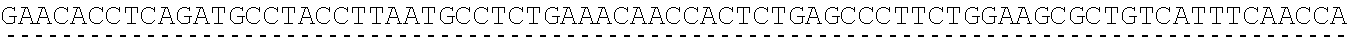

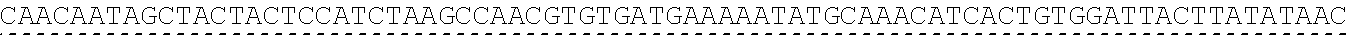

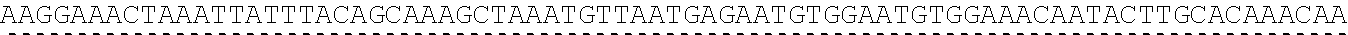

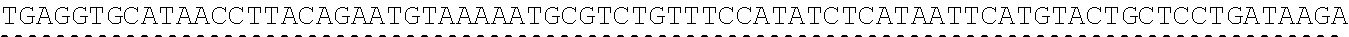

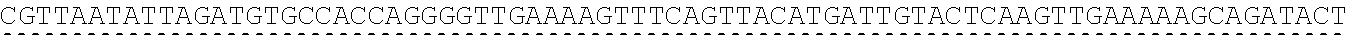

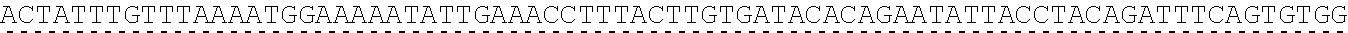

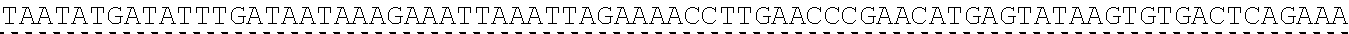

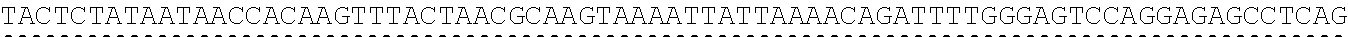

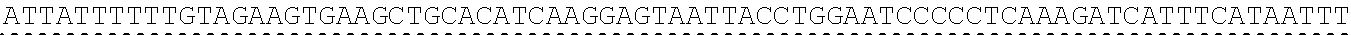

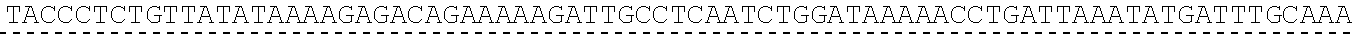

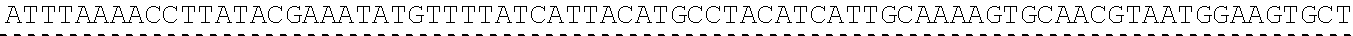

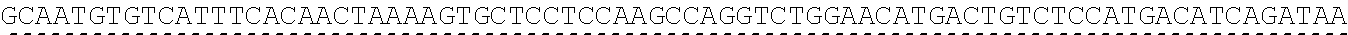

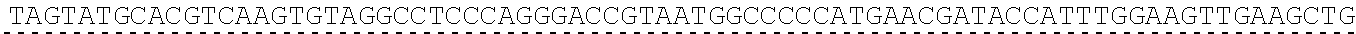

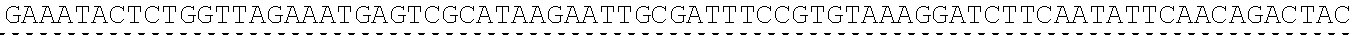

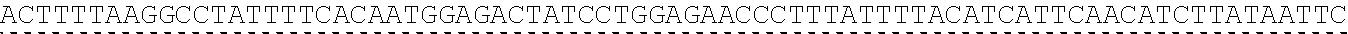

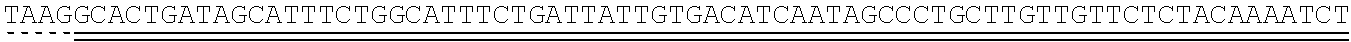

ATGATCTACATAAGAAAAGATCCTGCAATTGAGCGGCCGC

(SEQ ID NO: 126)

| | |
|---|---|
| MVAGSDAGRALGVLSVVCLLHCFGFISCFSQQIYGVVYGNVTFHVPSNVPLKEVLWKKQK | 60 |
| DKVAELENSEFRAFSSFKNRVYLDTVSGSLTIYNLISSDEDEYEMESPNITDTMKFFLYV | 120 |
| LESLPSPTLICALINGSIEVQCMIPEHYNSHRGLIMYSWDCPMEQCKRNSTSIYFKMEND | 180 |
| 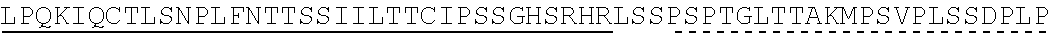 | 240 |
| 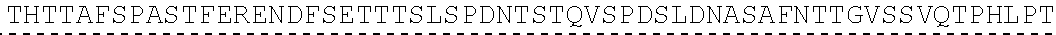 | 300 |
| 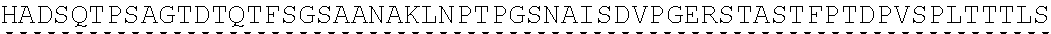 | 360 |
| 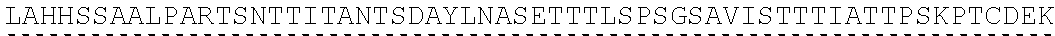 | 420 |
| 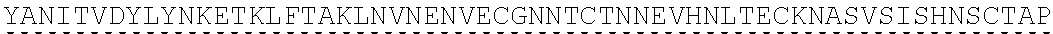 | 480 |
| 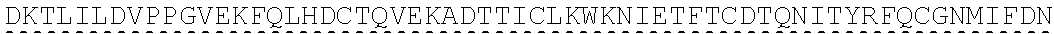 | 540 |
| 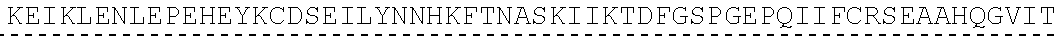 | 600 |
| 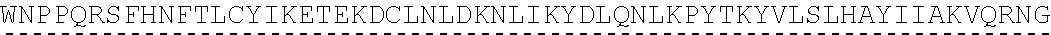 | 660 |
| 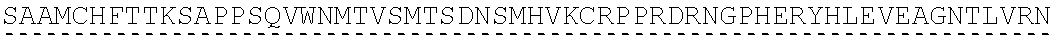 | 720 |
| 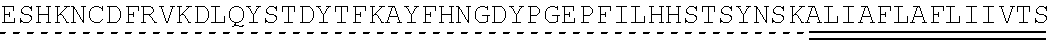 | 780 |
| IALLVVLYKIYDLHKKRSCN | 800 |

1944: mIEE-22 (FIG. 5G)
(SEQ ID NO: 127)

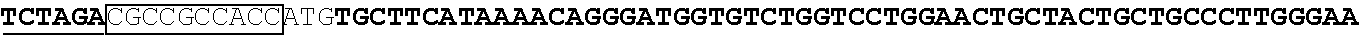

CTGGATTTCAAGGTCATTCAATACCAGATATAAATGCCACCACCGGCAGCAATGTAACCCTGAAAATCCATAAGGAC
CCACTTGGACCATATAAACGTATCACCTGGCTTCATACTAAAAATCAGAAGATTTTAGAGTACAACTATAATAGTAC
AAAGACAATCTTCGAGTCTGAATTTAAAGGCAGGGTTTATCTTGAAGAAAACAATGGTGCACTTCATATCTCTAATG
TCCGGAAAGAGGACAAAGGTACCTACTACATGAGAGTGCTGCGTGAAACTGAGAACGAGTTGAAGATAACCCTGGAA
GTATTTGATCCTGTGCCCAAGCCTTCCATAGAAATCAATAAGACTGAAGCCTCCACTGATTCCTGTCACCTGAGGCT
ATCGTGTGAGGTAAAGGACCAGCATGTTGACTATACTTGGTATGAGAGCAGCGGACCTTTCCCCAAAAAGAGTCCAG
GATATGTGCTCGATCTCATCGTCACACCACAGAACAAGTCTACATTTTACACCTGCCAAGTCAGCAATCCTGTAAGC

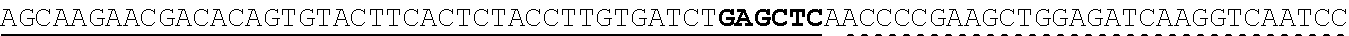

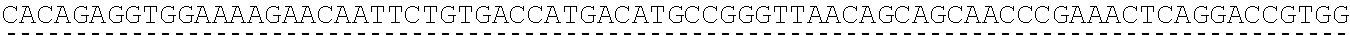

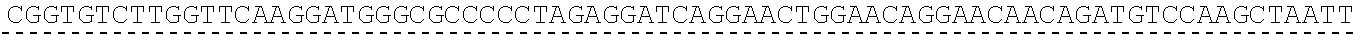

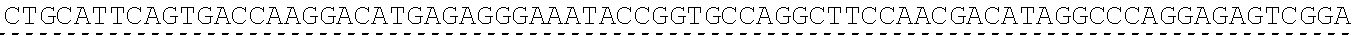

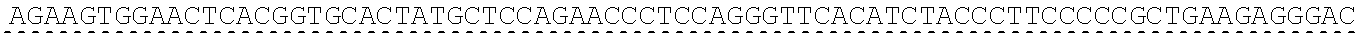

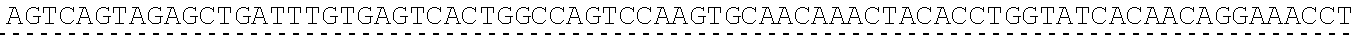

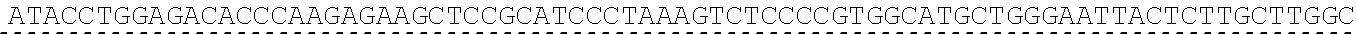

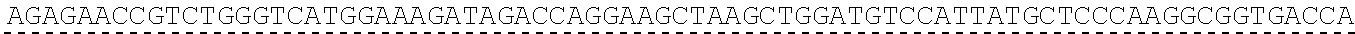

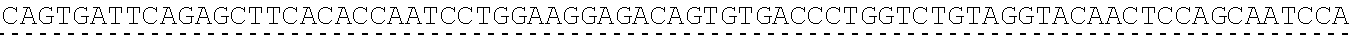

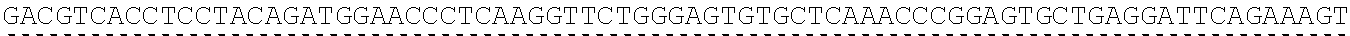

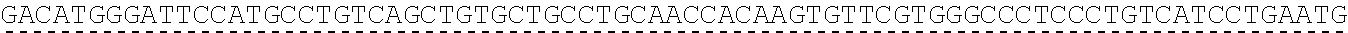

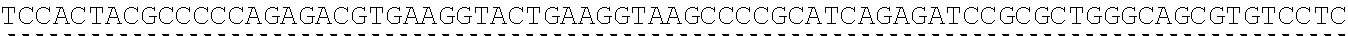

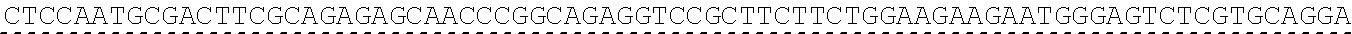

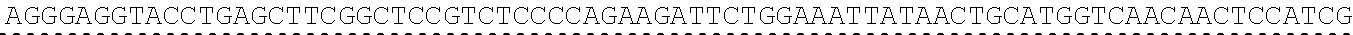

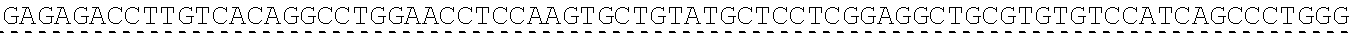

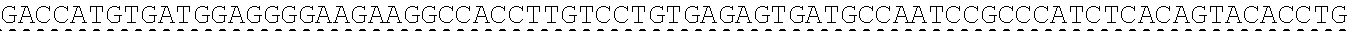

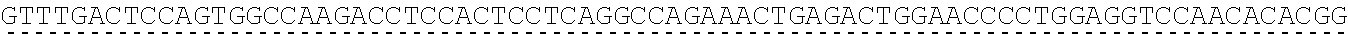

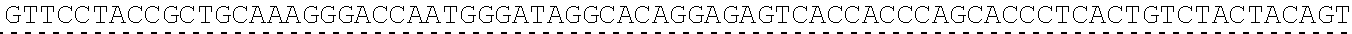

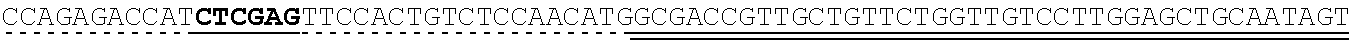

CACTGGAGCTGTGGTGGCTTTTGTGATGAAGATGAGAAGGAGAAACACAGGTGGAAAAGGAGGGGACTATGCTCTGG
CTCCAGGCTCCCAGACCTCTGATCTGTCTCTCCCAGATTGTAAAGTGATGGTTCATGACCCTCATTCTCTAGCGTGA
GCGGCCGC

(SEQ ID NO: 128)

| | |
|---|---|
| MCFIKQGWCLVLELLLLPLGTGFQGHSIPDINATTGSNVTLKIHKDPLGPYKRITWLHTK | 60 |
| NQKILEYNYNSTKTIFESEFKGRVYLEENNGALHISNVRKEDKGTYYMRVLRETENELKI | 120 |
| TLEVFDPVPKPSIEINKTEASTDSCHLRLSCEVKDQHVDYTWYESSGPFPKKSPGYVLDL | 180 |
| IVTPQNKSTFYTCQVSNPVSSKNDTVYFTLPCDLSSTPKLEIKVNPTEVEKNNSVTMTCR | 240 |
| VNSSNPKLRTVAVSWFKDGRPLEDQELEQEQQMSKLILHSVTKDMRGKYRCQASNDIGPG | 300 |
| ESEEVELTVHYAPEPSRVHIYPSPAEEGOSVELICESLASPSATNYTWYHNRKPIPGDTQ | 360 |
| EKLRIPKVSPWHAGNYSCLAENRLGHGKIDQEAKLDVHYAPKAVTTVIQSFTPILEGDSV | 420 |
| TLVCRYNSSNPDVTSYRWNPQGSGSVLKPGVLRIQKVTWDSMPVSCAACNHKCSWALPVI | 480 |
| GSVSPEDSGNYNCMVNNSIGETLSQAWNLQVLYAPRRLRVSISPGDHVMEGKKATLSCES | 600 |
| DANPPISQYTWFDSSGODLHSSGQKLRLEPLEVQHTGSYRCKGTNGIGTGESPPSTLTVY | 660 |
| 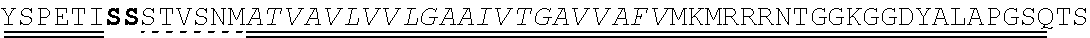 | 720 |
| DLSLPDCKVMVHDPHSLA | 738 |

1945: mIEE-45 (FIG. 5G)
(SEQ ID NO: 129)

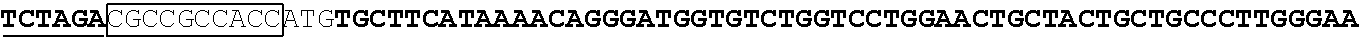

CTGGATTTCAAGGTCATTCAATACCAGATATAAATGCCACCACCGGCAGCAATGTAACCCTGAAAATCCATAAGGAC
CCACTTGGACCATATAAACGTATCACCTGGCTTCATACTAAAAATCAGAAGATTTTAGAGTACAACTATAATAGTAC
AAAGACAATCTTCGAGTCTGAATTTAAAGGCAGGGTTTATCTTGAAGAAAACAATGGTGCACTTCATATCTCTAATG
TCCGGAAAGAGGACAAAGGTACCTACTACATGAGAGTGCTGCGTGAAACTGAGAACGAGTTGAAGATAACCCTGGAA
GTATTTGATCCTGTGCCCAAGCCTTCCATAGAAATCAATAAGACTGAAGCCTCCACTGATTCCTGTCACCTGAGGCT
ATCGTGTGAGGTAAAGGACCAGCATGTTGACTATACTTGGTATGAGAGCAGCGGACCTTTCCCCAAAAAGAGTCCAG
GATATGTGCTCGATCTCATCGTCACACCACAGAACAAGTCTACATTTTACACCTGCCAAGTCAGCAATCCTGTAAGC

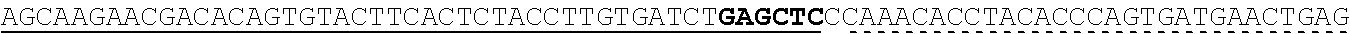

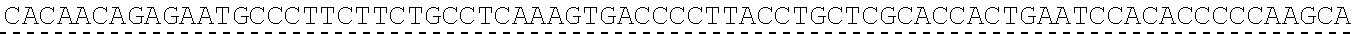

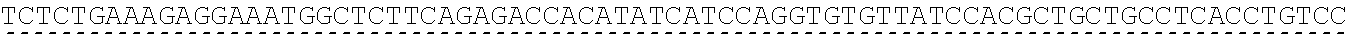

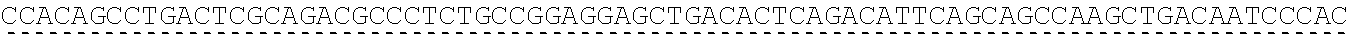

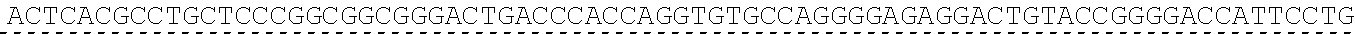

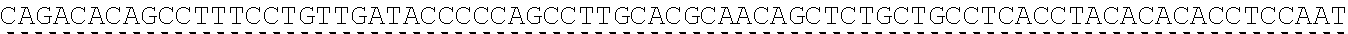

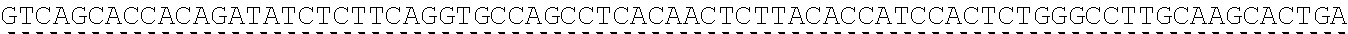

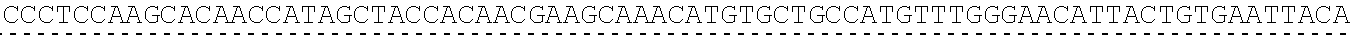

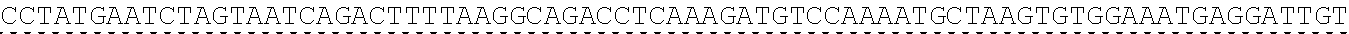

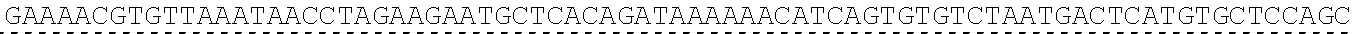

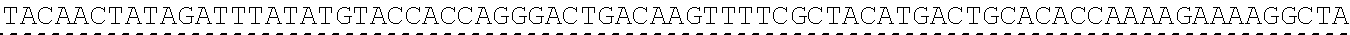

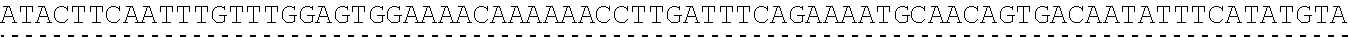

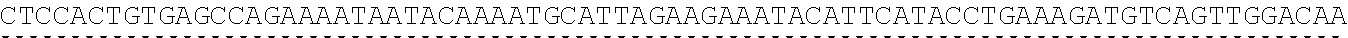

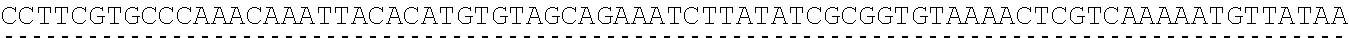

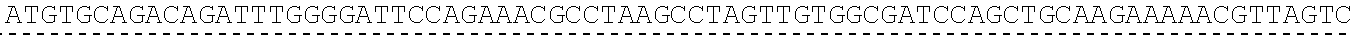

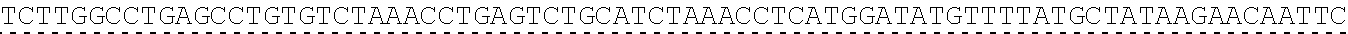

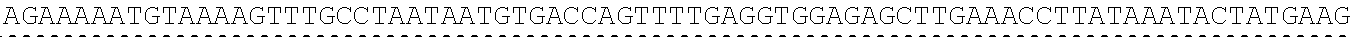

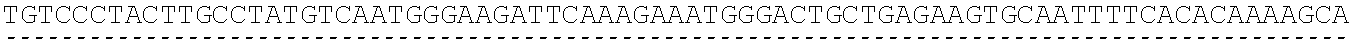

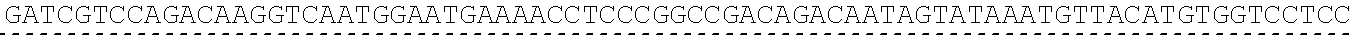

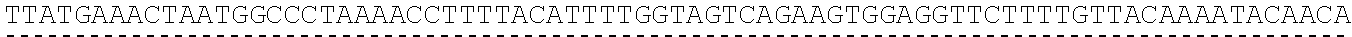

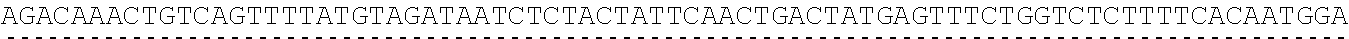

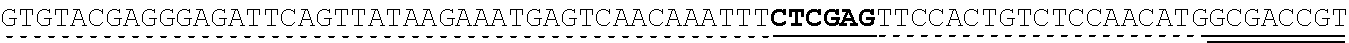

TGCTGTTCTGGTTGTCCTTGGAGCTGCAATAGTCACTGGAGCTGTGGTGGCTTTTGTGATGAAGATGAGAAGGAGAA
ACACAGGTGGAAAAGGAGGGGACTATGCTCTGGCTCCAGGCTCCCAGACCTCTGATCTGTCTCTCCCAGATTGTAAA
GTGATGGTTCATGACCCTCATTCTCTAGCGTGAGCGGCCGC

(SEQ ID NO: 130)

| | |
|---|---|
| MCFIKQGWCLVLELLLLPLGTGFQGHSIPDINATTGSNVTLKIHKDPLGPYKRITWLHTK | 60 |
| NQKILEYNYNSTKTIFESEFKGRVYLEENNGALHISNVRKEDKGTYYMRVLRETENELKI | 120 |
| TLEVFDPVPKPSIEINKTEASTDSCHLRLSCEVKDQHVDYTWYESSGPFPKKSPGYVLDL | 180 |
| 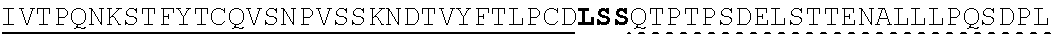 | 240 |
| 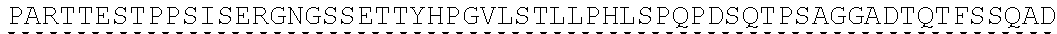 | 300 |
| 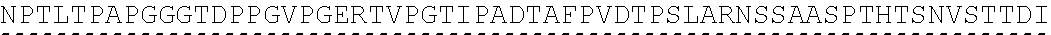 | 360 |
| 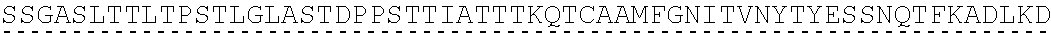 | 420 |
| 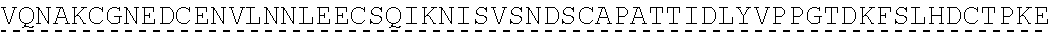 | 480 |
| 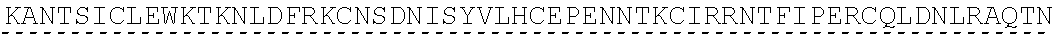 | 540 |
| 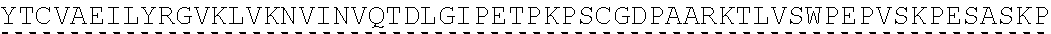 | 600 |
| 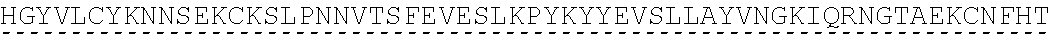 | 660 |
| 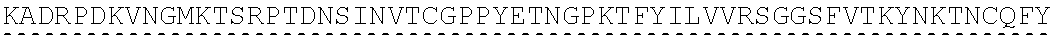 | 720 |
| 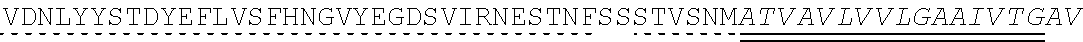 | 780 |
| VAFVMKMRRRNTGGKGGDYALZlPGSOTSDLSLPDCKVMVHDPHSLA | 826 |

1945: mIEE-45 (FIG. 5G)
(SEQ ID NO: 131)

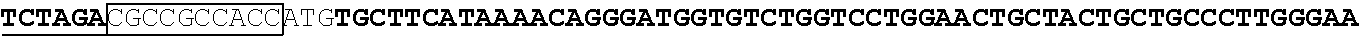

CTGGATTTCAAGGTCATTCAATACCAGATATAAATGCCACCACCGGCAGCAATGTAACCCTGAAAATCCATAAGGAC
CCACTTGGACCATATAAACGTATCACCTGGCTTCATACTAAAAATCAGAAGATTTTAGAGTACAACTATAATAGTAC
AAAGACAATCTTCGAGTCTGAATTTAAAGGCAGGGTTTATCTTGAAGAAAACAATGGTGCACTTCATATCTCTAATG
TCCGGAAAGAGGACAAAGGTACCTACTACATGAGAGTGCTGCGTGAAACTGAGAACGAGTTGAAGATAACCCTGGAA
GTATTTGATCCTGTGCCCAAGCCTTCCATAGAAATCAATAAGACTGAAGCCTCCACTGATTCCTGTCACCTGAGGCT
ATCGTGTGAGGTAAAGGACCAGCATGTTGACTATACTTGGTATGAGAGCAGCGGACCTTTCCCCAAAAAGAGTCCAG
GATATGTGCTCGATCTCATCGTCACACCACAGAACAAGTCTACATTTTACACCTGCCAAGTCAGCAATCCTGTAAGC

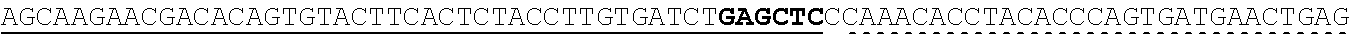

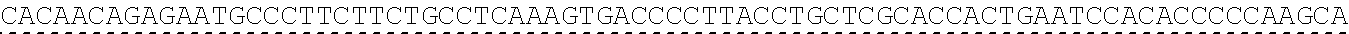

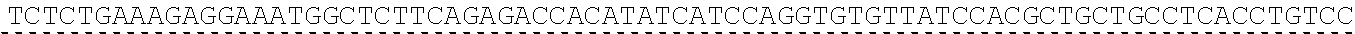

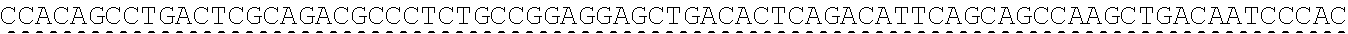

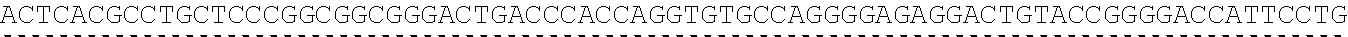

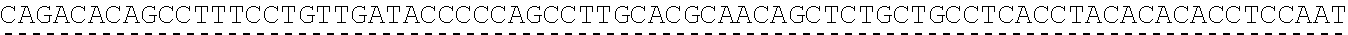

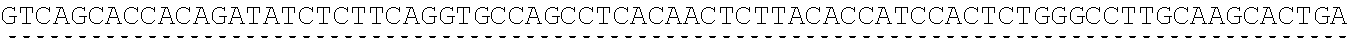

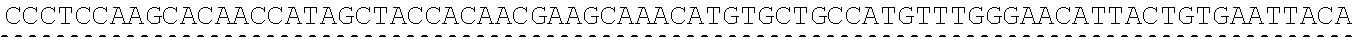

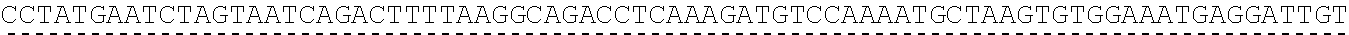

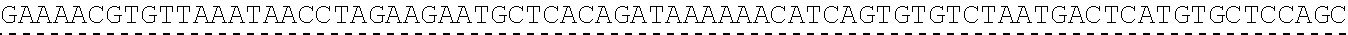

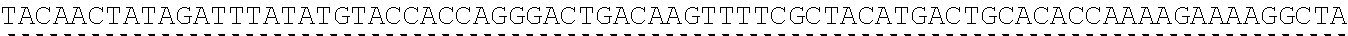

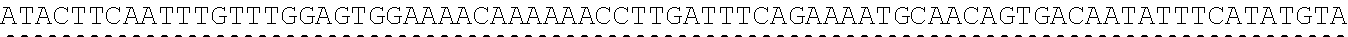

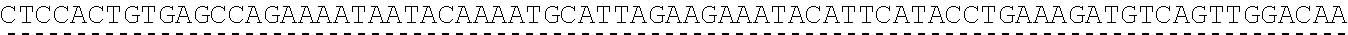

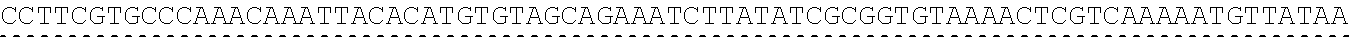

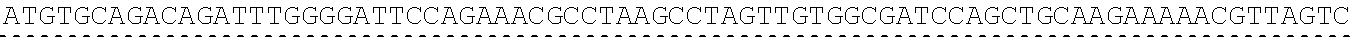

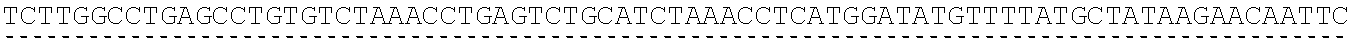

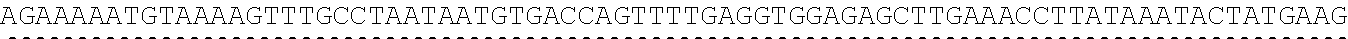

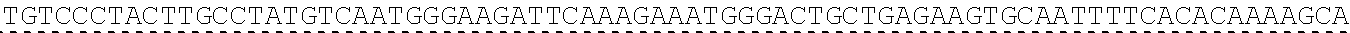

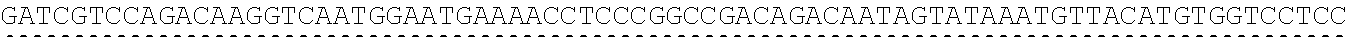

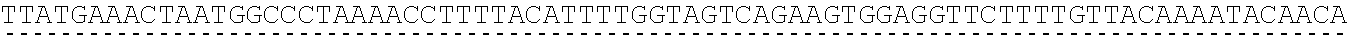

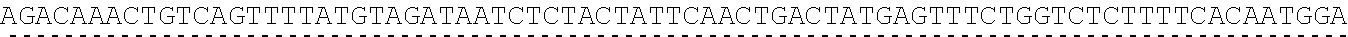

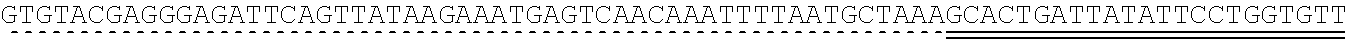

TCTGATTATTGTGACATCAATAGCCTTGCTTGTTGTTTTGTATAAAATCTATGATCTGCGCAAGAAAAGATCCAGCA
ATTGAGCGGCCGC

(SEQ ID NO: 132)

| | |
|---|---|
| 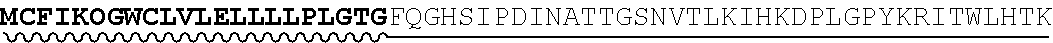 | 60 |
| NQKILEYNYNSTKTIFESEFKGRVYLEENNGALHISNVRKEDKGTYYMRVLRETENELKI | 120 |
| TLEVEDPVPKPSIEINKTEASTDSCHLRLSCEVKDQHVDYTWYESSGPFPKKSPGYVLDL | 180 |
| 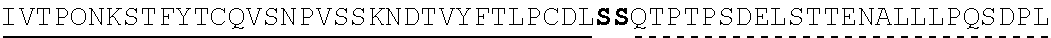 | 240 |
| 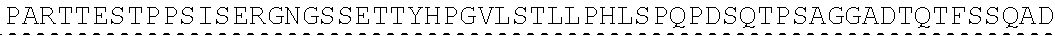 | 300 |
| 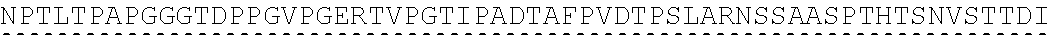 | 360 |
| 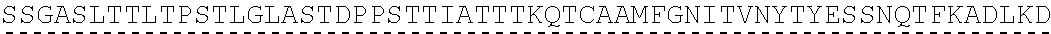 | 420 |
| 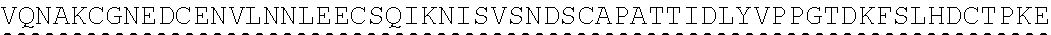 | 480 |
| 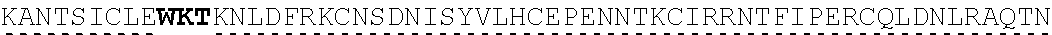 | 540 |
| 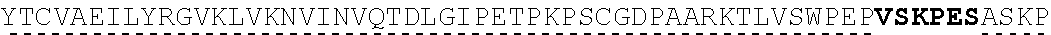 | 600 |
| 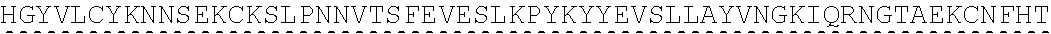 | 660 |
| 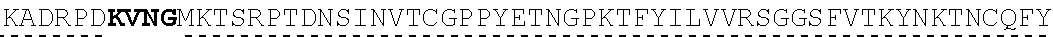 | 720 |
| 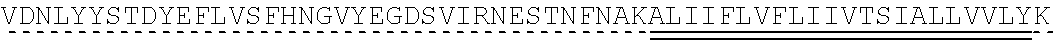 | 780 |
| 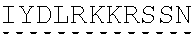 | 791 |

1961: hIEE1 (-) 22 (FIG. 5K)
(SEQ ID NO: 133)

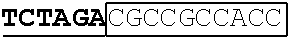

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA

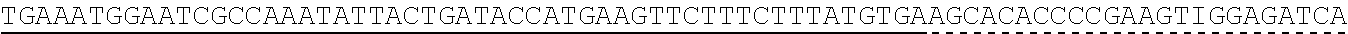

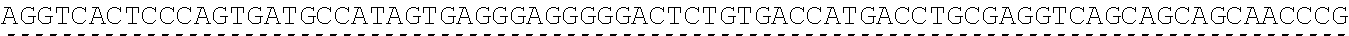

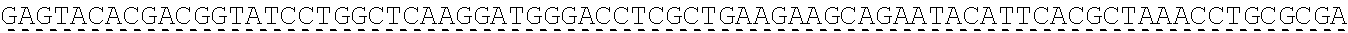

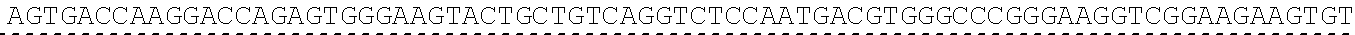

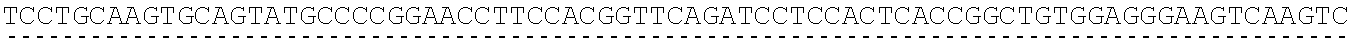

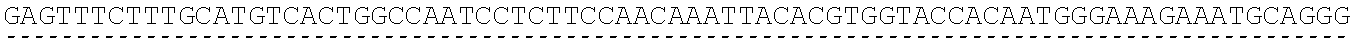

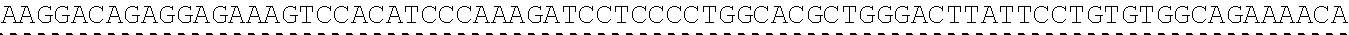

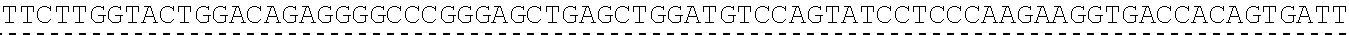

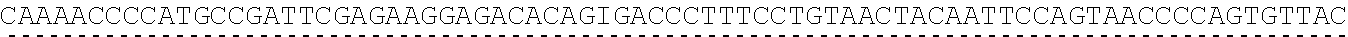

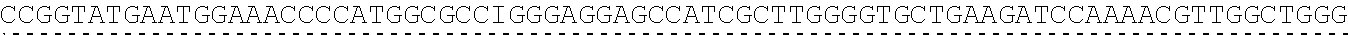

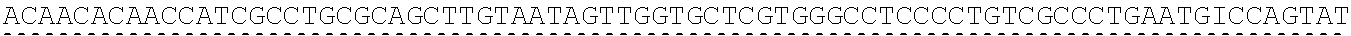

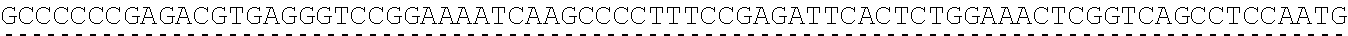

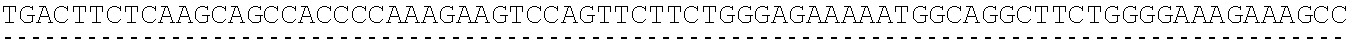

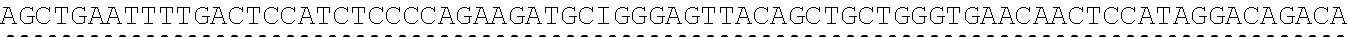

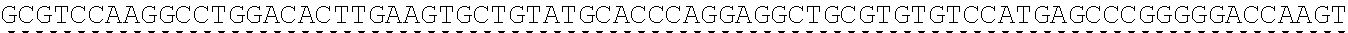

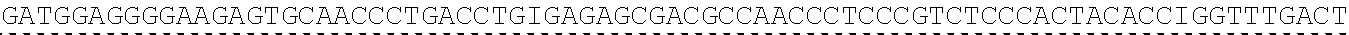

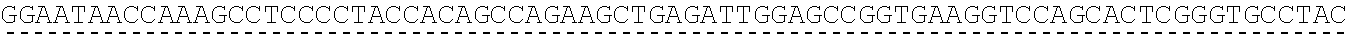

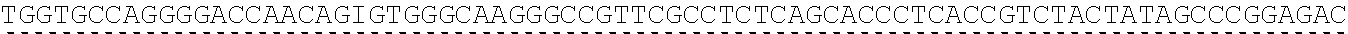

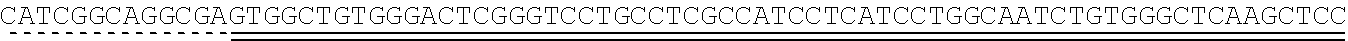

AGCGACGTIGGAAGAGGACACAGAGCCAGCAGGGGTGAGCGGCCGC

(SEQ ID NO: 134)

MVAGSDAGRALGVLSVVCLLHCFGFISCFSQQIYGVVYGNVTFHVPSNVPLKEVLWKKQKDKVAELENSEFRAFSSF

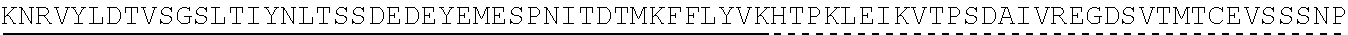

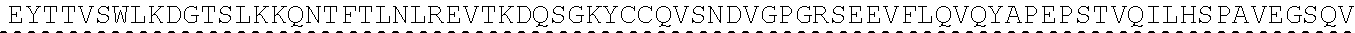

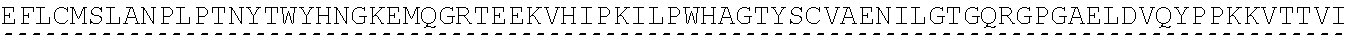

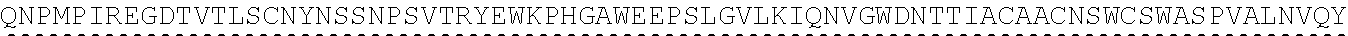

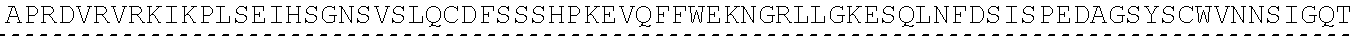

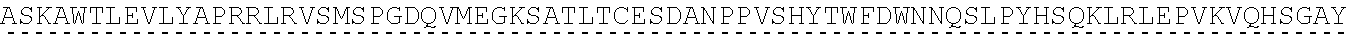

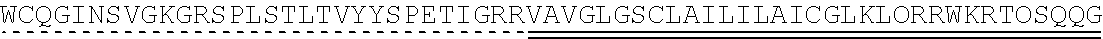

1963: hIEE1 (−)RO (FIG. 5K)
(SEQ ID NO: 135)

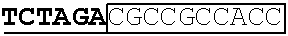

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA

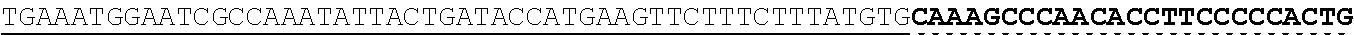

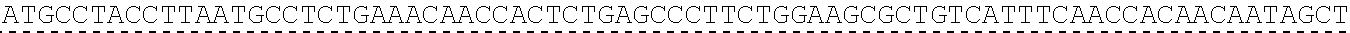

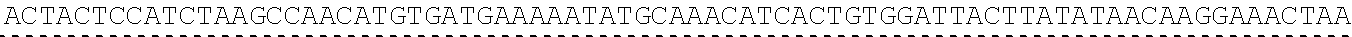

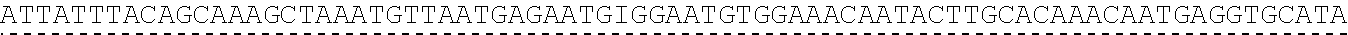

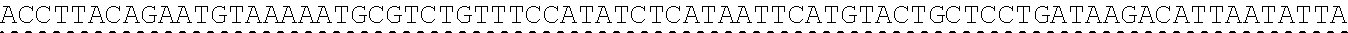

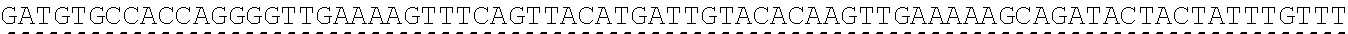

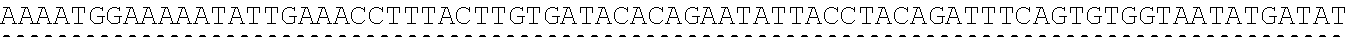

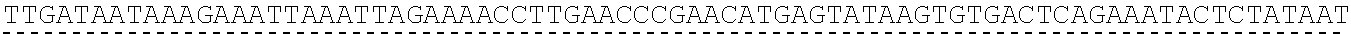

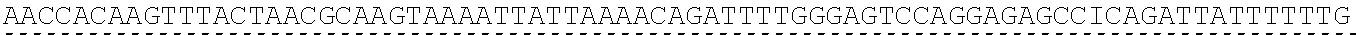

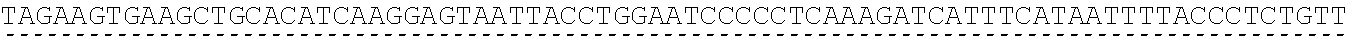

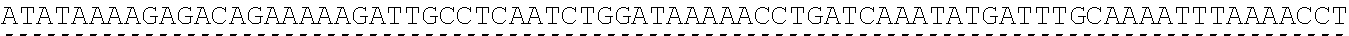

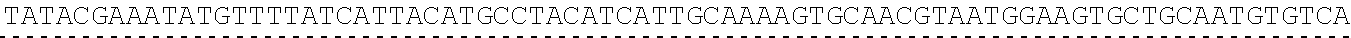

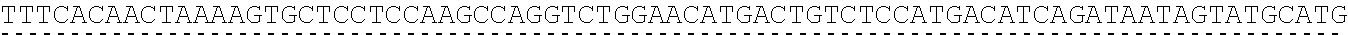

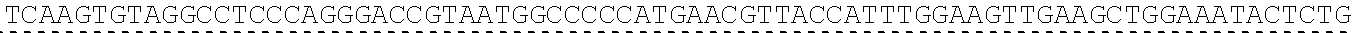

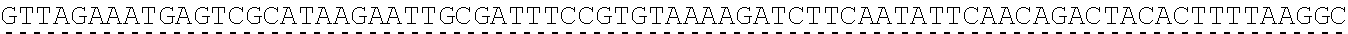

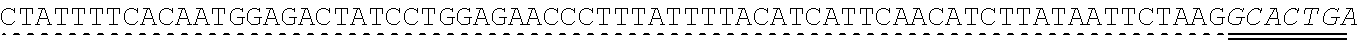

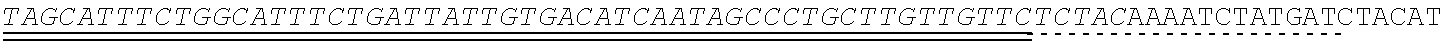

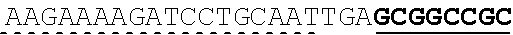

(SEQ ID NO: 136)

MVAGSDAGRALGVLSVVCLLHCFGFISCFSQQIYGVVYGNVTFHVPSNVPLKEVLWKKQKDKVAELENSEFRAFSSF

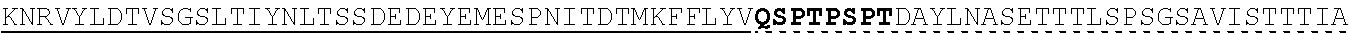

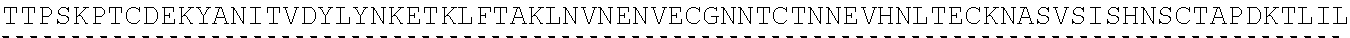

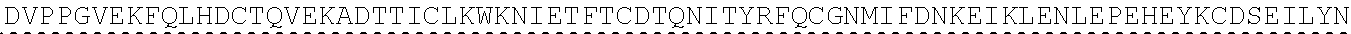

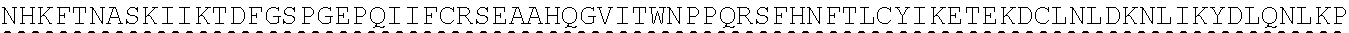

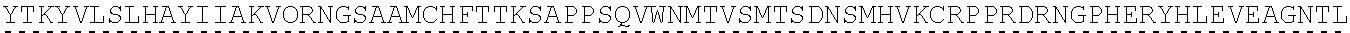

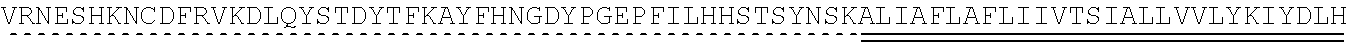

KKRSCN

1965: hIEE1 (−)ABC (FIG. 5K)
(SEQ ID NO: 137)

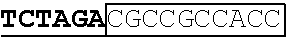

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA

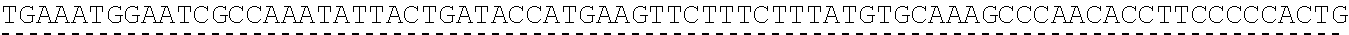

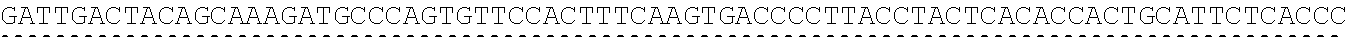

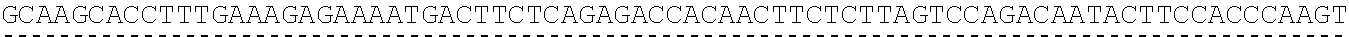

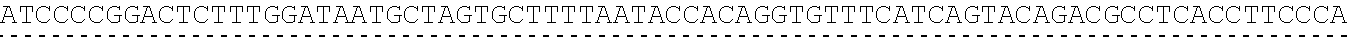

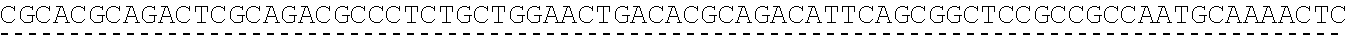

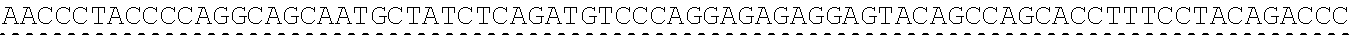

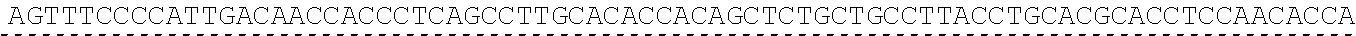

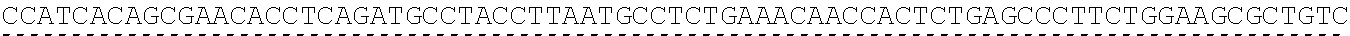

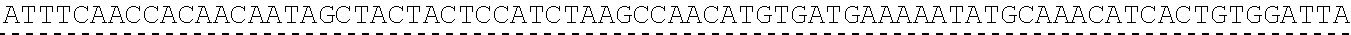

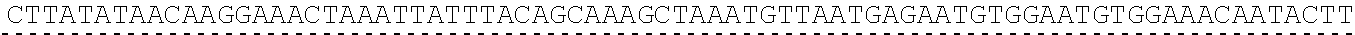

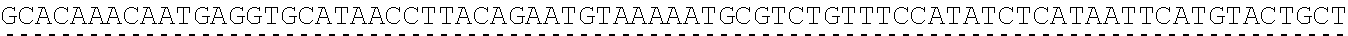

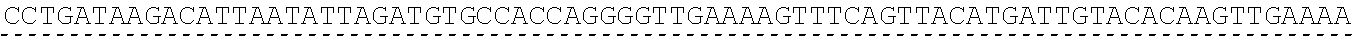

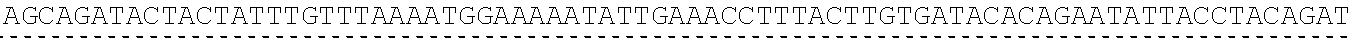

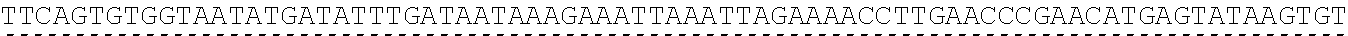

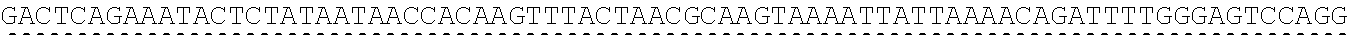

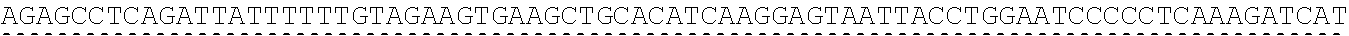

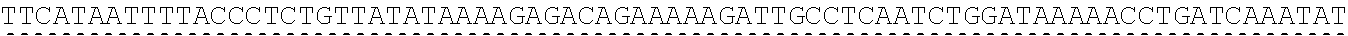

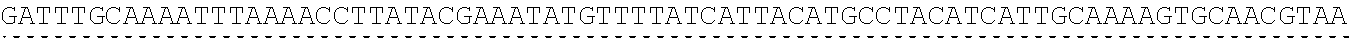

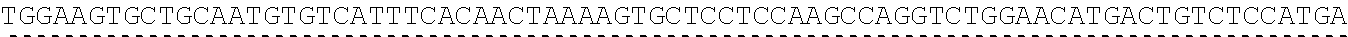

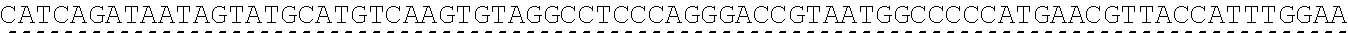

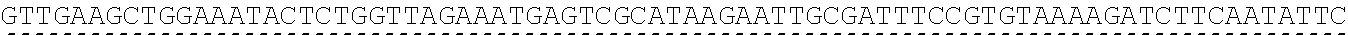

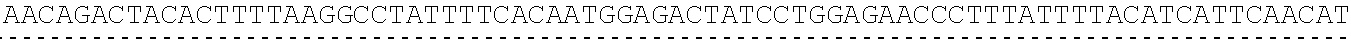

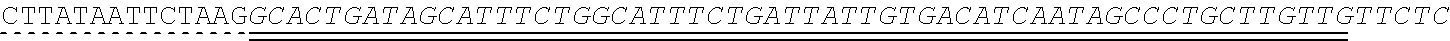

TACAAAATCTATGATCTACATAAGAAAAGATCCTGCAATTGAGCGGCCGC

(SEQ ID NO: 138)

MVAGSDAGRALGVLSVVCLLHCFGFISCFSQQIYGVVYGNVTFHVPSNVPLKEVLWKKQKDKVAELENSEFRAFSSF

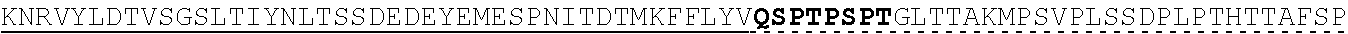

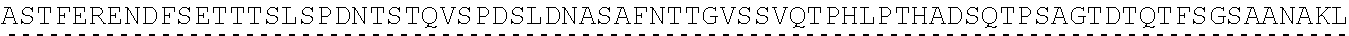

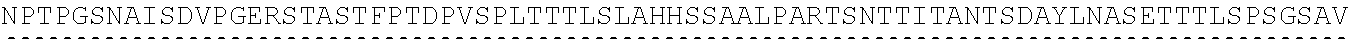

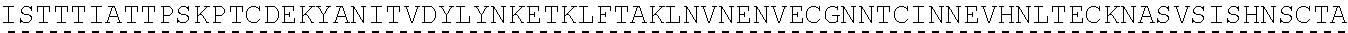

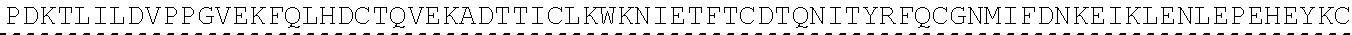

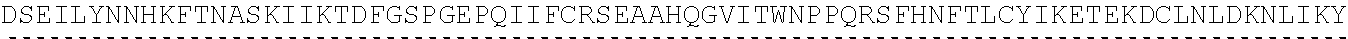

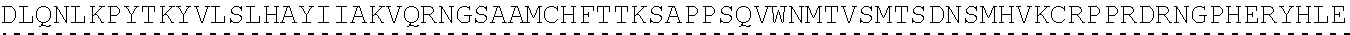

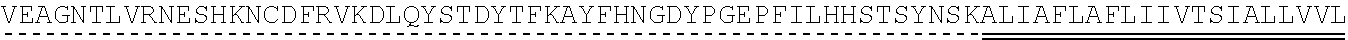

YKIYDLHKKRSCN

1962: hIEE1 (+) 22 (FIG. 5K)
(SEQ ID NO: 139)

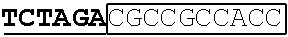

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA

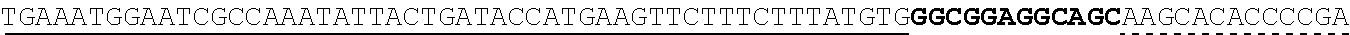

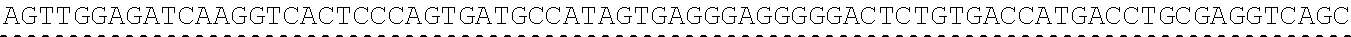

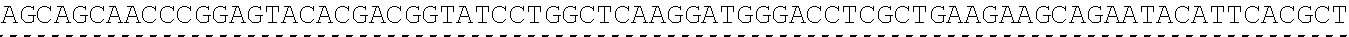

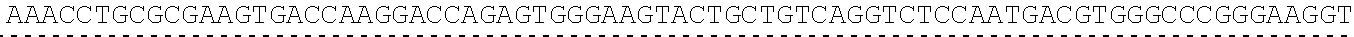

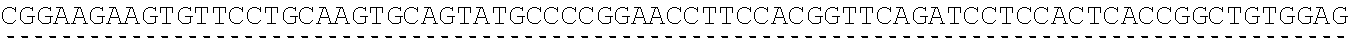

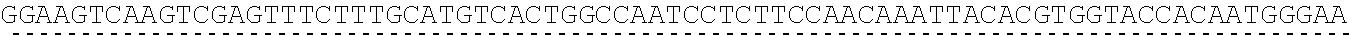

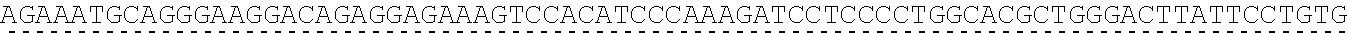

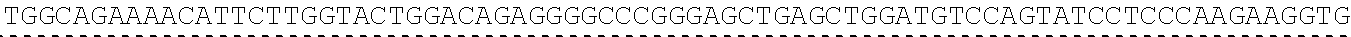

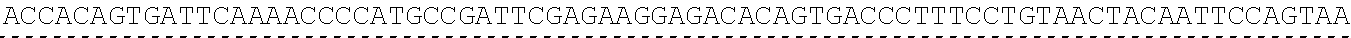

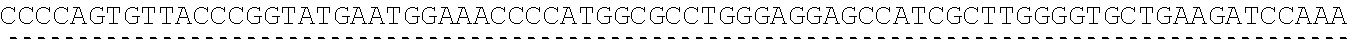

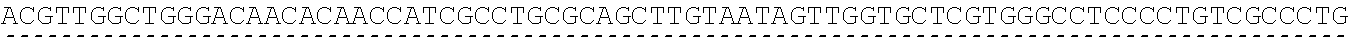

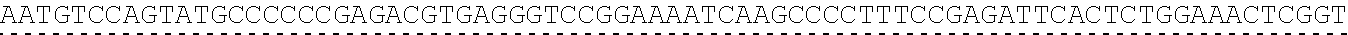

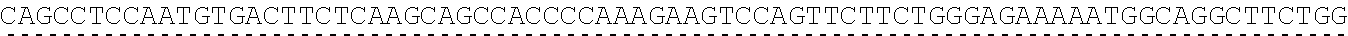

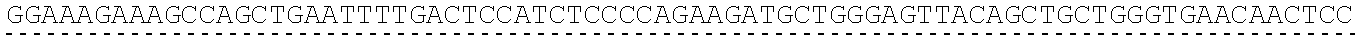

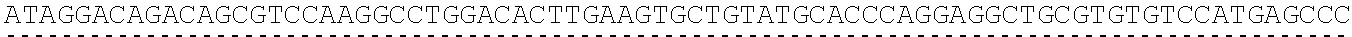

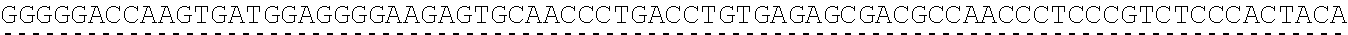

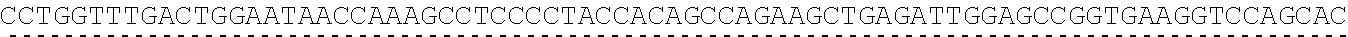

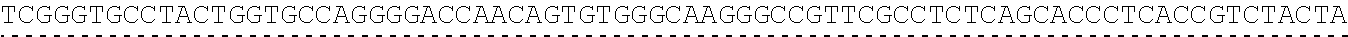

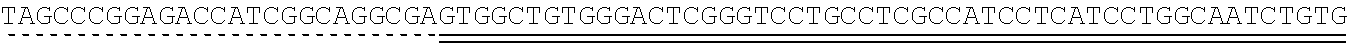

GGCTCAAGCTCCAGCGACGTTGGAAGAGGACACAGAGCCAGCAGGGGTGAGCGGCCGC

(SEQ ID NO: 140)

MVAGSDAGRALGVLSVVCLLHCFGFISCFSQQIYGVVYGNVTFHVPSNVPLKEVLWKKQKDKVAELENSEFRAFSSF

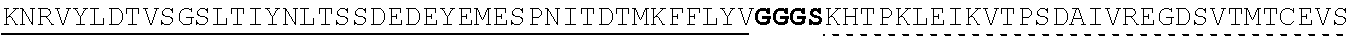

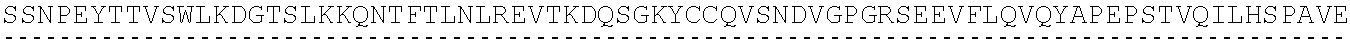

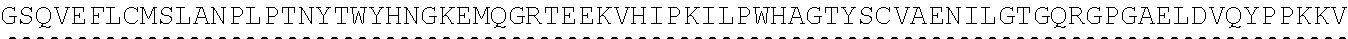

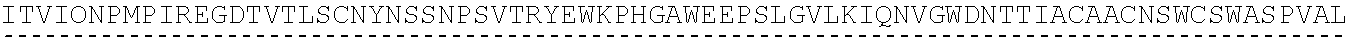

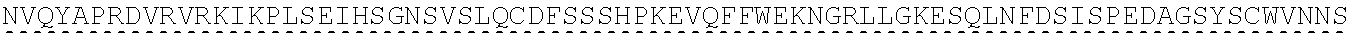

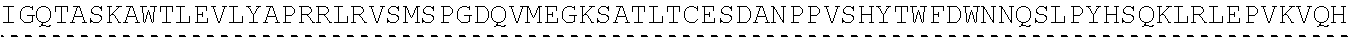

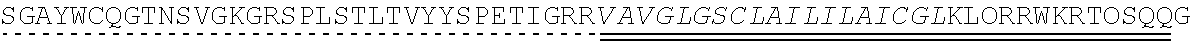

1964: hIEE1 (+) RO (FIG. 5K)
(SEQ ID NO: 141)

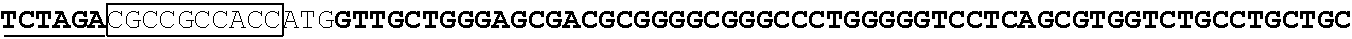

ACTGCTTTGGTTTCATCAGCTGTTTTTCCCAACAAATATATGGTGTTGTGTATGGGAATGTAACTTTCCATGTACCA
AGCAATGTGCCTTTAAAAGAGGTCCTATGGAAAAAACAAAAGGATAAAGTTGCAGAACTGGAAAATTCTGAGTTCAG
AGCTTTCTCATCTTTTAAAAATAGGGTTTATTTAGACACTGTGTCAGGTAGCCTCACTATCTACAACTTAACATCAT
CAGATGAAGATGAGTATGAAATGGAATCGCCAAATATTACTGATACCATGAAGTTCTTTCTTTATGTGGGCGGAGGC

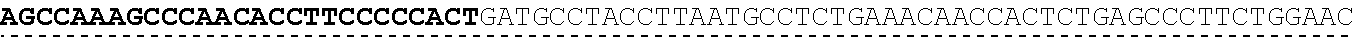

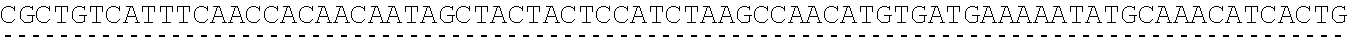

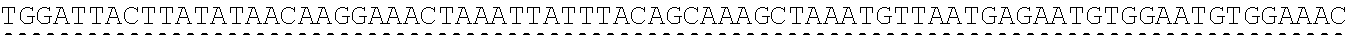

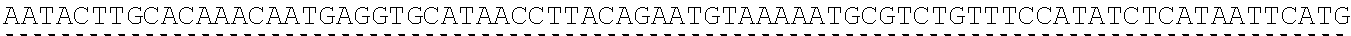

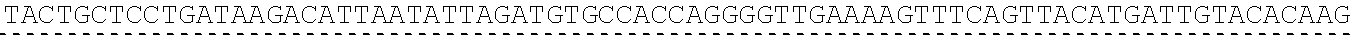

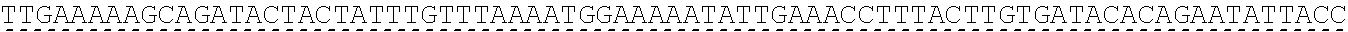

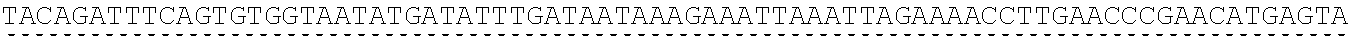

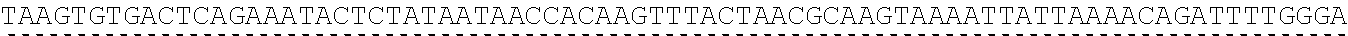

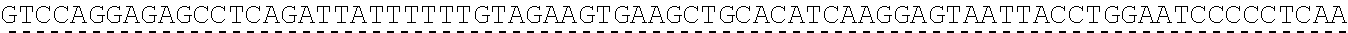

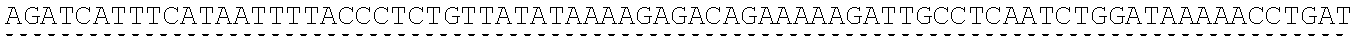

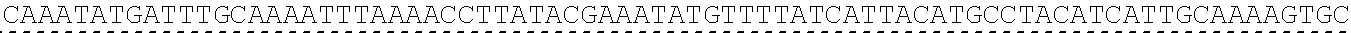

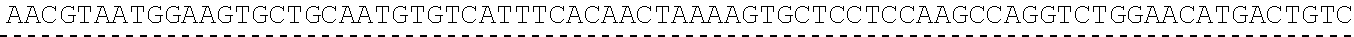

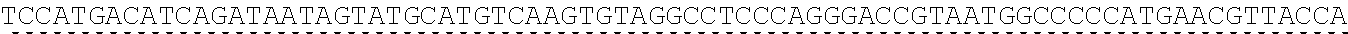

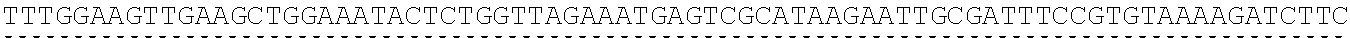

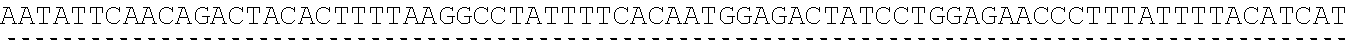

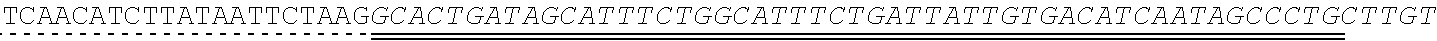

TGTTCTCTACAAAATCTATGATCTACATAAGAAAAGATCCTGCAATTGAGCGGCCGC

(SEQ ID NO: 142)

MVAGSDAGRALGVLSVVCLLHCFGFISCFSQQIYGVVYGNVTFHVPSNVPLKEVLWKKQKDKVAELENSEFRAFSSF

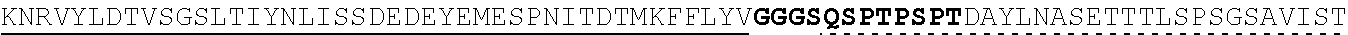

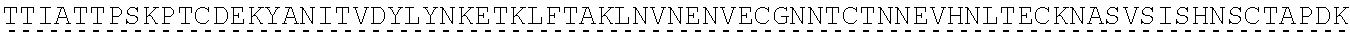

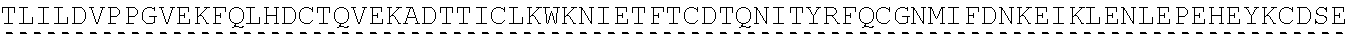

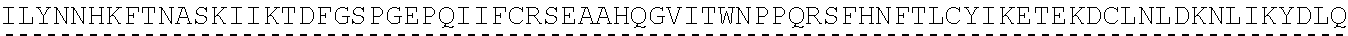

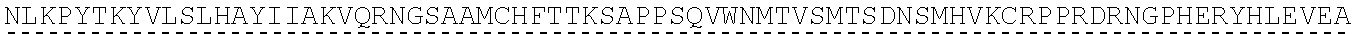

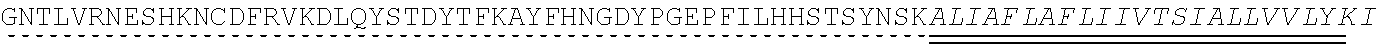

YDLHKKRSCN

(SEQ ID NO: 143)

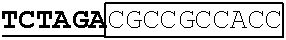

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA

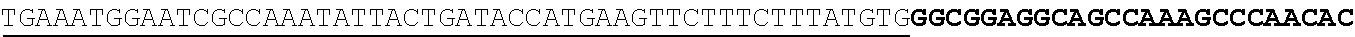

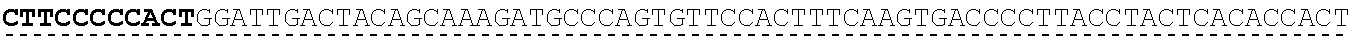

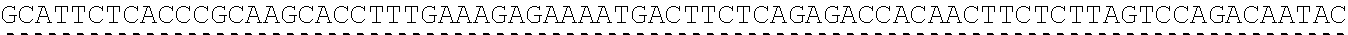

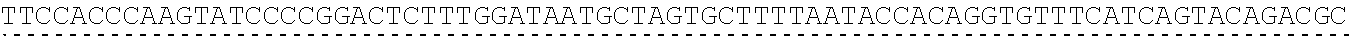

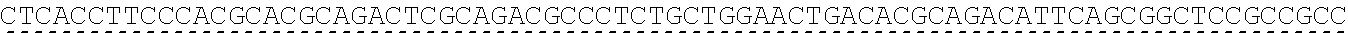

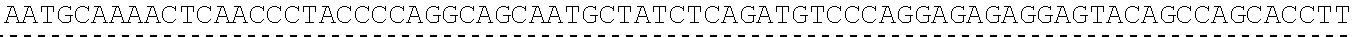

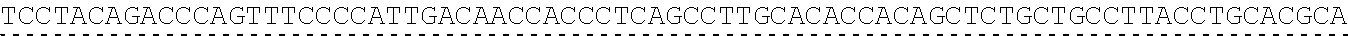

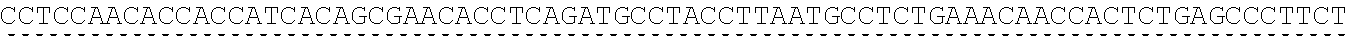

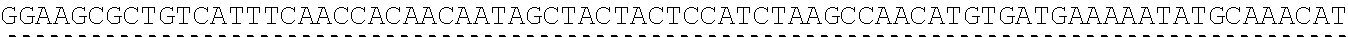

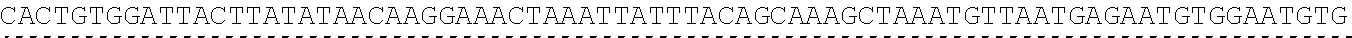

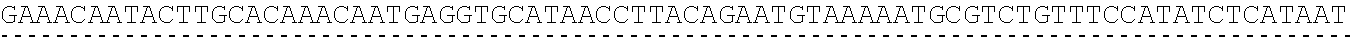

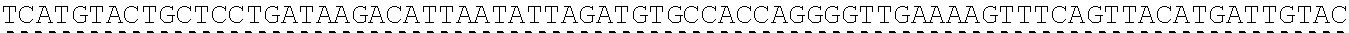

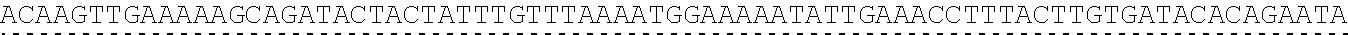

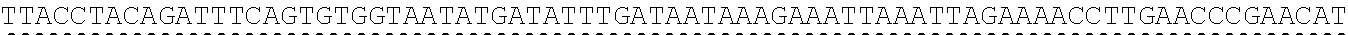

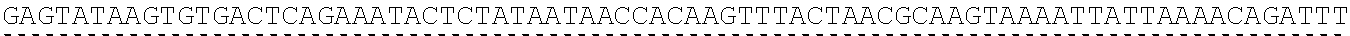

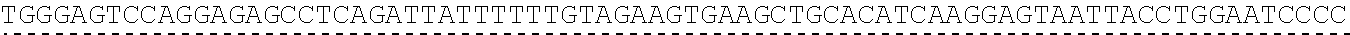

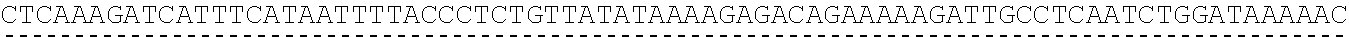

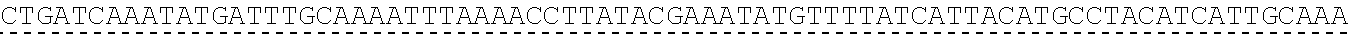

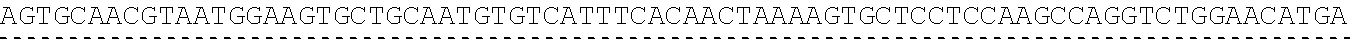

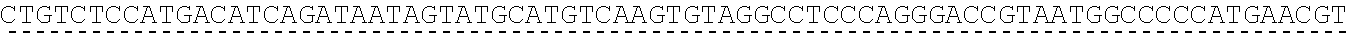

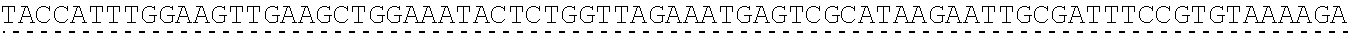

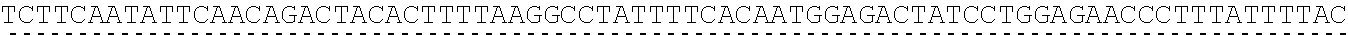

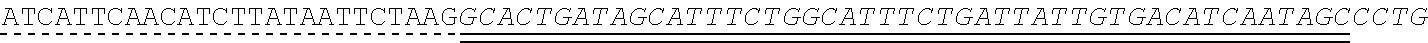

CTTGTTGTTCTCTACAAAATCTATGATCTACATAAGAAAAGATCCTGCAATTGAGCGGCCGC

(SEQ ID NO: 144)

MVAGSDAGRALGVLSVVCLLHCFGFISCFSQQIYGVVYGNVTFHVPSNVPLKEVLWKKQKDKVAELENSEFRAFSSF

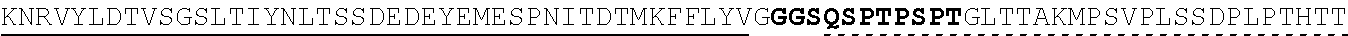

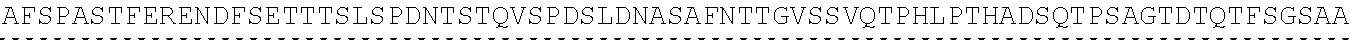

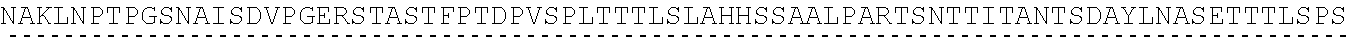

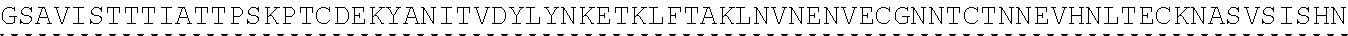

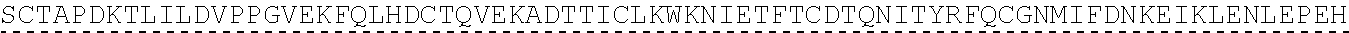

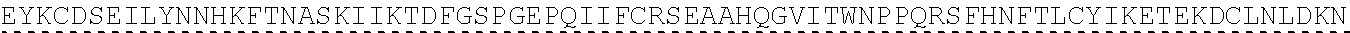

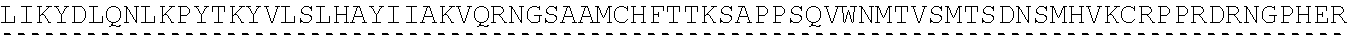

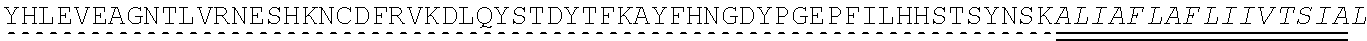

LVVLYKIYDLHKKRSCN

1967: hIEE2 (-) 22 (FIG. 5K)
(SEQ ID NO: 145)

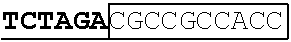

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA
TGAAATGGAATCGCCAAATATTACTGATACCATGAAGTTCTTTCTTTATGTGCTTGAGTCTCTTCCATCTCCCACAC
TAACTTGTGCATTGACTAATGGAAGCATTGAAGTCCAATGCATGATACCAGAGCATTACAACAGCCATCGAGGACTT
ATAATGTACTCATGGGATTGTCCTATGGAGCAATGTAAACGTAACTCAACCAGTATATATTTTAAGATGGAAAATGA
TCTTCCACAAAAAATACAGTGTACTCTTAGCAATCCATTATTTAATACAACATCATCAATCATTTTGACAACCTGTA

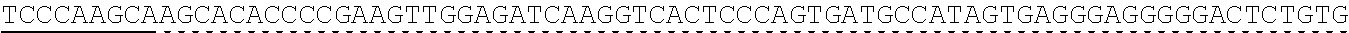

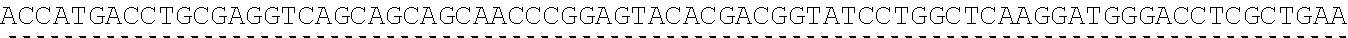

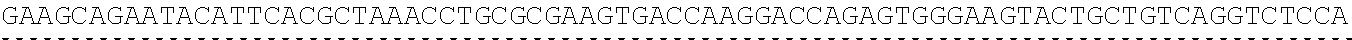

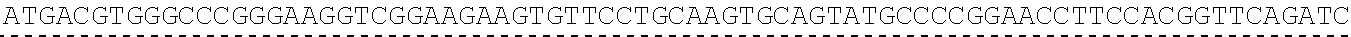

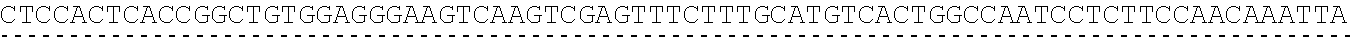

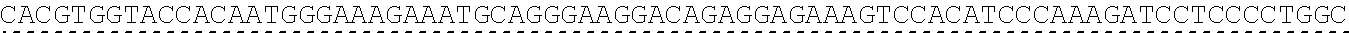

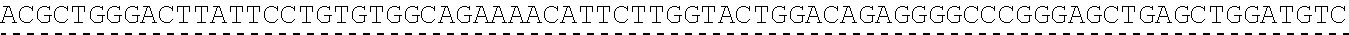

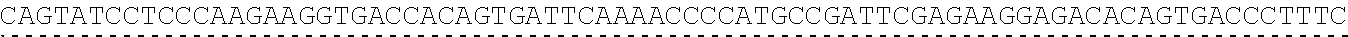

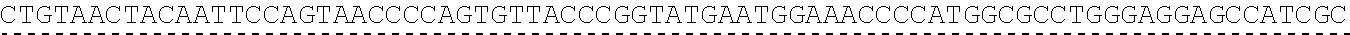

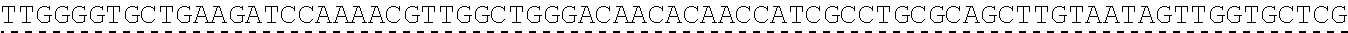

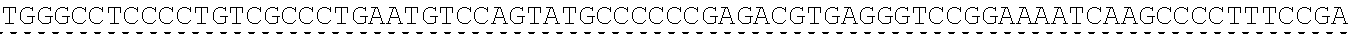

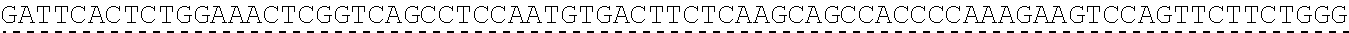

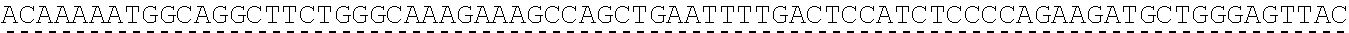

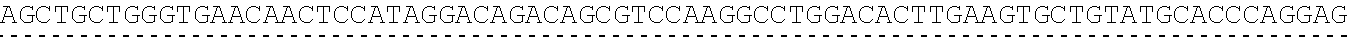

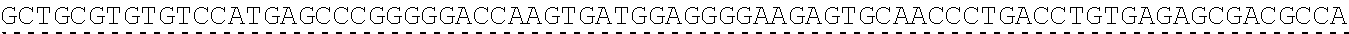

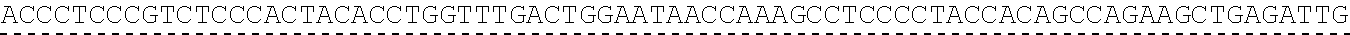

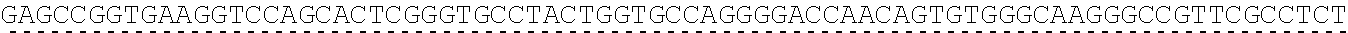

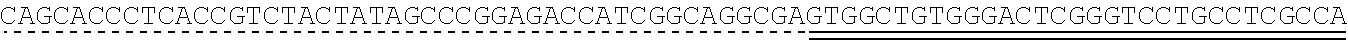

TCCTCATCCTGGCAATCTGTGGGCTCAAGCTCCAGCGACGTTGGAAGAGGACACAGAGCCAGCAGGGGTGAGCGGCC
GC

(SEQ ID NO: 146)

MVAGSDAGRALGVLSVVCLLHCFGFISCFSQQIYGVVYGNVTFHVPSNVPLKEVLWKKQKDKVAELENSEFRAFSSF
KNRVYLDTVSGSLTIYNLTSSDEDEYEMESPNITDTMKFFLYVLESLPSPTLTCALINGSIEVQCMIPEHYNSHRGL

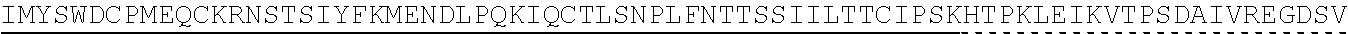

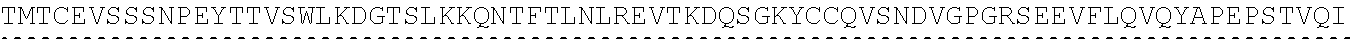

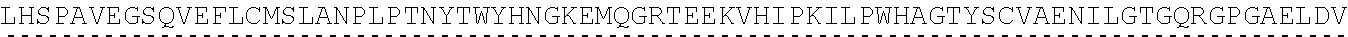

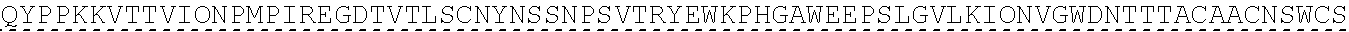

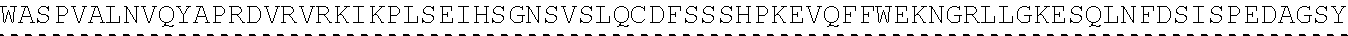

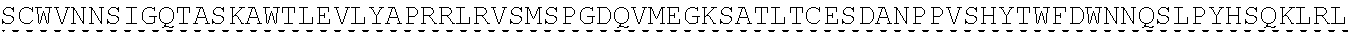

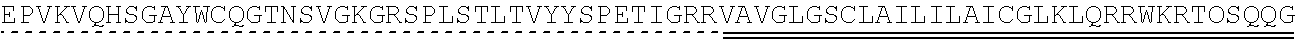

1969: hIEE2 (-) RO (FIG. 5K)
(SEQ ID NO: 147)

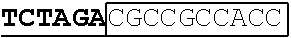

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA
TGAAATGGAATCGCCAAATATTACTGATACCATGAAGTTCTTTCTTTATGTGCTTGAGTCTCTTCCATCTCCCACAC
TAACTTGTGCATTGACTAATGGAAGCATTGAAGTCCAATGCATGATACCAGAGCATTACAACAGCCATCGAGGACTT
ATAATGTACTCATGGGATTGTCCTATGGAGCAATGTAAACGTAACTCAACCAGTATATATTTTAAGATGGAAAATGA
TCTTCCACAAAAAATACAGTGTACTCTTAGCAATCCATTATTTAATACAACATCATCAATCATTTTGACAACCTGTA

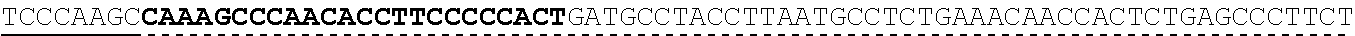

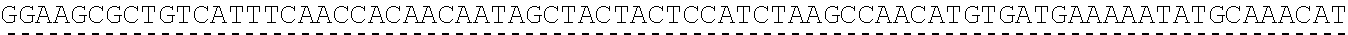

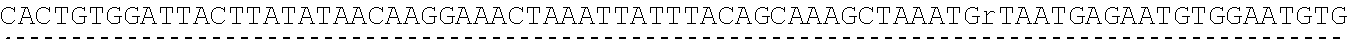

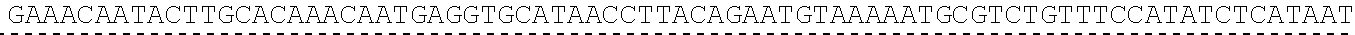

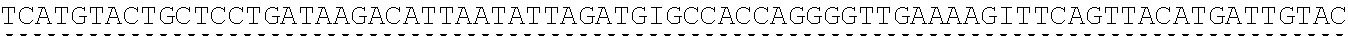

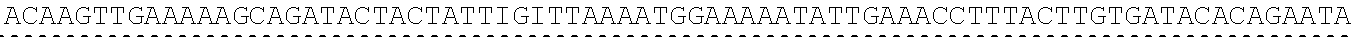

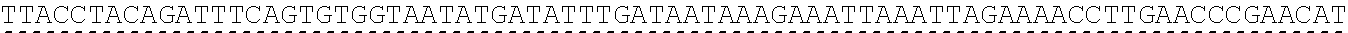

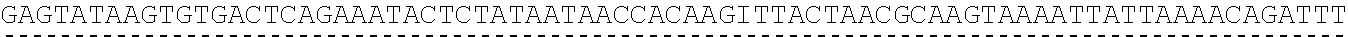

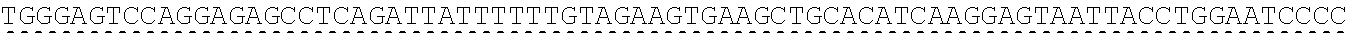

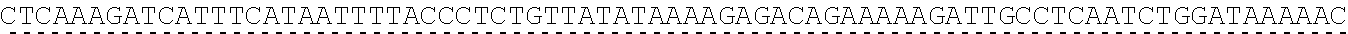

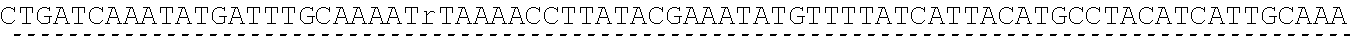

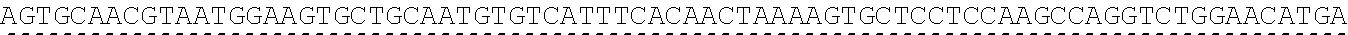

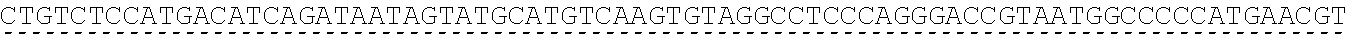

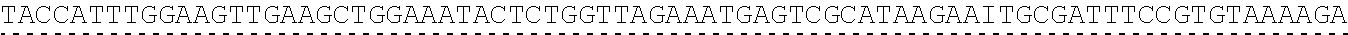

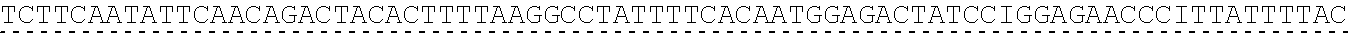

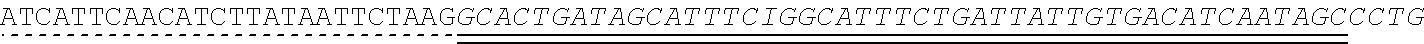

CTTGTTGTTCTCTACAAAATCTATGATCTACATAAGAAAAGATCCTGCAATTGAGCGGCCGC

(SEQ ID NO: 148)

MVAGSDAGRALGVLSVVCLLHCFGFISCFSQQIYGVVYGNVTFHVPSNVPLKEVLWKKQKDKVAELENSEFRAFSSF
KNRVYLDTVSGSLTIYNLTSSDEDEYEMESPNITDTMKFFLYVLESLPSPTLTCALTINGSIEVQCMIPEHYNSHRGL

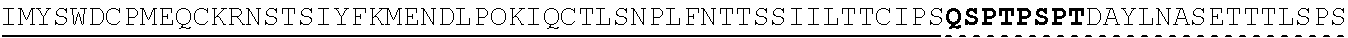

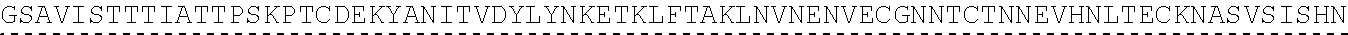

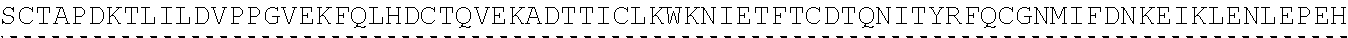

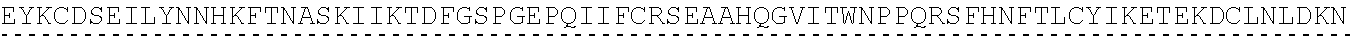

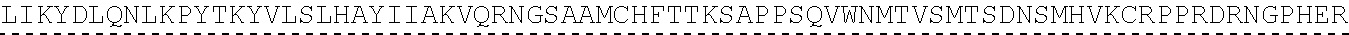

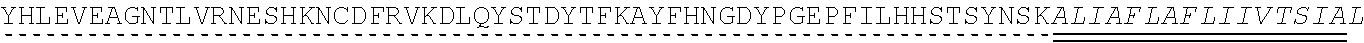

LVVLYKIYDLHKKRSCN

1971: hIEE2 (−)ABC (FIG. 5K)
(SEQ ID NO: 149)

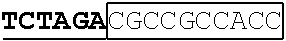

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA
TGAAATGGAATCGCCAAATATTACTGATACCATGAAGTTCTTTCTTTATGTGCTTGAGTCTCTTCCATCTCCCACAC
TAACTTGTGCATTGACTAATGGAAGCATTGAAGTCCAATGCATGATACCAGAGCATTACAACAGCCATCGAGGACTT
ATAATGTACTCATGGGATTGTCCTATGGAGCAATGTAAACGTAACTCAACCAGTATATATTTTAAGATGGAAAATGA
TCTTCCACAAAAAATACAGTGTACTCTTAGCAATCCATTATTTAATACAACATCATCAATCATTTTGACAACCTGTA

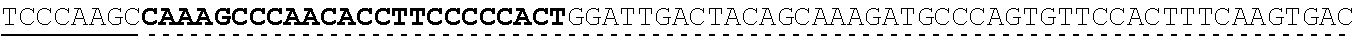

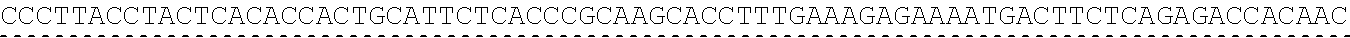

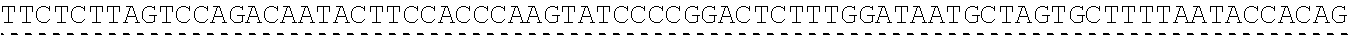

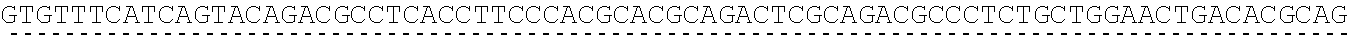

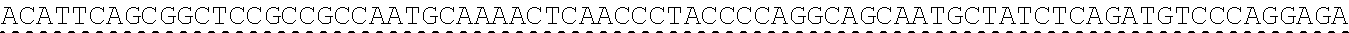

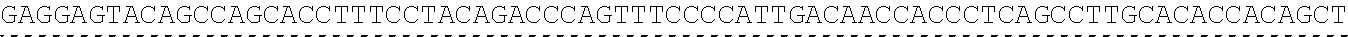

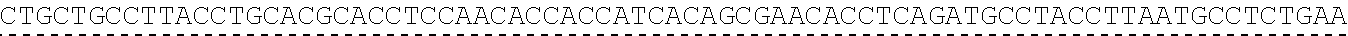

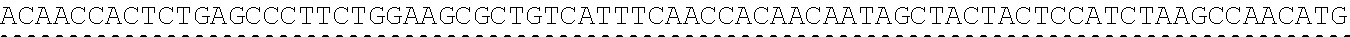

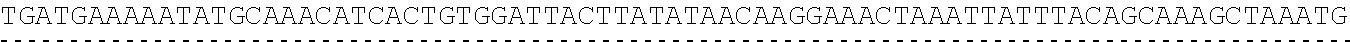

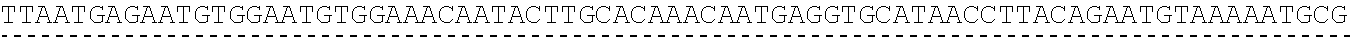

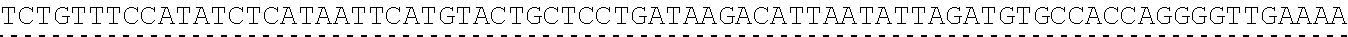

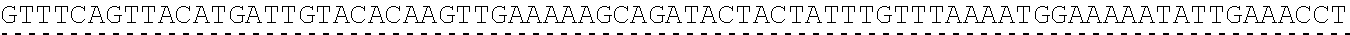

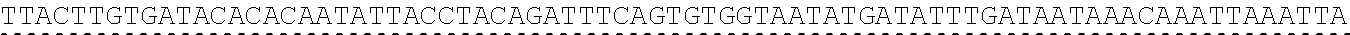

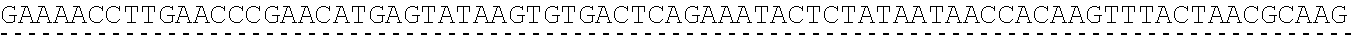

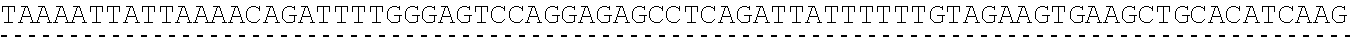

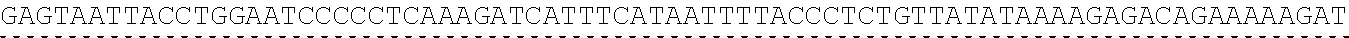

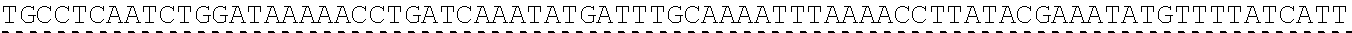

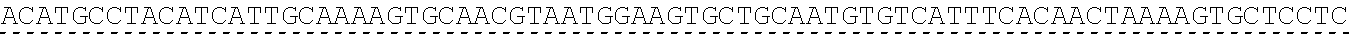

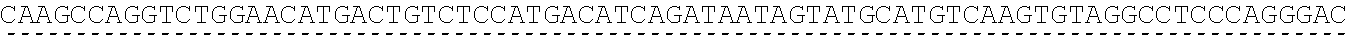

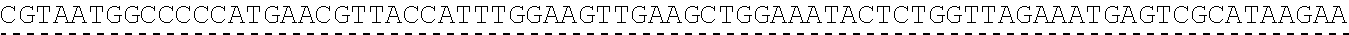

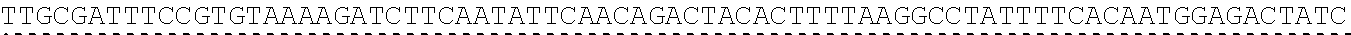

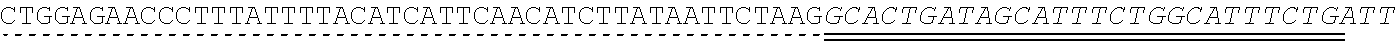

ATTGIGACATCAATAGCCCTGCTTGTTGTTCTCTACAAAATCTATGATCTACATAAGAAAAGATCCTGCAATTGAGC
GGCCGC

(SEQ ID NO: 150)

MVAGSDAGRALGVLSVVCLLHCFGFISCFSQQIYGVVYGNVTFHVPSNVPLKEVLWKKQKDKVAELENSEFRAFSSF
KNRVYLDTVSGSITIYNLTSSDEDEYEMESPNITDTMKFFLYVLESLPSPTLTCALTNGSIEVQCMIPEHYNSHFGL

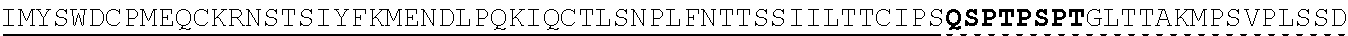

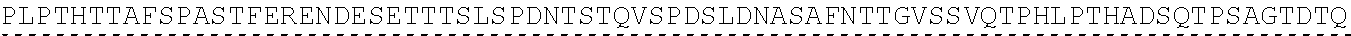

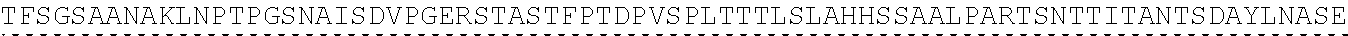

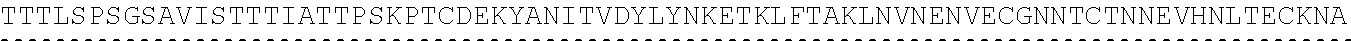

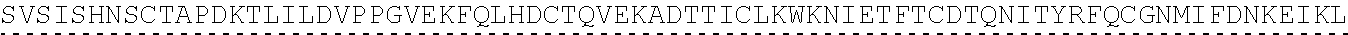

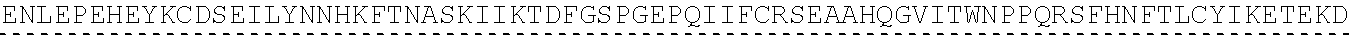

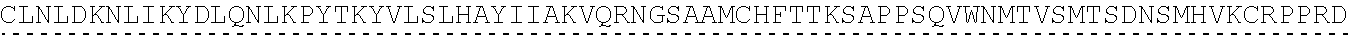

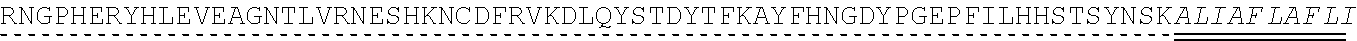

IVTSIALLVVLYKIYDLHKKRSCN

1968: hIEE2 (+) 22 (FIG. 5K)
(SEQ ID NO: 151)

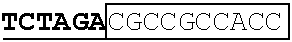

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA
TGAAATGGAATCGCCAAATATTACTGATACCATGAAGTTCTTTCTTTATGTGCTTGAGTCTCTTCCATCTCCCACAC
TAACTTGTGCATTGACTAATGGAAGCATTGAAGTCCAATGCATGATACCAGAGCATTACAACAGCCATCGAGGACTT
ATAATGTACTCATGGGATTGTCCTATGGAGCAATGTAAACGTAACTCAACCAGTATATATTTTAAGATGGAAAATGA
TCTTCCACAAAAAATACAGTGTACTCTTAGCAATCCATTATTTAATACAACATCATCAATCATTTTGACAACCTGTA

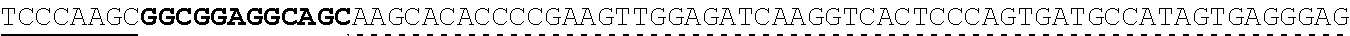

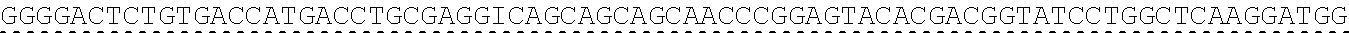

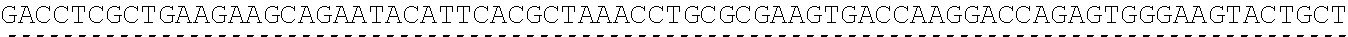

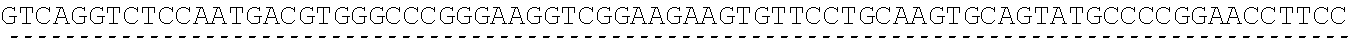

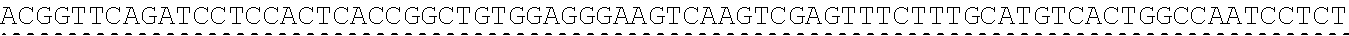

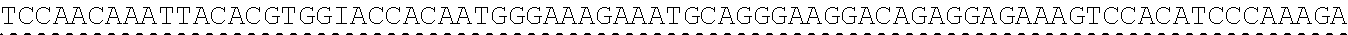

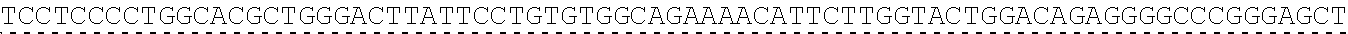

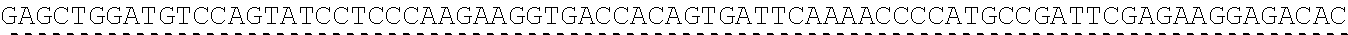

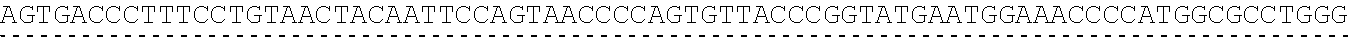

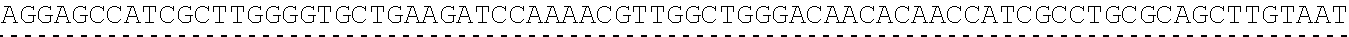

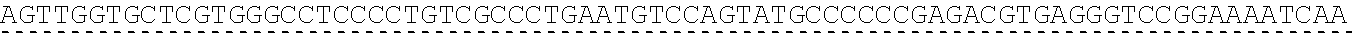

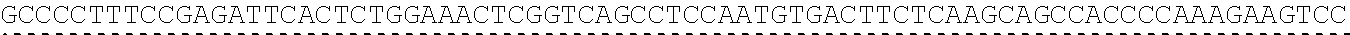

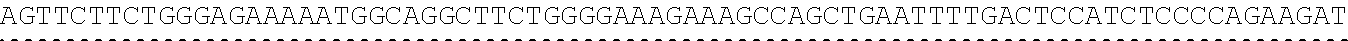

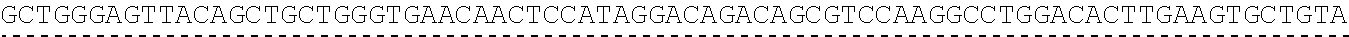

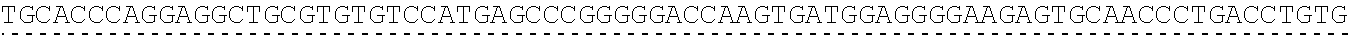

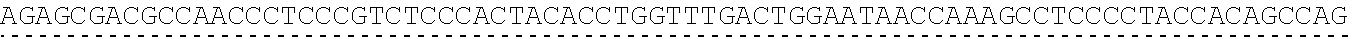

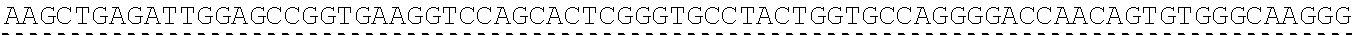

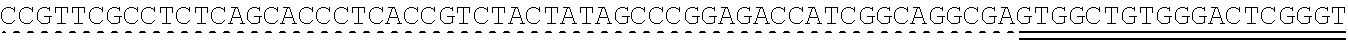

CCTGCCTCGCCATCCTCATCCTGGCAATCTGTGGGCTCAAGCTCCAGCGACGTTGGAAGAGGACACAGAGCCAGCAG
GGGTGAGCGGCCGC

(SEQ ID NO: 152)

MVAGSDAGRALGVLSVVCLLHCFGFISCFSQQIYGVVYGNVTFHVPSNVPLKEVLWKKQKDKVAELENSEFRAFSSF
KNRVYLDTVSGSLTIYNLTSSDKDEYEMESPNITDTMKFFLYVLESLPSPTLTCALrNGSIEVQCMIPEHYNSHRGL

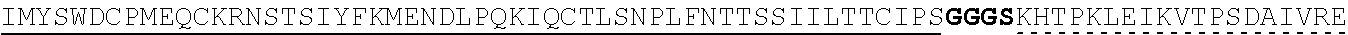

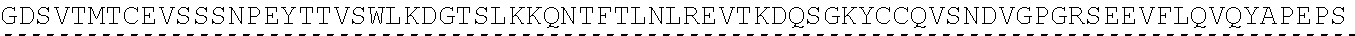

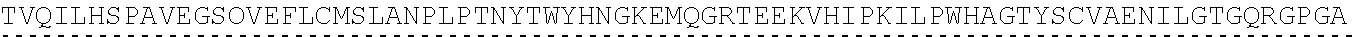

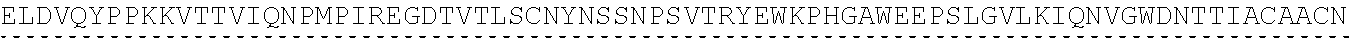

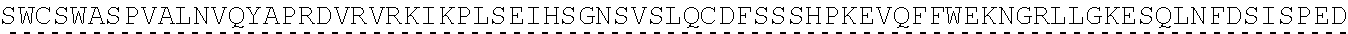

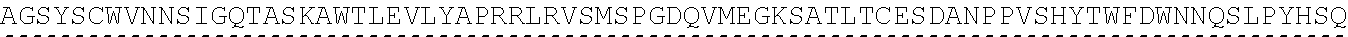

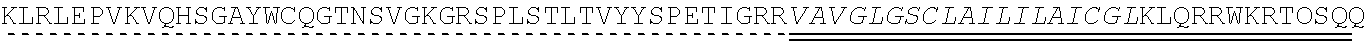

G

1970: hIEE2 (+) RO (FIG. 5K)

(SEQ ID NO: 153)

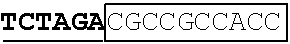

ATGGTTGCTGGGAGCGACGCGGGGCGGGCCCTGGGGGTCCTCAGCGTGGTCTGCCTGCTGCACTGCTTTGGTTTCAT
CAGCTGTTTTTCCCAACAAATATATGGTGTTGTGTATGGGAATGTAACTTTCCATGTACCAAGCAATGTGCCTTTAA
AAGAGGTCCTATGGAAAAAACAAAAGGATAAAGTTGCAGAACTGGAAAATTCTGAGTTCAGAGCTTTCTCATCTTTT
AAAAATAGGGTTTATTTAGACACTGTGTCAGGTAGCCTCACTATCTACAACTTAACATCATCAGATGAAGATGAGTA
TGAAATGGAATCGCCAAATATTACTGATACCATGAAGTTCTTTCTTTATGTGCTTGAGTCTCTTCCATCTCCCACAC
TAACTTGTGCATTGACTAATGGAAGCATTGAAGTCCAATGCATGATACCAGAGCATTACAACAGCCATCGAGGACTT
ATAATGTACTCATGGGATTGTCCTATGGAGCAATGTAAACGTAACTCAACCAGTATATATTTTAAGATGGAAAATGA
TCTTCCACAAAAAATACAGTGTACTCTTAGCAATCCATTATTTAATACAACATCATCAATCATTTTGACAACCTGTA

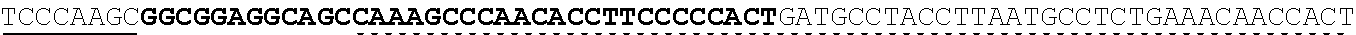

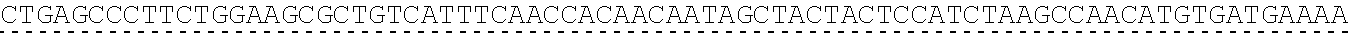

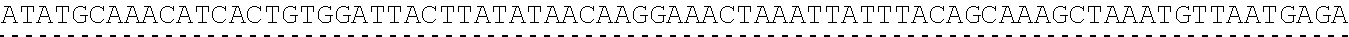

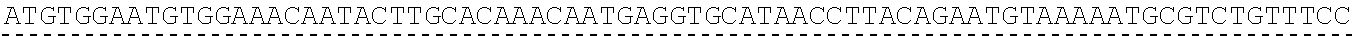

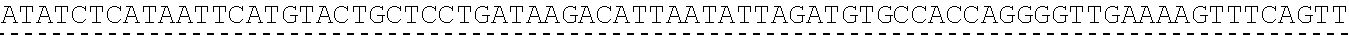

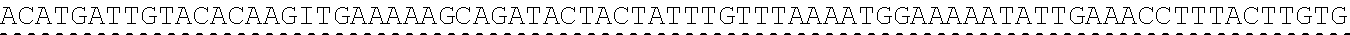

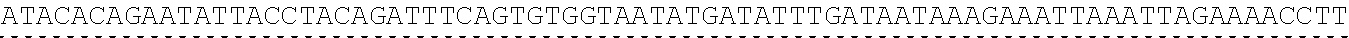

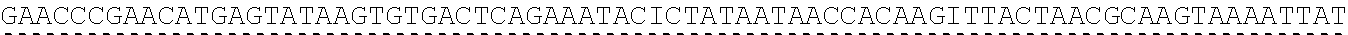

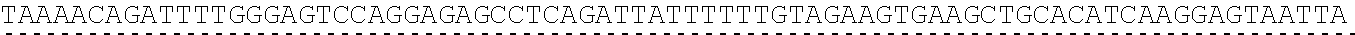

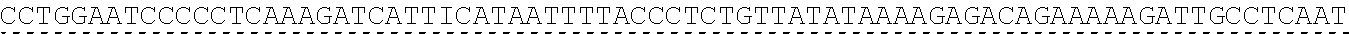

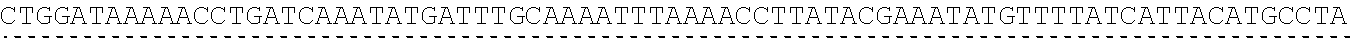

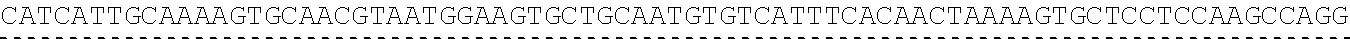

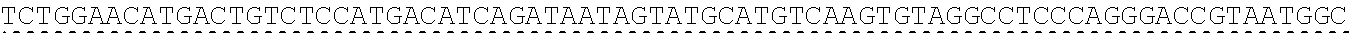

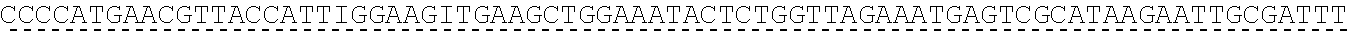

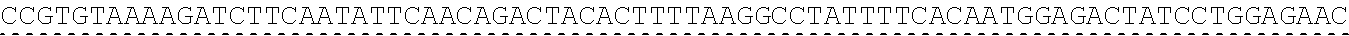

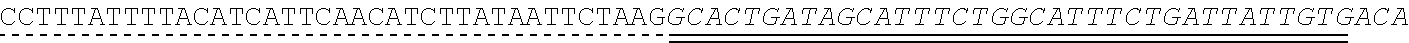

TCAATAGCCCTGCTTGTTGTTCTCTACAAAATCTATGATCTACATAAGAAAAGATCCTGCAATTGAGCGGCCGC

(SEQ ID NO: 154)

MVAGSDAGRALGVLSVVCLLHCFGFISCFSQQIYGVVYGNVTFHVPSNVPLKEVLWKKQKDKVAELENSEFRAFSSF
KNRVYLDTVSGSLTIYNLTSSDEDEYEMESPNITDTMKFFLYVLESLPSPTLTCALINGSIEVQCMIPEHYNSHRGI

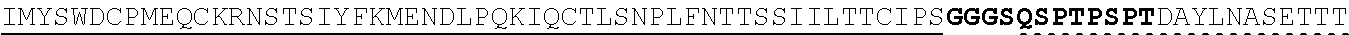

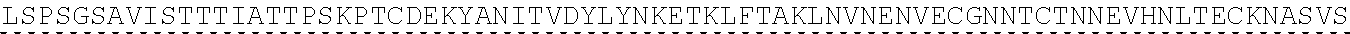

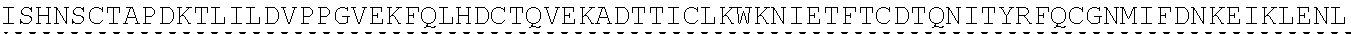

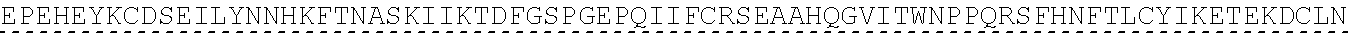

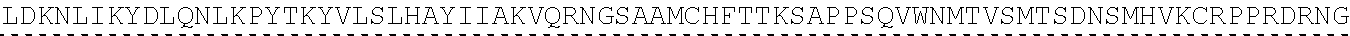

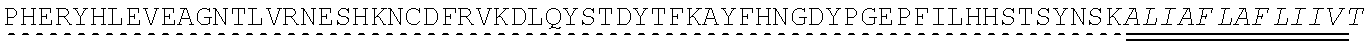

SIALLVVLYKIYDLHKKRSCN

1972: hIEE2 (+) ABC (FIG. 5K)
(SEQ ID NO: 155)

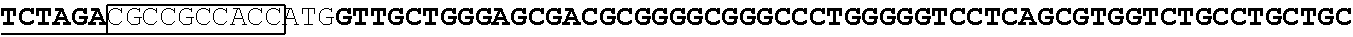

ACTGCTTTGGTTTCATCAGCTGTTTTTCCCAACAAATATATGGTGTTGTGTATGGGAATGTAACTTTCCATGTACCA
AGCAATGTGCCTTTAAAAGAGGTCCTATGGAAAAAACAAAAGGATAAAGTTGCAGAACTGGAAAATTCTGAGTTCAG
AGCTTTCTCATCTTTTAAAAATAGGGTTTATTTAGACACTGTGTCAGGTAGCCTCACTATCTACAACTTAACATCAT
CAGATGAAGATGAGTATGAAATGGAATCGCCAAATATTACTGATACCATGAAGTTCTTTCTTTATGTGCTTGAGTCT
CTTCCATCTCCCACACTAACTTGTGCATTGACTAATGGAAGCATTGAAGTCCAATGCATGATACCAGAGCATTACAA
CAGCCATCGAGGACTTATAATGTACTCATGGGATTGTCCTATGGAGCAATGTAAACGTAACTCAACCAGTATATATT
TTAAGATGGAAAATGATCTTCCACAAAAAATACAGTGTACTCTTAGCAATCCATTATTTAATACAACATCATCAATC

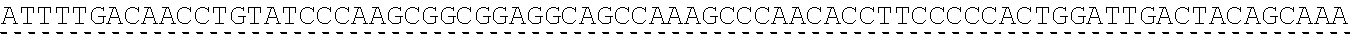

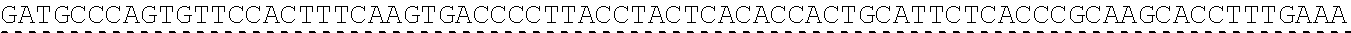

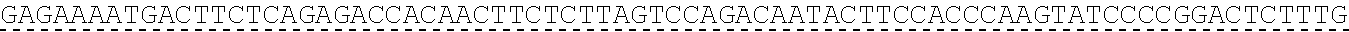

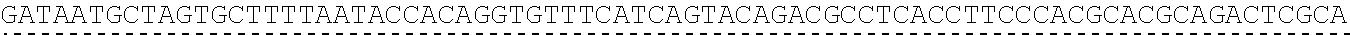

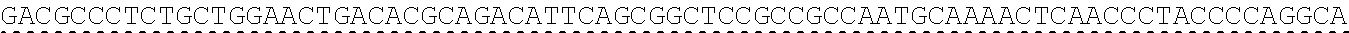

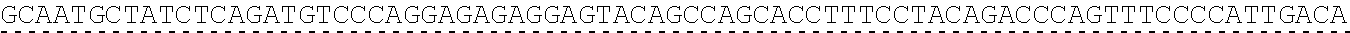

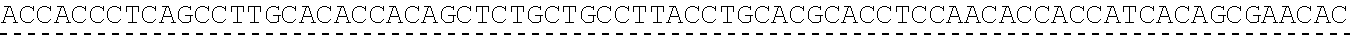

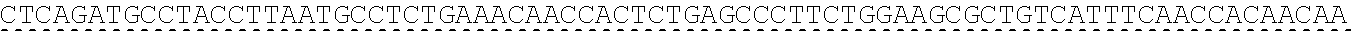

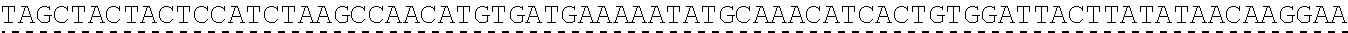

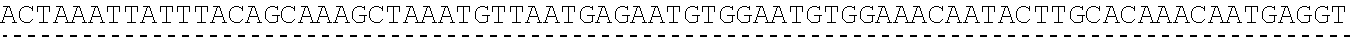

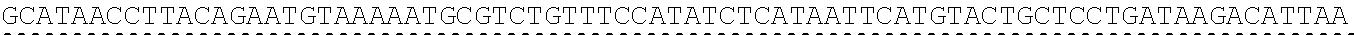

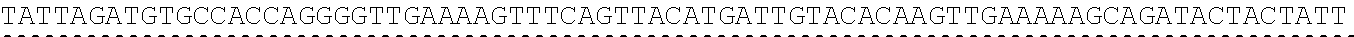

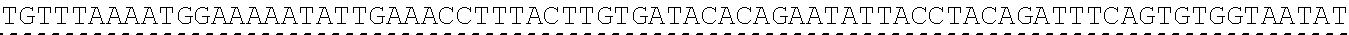

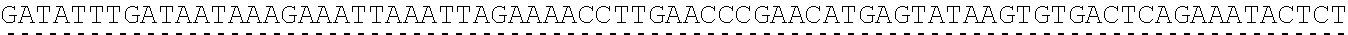

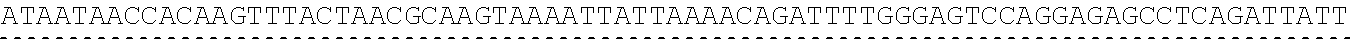

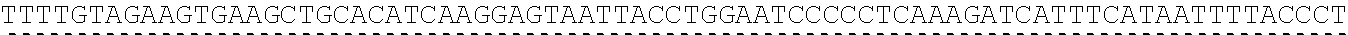

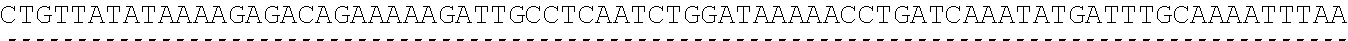

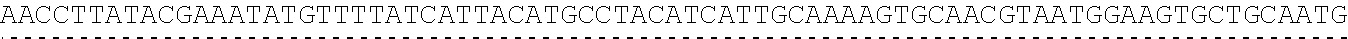

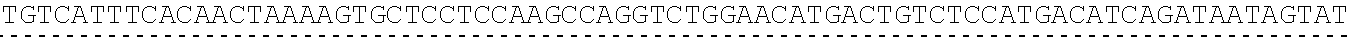

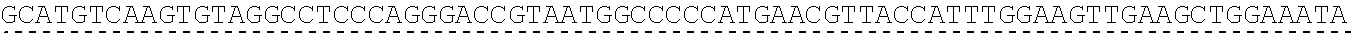

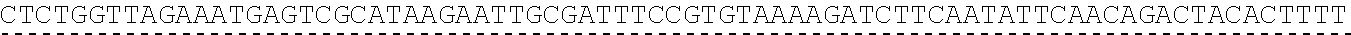

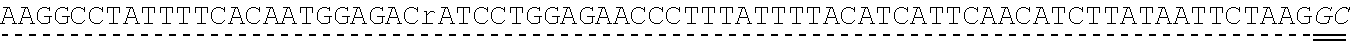

ACTGATAGCATTTCTGGCATTTCTGATTATTGIGACATCAATAGCCCTGCTTGTTGTTCTCTACAAAATCTATGATC
TACATAAGAAAAGATCCTGCAATTGAGCGGCCGC

(SEQ ID NO: 156)

MVAGSDAGRALGVLSVVCLLHCFGFISCFSQQIYGVVYGNVTFHVPSNVPLKEVLWKKQKDKVAELENSEFRAFSSF
KNRVYLDTVSGSLTIYNLTSSDEDEYEMESPNITDTMKFFLYVLESLPSPTLTCALrNGSIEVQCMIPEHYNSHRGL

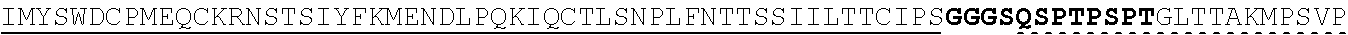

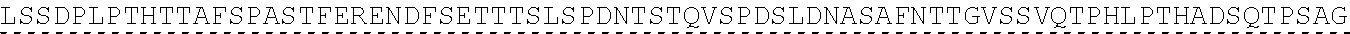

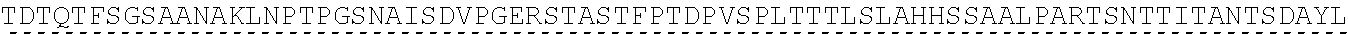

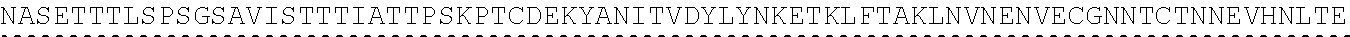

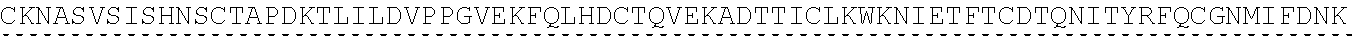

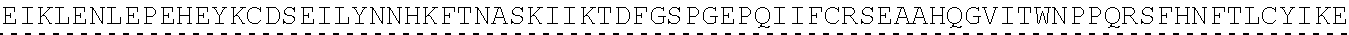

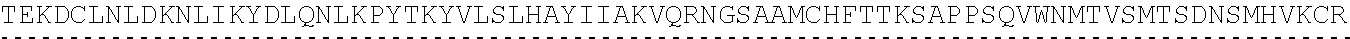

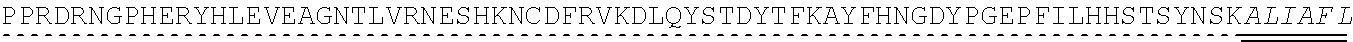

AFLIIVTSIALLVVLYKIYDLHKKRSCN

1974: hIEE-Fv-8-22 (FIG. 5L)
(SEQ ID NO: 157)

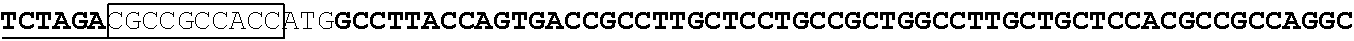

CGGACGTGGTGATGACCCAGAGCCCCCCCAGCCTGCTGGTGACCCTGGGCCAGCCCGCCAGCATCAGCTGCAGAAGC
AGCCAGAGCCTGCTGCACAGCAGCGGCAACACCTACCTGAACTGGCTGCTGCAGAGACCCGGCCAGAGCCCCCAGCC
CCTGATCTACCTGGTGAGCAAGCTGGAGAGCGGCGTGCCCGACAGATTCAGCGGCAGCGGCAGCGGCACCGACTTCA
CCCTGAAGATCAGCGGCGTGGAGGCCGAGGACGTGGGCGTGTACTACTGCATGCAGTTCACCCACTACCCCTACACC
TTCGGCCAGGGCACCAAGCTGGAGATCAAGGGCGGCGGCGGCAGCGGCGGCGGCGGCAGCGGCGGCGGCGGCAGCCA
GGTGCAGCTGGTGCAGAGCGGCGCCGAGGTGAAGAAGCCCGGCGCCAGCGTGAAGGTGAGCTGCAAGGCCAGCGGCT
ACACCTTCACCGAGTACTACATGTACTGGGTGAGACAGGCCCCCGGCCAGGGCCTGGAGCTGATGGGCAGAATCGAC
CCCGAGGACGGCAGCATCGACTACGTGGAGAAGTTCAAGAAGAAGGTGACCCTGACCGCCGACACCAGCAGCAGCAC
CGCCTACATGGAGCTGAGCAGCCTGACCAGCGACGACACCGCCGTGTACTACTGCGCCAGAGGCAAGTTCAACTACA
GATTCGCCTACTGGGGCCAGGGCACCCTGGTGACCGTGAGCAGCACCACTACCCCAGCACCGAGGCCACCCACCCCG
GCTCCTACCATCGCCTCCCAGCCTCTGTCCCTGCGTCCGGAGGCATGTAGACCCGCAGCTGGTGGGGCCGTGCATAC

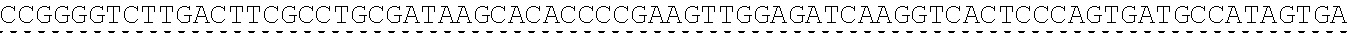

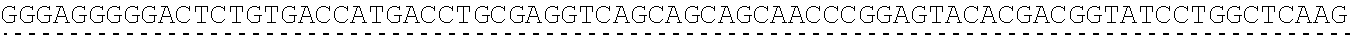

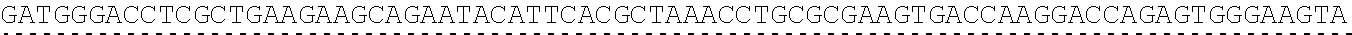

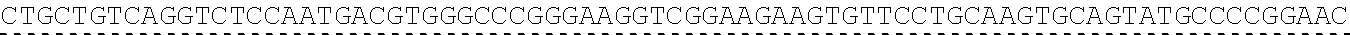

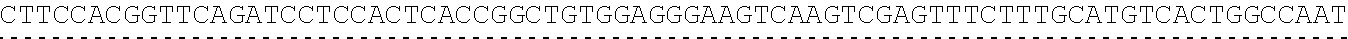

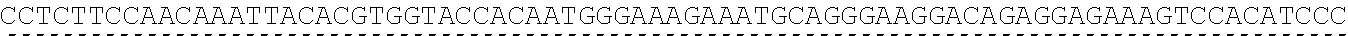

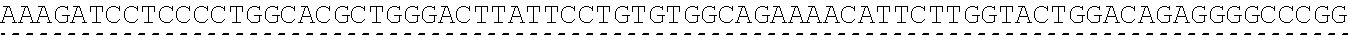

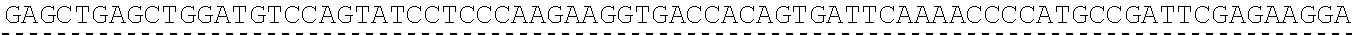

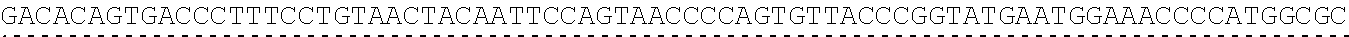

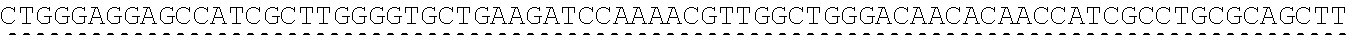

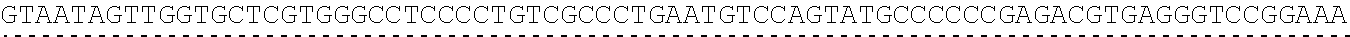

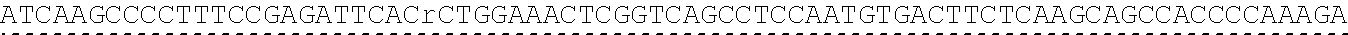

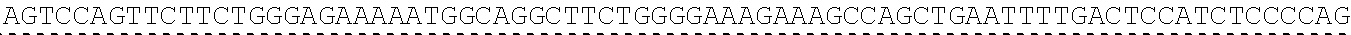

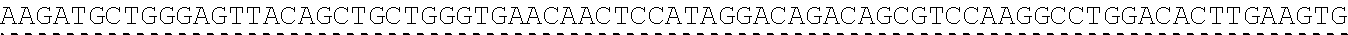

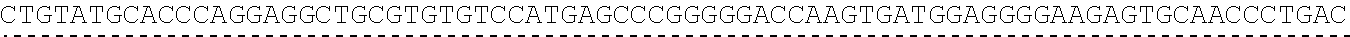

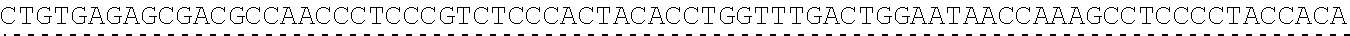

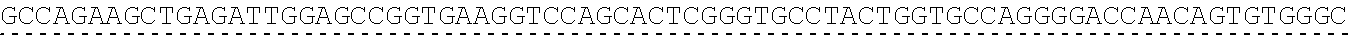

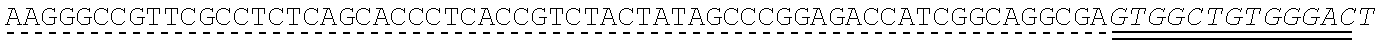

CGGGTCCTGCCTCGCCATCCTCATCCTGGCAATCTGTGGGCTCAAGCTCCAGCGACGTTGGAAGAGGACACAGAGCC
AGCAGGGGTGAGCGGCCGC

(SEQ ID NO: 158)

MALPVTALLLPLALLLHAARPDVVMTQSPPSLLVTLGQPASISCRSSQSLLHSSGNTYLNWLLQRPGQSPQPLIYLV
SKLESGVPDRFSGSGSGTDFTLKISGVEAEDVGVYYCMQFTHYPYTFGQGTKLEIKGGGGSGGGGSGGGGSQVQLVQ
SGAEVKKPGASVKVSCKASGYTFTEYYMYWVROAPGQGLELMGRIDPEDGSIDYVEKFKKKVTLTADTSSSTAYMEI
SSLTSDDTAVYYCARGKFNYRFAYWGQGTLVTVSSTTTPAPRPPTPAPTIASQPLSLRPEACRPAAGGAVHTRGLDF

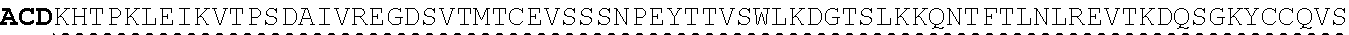

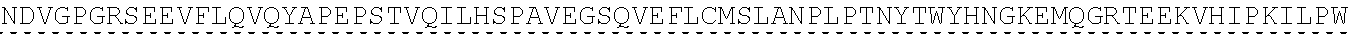

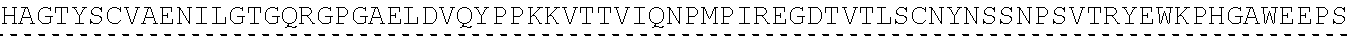

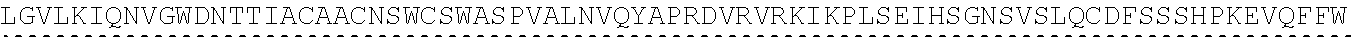

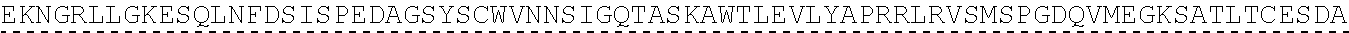

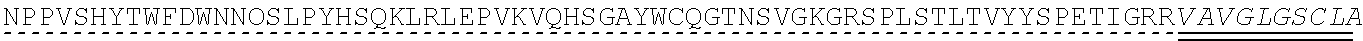

ILILAICGLKLORRWKRTOSQQG

1976: hIEE-Fv-8-RO (FIG. 5L)
(SEQ ID NO: 159)

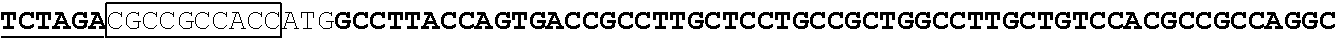

CGGACGTGGTGATGACCCAGAGCCCCCCCAGCCTGCTGGTGACCCTGGGCCAGCCCGCCAGCATCAGCTGCAGAAGC
AGCCAGAGCCTGCTGCACAGCAGCGGCAACACCTACCTGAACTGGCTGCTGCAGAGACCCGGCCAGAGCCCCCAGCC
CCTGATCTACCTGGTGAGCAAGCTGGAGAGCGGCGTGCCCGACAGATTCAGCGGCAGCGGCAGCGGCACCGACTTCA
CCCTGAAGATCAGCGGCGTGGAGGCCGAGGACGTGGGCGTGTACTACTGCATGCAGTTCACCCACTACCCCTACACC
TTCGGCCAGGGCACCAAGCTGGAGATCAAGGGCGGCGGCGGCAGCGGCGGCGGCGGCAGCGGCGGCGGCGGCAGCCA
GGTGCAGCTGGTGCAGAGCGGCGCCGAGGTGAAGAAGCCCGGCGCCAGCGTGAAGGTGAGCTGCAAGGCCAGCGGCT
ACACCTTCACCGAGTACTACATGTACTGGGTGAGACAGGCCCCCGGCCAGGGCCTGGAGCTGATGGGCAGAATCGAC
CCCGAGGACGGCAGCATCGACTACGTGGAGAAGTTCAAGAAGAAGGTGACCCTGACCGCCGACACCAGCAGCAGCAC
CGCCTACATGGAGCTGAGCAGCCTGACCAGCGACGACACCGCCGTGTACTACTGCGCCAGAGGCAAGTTCAACTACA
GATTCGCCTACTGGGGCCAGGGCACCCTGGTGACCGTGAGCAGCACCACTACCCCAGCACCGAGGCCACCCACCCCG
GCTCCTACCATCGCCTCCCAGCCTCTGTCCCTGCGTCCGGAGGCATGTAGACCCGCAGCTGGTGGGGCCGTGCATAC

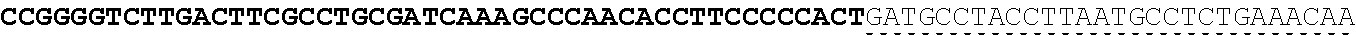

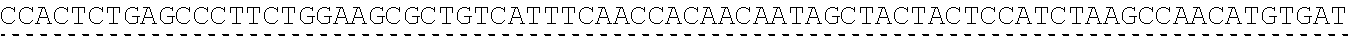

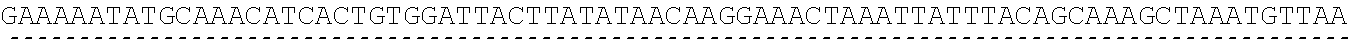

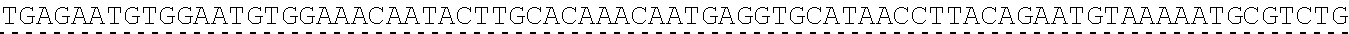

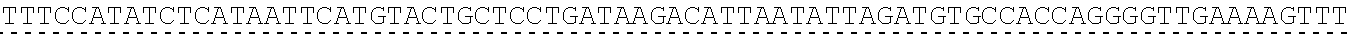

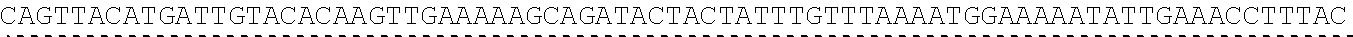

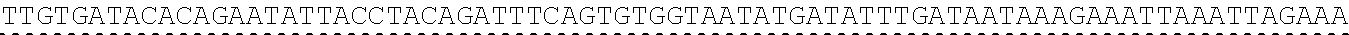

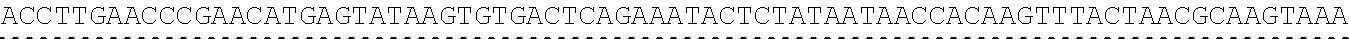

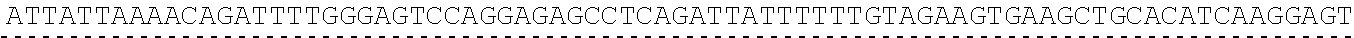

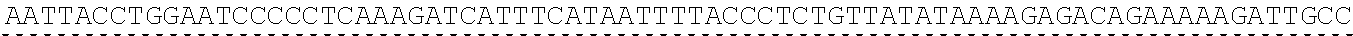

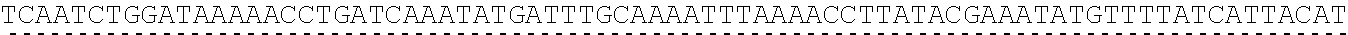

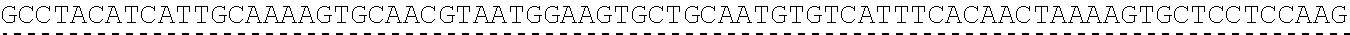

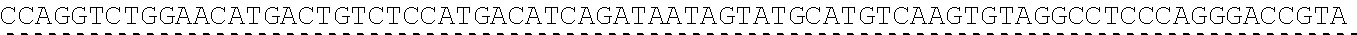

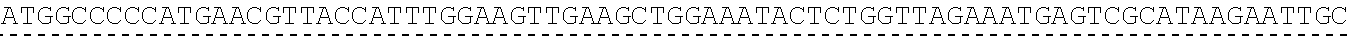

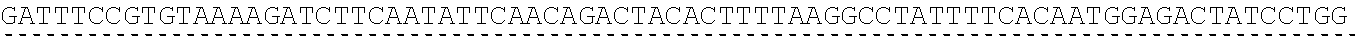

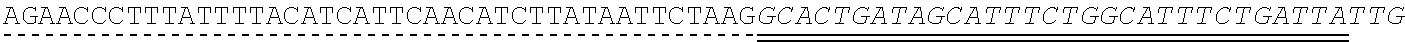

TGACATCAATAGCCCTGCTTGITGTTCTCTACAAAATCTATGATCTACATAAGAAAAGATCCTGCAATTGAGCGGCC
GC

(SEQ ID NO: 160)

MALPVTALLLPLALLLHAARPDVVMTQSPPSLLVTLGQPASISCRSSQSLLHSSGNTYLNWLLQRPGQSPQPLIYLV
SKLESGVPDRFSGSGSGTDFTLKISGVEAEDVGVYYCMQFTHYPYTFGQGTKLEIKGGGGSGGGGSGGGGSQVQLVQ
SGAEVKKPGASVKVSCKASGYTFTEYYMYWVROAPGOGLELMGRIDPEDGSIDYVEKFKKKVTLTADTSSSTAYMEL
SSLTSDDTAVYYCARGKFNYRFAYWGQGTLVTVSSTTTPAPRPPTPAPTIASQPLSLRPEACRPAAGGAVHTRGLDF

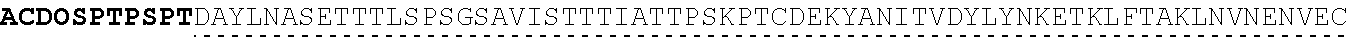

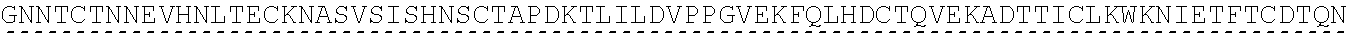

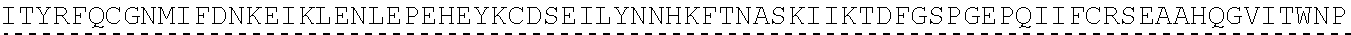

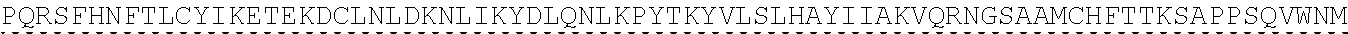

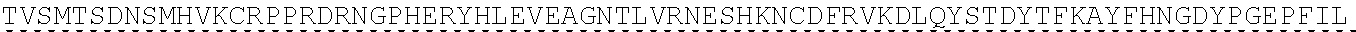

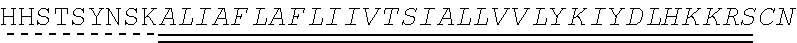

1978: hIEE-Fv-8-ABC (FIG. 5L)
(SEQ ID NO: 161)

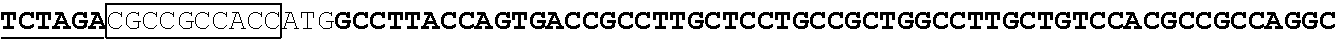

CGGACGTGGTGATGACCCAGAGCCCCCCCAGCCTGCTGGTGACCCTGGGCCAGCCCGCCAGCATCAGCTGCAGAAGC
AGCCAGAGCCTGCTGCACAGCAGCGGCAACACCTACCTGAACTGGCTGCTGCAGAGACCCGGCCAGAGCCCCCAGCC
CCTGATCTACCTGGTGAGCAAGCTGGAGAGCGGCGTGCCCGACAGATTCAGCGGCAGCGGCAGCGGCACCGACTTCA
CCCTGAAGATCAGCGGCGTGGAGGCCGAGGACGTGGGCGTGTACTACTGCATGCAGTTCACCCACTACCCCTACACC
TTCGGCCAGGGCACCAAGCTGGAGATCAAGGGCGGCGGCGGCAGCGGCGGCGGCGGCAGCGGCGGCGGCGGCAGCCA
GGTGCAGCTGGTGCAGAGCGGCGCCGAGGTGAAGAAGCCCGGCGCCAGCGTGAAGGTGAGCTGCAAGGCCAGCGGCT
ACACCTTCACCGAGTACTACATGTACTGGGTGAGACAGGCCCCCGGCCAGGGCCTGGAGCTGATGGGCAGAATCGAC
CCCGAGGACGGCAGCATCGACTACGTGGAGAAGTTCAAGAAGAAGGTGACCCTGACCGCCGACACCAGCAGCAGCAC
CGCCTACATGGAGCTGAGCAGCCTGACCAGCGACGACACCGCCGTGTACTACTGCGCCAGAGGCAAGTTCAACTACA
GATTCGCCTACTGGGGCCAGGGCACCCTGGTGACCGTGAGCAGCACCACTACCCCAGCACCGAGGCCACCCACCCCG
GCTCCTACCATCGCCTCCCAGCCTCTGTCCCTGCGTCCGGAGGCATGTAGACCCGCAGCTGGTGGGGCCGTGCATAC

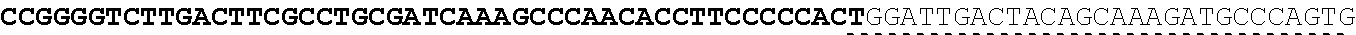

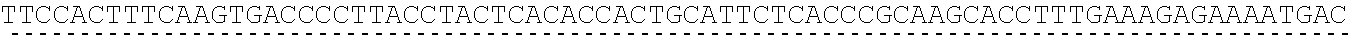

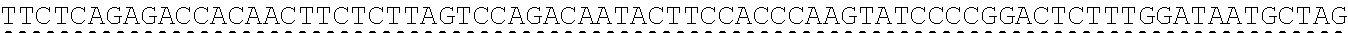

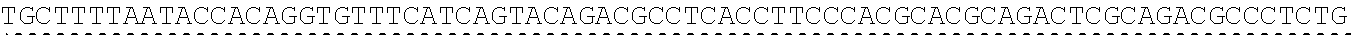

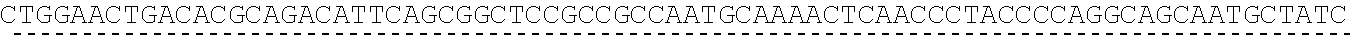

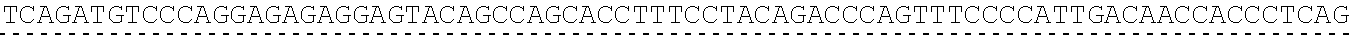

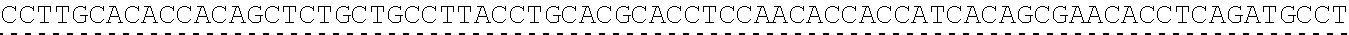

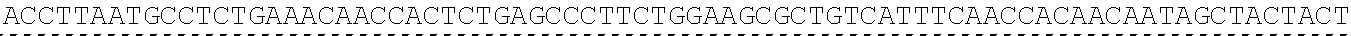

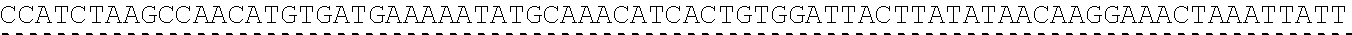

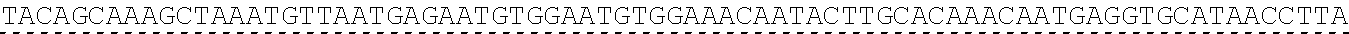

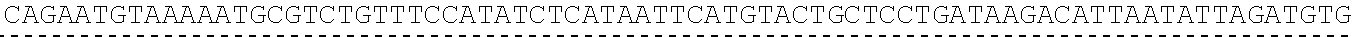

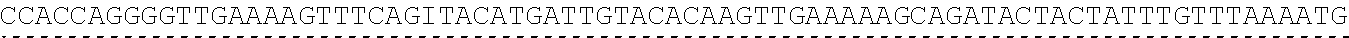

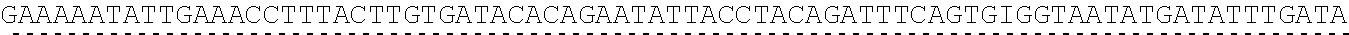

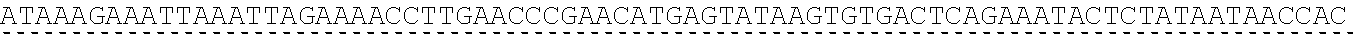

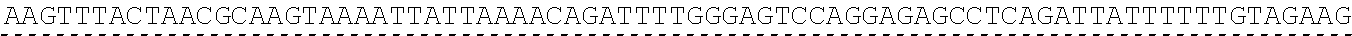

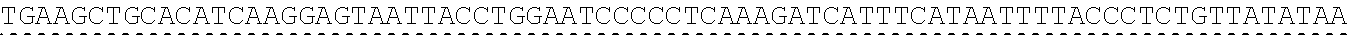

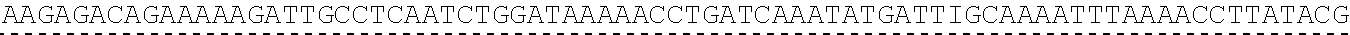

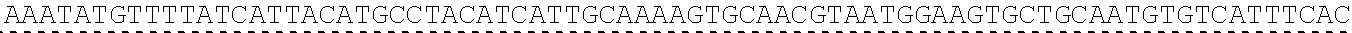

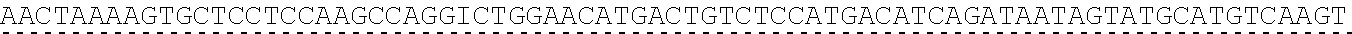

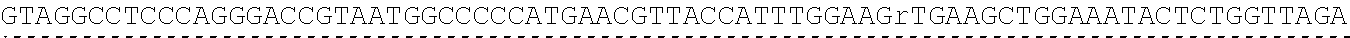

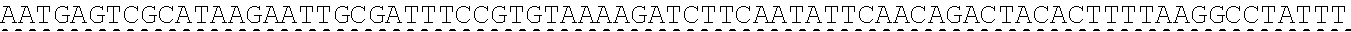

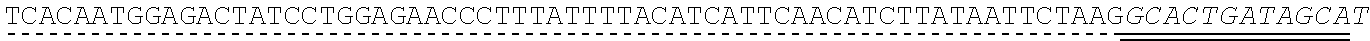

TTCTGGCATTTCTGATTATTGTGACATCAATAGCCCTGCTTGTTGTTCTCTACAAAATCTATGATCTACATAAGAAA
AGATCCTGCAATTGAGCGGCCGC

(SEQ ID NO: 162)

MALPVTALLLPLALLLHAARPDVVMTQSPPSLLVTLGQPASISCRSSQSLLHSSGNTYLNWLLQRPGQSPQPLIYLV
SKLESGVPDRFSGSGSGTDFTLKISGVEAEDVGVYYCMQFTHYPYTFGQGTKLEIKGGGGSGGGGSGGGGSQVQLVQ
SGAEVKKPGASVKVSCKASGYTFTEYYMYWVRQAPGOGLELMGRIDPEDGSIDYVEKFKKKVTLTADTSSSTAYMEL
SSLTSDDTAVYYCARGKFNYRFAYWGQGTLVTVSSTTTPAPRPPTPAPTIASQPLSLRPEACRPAAGGAVHTRGLDF

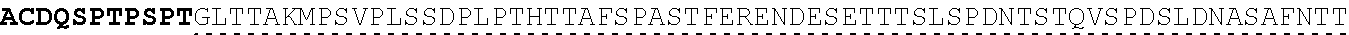

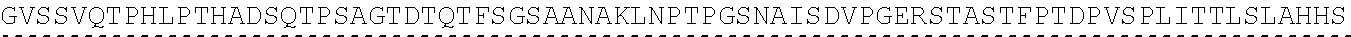

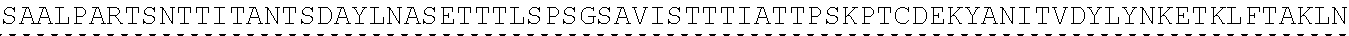

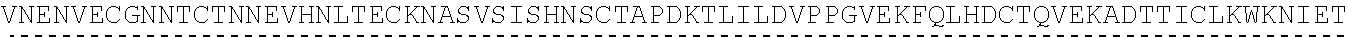

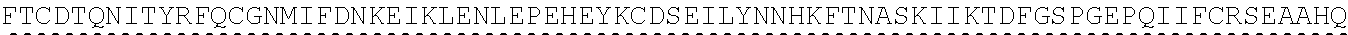

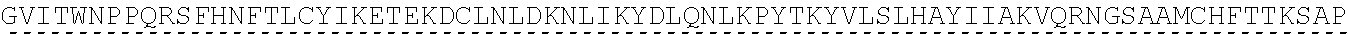

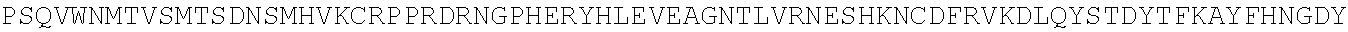

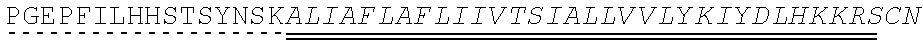

1973: hIEE-Fv-Li-22 (FIG. 5L)
(SEQ ID NO: 163)

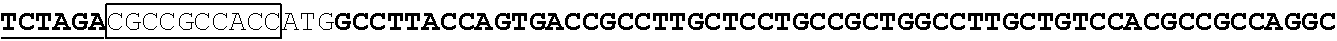

CGGACGTGGTGATGACCCAGAGCCCCCCCAGCCTGCTGGTGACCCTGGGCCAGCCCGCCAGCATCAGCTGCAGAAGC
AGCCAGAGCCTGCTGCACAGCAGCGGCAACACCTACCTGAACTGGCTGCTGCAGAGACCCGGCCAGAGCCCCCAGCC
CCTGATCTACCTGGTGAGCAAGCTGGAGAGCGGCGTGCCCGACAGATTCAGCGGCAGCGGCAGCGGCACCGACTTCA
CCCTGAAGATCAGCGGCGTGGAGGCCGAGGACGTGGGCGTGTACTACTGCATGCAGTTCACCCACTACCCCTACACC
TTCGGCCAGGGCACCAAGCTGGAGATCAAGGGCGGCGGCGGCAGCGGCGGCGGCGGCAGCGGCGGCGGCGGCAGCCA
GGTGCAGCTGGTGCAGAGCGGCGCCGAGGTGAAGAAGCCCGGCGCCAGCGTGAAGGTGAGCTGCAAGGCCAGCGGCT
ACACCTTCACCGAGTACTACATGTACTGGGTGAGACAGGCCCCCGGCCAGGGCCTGGAGCTGATGGGCAGAATCGAC
CCCGAGGACGGCAGCATCGACTACGTGGAGAAGTTCAAGAAGAAGGTGACCCTGACCGCCGACACCAGCAGCAGCAC
CGCCTACATGGAGCTGAGCAGCCTGACCAGCGACGACACCGCCGTGTACTACTGCGCCAGAGGCAAGTTCAACTACA

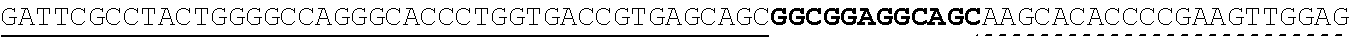

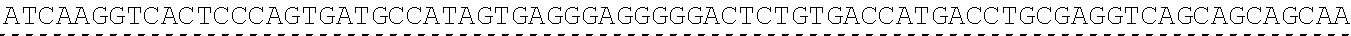

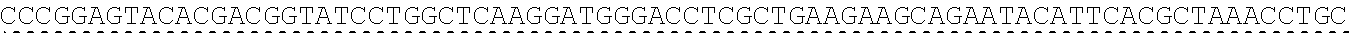

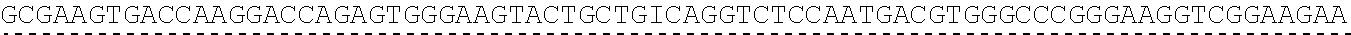

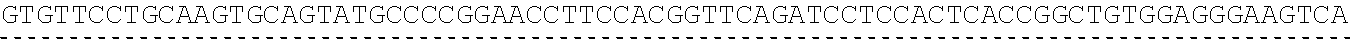

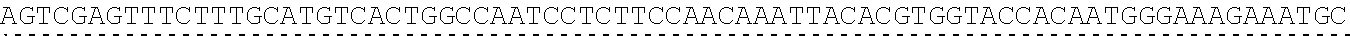

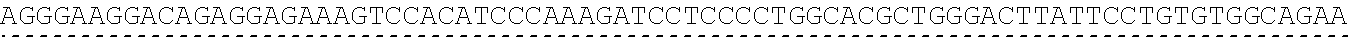

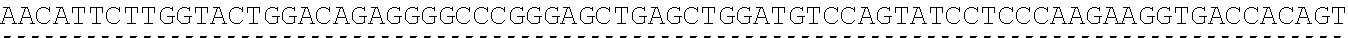

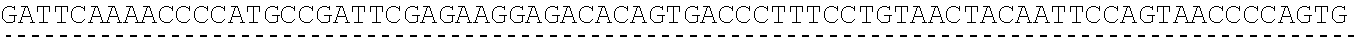

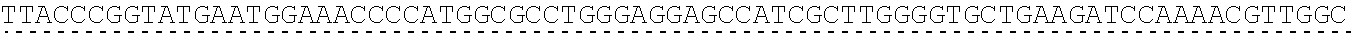

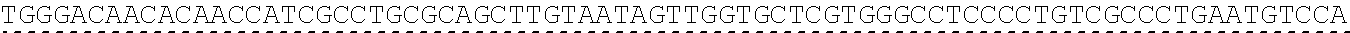

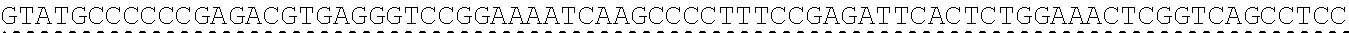

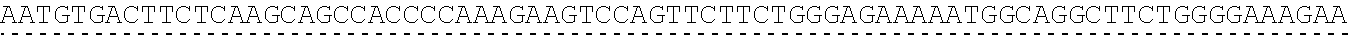

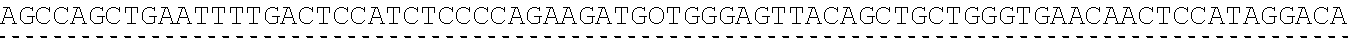

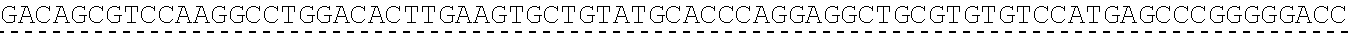

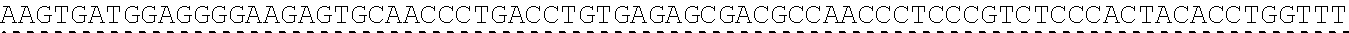

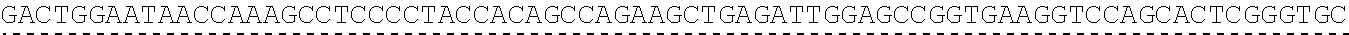

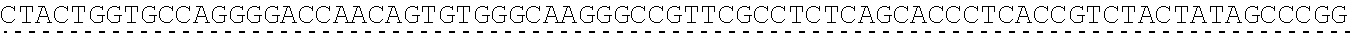

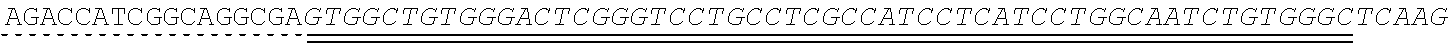

CTCCAGCGACGTTGGAAGAGGACACAGAGCCAGCAGGGGTGAGCGGCCGC

(SEQ ID NO: 164)

MALPVTALLLPLALLLHAARPDVVMTQSPPSLLVTLGQPASISCRSSQSLLHSSGNTYLNWLLQRPGQSPQPLIYLV
SKLESGVPDRFSGSGSGTDFTLKISGVEAEDVGVYYCMQFTHYPYTFGQGTKLEIKGGGGSGGGGSGGGGSQVQLVQ
SGAEVKKPGASVKVSCKASGYTFTEYYMYWVROAPGOGLELMGRIDPEDGSIDYVEKFKKKVTLTADTSSSTAYMEL

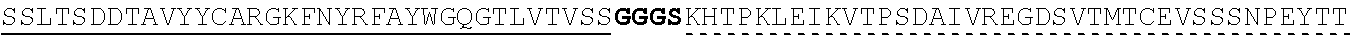

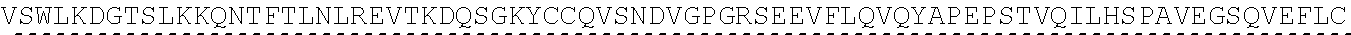

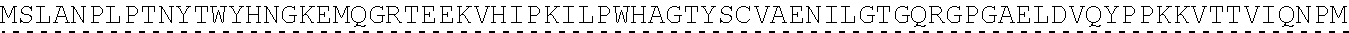

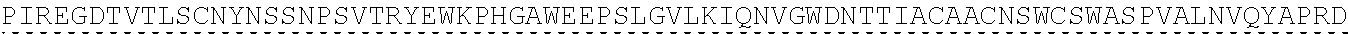

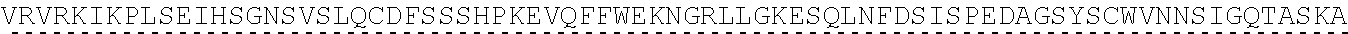

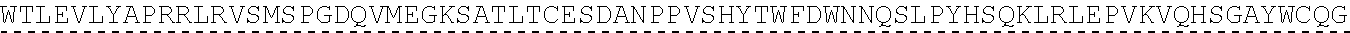

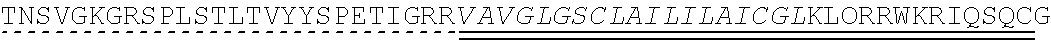

1975: hIEE-Fv-Li-RO (FIG. 5L)
(SEQ ID NO: 165)

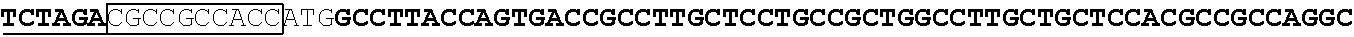

CGGACGTGGTGATGACCCAGAGCCCCCCCAGCCTGCTGGTGACCCTGGGCCAGCCCGCCAGCATCAGCTGCAGAAGC
AGCCAGAGCCTGCTGCACAGCAGCGGCAACACCTACCTGAACTGGCTGCTGCAGAGACCCGGCCAGAGCCCCCAGCC
CCTGATCTACCTGGTGAGCAAGCTGGAGAGCGGCGTGCCCGACAGATTCAGCGGCAGCGGCAGCGGCACCGACTTCA
CCCTGAAGATCAGCGGCGTGGAGGCCGAGGACGTGGGCGTGTACTACTGCATGCAGTTCACCCACTACCCCTACACC
TTCGGCCAGGGCACCAAGCTGGAGATCAAGGGCGGCGGCGGCAGCGGCGGCGGCGGCAGCGGCGGCGGCGGCAGCCA
GGTGCAGCTGGTGCAGAGCGGCGCCGAGGTGAAGAAGCCCGGCGCCAGCGTGAAGGTGAGCTGCAAGGCCAGCGGCT
ACACCTTCACCGAGTACTACATGTACTGGGTGAGACAGGCCCCCGGCCAGGGCCTGGAGCTGATGGGCAGAATCGAC
CCCGAGGACGGCAGCATCGACTACGTGGAGAAGTTCAAGAAGAAGGTGACCCTGACCGCCGACACCAGCAGCAGCAC
CGCCTACATGGAGCTGAGCAGCCTGACCAGCGACGACACCGCCGTGTACTACTGCGCCAGAGGCAAGTTCAACTACA
GATTCGCCTACTGGGGCCAGGGCACCCTGGTGACCGTGAGCAGCGGCGGAGGCAGCCAAAGCCCAACACCTTCCCCC

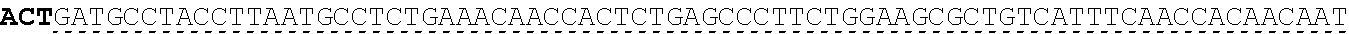

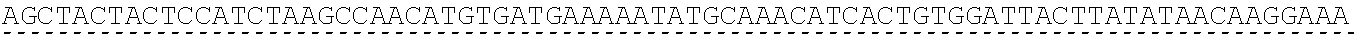

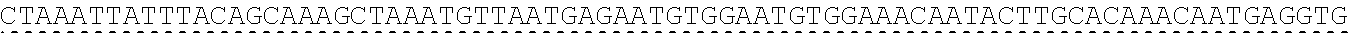

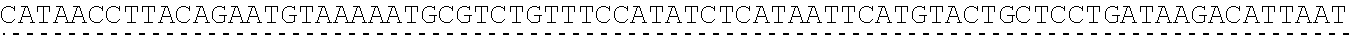

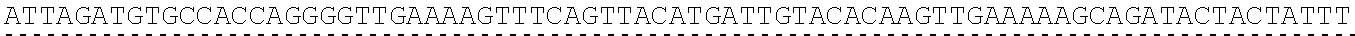

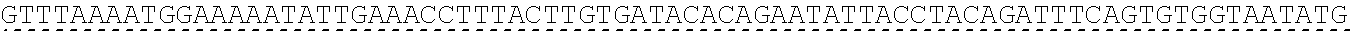

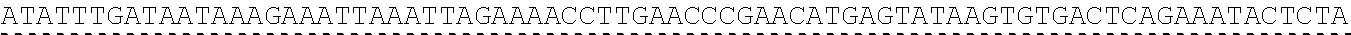

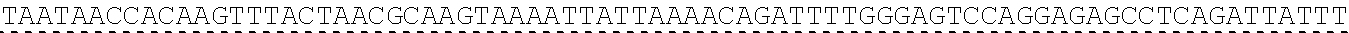

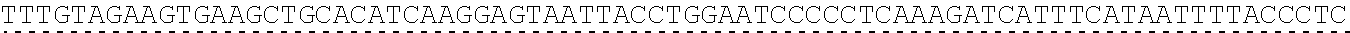

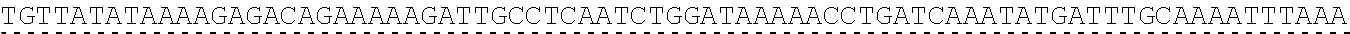

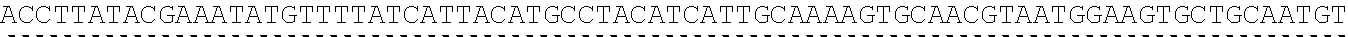

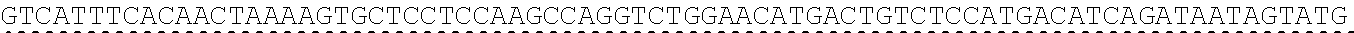

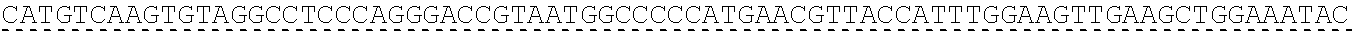

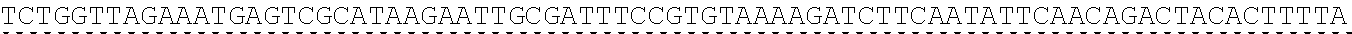

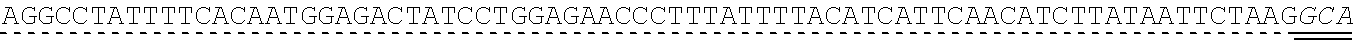

CTGATAGCATTTCIGGCATTTCTGATTATTGIGACATCAATAGCCCTGCTTGTTGTTCTCTACAAAATCTATGATCT
ACATAAGAAAAGATCCTGCAATTGAGCGGCCGC

(SEQ ID NO: 166)

MALPVTALLLPLALLLHAARPDVVMTQSPPSLLVTLGQPASISCRSSQSLLHSSGNTYLNWLLQRPGQSPQPLIYLV
SKLESGVPDRFSGSGSGTDFTLKISGVEAEDVGVYYCMQFTHYPYTFGQGTKLEIKGGGGSGGGGSGGGGSQVQLVQ
SGAEVKKPGASVKVSCKASGYTFTEYYMYWVROAPGQGLELMGRIDPEDGSIDYVEKFKKKVTLTADTSSSTAYMEL

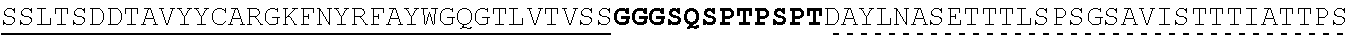

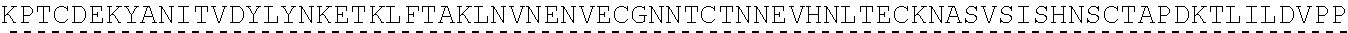

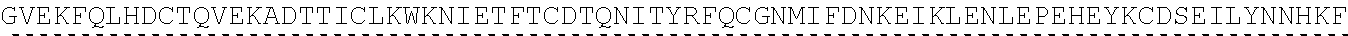

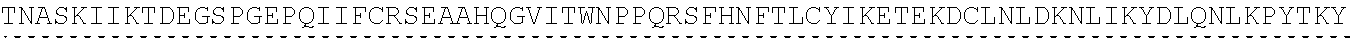

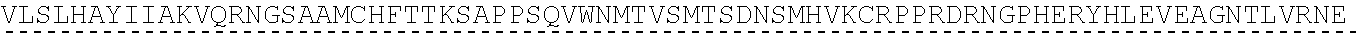

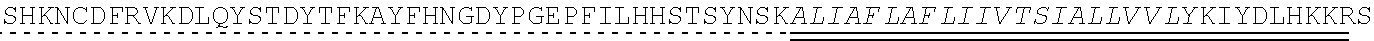

CN

1977: hIEE-Fv-Li-ABC (FIG. 5L)
(SEQ ID NO: 167)

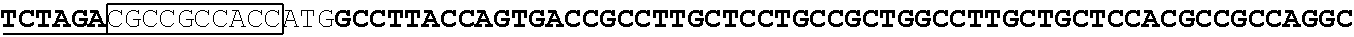

CGGACGTGGTGATGACCCAGAGCCCCCCCAGCCTGCTGGTGACCCTGGGCCAGCCCGCCAGCATCAGCTGCAGAAGC
AGCCAGAGCCTGCTGCACAGCAGCGGCAACACCTACCTGAACTGGCTGCTGCAGAGACCCGGCCAGAGCCCCCAGCC
CCTGATCTACCTGGTGAGCAAGCTGGAGAGCGGCGTGCCCGACAGATTCAGCGGCAGCGGCAGCGGCACCGACTTCA
CCCTGAAGATCAGCGGCGTGGAGGCCGAGGACGTGGGCGTGTACTACTGCATGCAGTTCACCCACTACCCCTACACC
TTCGGCCAGGGCACCAAGCTGGAGATCAAGGGCGGCGGCGGCAGCGGCGGCGGCGGCAGCGGCGGCGGCGGCAGCCA
GGTGCAGCTGGTGCAGAGCGGCGCCGAGGTGAAGAAGCCCGGCGCCAGCGTGAAGGTGAGCTGCAAGGCCAGCGGCT
ACACCTTCACCGAGTACTACATGTACTGGGTGAGACAGGCCCCCGGCCAGGGCCTGGAGCTGATGGGCAGAATCGAC
CCCGAGGACGGCAGCATCGACTACGTGGAGAAGTTCAAGAAGAAGGTGACCCTGACCGCCGACACCAGCAGCAGCAC
CGCCTACATGGAGCTGAGCAGCCTGACCAGCGACGACACCGCCGTGTACTACTGCGCCAGAGGCAAGTTCAACTACA
GATTCGCCTACTGGGGCCAGGGCACCCTGGTGACCGTGAGCAGCGGCGGAGGCAGCCAAAGCCCAACACCTTCCCCC

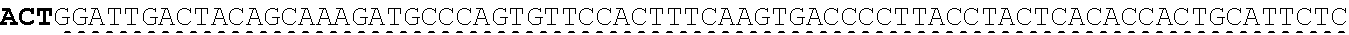

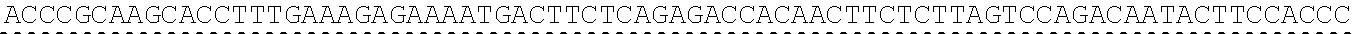

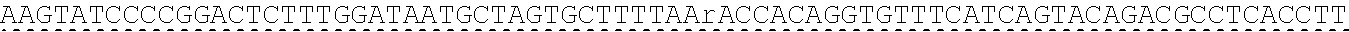

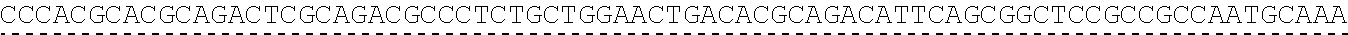

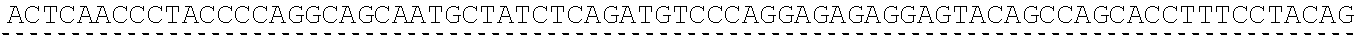

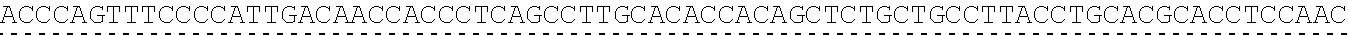

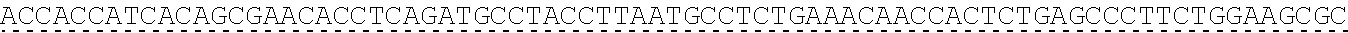

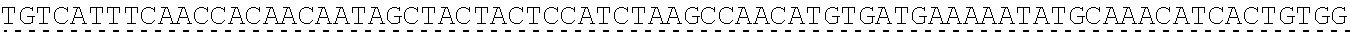

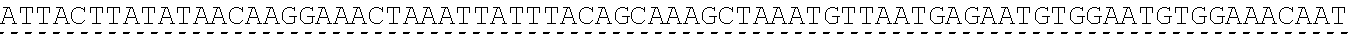

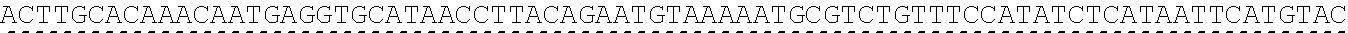

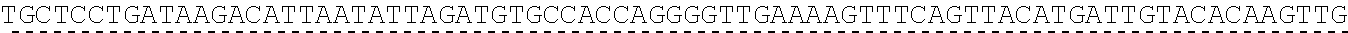

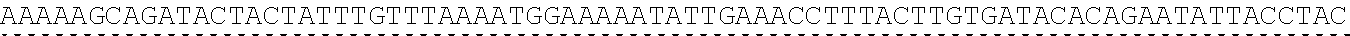

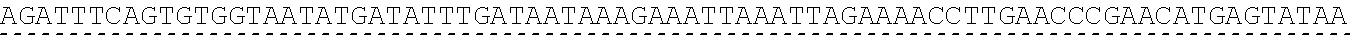

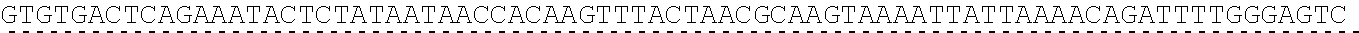

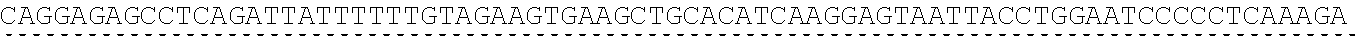

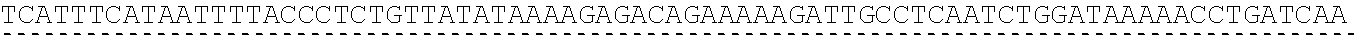

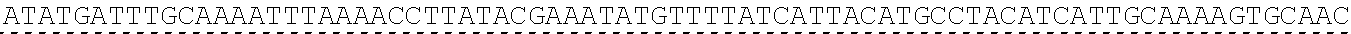

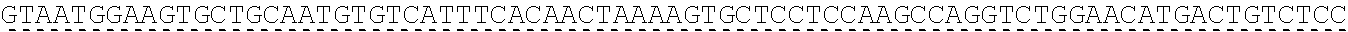

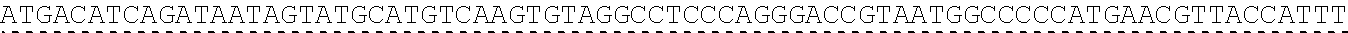

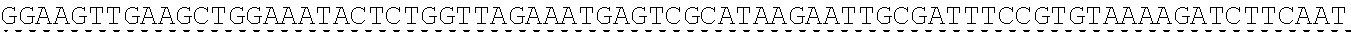

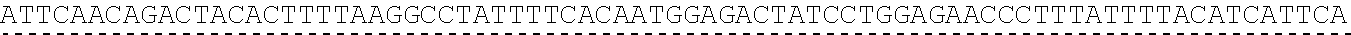

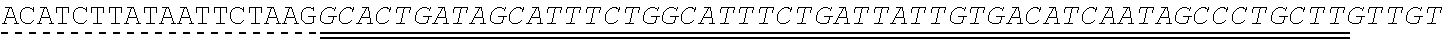

TCTCTACAAAATCTATGATCTACATAAGAAAAGATCCTGCAATTGAGCGGCCGC

(SEQ ID NO: 168)

MALPVTALLLPLALLLHAARPDVVMTQSPPSLLVTLGQPASISCRSSQSLLHSSGNTYLNWLLQRPGQSPQPLIYLV
SKLESGVPDRFSGSGSGTDFTLKISGVEAEDVGVYYCMQFTHYPYTFGQGTKLEIKGGGGSGGGGSGGGGSQVQLVQ
SGAEVKKPGASVKVSCKASGYTFTEYYMYWVROAPGOGLELMGRIDPEDGSIDYVEKFKKKVTLTADTSSSTAYMEL

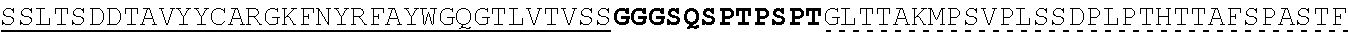

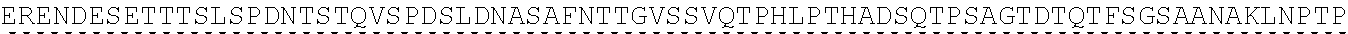

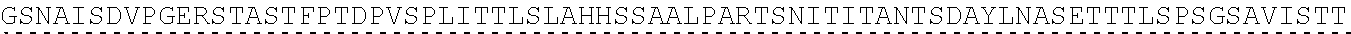

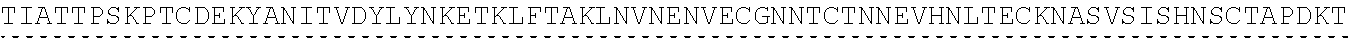

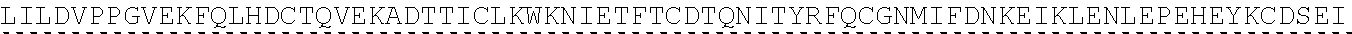

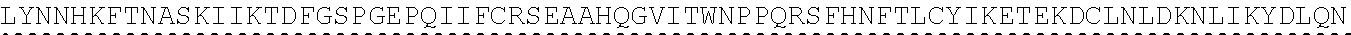

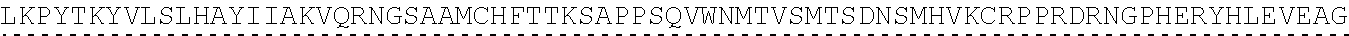

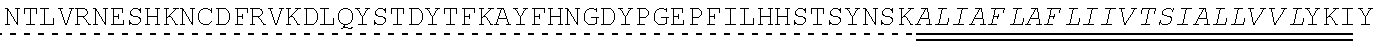

DLHKKRSCN

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

### REFERENCES

1. Yang Y, Jacoby E, Fry T J. Challenges and opportunities of allogeneic donor-derived CAR T cells. Curr Opin Hematol. 2015; 22:509-15.
2. Qasim W. Allogeneic CAR T cell therapies for leukemia. Am J Hematol [Internet]. Am J Hematol; 2019 [cited 2020 Jul. 13]; 94:550-4. Available from: http://www.ncbi.nlm.nih.gov/pubmed/30632623
3. Kim D W, Cho J-Y. Recent advances in allogeneic CAR-T cells. Biomolecules. 2020; 10.
4. Depil S, Duchateau P, Grupp S A, Mufti G, Poirot L. ‘Off-the-shelf’ allogeneic CAR T cells: development and challenges. Nat Rev Drug Discov [Internet]. Nature Publishing Group; 2020 [cited 2020 Jul. 13]; 19:185-99. Available from: http://www.nature.com/articles/s41573-019-0051-2
5. MacDonald K N, Piret J M, Levings M K. Methods to manufacture regulatory T cells for cell therapy. Clin Exp Immunol [Internet]. John Wiley & Sons, Ltd; 2019 [cited 2020 Jul. 14]; 197:cei.13297. Available from: https://onlinelibrary.wiley.com/doi/abs/10.1111/cei.13297
6. Ferreira LMR, Muller Y D, Bluestone J A, Tang Q. Next-generation regulatory T cell therapy. Nat Rev Drug Discov [Internet]. Nature Publishing Group; 2019 [cited 2019 Oct. 2]; 18:749-69. Available from: http://www.nature.com/articles/s41573-019-0041-4
7. Fritsche E, Volk H-D, Reinke P, Abou-El-Enein M. Toward an Optimized Process for Clinical Manufacturing of CAR-Treg Cell Therapy. Trends Biotechnol [Internet]. Trends Biotechnol; 2020 [cited 2020 Jul. 14]; Available from: http://www.ncbi.nlm.nih.gov/pubmed/31982150
8. Raffin C, Vo L T, Bluestone J A. Treg cell-based therapies: challenges and perspectives.

Nat Rev Immunol. 2020; 20:158-72.
9. Wang D, Quan Y, Yan Q, Morales J E, Wetsel R A. Targeted Disruption of the β2-Microglobulin Gene Minimizes the Immunogenicity of Human Embryonic Stem Cells. Stem Cells Transl Med [Internet]. Stem Cells Transl Med; 2015 [cited 2020 Jul. 14]; 4:1234-45. Available from: http://www.ncbi.nlm.nih.gov/pubmed/26285657
10. Gornalusse G G, Hirata R K, Funk S E, Riolobos L, Lopes V S, Manske G, et al. HLA-E-expressing pluripotent stem cells escape allogeneic responses and lysis by N K cells. Nat Biotechnol [Internet]. Nat Biotechnol; 2017 [cited 2020 Jul. 14]; 35:765-72. Available from: http://www.ncbi.nlm.nih.gov/pubmed/28504668
11. Torikai H, Reik A, Soldner F, Warren E H, Yuen C, Zhou Y, et al. Toward eliminating HLA class I expression to generate universal cells from allogeneic donors. Blood [Internet]. Affiliation: Division of Pediatrics, The University of Texas M D Anderson Cancer Center, Houston, Tex. 77030, USA.; 2013; 122:1341-9. Available from: http://www.scopus.com/inward/record.url?eid=2-s2.0-84886849781&partnerlD=40&md5=5053221e7f995f2a8deec5da4el6bble
12. Xu H, Wang B, Ono M, Kagita A, Fujii K, Sasakawa N, et al. Targeted Disruption of HLA Genes via CRISPR-Cas9 Generates iPSCs with Enhanced Immune Compatibility. Cell Stem Cell [Internet]. Cell Stem Cell; 2019 [cited 2020 Jul. 14]; 24:566-578.e7. Available from: http://www.ncbi.nlm.nih.gov/pubmed/30853558
13. Krawczyk M, Peyraud N, Rybtsova N, Masternak K, Bucher P, Barras E, et al. Long distance control of MHC class II expression by multiple distal enhancers regulated by regulatory factor X complex and CIITA. J Immunol [Internet]. American Association of Immunologists; 2004 [cited 2020 Jul. 14]; 173:6200-10. Available from: http://www.ncbi.nlm.nih.gov/pubmed/15528357
14. Crivello P, Ahci M, Maa8en F, Wossidlo N, Arrieta-Bolanos E, Heinold A, et al. Multiple Knockout of Classical HLA Class II β-Chains by CRISPR/Cas9 Genome Editing Driven by a Single Guide RNA. J Immunol [Internet]. J Immunol; 2019 [cited 2020 Jul. 14]; 202:1895-903. Available from: http://www.ncbi.nlm.nih.gov/pubmed/30700588
15. Poirot L, Philip B, Schiffer-Mannioui C, Le Clerre D, Chion-Sotinel I, Derniame S, et al. Multiplex genome edited T-cell manufacturing platform for “off-the-shelf” adoptive T-cell immunotherapies. Cancer Res [Internet]. 2015 [cited 2015 Jul. 18]; 75:3853-64. Available from: http://www.ncbi.nlm.nih.gov/pubmed/26183927
16. Qasim W, Zhan H, Samarasinghe S, Adams S, Amrolia P, Stafford S, et al. Molecular remission of infant B-ALL after infusion of universal TALEN gene-edited CAR T cells. Sci Transl Med [Internet]. 2017 [cited 2017 Jan. 26]; 9:eaaj2013. Available from: http://stm.sciencemag.org/content/9/374/eaaj20l3.abstract
17. Valton J, Guyot V, Marechal A, Filhol J-M, Juillerat A, Duclert A, et al. A Multidrug-resistant Engineered CAR T Cell for Allogeneic Combination Immunotherapy. Mol Ther [Internet]. American Society of Gene & Cell Therapy; 2015 [cited 2016 Sep. 15]; 23:1507-18. Available from: http://dx.doi.org/10.1038/mt.2015.104
18. Han X, Wang M, Duan S, Franco P J, Kenty J H-R, Hedrick P, et al. Generation of hypoimmunogenic human pluripotent stem cells. Proc Natl Acad Sci USA [Internet]. National Academy of Sciences; 2019 [cited 2020 Jul. 31]; 116:10441-6. Available from: http://www.ncbi.nlm.nih.gov/pubmed/31040209
19. Malik N N, Jenkins A M, Mellon J, Bailey G. Engineering strategies for generating hypoimmunogenic cells with high clinical and commercial value. Regen Med. 2019; 14:983-9.
20. Davis S J, van der Merwe P A. The structure and ligand interactions of CD2: implications for T-cell function. Immunol Today [Internet]. 1996 [cited 2019 Mar. 16]; 17:177-87. Available from: http://linkinghub.elsevier.com/retrieve/pii/0167569996806177
21. Anton van der Merwe P, Davis S J, Shaw A S, Dustin M L. Cytoskeletal polarization and redistribution of cell-surface molecules during T cell antigen recognition. Semin Immunol [Internet]. Sir William Dunn School of Pathology, University of Oxford, U K.; 2000; 12:5-21. Available from: http://www.ncbi.nlm.nih.gov/entrez/query.fcgi?cmd=Retrieve&db=PubMed&dopt=Citation&list _uids=10723794
22. Davis S J, van der Merwe P A. The kinetic-segregation model: TCR triggering and beyond. Nat Immunol [Internet]. 2006 [cited 2017 Feb. 5]; 7:803-9. Available from: http://www.ncbi.nlm.nih.gov/pubmed/16855606
23. Chang V T, Fernandes R A, Ganzinger K A, Lee S F, Siebold C, McColl J, et al. Initiation of T cell signaling by CD45 segregation at “close contacts”. Nat Immunol [Internet]. 2016 [cited 2017 Jan. 27]; 17:574-82. Available from: http://www.pubmedcentral.nih.gov/articlerender.fcgi?artid=4839504&tool=pmcentrez&renderty pe=abstract
24. Binder C, Cvetkovski F, Sellberg F, Berg S, Paternina Visbal H, Sachs D H, et al. CD2 Immunobiology. Front Immunol [Internet]. Frontiers; 2020 [cited 2020 Jul. 31]; 11:1090. Available from: https://www.frontiersin.org/article/10.3389/fimmu.2020.01090/full
25. Wild M K, Cambiaggi A, Brown M H, Davies E A, Ohno H, Saito T, et al. Dependence of T cell antigen recognition on the dimensions of an accessory receptor-ligand complex. J Exp Med [Internet]. 1999 [cited 2017 Feb. 6]; 190:31-41. Available from: http://www.pubmedcentral.nih.gov/articlerender.fcgi?artid=2195552&tool=pmcentrez&renderty pe=abstract
26. Irles C, Symons A, Michel F, Bakker T R, van der Merwe P A, Acuto O. CD45 ectodomain controls interaction with GEMs and Lck activity for optimal TCR signaling. Nat Immunol. 2003; 4:189-97.
27. Choudhuri K, Wiseman D, Brown M H, Gould K, van der Merwe P A. T-cell receptor triggering is critically dependent on the dimensions of its peptide-MHC ligand. Nature [Internet]. 2005 [cited 2017 Jan. 28]; 436:578-82. Available from: http://www.ncbi.nlm.nih.gov/pubmed/16049493
28. James J R, Vale R D. Biophysical mechanism of T-cell receptor triggering in a reconstituted system. Nature [Internet]. 2012 [cited 2017 Jan. 27]; 487:64-9. Available from: http://www.pubmedcentral.nih.gov/articlerender.fcgi?artid=3393772&tool=pmcentrez&renderty pe=abstract
29. Cordoba S-P, Choudhuri K, Zhang H, Bridge M, Basat A B, Dustin M L, et al. The large ectodomains of CD45 and CD148 regulate their segregation from and inhibition of ligated T-cell receptor. Blood [Internet]. 2013 [cited 2017 Jan. 25]; 121:4295-302. Available from: http://www.pubmedcentral.nih.gov/articlerender.fcgi?artid=3663424&tool=pmcentrez&renderty pe=abstract
30. Schmid E M, Bakalar M H, Choudhuri K, Weichsel J, Ann H, Geissler P L, et al. Size-dependent protein segregation at membrane interfaces. Nat Phys [Internet]. 2016 [cited 2017 Jan. 27]; 12:704-11. Available from: http://www.pubmedcentral.nih.gov/articlerender.fcgi?artid=5152624&tool=pmcentrez&renderty pe=abstract
31. Aricescu A R, Jones E Y. Immunoglobulin superfamily cell adhesion molecules: zippers and signals. Curr Opin Cell Biol [Internet]. 2007 [cited 2016 Aug. 8]; 19:543-50. Available from: http://www.scopus.com/inward/record.url?eid=2-s2.0-35548982621&partnerlD=tZOtx3y1
32. Okumura M, Matthews R J, Robb B, Litman G W, Bork P, Thomas M L. Comparison of CD45 extracellular domain sequences from divergent vertebrate species suggests the conservation of three fibronectin type III domains. J Immunol [Internet]. 1996 [cited 2017 Feb. 21]; 157:1569-75. Available from: http://www.ncbi.nlm.nih.gov/pubmed/8759740
33. Brown M H, Cantrell D A, Brattsand G, Crumpton M J, Gullberg M. The CD2 antigen associates with the T-cell antigen receptor CD3 antigen complex on the surface of human T lymphocytes. Nature [Internet]. Nature Publishing Group; 1989 [cited 2017 Feb. 20]; 339:551-3. Available from: http://www.nature.com/doifinder/10.1038/339551a0
34. Dustin M L, Olszowy M W, Holdorf A D, Li J, Bromley S, Desai N, et al. A Novel Adaptor Protein Orchestrates Receptor Patterning and Cytoskeletal Polarity in T-Cell Contacts. Cell [Internet]. 1998 [cited 2017 Feb. 13]; 94:667-77. Available from: http://www.sciencedirect.com/science/article/pii/S0092867400816086
35. Penninger J M, Crabtree G R. The actin cytoskeleton and lymphocyte activation. Cell [Internet]. 1999 [cited 2017 Feb. 14]; 96:9-12. Available from: http://www.ncbi.nlm.nih.gov/pubmed/9989492
36. Milstein O, Tseng S-Y, Starr T, Llodra J, Nans A, Liu M, et al. Nanoscale increases in CD2-CD48-mediated intermembrane spacing decrease adhesion and reorganize the immunological synapse. J Biol Chem [Internet]. 2008 [cited 2017 Feb. 20]; 283:34414-22. Available from: http://www.jbc.org/cgi/doi/10.1074/jbc.M804756200
37. Bromley S K, Burack W R, Johnsonn K G, Somersalo K, Sims T N, Sumen C, et al. The immunological synapse. Annu. Rev. Immunol. 2001.
38. Van der Merwe P A. Formation and function of the immunological synapse. Curr Opin Immunol. 2002; 14:293-8.
39. Orange J S. Formation and function of the lytic N K-cell immunological synapse. Nat Rev Immunol [Internet]. Nature Publishing Group; 2008 [cited 2020 Aug. 2]; 8:713-25. Available from: http://www.nature.com/articles/nri2381
40. Dustin M L, Long E O. Cytotoxic immunological synapses. Immunol Rev [Internet]. NIH Public Access; 2010 [cited 2019 Mar. 15]; 235:24-34. Available from: http://www.ncbi.nlm.nih.gov/pubmed/20536553
41. Brzostek J, Chai J-G, Gebhardt F, Busch D H, Zhao R, van der Merwe P A, et al. Ligand dimensions are important in controlling N K-cell responses. Eur J Immunol [Internet]. Eur J Immunol; 2010 [cited 2020 Aug. 1]; 40:2050-9. Available from: http://www.ncbi.nlm.nih.gov/pubmed/20432238
42. Orange J S, Harris K E, Andzelm M M, Valter M M, Geha R S, Strominger J L. The mature activating natural killer cell immunologic synapse is formed in distinct stages. Proc Natl Acad Sci USA [Internet]. Proc Natl Acad Sci USA; 2003 [cited 2020 Aug. 2]; 100:14151-6. Available from: http://www.ncbi.nlm.nih.gov/pubmed/14612578
43. Bierer B E, Golan D E, Brown C S, Herrmann S H, Burakoff S J. A monoclonal antibody to LFA-3, the CD2 ligand, specifically immobilizes major histocompatibility complex proteins. Eur J Immunol [Internet]. Eur J Immunol; 1989 [cited 2020 Jul. 10]; 19:661-5. Available from: http://www.ncbi.nlm.nih.gov/pubmed/2471647
44. Nielsen M-B, Gerwien J, Nielsen M, Geisler C, Ropke C, Svejgaard A, et al. MHC class II ligation induces CD58 (LFA-3)-mediated adhesion in human T cells. Exp Clin Immunogenet. 1998; 15:61-8.
45. Junghans V, Santos A M, Lui Y, Davis S J, JMnsson P. Dimensions and Interactions of Large T-Cell Surface Proteins. Front Immunol [Internet]. Frontiers Media S A; 2018 [cited 2020 Jul. 20]; 9. Available from: https://www.ncbi.nlm.nih.gov/pmc/articles/PMC6170634/
46. McKenna R M, Takemoto S K, Terasaki P I. Anti-HLA antibodies after solid organ transplantation. Transplantation. 2000; 69:319-26.
47. Themeli M, Riviere I, Sadelain M. New Cell Sources for T Cell Engineering and Adoptive Immunotherapy. Cell Stem Cell [Internet]. Cell Press; 2015 [cited 2018 Mar. 28]; 16:357-66. Available from: https://www.sciencedirect.com/science/article/pii/S1934590915001228?_rdoc=1&_fmt=high&_origin=gateway&_docanchor-&md5=b8429449ccfc9c30159a5f9aeaa92ffb
48. Riolobos L, Hirata R K, Turtle C J, Wang P-R, Gornalusse G G, Zavajlevski M, et al. HLA engineering of human pluripotent stem cells. Mol Ther [Internet]. Mol Ther; 2013 [cited 2020 Jul. 16]; 21:1232-41. Available from: http://www.ncbi.nlm.nih.gov/pubmed/23629003
49. Turner M, Leslie S, Martin N G, Peschanski M, Rao M, Taylor C J, et al. Toward the Development of a Global Induced Pluripotent Stem Cell Library. Cell Stem Cell [Internet]. 2013 [cited 2020 Jul. 17]; 13:382-4. Available from: http://www.ncbi.nlm.nih.gov/pubmed/24094319
50. Zhao W, Lei A, Tian L, Wang X, Correia C, Weiskittel T, et al. Strategies for Genetically Engineering Hypoimmunogenic Universal Pluripotent Stem Cells. iScience. 2020; 23.
51. Chen X, Zaro J L, Shen W-C. Fusion protein linkers: property, design and functionality. Adv Drug Deliv Rev [Internet]. 2013 [cited 2016 Aug. 18]; 65:1357-69. Available from: http://www.pubmedcentral.nih.gov/articlerender.fcgi?artid=3726540&tool=pmcentrez&renderty pe=abstract
52. Matuskova M, Durinikov E. Retroviral Vectors in Gene Therapy. Adv Mol Retrovirology [Internet]. InTech; 2016 [cited 2019 Mar. 21]. Available from: http://www.intechopen.com/books/advances-in-molecular-retrovirology/retroviral-vectors-in-gene-therapy
53. Izsvik Z, Ivics Z. Sleeping Beauty Transposition: Biology and Applications for Molecular Therapy. Mol Ther [Internet]. 2004 [cited 2019 May 27]; 9:147-56. Available from: http://www.ncbi.nlm.nih.gov/pubmed/14759798
54. Miller A D, Law M F, Verma I M. Generation of helper-free amphotropic retroviruses that transduce a dominant-acting, methotrexate-resistant dihydrofolate reductase gene. Mol Cell Biol [Internet]. American Society for Microbiology (ASM); 1985 [cited 2020 Mar. 7]; 5:431-7. Available from: http://www.ncbi.nlm.nih.gov/pubmed/2985952
55. Miller A D, Buttimore C. Redesign of retrovirus packaging cell lines to avoid recombination leading to helper virus production. Mol Cell Biol [Internet]. American Society for Microbiology (ASM); 1986 [cited 2019 Aug. 28]; 6:2895-902. Available from: http://www.ncbi.nlm.nih.gov/pubmed/3785217
56. Danos O, Mulligan R C. Safe and Efficient Generation of Recombinant Retroviruses with Amphotropic and Ecotropic Host Ranges [Internet]. Proc. Natl. Acad. Sci. U.S.A National Academy of Sciences; 1988 [cited 2019 Aug. 28]. p. 6460-4. Available from: https://www.jstor.org/stable/32039
57. Bregni M, Magni M, Siena S, Di Nicola M, Bonadonna G, Gianni A. Human peripheral blood hematopoietic progenitors are optimal targets of retroviral-mediated gene transfer. Blood. 1992; 80:1418-22.
58. Xu L, Stahl S K, Dave H P, Schiffmann R, Correll P H, Kessler S, et al. Correction of the enzyme deficiency in hematopoietic cells of Gaucher patients using a clinically acceptable retroviral supernatant transduction protocol. Exp Hematol [Internet]. 1994 [cited 2020 Mar. 7]; 22:223-30. Available from: http://www.ncbi.nlm.nih.gov/pubmed/8299741
59. Hughes P F, Thacker J D, Hogge D, Sutherland H J, Thomas T E, Lansdorp P M, et al. Retroviral gene transfer to primitive normal and leukemic hematopoietic cells using clinically applicable procedures. J Clin Invest [Internet]. American Society for Clinical Investigation; 1992 [cited 2019 Aug. 28]; 89:1817-24. Available from: http://www.ncbi.nlm.nih.gov/pubmed/1601991

What is claimed is:

1. An alloreactive T cell-distancing device comprising:
(a) an extracellular membrane-distal domain comprising a binding domain capable of binding a member of a central supramolecular activation cluster (SMAC) of the immunological synapse or a member closely associated therewith;
(b) an extracellular elongation domain comprising at least one rigid protein module;
(c) a transmembrane domain; and optionally
(d) an extracellular membrane-proximal domain, optionally less than 5 nm in length and/or lacking a glycosylphosphatidylinositol (GPI) anchor; and/or
(e) an intracellular domain optionally capable of associating, or co-clustering with, MHC molecules,
wherein (a)-(c) are connected from N-terminus to C-terminus in the following order via one or more hinges: transmembrane domain, extracellular elongation domain, and extracellular membrane-distal domain; intracellular domain, transmembrane domain, extracellular elongation domain, and extracellular membrane-distal domain; or intracellular domain, transmembrane domain, extracellular membrane-proximal domain, extracellular elongation domain, and extracellular membrane-distal domain.

2. A nucleic acid molecule comprising a nucleotide sequence encoding the alloreactive T cell-distancing device of claim 1, or an alloreactive T cell-distancing device comprising (a) an extracellular membrane-distal domain comprising a binding domain capable of binding a member of a central supramolecular activation cluster (SMAC) of the immunological synapse or a member closely associated therewith; and (b) an elongation domain comprising at least one rigid protein module, wherein said membrane-distal domain is linked via a membrane-proximal domain and a transmembrane domain to an intracellular domain optionally capable of associating, or co-clustering, with, MHC molecules.

3. The alloreactive T cell-distancing device of claim 1 or the nucleic acid molecule of claim 2, wherein the member of the central SMAC is selected from CD2, CD8, CD4, a signaling lymphocytic activation molecule (SLAM) and a CD28 family member.

4. The alloreactive T cell-distancing device or the nucleic acid molecule of claim 3, wherein the CD28 family member is selected from CD28, ICOS, BTLA, CTLA-4 and PD-1.

5. The alloreactive T cell-distancing device or the nucleic acid molecule of claim 4, wherein the binding domain is a CD2-binding domain selected from an LFA-3 (CD58) CD2-binding domain and a synthetic anti-CD2 antibody.

6. The alloreactive T cell-distancing device of claim 1 or the nucleic acid molecule of claim 2, wherein the at least one rigid protein module comprises an α-helix-forming peptide sequence, such as (EAAAK)n (SEQ ID NO: 171); or a proline-rich peptide sequence, such as (XP)n, with X designating any amino acid, e.g., Ala, Lys, or Glu.

7. The alloreactive T cell-distancing device of claim 1 or the nucleic acid molecule of claim 2, wherein the at least one rigid protein module is a fibronectin type III repeat or an Ig domain harboring the typical motifs of the Ig fold (Ig-like domain).

8. The alloreactive T cell-distancing device or the nucleic acid molecule of claim 7, wherein the elongation domain comprises at least two Ig-like domains and/or at least three fibronectin type III repeats.

9. The alloreactive T cell-distancing device or the nucleic acid molecule of claim 8, wherein the rigid elongation domain comprises the complete extracellular domain of LFA-3 (containing two Ig-like domains), CD22 (containing seven Ig-like domains), CD45 (comprising three fibronectin type III repeats), or CD148 (comprising five fibronectin type III repeats) or any combination of Ig-like domains and/or fibronectin type III domains.

10. The alloreactive T cell-distancing device or the nucleic acid molecule of claim 9, wherein the complete extracellular domain of CD45 is the complete extracellular domain of the CD45 isoform CD45RO, CD45RAB or CD45RABC.

11. The alloreactive T cell-distancing device of claim 1 or the nucleic acid molecule of claim 2, wherein the membrane-proximal domain comprises an Ig-like domain (such as an LFA-3 Ig-like domain) or a fibronectin type III repeat.

12. The alloreactive T cell-distancing device of claim 1 or the nucleic acid molecule of claim 2, wherein the transmembrane domain and/or intracellular domain is the transmembrane domain and/or intracellular domain of LFA-3.

13. The alloreactive T cell-distancing device of claim 1 or the nucleic acid molecule of claim 2, wherein the member of the central SMAC is selected from CD2, CD8, CD4, a signaling lymphocytic activation molecule (SLAM), and a CD28 family member; the at least one rigid protein module comprises an α-helix-forming peptide sequence (such as (EAAAK)n (SEQ ID NO: 171)), a proline-rich peptide sequence (such as (XP)n, with X designating any amino acid), a fibronectin type III repeat or an Ig domain harboring the typical motifs of the Ig fold (Ig-like domain); the membrane-proximal domain comprises an Ig-like domain (such as an LFA-3 Ig-like domain) or a fibronectin type III repeat; and the transmembrane domain and/or intracellular domain is the transmembrane domain and/or intracellular domain of LFA-3.

14. The alloreactive T cell-distancing device or the nucleic acid molecule of claim 13, wherein the binding domain is a CD2-binding domain selected from an LFA-3 (CD58) CD2-binding domain or a synthetic anti-CD2 antibody; the CD28 family member is selected from CD28, ICOS, BTLA, CTLA-4 and PD-1; and the elongation domain comprises at least two Ig-like domains and/or at least three fibronectin type III repeats.

15. The alloreactive T cell-distancing device or the nucleic acid molecule of claim 14, wherein the rigid elongation domain comprises the complete extracellular domain of LFA-3 (containing two Ig-like domains), CD22 (containing seven Ig-like domains), CD45 (comprising three fibronectin type III repeats), or CD148 (comprising five fibronectin type III repeats) or any combination of Ig-like domains and/or fibronectin type III domains.

16. The alloreactive T cell-distancing device or the nucleic acid molecule of claim 15, wherein the complete extracellular domain of CD45 is the complete extracellular domain of the CD45 isoform CD45RO, CD45RAB or CD45RABC.

17. The alloreactive T cell-distancing device or the nucleic acid molecule of any one of claims 1 to 16, wherein the alloreactive T cell-distancing device comprises an LFA-3 CD2-binding domain; a rigid elongation domain comprising at least two CD22 Ig-like domains and at least one LFA-3 Ig-like domain; or a complete extracellular CD45 domain and at least one LFA-3 Ig-like domain; an LFE-3 Ig-like membrane-proximal domain, and an LFE-3 transmembrane and intracellular domain.

18. The alloreactive T cell-distancing device or the nucleic acid molecule of claim 17, wherein said rigid elongation domain comprises a complete extracellular CD45 domain selected from that of CD45RO, CD45RAB and CD45RABC and one LFA-3 Ig-like domain, and the complete extracellular CD45 domain is located between the LFE-3 Ig-like membrane-proximal domain and the LFA-3 Ig-like rigid elongation domain.

19. A vector comprising the nucleic acid molecule of any one of claims 2 to 18.

20. The vector of claim 19, which is a DNA vector, such as a plasmid or viral vector; or a non-viral vector, such as a polymer nanoparticle, lipid, calcium phosphate, DNA-coated microparticle or transposon.

21. A method for producing a donor-derived allogeneic cell, cell-line or stem cell-line expressing an alloreactive T cell-distancing device, said method comprising contacting a donor-derived allogeneic cell, cell-line or stem cell-line with the nucleic acid molecule of any one of claims 2-18 or the vector of claims 19 or 20.

22. The method of claim 21, wherein said vector is a DNA vector, such as a plasmid or viral vector; or a non-viral vector, such as a polymer nanoparticle, lipid, calcium phosphate, DNA-coated microparticle or transposon.

23. A donor-derived allogeneic cell, cell-line or stem cell-line or a differentiated cell, organ or tissue derived from stem cells, expressing the alloreactive T cell-distancing device of claim 1 or any one of claims 3-18, or comprising the nucleic acid molecule of any one of claims 2-18 or the vector of claims 19, 20 or 22,
wherein said donor-derived allogeneic cell, cell-line or stem cell-line is protected from allorejection in adoptive cell therapy or stem cell transplantation, and a differentiated cell, organ or tissue derived from said stem cell-line is protected from allorejection in cell, organ or tissue transplantation.

24. The donor-derived allogeneic cell of claim 23, which is an immune cell, such as a cytotoxic T cell, regulatory T cell (Treg), B cell or NK cell; or a hematopoietic stem cell.

25. The donor-derived allogeneic cell of claim 24, wherein said immune cell is further expressing a chimeric antigen receptor (CAR).

26. The donor-derived allogeneic cell-line of claim 25, which is an induced pluripotent stem cell-line.

27. The donor-derived allogeneic cell of claim 26, wherein said differentiated cell derived from an induced pluripotent stem cell-line is a retinal pigment epithelial cell, cardiac cell or neural cell.

28. A method of transplantation therapy in a subject in need thereof, said method comprising administering to said subject in need a donor-derived allogeneic cell, cell-line or stem cell-line or a differentiated cell, organ or tissue derived from stem cells of any one of claims 23 to 27.

29. A method comprising administering to a subject the donor-derived allogeneic cell of any one of claims 23-27.


SEQUENCE LISTING (171 sequences)

SEQ ID NO: 1 - DNA, 642 bp, Artificial Sequence
  Synthetic
gttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacaga

SEQ ID NO: 2 - DNA, 561 bp, Artificial Sequence
  Synthetic
ttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacaga

SEQ ID NO: 3 - DNA, 276 bp, Artificial Sequence
  Synthetic
ttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtg

SEQ ID NO: 4 - DNA, 540 bp, Artificial Sequence
  Synthetic
ttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagc

SEQ ID NO: 5 - DNA, 638 bp, Artificial Sequence
  Synthetic
tgcttcataaaacagggatggtgtctggtcctggaactgctactgctgcccttgggaactggatttcaaggtcattcaataccagatataaatgccaccaccggcagcaatgtaaccctgaaaatccataaggacccacttggaccatataaacgtatcacctggcttcatactaaaaatcagaagattttagagtacaactataatagtacaaagacaatcttcgagtctgaatttaaaggcagggtttatcttgaagaaaacaatggtgcacttcatatctctaatgtccggaaagaggacaaaggtacctactacatgagagtgctgcgtgaaactgagaacgagttgaagataaccctggaagtatttgatcctgtgcccaagccttccatagaaatcaataagactgaagcctccactgattcctgtcacctgaggctatcgtgtgaggtaaaggaccagcatgttgactatacttggtatgagagcagcggacctttccccaaaaagagtccaggatatgtgctcgatctcatcgtcacaccacagaacaagtctacattttacacctgccaagtcagcaatcctgtaagcagcaagaacgacacagtgtacttcactctaccttgtgatct

SEQ ID NO: 6 - DNA, 575 bp, Artificial Sequence
  Synthetic
tttcaaggtcattcaataccagatataaatgccaccaccggcagcaatgtaaccctgaaaatccataaggacccacttggaccatataaacgtatcacctggcttcatactaaaaatcagaagattttagagtacaactataatagtacaaagacaatcttcgagtctgaatttaaaggcagggtttatcttgaagaaaacaatggtgcacttcatatctctaatgtccggaaagaggacaaaggtacctactacatgagagtgctgcgtgaaactgagaacgagttgaagataaccctggaagtatttgatcctgtgcccaagccttccatagaaatcaataagactgaagcctccactgattcctgtcacctgaggctatcgtgtgaggtaaaggaccagcatgttgactatacttggtatgagagcagcggacctttccccaaaaagagtccaggatatgtgctcgatctcatcgtcacaccacagaacaagtctacattttacacctgccaagtcagcaatcctgtaagcagcaagaacgacacagtgtacttcactctaccttgtgatct

SEQ ID NO: 7 - DNA, 642 bp, Artificial Sequence; identical to SEQ ID NO: 1
  Synthetic

SEQ ID NO: 8 - DNA, 561 bp, Artificial Sequence; identical to SEQ ID NO: 2
  Synthetic

SEQ ID NO: 9 - DNA, 357 bp, Artificial Sequence
  Synthetic
gttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtg

SEQ ID NO: 10 - DNA, 276 bp, Artificial Sequence; identical to SEQ ID NO: 3
  Synthetic

SEQ ID NO: 11 - DNA, 621 bp, Artificial Sequence
  Synthetic
gttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagc

SEQ ID NO: 12 - DNA, 540 bp, Artificial Sequence; identical to SEQ ID NO: 4
  Synthetic

SEQ ID NO: 13 - DNA, 795 bp, Artificial Sequence
  Synthetic
gccttaccagtgaccgccttgctcctgccgctggccttgctgctccacgccgccaggccggacgtggtgatgacccagagcccccccagcctgctggtgaccctgggccagcccgccagcatcagctgcagaagcagccagagcctgctgcacagcagcggcaacacctacctgaactggctgctgcagagacccggccagagcccccagcccctgatctacctggtgagcaagctggagagcggcgtgcccgacagattcagcggcagcggcagcggcaccgacttcaccctgaagatcagcggcgtggaggccgaggacgtgggcgtgtactactgcatgcagttcacccactacccctacaccttcggccagggcaccaagctggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgagtactacatgtactgggtgagacaggcccccggccagggcctggagctgatgggcagaatcgaccccgaggacggcagcatcgactacgtggagaagttcaagaagaaggtgaccctgaccgccgacaccagcagcagcaccgcctacatggagctgagcagcctgaccagcgacgacaccgccgtgtactactgcgccagaggcaagttcaactacagattcgcctactggggccagggcaccctggtgaccgtgagcagc

SEQ ID NO: 14 - DNA, 735 bp, Artificial Sequence
  Synthetic
gacgtggtgatgacccagagcccccccagcctgctggtgaccctgggccagcccgccagcatcagctgcagaagcagccagagcctgctgcacagcagcggcaacacctacctgaactggctgctgcagagacccggccagagcccccagcccctgatctacctggtgagcaagctggagagcggcgtgcccgacagattcagcggcagcggcagcggcaccgacttcaccctgaagatcagcggcgtggaggccgaggacgtgggcgtgtactactgcatgcagttcacccactacccctacaccttcggccagggcaccaagctggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgagtactacatgtactgggtgagacaggcccccggccagggcctggagctgatgggcagaatcgaccccgaggacggcagcatcgactacgtggagaagttcaagaagaaggtgaccctgaccgccgacaccagcagcagcaccgcctacatggagctgagcagcctgaccagcgacgacaccgccgtgtactactgcgccagaggcaagttcaactacagattcgcctactggggccagggcaccctggtgaccgtgagcagc

SEQ ID NO: 15 - DNA, 810 bp, Artificial Sequence
  Synthetic
ccgtcgacccccaagaaggtgaccacagtgattcaaaaccccatgccgattcgagaaggagacacagtgaccctttcctgtaactacaattccagtaaccccagtgttacccggtatgaatggaaacctcatggggcctgggaggagccatcgcttggggtgctgaagatccaaaacgtaggctgggacaacacaaccatcgcctgcgcagcttgtaatagttggtgctcttgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcgccggagaccatctcg

SEQ ID NO: 16 - DNA, 1071 bp, Artificial Sequence
  Synthetic
tcgaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcactcg

SEQ ID NO: 17 - DNA, 1173 bp, Artificial Sequence
  Synthetic
caaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaag

SEQ ID NO: 18 - DNA, 1632 bp, Artificial Sequence
  Synthetic
tcgacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcactcg

SEQ ID NO: 19 - DNA, 1317 bp, Artificial Sequence
  Synthetic
tccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaaatactgctgtcaggtctccaatgacgtgggaccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtatcacaatgggaaagaaatgcagggaaggacagaggagaaagtccacatcccaaagatcctcccctggcacgctgggacttattcctgtgtggcagaaaacattcttggtactggacagaggggaccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaacctcatggggcctgggaggagccatcgcttggggtgctgaagatccaaaacgtaggctgggacaacacaaccatcgcctgcgcagcttgtaatagttggtgctcttgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactac

SEQ ID NO: 20 - DNA, 1349 bp, Artificial Sequence
  Synthetic
accccgaagctggagatcaaggtcaatcccacagaggtggaaaagaacaattctgtgaccatgacatgccgggttaacagcagcaacccgaaactcaggaccgtggcggtgtcttggttcaaggatgggcgccccctagaggatcaggaactggaacaggaacaacagatgtccaagctaattctgcattcagtgaccaaggacatgagagggaaataccggtgccaggcttccaacgacataggcccaggagagtcggaagaagtggaactcacggtgcactatgctccagaaccctccagggttcacatctacccttcccccgctgaagagggacagtcagtagagctgatttgtgagtcactggccagtccaagtgcaacaaactacacctggtatcacaacaggaaacctatacctggagacacccaagagaagctccgcatccctaaagtctccccgtggcatgctgggaattactcttgcttggcagagaaccgtctgggtcatggaaagatagaccaggaagctaagctggatgtccattatgctcccaaggcggtgaccacagtgattcagagcttcacaccaatcctggaaggagacagtgtgaccctggtctgtaggtacaactccagcaatccagacgtcacctcctacagatggaaccctcaaggttctgggagtgtgctcaaacccggagtgctgaggattcagaaagtgacatgggattccatgcctgtcagctgtgctgcctgcaaccacaagtgttcgtgggccctccctgtcatcctgaatgtccactacgcccccagagacgtgaaggtactgaaggtaagccccgcatcagagatccgcgctgggcagcgtgtcctcctccaatgcgacttcgcagagagcaacccggcagaggtccgcttcttctggaagaagaatgggagtctcgtgcaggaagggaggtacctgagcttcggctccgtctccccagaagattctggaaattataactgcatggtcaacaactccatcggagagaccttgtcacaggcctggaacctccaagtgctgtatgctcctcggaggctgcgtgtgtccatcagccctggggaccatgtgatggaggggaagaaggccaccttgtcctgtgagagtgatgccaatccgcccatctcacagtacacctggtttgactccagtggccaagacctccactcctcaggccagaaactgagactggaacccctggaggtccaacacacgggttcctaccgctgcaaagggaccaatgggataggcacaggagagtcaccacccagcaccctcactgtctactacagtccagagaccat

SEQ ID NO: 21 - DNA, 1347 bp, Artificial Sequence
  Synthetic
aagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcga

SEQ ID NO: 22 - DNA, 1626 bp, Artificial Sequence
  Synthetic
tcaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcac

SEQ ID NO: 23 - DNA, 1613 bp, Artificial Sequence
  Synthetic
caaacacctacacccagtgatgaactgagcacaacagagaatgcccttcttctgcctcaaagtgaccccttacctgctcgcaccactgaatccacacccccaagcatctctgaaagaggaaatggctcttcagagaccacatatcatccaggtgtgttatccacgctgctgcctcacctgtccccacagcctgactcgcagacgccctctgccggaggagctgacactcagacattcagcagccaagctgacaatcccacactcacgcctgctcccggcggcgggactgacccaccaggtgtgccaggggagaggactgtaccggggaccattcctgcagacacagcctttcctgttgatacccccagccttgcacgcaacagctctgctgcctcacctacacacacctccaatgtcagcaccacagatatctcttcaggtgccagcctcacaactcttacaccatccactctgggccttgcaagcactgaccctccaagcacaaccatagctaccacaacgaagcaaacatgtgctgccatgtttgggaacattactgtgaattacacctatgaatctagtaatcagacttttaaggcagacctcaaagatgtccaaaatgctaagtgtggaaatgaggattgtgaaaacgtgttaaataacctagaagaatgctcacagataaaaaacatcagtgtgtctaatgactcatgtgctccagctacaactatagatttatatgtaccaccagggactgacaagttttcgctacatgactgcacaccaaaagaaaaggctaatacttcaatttgtttggagtggaaaacaaaaaaccttgatttcagaaaatgcaacagtgacaatatttcatatgtactccactgtgagccagaaaataatacaaaatgcattagaagaaatacattcatacctgaaagatgtcagttggacaaccttcgtgcccaaacaaattacacatgtgtagcagaaatcttatatcgcggtgtaaaactcgtcaaaaatgttataaatgtgcagacagatttggggattccagaaacgcctaagcctagttgtggcgatccagctgcaagaaaaacgttagtctcttggcctgagcctgtgtctaaacctgagtctgcatctaaacctcatggatatgttttatgctataagaacaattcagaaaaatgtaaaagtttgcctaataatgtgaccagttttgaggtggagagcttgaaaccttataaatactatgaagtgtccctacttgcctatgtcaatgggaagattcaaagaaatgggactgctgagaagtgcaattttcacacaaaagcagatcgtccagacaaggtcaatggaatgaaaacctcccggccgacagacaatagtataaatgttacatgtggtcctccttatgaaactaatggccctaaaaccttttacattttggtagtcagaagtggaggttcttttgttacaaaatacaacaagacaaactgtcagttttatgtagataatctctactattcaactgactatgagtttctggtctcttttcacaatggagtgtacgagggagattcagttataagaaatgagtcaacaaattt

SEQ ID NO: 24 - DNA, 1656 bp, Artificial Sequence
  Synthetic
caaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaag

SEQ ID NO: 25 - DNA, 1749 bp, Artificial Sequence
  Synthetic
tcaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaat

SEQ ID NO: 26 - DNA, 1725 bp, Artificial Sequence
  Synthetic
caaacacctacacccagtgatgaactgagcacaacagagaatgcccttcttctgcctcaaagtgaccccttacctgctcgcaccactgaatccacacccccaagcatctctgaaagaggaaatggctcttcagagaccacatatcatccaggtgtgttatccacgctgctgcctcacctgtccccacagcctgactcgcagacgccctctgccggaggagctgacactcagacattcagcagccaagctgacaatcccacactcacgcctgctcccggcggcgggactgacccaccaggtgtgccaggggagaggactgtaccggggaccattcctgcagacacagcctttcctgttgatacccccagccttgcacgcaacagctctgctgcctcacctacacacacctccaatgtcagcaccacagatatctcttcaggtgccagcctcacaactcttacaccatccactctgggccttgcaagcactgaccctccaagcacaaccatagctaccacaacgaagcaaacatgtgctgccatgtttgggaacattactgtgaattacacctatgaatctagtaatcagacttttaaggcagacctcaaagatgtccaaaatgctaagtgtggaaatgaggattgtgaaaacgtgttaaataacctagaagaatgctcacagataaaaaacatcagtgtgtctaatgactcatgtgctccagctacaactatagatttatatgtaccaccagggactgacaagttttcgctacatgactgcacaccaaaagaaaaggctaatacttcaatttgtttggagtggaaaacaaaaaaccttgatttcagaaaatgcaacagtgacaatatttcatatgtactccactgtgagccagaaaataatacaaaatgcattagaagaaatacattcatacctgaaagatgtcagttggacaaccttcgtgcccaaacaaattacacatgtgtagcagaaatcttatatcgcggtgtaaaactcgtcaaaaatgttataaatgtgcagacagatttggggattccagaaacgcctaagcctagttgtggcgatccagctgcaagaaaaacgttagtctcttggcctgagcctgtgtctaaacctgagtctgcatctaaacctcatggatatgttttatgctataagaacaattcagaaaaatgtaaaagtttgcctaataatgtgaccagttttgaggtggagagcttgaaaccttataaatactatgaagtgtccctacttgcctatgtcaatgggaagattcaaagaaatgggactgctgagaagtgcaattttcacacaaaagcagatcgtccagacaaggtcaatggaatgaaaacctcccggccgacagacaatagtataaatgttacatgtggtcctccttatgaaactaatggccctaaaaccttttacattttggtagtcagaagtggaggttcttttgttacaaaatacaacaagacaaactgtcagttttatgtagataatctctactattcaactgactatgagtttctggtctcttttcacaatggagtgtacgagggagattcagttataagaaatgagtcaacaaattttaatgctaaagcactgattatattcctggtgtttctgattattgtgacatcaatagccttgcttgttgttttgtataaaatctatgatctgcgcaagaaaagatccagcaat

SEQ ID NO: 27 - DNA, 1446 bp, Artificial Sequence
  Synthetic
aagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcagggg

SEQ ID NO: 28 - DNA, 1275 bp, Artificial Sequence
  Synthetic
caaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaat

SEQ ID NO: 29 - DNA, 1251 bp, Artificial Sequence
  Synthetic
gatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaat

SEQ ID NO: 30 - DNA, 1758 bp, Artificial Sequence
  Synthetic
caaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaat

SEQ ID NO: 31 - DNA, 1734 bp, Artificial Sequence
  Synthetic
ggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaat

SEQ ID NO: 32 - DNA, 18 bp, Artificial Sequence
  Synthetic
cagcccaccatccccatc

SEQ ID NO: 33 - DNA, 36 bp, Artificial Sequence
  Synthetic
tttattttacatcattcaacatcttataattctaag

SEQ ID NO: 34 - DNA, 18 bp, Artificial Sequence
  Synthetic
tccactgtctccaacatg

SEQ ID NO: 35 - DNA, 12 bp, Artificial Sequence
  Synthetic
tcgagacacaga

SEQ ID NO: 36 - DNA, 24 bp, Artificial Sequence
  Synthetic
tcgagccagcccaccatccccatc

SEQ ID NO: 37 - DNA, 114 bp, Artificial Sequence
  Synthetic
agacacagatatgcacttatacccataccattagcagtaattacaacatgtattgtgctgtatatgaatggtattctgaaatgtgacagaaaaccagacagaaccaactccaat

SEQ ID NO: 38 - DNA, 189 bp, Artificial Sequence
  Synthetic
cagcccaccatccccatcgtgggcatcattgctggcctggttctctttggagctgtgatcactggagctgtggtcgctgctgtgatgtggaggaggaaaagctcagatagaaaaggagggagctactctcaggctgcaagcagtgacagtgcccagggctctgatgtgtctctcacagcttgtaaagtg

SEQ ID NO: 39 - DNA, 171 bp, Artificial Sequence
  Synthetic
gtgggcatcattgctggcctggttctctttggagctgtgatcactggagctgtggtcgctgctgtgatgtggaggaggaaaagctcagatagaaaaggagggagctactctcaggctgcaagcagtgacagtgcccagggctctgatgtgtctctcacagcttgtaaagtg

SEQ ID NO: 40 - DNA, 210 bp, Artificial Sequence
  Synthetic
tccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcg

SEQ ID NO: 41 - DNA, 192 bp, Artificial Sequence
  Synthetic
gcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcg

SEQ ID NO: 42 - DNA, 138 bp, Artificial Sequence
  Synthetic
tttattttacatcattcaacatcttataattctaaggcactgatagcatttctggcatttctgattattgtgacatcaatagccctgcttgttgttctctacaaaatctatgatctacataagaaaagatcctgcaat

SEQ ID NO: 43 - DNA, 72 bp, Artificial Sequence
  Synthetic
gtgggcatcattgctggcctggttctctttggagctgtgatcactggagctgtggtcgctgctgtgatgtgg

SEQ ID NO: 44 - DNA, 69 bp, Artificial Sequence
  Synthetic
gcgaccgttgctgttctggttgtccttggagctgcaatagtcactggagctgtggtggcttttgtgatg

SEQ ID NO: 45 - DNA, 66 bp, Artificial Sequence
  Synthetic
gcactgatagcatttctggcatttctgattattgtgacatcaatagccctgcttgttgttctctac

SEQ ID NO: 46 - DNA, 63 bp, Artificial Sequence
  Synthetic
gcactgatagcatttctggcatttctgattattgtgacatcaatagccctgcttgttgttctc

SEQ ID NO: 47 - DNA, 57 bp, Artificial Sequence
  Synthetic
gtggctgtgggactcgggtcctgcctcgccatcctcatcctggcaatctgtgggctc

SEQ ID NO: 48 - DNA, 66 bp, Artificial Sequence; identical to SEQ ID NO: 45
  Synthetic

SEQ ID NO: 49 - DNA, 135 bp, Artificial Sequence
  Synthetic
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 50 - DNA, 12 bp, Artificial Sequence
  Synthetic
ggcggaggcagc

SEQ ID NO: 51 - DNA, 81 bp, Artificial Sequence
  Synthetic
gttgctgggagcgacgcggggcgggccctgggggtcctcagcgtggtctgcctgctgcactgctttggtttcatcagctgt

SEQ ID NO: 52 - DNA, 63 bp, Artificial Sequence
  Synthetic
tgcttcataaaacagggatggtgtctggtcctggaactgctactgctgcccttgggaactgga

SEQ ID NO: 53 - DNA, 60 bp, Artificial Sequence
  Synthetic
gccttaccagtgaccgccttgctcctgccgctggccttgctgctccacgccgccaggccg

SEQ ID NO: 54 - DNA, 42 bp, Artificial Sequence
  Synthetic
gggtcctacccctacgacgttcccgactacgctgggagctcg

SEQ ID NO: 55 - DNA, 1638 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagagggtcctacccctacgacgttcccgactacgctgggagctcgccgtcgacccccaagaaggtgaccacagtgattcaaaaccccatgccgattcgagaaggagacacagtgaccctttcctgtaactacaattccagtaaccccagtgttacccggtatgaatggaaacctcatggggcctgggaggagccatcgcttggggtgctgaagatccaaaacgtaggctgggacaacacaaccatcgcctgcgcagcttgtaatagttggtgctcttgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcgccggagaccatctcgagacacagatatgcacttatacccataccattagcagtaattacaacatgtattgtgctgtatatgaatggtattctgaaatgtgacagaaaaccagacagaaccaactccaattgagcggccgc

SEQ ID NO: 56 - protein, 215 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 57 - protein, 187 aa, Artificial Sequence
  Synthetic
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 58 - protein, 92 aa, Artificial Sequence
  Synthetic
FSQQIYGVVYGNVTFHVPSNVPLKEVLWKKQKDKVAELENSEFRAFSSFKNRVYLDTVSGSLTIYNLTSSDEDEYEMESPNITDTMKFFLYV

SEQ ID NO: 59 - protein, 180 aa, Artificial Sequence
  Synthetic
FSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPS

SEQ ID NO: 60 - protein, 217 aa, Artificial Sequence
  Synthetic
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARS

SEQ ID NO: 61 - protein, 192 aa, Artificial Sequence
  Synthetic
FQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDL

SEQ ID NO: 62 - protein, 215 aa, Artificial Sequence; identical to SEQ ID NO: 56
  Synthetic

SEQ ID NO: 63 - protein, 187 aa, Artificial Sequence; identical to SEQ ID NO: 57
  Synthetic

SEQ ID NO: 64 - protein, 120 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYV

SEQ ID NO: 65 - protein, 92 aa, Artificial Sequence; identical to SEQ ID NO: 58
  Synthetic

SEQ ID NO: 66 - protein, 208 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPS

SEQ ID NO: 67 - protein, 180 aa, Artificial Sequence; identical to SEQ ID NO: 59
  Synthetic

SEQ ID NO: 68 - protein, 266 aa, Artificial Sequence
  Synthetic
MALPVTALLLPLALLLHAARPDVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKGGGGSGGGGSGGGGSQVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSS

SEQ ID NO: 69 - protein, 245 aa, Artificial Sequence
  Synthetic
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKGGGGSGGGGSGGGGSQVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSS

SEQ ID NO: 70 - protein, 270 aa, Artificial Sequence
  Synthetic
PSTPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIS

SEQ ID NO: 71 - protein, 449 aa, Artificial Sequence
  Synthetic
KHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 72 - protein, 357 aa, Artificial Sequence
  Synthetic
STTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHS

SEQ ID NO: 73 - protein, 544 aa, Artificial Sequence
  Synthetic
STPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHS

SEQ ID NO: 74 - protein, 438 aa, Artificial Sequence
  Synthetic
PKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYY

SEQ ID NO: 75 - protein, 450 aa, Artificial Sequence
  Synthetic
TPKLEIKVNPTEVEKNNSVTMTCRVNSSNPKLRTVAVSWFKDGRPLEDQELEQEQQMSKLILHSVTKDMRGKYRCQASNDIGPGESEEVELTVHYAPEPSRVHIYPSPAEEGQSVELICESLASPSATNYTWYHNRKPIPGDTQEKLRIPKVSPWHAGNYSCLAENRLGHGKIDQEAKLDVHYAPKAVTTVIQSFTPILEGDSVTLVCRYNSSNPDVTSYRWNPQGSGSVLKPGVLRIQKVTWDSMPVSCAACNHKCSWALPVILNVHYAPRDVKVLKVSPASEIRAGQRVLLQCDFAESNPAEVRFFWKKNGSLVQEGRYLSFGSVSPEDSGNYNCMVNNSIGETLSQAWNLQVLYAPRRLRVSISPGDHVMEGKKATLSCESDANPPISQYTWFDSSGQDLHSSGQKLRLEPLEVQHTGSYRCKGTNGIGTGESPPSTLTVYYSPETI

SEQ ID NO: 76 - protein, 541 aa, Artificial Sequence
  Synthetic
PSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHH

SEQ ID NO: 77 - protein, 538 aa, Artificial Sequence
  Synthetic
QTPTPSDELSTTENALLLPQSDPLPARTTESTPPSISERGNGSSETTYHPGVLSTLLPHLSPQPDSQTPSAGGADTQTFSSQADNPTLTPAPGGGTDPPGVPGERTVPGTIPADTAFPVDTPSLARNSSAASPTHTSNVSTTDISSGASLTTLTPSTLGLASTDPPSTTIATTTKQTCAAMFGNITVNYTYESSNQTFKADLKDVQNAKCGNEDCENVLNNLEECSQIKNISVSNDSCAPATTIDLYVPPGTDKFSLHDCTPKEKANTSICLEWKTKNLDFRKCNSDNISYVLHCEPENNTKCIRRNTFIPERCQLDNLRAQTNYTCVAEILYRGVKLVKNVINVQTDLGIPETPKPSCGDPAARKTLVSWPEPVSKPESASKPHGYVLCYKNNSEKCKSLPNNVTSFEVESLKPYKYYEVSLLAYVNGKIQRNGTAEKCNFHTKADRPDKVNGMKTSRPTDNSINVTCGPPYETNGPKTFYILVVRSGGSFVTKYNKTNCQFYVDNLYYSTDYEFLVSFHNGVYEGDSVIRNESTNF

SEQ ID NO: 78 - protein, 551 aa, Artificial Sequence
  Synthetic
QSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNS

SEQ ID NO: 79 - protein, 391 aa, Artificial Sequence
  Synthetic
QSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSK

SEQ ID NO: 80 - protein, 582 aa, Artificial Sequence
  Synthetic
PSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 81 - protein, 575 aa, Artificial Sequence
  Synthetic
QTPTPSDELSTTENALLLPQSDPLPARTTESTPPSISERGNGSSETTYHPGVLSTLLPHLSPQPDSQTPSAGGADTQTFSSQADNPTLTPAPGGGTDPPGVPGERTVPGTIPADTAFPVDTPSLARNSSAASPTHTSNVSTTDISSGASLTTLTPSTLGLASTDPPSTTIATTTKQTCAAMFGNITVNYTYESSNQTFKADLKDVQNAKCGNEDCENVLNNLEECSQIKNISVSNDSCAPATTIDLYVPPGTDKFSLHDCTPKEKANTSICLEWKTKNLDFRKCNSDNISYVLHCEPENNTKCIRRNTFIPERCQLDNLRAQTNYTCVAEILYRGVKLVKNVINVQTDLGIPETPKPSCGDPAARKTLVSWPEPVSKPESASKPHGYVLCYKNNSEKCKSLPNNVTSFEVESLKPYKYYEVSLLAYVNGKIQRNGTAEKCNFHTKADRPDKVNGMKTSRPTDNSINVTCGPPYETNGPKTFYILVVRSGGSFVTKYNKTNCQFYVDNLYYSTDYEFLVSFHNGVYEGDSVIRNESTNFNAKALIIFLVFLIIVTSIALLVVLYKIYDLRKKRSSN

SEQ ID NO: 82 - protein, 482 aa, Artificial Sequence
  Synthetic
KHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQG

SEQ ID NO: 83 - protein, 425 aa, Artificial Sequence
  Synthetic
QSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 84 - protein, 417 aa, Artificial Sequence
  Synthetic
DAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 85 - protein, 586 aa, Artificial Sequence
  Synthetic
QSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 86 - protein, 578 aa, Artificial Sequence
  Synthetic
GLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 87 - protein, 6 aa, Artificial Sequence
  Synthetic
QPTIPI

SEQ ID NO: 88 - protein, 9 aa, Artificial Sequence
  Synthetic
HHSTSYNSK

SEQ ID NO: 89 - protein, 6 aa, Artificial Sequence
  Synthetic
STVSNM

SEQ ID NO: 90 - protein, 4 aa, Artificial Sequence
  Synthetic
SRHR

SEQ ID NO: 91 - protein, 8 aa, Artificial Sequence
  Synthetic
SSQPTIPI

SEQ ID NO: 92 - protein, 38 aa, Artificial Sequence
  Synthetic
RHRYALIPIPLAVITTCIVLYMNGILKCDRKPDRTNSN

SEQ ID NO: 93 - protein, 63 aa, Artificial Sequence
  Synthetic
QPTIPIVGIIAGLVLFGAVITGAVVAAVMWRRKSSDRKGGSYSQAASSDSAQGSDVSLTACKV

SEQ ID NO: 94 - protein, 57 aa, Artificial Sequence
  Synthetic
VGIIAGLVLFGAVITGAVVAAVMWRRKSSDRKGGSYSQAASSDSAQGSDVSLTACKV

SEQ ID NO: 95 - protein, 70 aa, Artificial Sequence
  Synthetic
STVSNMATVAVLVVLGAAIVTGAVVAFVMKMRRRNTGGKGGDYALAPGSQTSDLSLPDCKVMVHDPHSLA

SEQ ID NO: 96 - protein, 64 aa, Artificial Sequence
  Synthetic
ATVAVLVVLGAAIVTGAVVAFVMKMRRRNTGGKGGDYALAPGSQTSDLSLPDCKVMVHDPHSLA

SEQ ID NO: 97 - protein, 43 aa, Artificial Sequence
  Synthetic
HHSTSYNSKALIAFLAFLIIVTSIALLVVLYKIYDLHKKRSCN

SEQ ID NO: 98 - protein, 24 aa, Artificial Sequence
  Synthetic
VGIIAGLVLFGAVITGAVVAAVMW

SEQ ID NO: 99 - protein, 23 aa, Artificial Sequence
  Synthetic
ATVAVLVVLGAAIVTGAVVAFVM

SEQ ID NO: 100 - protein, 22 aa, Artificial Sequence
  Synthetic
ALIAFLAFLIIVTSIALLVVLY

SEQ ID NO: 101 - protein, 21 aa, Artificial Sequence
  Synthetic
ALIAFLAFLIIVTSIALLVVL

SEQ ID NO: 102 - protein, 19 aa, Artificial Sequence
  Synthetic
VAVGLGSCLAILILAICGL

SEQ ID NO: 103 - protein, 22 aa, Artificial Sequence
  Synthetic
ALIAFLAFLIIVTSIALLVVLY

SEQ ID NO: 104 - protein, 45 aa, Artificial Sequence
  Synthetic
TTTPAPRPPTPAPTIASQPLSLRPEACRPAAGGAVHTRGLDFACD

SEQ ID NO: 105 - protein, 4 aa, Artificial Sequence
  Synthetic
GGGS

SEQ ID NO: 106 - protein, 28 aa, Artificial Sequence
  Synthetic
MVAGSDAGRALGVLSVVCLLHCFGFISC

SEQ ID NO: 107 - protein, 22 aa, Artificial Sequence
  Synthetic
MCFIKQGWCLVLELLLLPLGTG

SEQ ID NO: 108 - protein, 21 aa, Artificial Sequence
  Synthetic
MALPVTALLLPLALLLHAARP

SEQ ID NO: 109 - protein, 14 aa, Artificial Sequence
  Synthetic
GSYPYDVPDYAGSS

SEQ ID NO: 110 - protein, 537 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRGSYPYDVPDYAGSSPSTPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETISRHRYALIPIPLAVITTCIVLYMNGILKCDRKPDRTNSN

SEQ ID NO: 111 - DNA, 1899 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagagggtcctacccctacgacgttcccgactacgctgggagctcgtcgaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcactcgagacacagatatgcacttatacccataccattagcagtaattacaacatgtattgtgctgtatatgaatggtattctgaaatgtgacagaaaaccagacagaaccaactccaattgagcggccgc

SEQ ID NO: 112 - protein, 624 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRGSYPYDVPDYAGSSSTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSRHRYALIPIPLAVITTCIVLYMNGILKCDRKPDRTNSN

SEQ ID NO: 113 - DNA, 2460 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagagggtcctacccctacgacgttcccgactacgctgggagctcgtcgacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcactcgagacacagatatgcacttatacccataccattagcagtaattacaacatgtattgtgctgtatatgaatggtattctgaaatgtgacagaaaaccagacagaaccaactccaattgagcggccgc

SEQ ID NO: 114 - protein, 811 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRGSYPYDVPDYAGSSSTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSRHRYALIPIPLAVITTCIVLYMNGILKCDRKPDRTNSN

SEQ ID NO: 115 - DNA, 1644 bp, Artificial Sequence
  Synthetic
tctagacgccgccaccatgtgcttcataaaacagggatggtgtctggtcctggaactgctactgctgcccttgggaactggatttcaaggtcattcaataccagatataaatgccaccaccggcagcaatgtaaccctgaaaatccataaggacccacttggaccatataaacgtatcacctggcttcatactaaaaatcagaagattttagagtacaactataatagtacaaagacaatcttcgagtctgaatttaaaggcagggtttatcttgaagaaaacaatggtgcacttcatatctctaatgtccggaaagaggacaaaggtacctactacatgagagtgctgcgtgaaactgagaacgagttgaagataaccctggaagtatttgatcctgtgcccaagccttccatagaaatcaataagactgaagcctcgactgattcctgtcacctgaggctatcgtgtgaggtaaaggaccagcatgttgactatacttggtatgagagcagcggacctttccccaaaaagagtccaggatatgtgctcgatctcatcgtcacaccacagaacaagtctacattttacacctgccaagtcagcaatcctgtaagcagcaagaacgacacagtgtacttcactctaccttgtgatctagccagatctgggtcctacccctacgacgttcccgactacgctgggagctcgccgtcgacccccaagaaggtgaccacagtgattcaaaaccccatgccgattcgagaaggagacacagtgaccctttcctgtaactacaattccagtaaccccagtgttacccggtatgaatggaaacctcatggggcctgggaggagccatcgcttggggtgctgaagatccaaaacgtaggctgggacaacacaaccatcgcctgcgcagcttgtaatagttggtgctcttgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcgccggagaccatctcgagacacagatatgcacttatacccataccattagcagtaattacaacatgtattgtgctgtatatgaatggtattctgaaatgtgacagaaaaccagacagaaccaactccaattgagcggccgc

SEQ ID NO: 116 - protein, 539 aa, Artificial Sequence
  Synthetic
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARSGSYPYDVPDYAGSSPSTPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETISRHRYALIPIPLAVITTCIVLYMNGILKCDRKPDRTNSN

SEQ ID NO: 117 - DNA, 1905 bp, Artificial Sequence
  Synthetic
tctagacgccgccaccatgtgcttcataaaacagggatggtgtctggtcctggaactgctactgctgcccttgggaactggatttcaaggtcattcaataccagatataaatgccaccaccggcagcaatgtaaccctgaaaatccataaggacccacttggaccatataaacgtatcacctggcttcatactaaaaatcagaagattttagagtacaactataatagtacaaagacaatcttcgagtctgaatttaaaggcagggtttatcttgaagaaaacaatggtgcacttcatatctctaatgtccggaaagaggacaaaggtacctactacatgagagtgctgcgtgaaactgagaacgagttgaagataaccctggaagtatttgatcctgtgcccaagccttccatagaaatcaataagactgaagcctcgactgattcctgtcacctgaggctatcgtgtgaggtaaaggaccagcatgttgactatacttggtatgagagcagcggacctttccccaaaaagagtccaggatatgtgctcgatctcatcgtcacaccacagaacaagtctacattttacacctgccaagtcagcaatcctgtaagcagcaagaacgacacagtgtacttcactctaccttgtgatctagccagatctgggtcctacccctacgacgttcccgactacgctgggagctcgtcgaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcactcgagacacagatatgcacttatacccataccattagcagtaattacaacatgtattgtgctgtatatgaatggtattctgaaatgtgacagaaaaccagacagaaccaactccaattgagcggccgc

SEQ ID NO: 118 - protein, 626 aa, Artificial Sequence
  Synthetic
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARSGSYPYDVPDYAGSSSTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSRHRYALIPIPLAVITTCIVLYMNGILKCDRKPDRTNSN

SEQ ID NO: 119 - DNA, 2466 bp, Artificial Sequence
  Synthetic
tctagacgccgccaccatgtgcttcataaaacagggatggtgtctggtcctggaactgctactgctgcccttgggaactggatttcaaggtcattcaataccagatataaatgccaccaccggcagcaatgtaaccctgaaaatccataaggacccacttggaccatataaacgtatcacctggcttcatactaaaaatcagaagattttagagtacaactataatagtacaaagacaatcttcgagtctgaatttaaaggcagggtttatcttgaagaaaacaatggtgcacttcatatctctaatgtccggaaagaggacaaaggtacctactacatgagagtgctgcgtgaaactgagaacgagttgaagataaccctggaagtatttgatcctgtgcccaagccttccatagaaatcaataagactgaagcctcgactgattcctgtcacctgaggctatcgtgtgaggtaaaggaccagcatgttgactatacttggtatgagagcagcggacctttccccaaaaagagtccaggatatgtgctcgatctcatcgtcacaccacagaacaagtctacattttacacctgccaagtcagcaatcctgtaagcagcaagaacgacacagtgtacttcactctaccttgtgatctagccagatctgggtcctacccctacgacgttcccgactacgctgggagctcgtcgacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcactcgagacacagatatgcacttatacccataccattagcagtaattacaacatgtattgtgctgtatatgaatggtattctgaaatgtgacagaaaaccagacagaaccaactccaattgagcggccgc

SEQ ID NO: 120 - protein, 813 aa, Artificial Sequence
  Synthetic
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARSGSYPYDVPDYAGSSSTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSRHRYALIPIPLAVITTCIVLYMNGILKCDRKPDRTNSN

SEQ ID NO: 121 - DNA, 2190 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagactgagctccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaaatactgctgtcaggtctccaatgacgtgggaccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtatcacaatgggaaagaaatgcagggaaggacagaggagaaagtccacatcccaaagatcctcccctggcacgctgggacttattcctgtgtggcagaaaacattcttggtactggacagaggggaccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaacctcatggggcctgggaggagccatcgcttggggtgctgaagatccaaaacgtaggctgggacaacacaaccatcgcctgcgcagcttgtaatagttggtgctcttgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcgagccagcccaccatccccatcgtgggcatcattgctggcctggttctctttggagctgtgatcactggagctgtggtcgctgctgtgatgtggaggaggaaaagctcagatagaaaaggagggagctactctcaggctgcaagcagtgacagtgcccagggctctgatgtgtctctcacagcttgtaaagtgtgagcggccgc

SEQ ID NO: 122 - protein, 721 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRLSSPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSSQPTIPIVGIIAGLVLFGAVITGAVVAAVMWRRKSSDRKGGSYSQAASSDSAQGSDVSLTACKV

SEQ ID NO: 123 - DNA, 2499 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagactgagctcaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcactcgagccagcccaccatccccatcgtgggcatcattgctggcctggttctctttggagctgtgatcactggagctgtggtcgctgctgtgatgtggaggaggaaaagctcagatagaaaaggagggagctactctcaggctgcaagcagtgacagtgcccagggctctgatgtgtctctcacagcttgtaaagtgtgagcggccgc

SEQ ID NO: 124 - protein, 824 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRLSSPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSSQPTIPIVGIIAGLVLFGAVITGAVVAAVMWRRKSSDRKGGSYSQAASSDSAQGSDVSLTACKV

SEQ ID NO: 125 - DNA, 2427 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagactgagctcaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 126 - protein, 800 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRLSSPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 127 - DNA, 2241 bp, Artificial Sequence
  Synthetic
tctagacgccgccaccatgtgcttcataaaacagggatggtgtctggtcctggaactgctactgctgcccttgggaactggatttcaaggtcattcaataccagatataaatgccaccaccggcagcaatgtaaccctgaaaatccataaggacccacttggaccatataaacgtatcacctggcttcatactaaaaatcagaagattttagagtacaactataatagtacaaagacaatcttcgagtctgaatttaaaggcagggtttatcttgaagaaaacaatggtgcacttcatatctctaatgtccggaaagaggacaaaggtacctactacatgagagtgctgcgtgaaactgagaacgagttgaagataaccctggaagtatttgatcctgtgcccaagccttccatagaaatcaataagactgaagcctccactgattcctgtcacctgaggctatcgtgtgaggtaaaggaccagcatgttgactatacttggtatgagagcagcggacctttccccaaaaagagtccaggatatgtgctcgatctcatcgtcacaccacagaacaagtctacattttacacctgccaagtcagcaatcctgtaagcagcaagaacgacacagtgtacttcactctaccttgtgatctgagctcaaccccgaagctggagatcaaggtcaatcccacagaggtggaaaagaacaattctgtgaccatgacatgccgggttaacagcagcaacccgaaactcaggaccgtggcggtgtcttggttcaaggatgggcgccccctagaggatcaggaactggaacaggaacaacagatgtccaagctaattctgcattcagtgaccaaggacatgagagggaaataccggtgccaggcttccaacgacataggcccaggagagtcggaagaagtggaactcacggtgcactatgctccagaaccctccagggttcacatctacccttcccccgctgaagagggacagtcagtagagctgatttgtgagtcactggccagtccaagtgcaacaaactacacctggtatcacaacaggaaacctatacctggagacacccaagagaagctccgcatccctaaagtctccccgtggcatgctgggaattactcttgcttggcagagaaccgtctgggtcatggaaagatagaccaggaagctaagctggatgtccattatgctcccaaggcggtgaccacagtgattcagagcttcacaccaatcctggaaggagacagtgtgaccctggtctgtaggtacaactccagcaatccagacgtcacctcctacagatggaaccctcaaggttctgggagtgtgctcaaacccggagtgctgaggattcagaaagtgacatgggattccatgcctgtcagctgtgctgcctgcaaccacaagtgttcgtgggccctccctgtcatcctgaatgtccactacgcccccagagacgtgaaggtactgaaggtaagccccgcatcagagatccgcgctgggcagcgtgtcctcctccaatgcgacttcgcagagagcaacccggcagaggtccgcttcttctggaagaagaatgggagtctcgtgcaggaagggaggtacctgagcttcggctccgtctccccagaagattctggaaattataactgcatggtcaacaactccatcggagagaccttgtcacaggcctggaacctccaagtgctgtatgctcctcggaggctgcgtgtgtccatcagccctggggaccatgtgatggaggggaagaaggccaccttgtcctgtgagagtgatgccaatccgcccatctcacagtacacctggtttgactccagtggccaagacctccactcctcaggccagaaactgagactggaacccctggaggtccaacacacgggttcctaccgctgcaaagggaccaatgggataggcacaggagagtcaccacccagcaccctcactgtctactacagtccagagaccatctcgagttccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtgagcggccgc

SEQ ID NO: 128 - protein, 738 aa, Artificial Sequence
  Synthetic
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLSSTPKLEIKVNPTEVEKNNSVTMTCRVNSSNPKLRTVAVSWFKDGRPLEDQELEQEQQMSKLILHSVTKDMRGKYRCQASNDIGPGESEEVELTVHYAPEPSRVHIYPSPAEEGQSVELICESLASPSATNYTWYHNRKPIPGDTQEKLRIPKVSPWHAGNYSCLAENRLGHGKIDQEAKLDVHYAPKAVTTVIQSFTPILEGDSVTLVCRYNSSNPDVTSYRWNPQGSGSVLKPGVLRIQKVTWDSMPVSCAACNHKCSWALPVILNVHYAPRDVKVLKVSPASEIRAGQRVLLQCDFAESNPAEVRFFWKKNGSLVQEGRYLSFGSVSPEDSGNYNCMVNNSIGETLSQAWNLQVLYAPRRLRVSISPGDHVMEGKKATLSCESDANPPISQYTWFDSSGQDLHSSGQKLRLEPLEVQHTGSYRCKGTNGIGTGESPPSTLTVYYSPETISSSTVSNMATVAVLVVLGAAIVTGAVVAFVMKMRRRNTGGKGGDYALAPGSQTSDLSLPDCKVMVHDPHSLA

SEQ ID NO: 129 - DNA, 2505 bp, Artificial Sequence
  Synthetic
tctagacgccgccaccatgtgcttcataaaacagggatggtgtctggtcctggaactgctactgctgcccttgggaactggatttcaaggtcattcaataccagatataaatgccaccaccggcagcaatgtaaccctgaaaatccataaggacccacttggaccatataaacgtatcacctggcttcatactaaaaatcagaagattttagagtacaactataatagtacaaagacaatcttcgagtctgaatttaaaggcagggtttatcttgaagaaaacaatggtgcacttcatatctctaatgtccggaaagaggacaaaggtacctactacatgagagtgctgcgtgaaactgagaacgagttgaagataaccctggaagtatttgatcctgtgcccaagccttccatagaaatcaataagactgaagcctccactgattcctgtcacctgaggctatcgtgtgaggtaaaggaccagcatgttgactatacttggtatgagagcagcggacctttccccaaaaagagtccaggatatgtgctcgatctcatcgtcacaccacagaacaagtctacattttacacctgccaagtcagcaatcctgtaagcagcaagaacgacacagtgtacttcactctaccttgtgatctgagctcccaaacacctacacccagtgatgaactgagcacaacagagaatgcccttcttctgcctcaaagtgaccccttacctgctcgcaccactgaatccacacccccaagcatctctgaaagaggaaatggctcttcagagaccacatatcatccaggtgtgttatccacgctgctgcctcacctgtccccacagcctgactcgcagacgccctctgccggaggagctgacactcagacattcagcagccaagctgacaatcccacactcacgcctgctcccggcggcgggactgacccaccaggtgtgccaggggagaggactgtaccggggaccattcctgcagacacagcctttcctgttgatacccccagccttgcacgcaacagctctgctgcctcacctacacacacctccaatgtcagcaccacagatatctcttcaggtgccagcctcacaactcttacaccatccactctgggccttgcaagcactgaccctccaagcacaaccatagctaccacaacgaagcaaacatgtgctgccatgtttgggaacattactgtgaattacacctatgaatctagtaatcagacttttaaggcagacctcaaagatgtccaaaatgctaagtgtggaaatgaggattgtgaaaacgtgttaaataacctagaagaatgctcacagataaaaaacatcagtgtgtctaatgactcatgtgctccagctacaactatagatttatatgtaccaccagggactgacaagttttcgctacatgactgcacaccaaaagaaaaggctaatacttcaatttgtttggagtggaaaacaaaaaaccttgatttcagaaaatgcaacagtgacaatatttcatatgtactccactgtgagccagaaaataatacaaaatgcattagaagaaatacattcatacctgaaagatgtcagttggacaaccttcgtgcccaaacaaattacacatgtgtagcagaaatcttatatcgcggtgtaaaactcgtcaaaaatgttataaatgtgcagacagatttggggattccagaaacgcctaagcctagttgtggcgatccagctgcaagaaaaacgttagtctcttggcctgagcctgtgtctaaacctgagtctgcatctaaacctcatggatatgttttatgctataagaacaattcagaaaaatgtaaaagtttgcctaataatgtgaccagttttgaggtggagagcttgaaaccttataaatactatgaagtgtccctacttgcctatgtcaatgggaagattcaaagaaatgggactgctgagaagtgcaattttcacacaaaagcagatcgtccagacaaggtcaatggaatgaaaacctcccggccgacagacaatagtataaatgttacatgtggtcctccttatgaaactaatggccctaaaaccttttacattttggtagtcagaagtggaggttcttttgttacaaaatacaacaagacaaactgtcagttttatgtagataatctctactattcaactgactatgagtttctggtctcttttcacaatggagtgtacgagggagattcagttataagaaatgagtcaacaaatttctcgagttccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtgagcggccgc

SEQ ID NO: 130 - protein, 826 aa, Artificial Sequence
  Synthetic
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLSSQTPTPSDELSTTENALLLPQSDPLPARTTESTPPSISERGNGSSETTYHPGVLSTLLPHLSPQPDSQTPSAGGADTQTFSSQADNPTLTPAPGGGTDPPGVPGERTVPGTIPADTAFPVDTPSLARNSSAASPTHTSNVSTTDISSGASLTTLTPSTLGLASTDPPSTTIATTTKQTCAAMFGNITVNYTYESSNQTFKADLKDVQNAKCGNEDCENVLNNLEECSQIKNISVSNDSCAPATTIDLYVPPGTDKFSLHDCTPKEKANTSICLEWKTKNLDFRKCNSDNISYVLHCEPENNTKCIRRNTFIPERCQLDNLRAQTNYTCVAEILYRGVKLVKNVINVQTDLGIPETPKPSCGDPAARKTLVSWPEPVSKPESASKPHGYVLCYKNNSEKCKSLPNNVTSFEVESLKPYKYYEVSLLAYVNGKIQRNGTAEKCNFHTKADRPDKVNGMKTSRPTDNSINVTCGPPYETNGPKTFYILVVRSGGSFVTKYNKTNCQFYVDNLYYSTDYEFLVSFHNGVYEGDSVIRNESTNFSSSTVSNMATVAVLVVLGAAIVTGAVVAFVMKMRRRNTGGKGGDYALAPGSQTSDLSLPDCKVMVHDPHSLA

SEQ ID NO: 131 - DNA, 2400 bp, Artificial Sequence
  Synthetic
tctagacgccgccaccatgtgcttcataaaacagggatggtgtctggtcctggaactgctactgctgcccttgggaactggatttcaaggtcattcaataccagatataaatgccaccaccggcagcaatgtaaccctgaaaatccataaggacccacttggaccatataaacgtatcacctggcttcatactaaaaatcagaagattttagagtacaactataatagtacaaagacaatcttcgagtctgaatttaaaggcagggtttatcttgaagaaaacaatggtgcacttcatatctctaatgtccggaaagaggacaaaggtacctactacatgagagtgctgcgtgaaactgagaacgagttgaagataaccctggaagtatttgatcctgtgcccaagccttccatagaaatcaataagactgaagcctccactgattcctgtcacctgaggctatcgtgtgaggtaaaggaccagcatgttgactatacttggtatgagagcagcggacctttccccaaaaagagtccaggatatgtgctcgatctcatcgtcacaccacagaacaagtctacattttacacctgccaagtcagcaatcctgtaagcagcaagaacgacacagtgtacttcactctaccttgtgatctgagctcccaaacacctacacccagtgatgaactgagcacaacagagaatgcccttcttctgcctcaaagtgaccccttacctgctcgcaccactgaatccacacccccaagcatctctgaaagaggaaatggctcttcagagaccacatatcatccaggtgtgttatccacgctgctgcctcacctgtccccacagcctgactcgcagacgccctctgccggaggagctgacactcagacattcagcagccaagctgacaatcccacactcacgcctgctcccggcggcgggactgacccaccaggtgtgccaggggagaggactgtaccggggaccattcctgcagacacagcctttcctgttgatacccccagccttgcacgcaacagctctgctgcctcacctacacacacctccaatgtcagcaccacagatatctcttcaggtgccagcctcacaactcttacaccatccactctgggccttgcaagcactgaccctccaagcacaaccatagctaccacaacgaagcaaacatgtgctgccatgtttgggaacattactgtgaattacacctatgaatctagtaatcagacttttaaggcagacctcaaagatgtccaaaatgctaagtgtggaaatgaggattgtgaaaacgtgttaaataacctagaagaatgctcacagataaaaaacatcagtgtgtctaatgactcatgtgctccagctacaactatagatttatatgtaccaccagggactgacaagttttcgctacatgactgcacaccaaaagaaaaggctaatacttcaatttgtttggagtggaaaacaaaaaaccttgatttcagaaaatgcaacagtgacaatatttcatatgtactccactgtgagccagaaaataatacaaaatgcattagaagaaatacattcatacctgaaagatgtcagttggacaaccttcgtgcccaaacaaattacacatgtgtagcagaaatcttatatcgcggtgtaaaactcgtcaaaaatgttataaatgtgcagacagatttggggattccagaaacgcctaagcctagttgtggcgatccagctgcaagaaaaacgttagtctcttggcctgagcctgtgtctaaacctgagtctgcatctaaacctcatggatatgttttatgctataagaacaattcagaaaaatgtaaaagtttgcctaataatgtgaccagttttgaggtggagagcttgaaaccttataaatactatgaagtgtccctacttgcctatgtcaatgggaagattcaaagaaatgggactgctgagaagtgcaattttcacacaaaagcagatcgtccagacaaggtcaatggaatgaaaacctcccggccgacagacaatagtataaatgttacatgtggtcctccttatgaaactaatggccctaaaaccttttacattttggtagtcagaagtggaggttcttttgttacaaaatacaacaagacaaactgtcagttttatgtagataatctctactattcaactgactatgagtttctggtctcttttcacaatggagtgtacgagggagattcagttataagaaatgagtcaacaaattttaatgctaaagcactgattatattcctggtgtttctgattattgtgacatcaatagccttgcttgttgttttgtataaaatctatgatctgcgcaagaaaagatccagcaattgagcggccgc

SEQ ID NO: 132 - protein, 791 aa, Artificial Sequence
  Synthetic
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLSSQTPTPSDELSTTENALLLPQSDPLPARTTESTPPSISERGNGSSETTYHPGVLSTLLPHLSPQPDSQTPSAGGADTQTFSSQADNPTLTPAPGGGTDPPGVPGERTVPGTIPADTAFPVDTPSLARNSSAASPTHTSNVSTTDISSGASLTTLTPSTLGLASTDPPSTTIATTTKQTCAAMFGNITVNYTYESSNQTFKADLKDVQNAKCGNEDCENVLNNLEECSQIKNISVSNDSCAPATTIDLYVPPGTDKFSLHDCTPKEKANTSICLEWKTKNLDFRKCNSDNISYVLHCEPENNTKCIRRNTFIPERCQLDNLRAQTNYTCVAEILYRGVKLVKNVINVQTDLGIPETPKPSCGDPAARKTLVSWPEPVSKPESASKPHGYVLCYKNNSEKCKSLPNNVTSFEVESLKPYKYYEVSLLAYVNGKIQRNGTAEKCNFHTKADRPDKVNGMKTSRPTDNSINVTCGPPYETNGPKTFYILVVRSGGSFVTKYNKTNCQFYVDNLYYSTDYEFLVSFHNGVYEGDSVIRNESTNFNAKALIIFLVFLIIVTSIALLVVLYKIYDLRKKRSSN

SEQ ID NO: 133 - DNA, 1833 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggtgagcggccgc

SEQ ID NO: 134 - protein, 602 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQG

SEQ ID NO: 135 - DNA, 1662 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 136 - protein, 545 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 137 - DNA, 2145 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 138 - protein, 706 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 139 - DNA, 1845 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgggcggaggcagcaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggtgagcggccgc

SEQ ID NO: 140 - protein, 606 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVGGGSKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQG

SEQ ID NO: 141 - DNA, 1674 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgggcggaggcagccaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 142 - protein, 549 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVGGGSQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 143 - DNA, 2157 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgggcggaggcagccaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 144 - protein, 710 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVGGGSQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 145 - DNA, 2097 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggtgagcggccgc

SEQ ID NO: 146 - protein, 690 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQG

SEQ ID NO: 147 - DNA, 1926 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagccaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 148 - protein, 633 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 149 - DNA, 2409 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagccaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 150 - protein, 794 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 151 - DNA, 2109 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcggcggaggcagcaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggtgagcggccgc

SEQ ID NO: 152 - protein, 694 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSGGGSKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQG

SEQ ID NO: 153 - DNA, 1938 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcggcggaggcagccaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 154 - protein, 637 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSGGGSQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 155 - DNA, 2421 bp, Artificial Sequence
  Synthetic
tctagacgccgccaccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgagttcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcggcggaggcagccaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 156 - protein, 798 aa, Artificial Sequence
  Synthetic
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSGGGSQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 157 - DNA, 2406 bp, Artificial Sequence
  Synthetic
tctagacgccgccaccatggccttaccagtgaccgccttgctcctgccgctggccttgctgctccacgccgccaggccggacgtggtgatgacccagagcccccccagcctgctggtgaccctgggccagcccgccagcatcagctgcagaagcagccagagcctgctgcacagcagcggcaacacctacctgaactggctgctgcagagacccggccagagcccccagcccctgatctacctggtgagcaagctggagagcggcgtgcccgacagattcagcggcagcggcagcggcaccgacttcaccctgaagatcagcggcgtggaggccgaggacgtgggcgtgtactactgcatgcagttcacccactacccctacaccttcggccagggcaccaagctggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgagtactacatgtactgggtgagacaggcccccggccagggcctggagctgatgggcagaatcgaccccgaggacggcagcatcgactacgtggagaagttcaagaagaaggtgaccctgaccgccgacaccagcagcagcaccgcctacatggagctgagcagcctgaccagcgacgacaccgccgtgtactactgcgccagaggcaagttcaactacagattcgcctactggggccagggcaccctggtgaccgtgagcagcaccactaccccagcaccgaggccacccaccccggctcctaccatcgcctcccagcctctgtccctgcgtccggaggcatgtagacccgcagctggtggggccgtgcatacccggggtcttgacttcgcctgcgataagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggtgagcggccgc

SEQ ID NO: 158 - protein, 793 aa, Artificial Sequence
  Synthetic
MALPVTALLLPLALLLHAARPDVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKGGGGSGGGGSGGGGSQVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSSTTTPAPRPPTPAPTIASQPLSLRPEACRPAAGGAVHTRGLDFACDKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQG

SEQ ID NO: 159 - DNA, 2235 bp, Artificial Sequence
  Synthetic
tctagacgccgccaccatggccttaccagtgaccgccttgctcctgccgctggccttgctgctccacgccgccaggccggacgtggtgatgacccagagcccccccagcctgctggtgaccctgggccagcccgccagcatcagctgcagaagcagccagagcctgctgcacagcagcggcaacacctacctgaactggctgctgcagagacccggccagagcccccagcccctgatctacctggtgagcaagctggagagcggcgtgcccgacagattcagcggcagcggcagcggcaccgacttcaccctgaagatcagcggcgtggaggccgaggacgtgggcgtgtactactgcatgcagttcacccactacccctacaccttcggccagggcaccaagctggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgagtactacatgtactgggtgagacaggcccccggccagggcctggagctgatgggcagaatcgaccccgaggacggcagcatcgactacgtggagaagttcaagaagaaggtgaccctgaccgccgacaccagcagcagcaccgcctacatggagctgagcagcctgaccagcgacgacaccgccgtgtactactgcgccagaggcaagttcaactacagattcgcctactggggccagggcaccctggtgaccgtgagcagcaccactaccccagcaccgaggccacccaccccggctcctaccatcgcctcccagcctctgtccctgcgtccggaggcatgtagacccgcagctggtggggccgtgcatacccggggtcttgacttcgcctgcgatcaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 160 - protein, 736 aa, Artificial Sequence
  Synthetic
MALPVTALLLPLALLLHAARPDVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKGGGGSGGGGSGGGGSQVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSSTTTPAPRPPTPAPTIASQPLSLRPEACRPAAGGAVHTRGLDFACDQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 161 - DNA, 2718 bp, Artificial Sequence
  Synthetic
tctagacgccgccaccatggccttaccagtgaccgccttgctcctgccgctggccttgctgctccacgccgccaggccggacgtggtgatgacccagagcccccccagcctgctggtgaccctgggccagcccgccagcatcagctgcagaagcagccagagcctgctgcacagcagcggcaacacctacctgaactggctgctgcagagacccggccagagcccccagcccctgatctacctggtgagcaagctggagagcggcgtgcccgacagattcagcggcagcggcagcggcaccgacttcaccctgaagatcagcggcgtggaggccgaggacgtgggcgtgtactactgcatgcagttcacccactacccctacaccttcggccagggcaccaagctggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgagtactacatgtactgggtgagacaggcccccggccagggcctggagctgatgggcagaatcgaccccgaggacggcagcatcgactacgtggagaagttcaagaagaaggtgaccctgaccgccgacaccagcagcagcaccgcctacatggagctgagcagcctgaccagcgacgacaccgccgtgtactactgcgccagaggcaagttcaactacagattcgcctactggggccagggcaccctggtgaccgtgagcagcaccactaccccagcaccgaggccacccaccccggctcctaccatcgcctcccagcctctgtccctgcgtccggaggcatgtagacccgcagctggtggggccgtgcatacccggggtcttgacttcgcctgcgatcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 162 - protein, 897 aa, Artificial Sequence
  Synthetic
MALPVTALLLPLALLLHAARPDVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKGGGGSGGGGSGGGGSQVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSSTTTPAPRPPTPAPTIASQPLSLRPEACRPAAGGAVHTRGLDFACDQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 163 - DNA, 2283 bp, Artificial Sequence
  Synthetic
tctagacgccgccaccatggccttaccagtgaccgccttgctcctgccgctggccttgctgctccacgccgccaggccggacgtggtgatgacccagagcccccccagcctgctggtgaccctgggccagcccgccagcatcagctgcagaagcagccagagcctgctgcacagcagcggcaacacctacctgaactggctgctgcagagacccggccagagcccccagcccctgatctacctggtgagcaagctggagagcggcgtgcccgacagattcagcggcagcggcagcggcaccgacttcaccctgaagatcagcggcgtggaggccgaggacgtgggcgtgtactactgcatgcagttcacccactacccctacaccttcggccagggcaccaagctggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgagtactacatgtactgggtgagacaggcccccggccagggcctggagctgatgggcagaatcgaccccgaggacggcagcatcgactacgtggagaagttcaagaagaaggtgaccctgaccgccgacaccagcagcagcaccgcctacatggagctgagcagcctgaccagcgacgacaccgccgtgtactactgcgccagaggcaagttcaactacagattcgcctactggggccagggcaccctggtgaccgtgagcagcggcggaggcagcaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggtgagcggccgc

SEQ ID NO: 164 - protein, 752 aa, Artificial Sequence
  Synthetic
MALPVTALLLPLALLLHAARPDVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKGGGGSGGGGSGGGGSQVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSSGGGSKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQG

SEQ ID NO: 165 - DNA, 2112 bp, Artificial Sequence
  Synthetic
tctagacgccgccaccatggccttaccagtgaccgccttgctcctgccgctggccttgctgctccacgccgccaggccggacgtggtgatgacccagagcccccccagcctgctggtgaccctgggccagcccgccagcatcagctgcagaagcagccagagcctgctgcacagcagcggcaacacctacctgaactggctgctgcagagacccggccagagcccccagcccctgatctacctggtgagcaagctggagagcggcgtgcccgacagattcagcggcagcggcagcggcaccgacttcaccctgaagatcagcggcgtggaggccgaggacgtgggcgtgtactactgcatgcagttcacccactacccctacaccttcggccagggcaccaagctggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgagtactacatgtactgggtgagacaggcccccggccagggcctggagctgatgggcagaatcgaccccgaggacggcagcatcgactacgtggagaagttcaagaagaaggtgaccctgaccgccgacaccagcagcagcaccgcctacatggagctgagcagcctgaccagcgacgacaccgccgtgtactactgcgccagaggcaagttcaactacagattcgcctactggggccagggcaccctggtgaccgtgagcagcggcggaggcagccaaagcccaacaccttcccccactgatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 166 - protein, 695 aa, Artificial Sequence
  Synthetic
MALPVTALLLPLALLLHAARPDVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKGGGGSGGGGSGGGGSQVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSSGGGSQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 167 - DNA, 2595 bp, Artificial Sequence
  Synthetic
tctagacgccgccaccatggccttaccagtgaccgccttgctcctgccgctggccttgctgctccacgccgccaggccggacgtggtgatgacccagagcccccccagcctgctggtgaccctgggccagcccgccagcatcagctgcagaagcagccagagcctgctgcacagcagcggcaacacctacctgaactggctgctgcagagacccggccagagcccccagcccctgatctacctggtgagcaagctggagagcggcgtgcccgacagattcagcggcagcggcagcggcaccgacttcaccctgaagatcagcggcgtggaggccgaggacgtgggcgtgtactactgcatgcagttcacccactacccctacaccttcggccagggcaccaagctggagatcaagggcggcggcggcagcggcggcggcggcagcggcggcggcggcagccaggtgcagctggtgcagagcggcgccgaggtgaagaagcccggcgccagcgtgaaggtgagctgcaaggccagcggctacaccttcaccgagtactacatgtactgggtgagacaggcccccggccagggcctggagctgatgggcagaatcgaccccgaggacggcagcatcgactacgtggagaagttcaagaagaaggtgaccctgaccgccgacaccagcagcagcaccgcctacatggagctgagcagcctgaccagcgacgacaccgccgtgtactactgcgccagaggcaagttcaactacagattcgcctactggggccagggcaccctggtgaccgtgagcagcggcggaggcagccaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaattgagcggccgc

SEQ ID NO: 168 - protein, 856 aa, Artificial Sequence
  Synthetic
MALPVTALLLPLALLLHAARPDVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKGGGGSGGGGSGGGGSQVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSSGGGSQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCN

SEQ ID NO: 169 - protein, 3 aa, Artificial Sequence
  Synthetic
GGG

SEQ ID NO: 170 - protein, 7 aa, Artificial Sequence
  Synthetic
GGGSGGG

SEQ ID NO: 171 - protein, 30 aa, Artificial Sequence
  Synthetic
  misc_feature (16)..(30): may be absent
EAAAKEAAAKEAAAKEAAAKEAAAKEAAAK